SEQUENCE LISTING (985 sequences)

SEQ ID NO: 1 - protein, 227 aa, Severe acute respiratory syndrome coronavirus 2 (SARS-CoV-2)
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 325 aa, Severe acute respiratory syndrome coronavirus 2 (SARS-CoV-2)
STKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 3 - protein, 246 aa, Severe acute respiratory syndrome coronavirus 2 (SARS-CoV-2)
HKPSNTKVDKRVELKTPLGDTTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 383 aa, Severe acute respiratory syndrome coronavirus 2 (SARS-CoV-2)
PTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPRSTTFWAWSVLRVPAPPSPQPATYTCVVSHEDSRTLLNASRSLEVSYVTDHGPMK

SEQ ID NO: 5 - protein, 276 aa, Severe acute respiratory syndrome coronavirus 2 (SARS-CoV-2)
VTSTLTIKZSDWLGESMFTCRVDHRGLTFQQNASSMCVPDQDTAIRVFAIPPSFASIFLTKSTKLTCLVTDLTTYBSVTISWTREENGAVKTHTNISESHPNATFSAVGEASICEDBDWSGERFTCTVTHTDLPSPLKQTISRPKGVALHRPBVYLLPPARZZLNLRESATITCLVTGFSPADVFVEWMQRGEPLSPQKYVTSAPMPEPQAPGRYFAHSILTVSEEEWNTGGTYTCVVAHEALPNRVTERTVDKSTGKPTLYNVSLVMSDTAGTCY

SEQ ID NO: 6 - protein, 353 aa, Severe acute respiratory syndrome coronavirus 2 (SARS-CoV-2)
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 7 - protein, 222 aa, Severe acute respiratory syndrome coronavirus 2 (SARS-CoV-2)
ADPCDSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 8 - protein, 327 aa, Severe acute respiratory syndrome coronavirus 2 (SARS-CoV-2)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 9 - protein, 223 aa, Severe acute respiratory syndrome coronavirus 2 (SARS-CoV-2)
PPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPG

SEQ ID NO: 10 - protein, 227 aa, Severe acute respiratory syndrome coronavirus 2 (SARS-CoV-2)
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 227 aa, Severe acute respiratory syndrome coronavirus 2 (SARS-CoV-2)
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 227 aa, Severe acute respiratory syndrome coronavirus 2 (SARS-CoV-2)
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 227 aa, Severe acute respiratory syndrome coronavirus 2 (SARS-CoV-2)
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 365 aa, Severe acute respiratory syndrome coronavirus 2 (SARS-CoV-2)
MAVMAPRTLLLLLSGALALTQTWAGSHSMRYFFTSVSRPGRGEPRFIAVGYVDDTQFVRFDSDAASQKMEPRAPWIEQEGPEYWDQETRNMKAHSQTDRANLGTLRGYYNQSEDGSHTIQIMYGCDVGPDGRFLRGYRQDAYDGKDYIALNEDLRSWTAADMAAQITKRKWEAVHAAEQRRVYLEGRCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 15 - protein, 365 aa, Severe acute respiratory syndrome coronavirus 2 (SARS-CoV-2)
MAVMAPRTLLLLLSGALALTQTWAGSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGRCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 16 - protein, 365 aa, Severe acute respiratory syndrome coronavirus 2 (SARS-CoV-2) [sequence omitted]

SEQ ID NO: 17 - protein, 365 aa, Severe acute respiratory syndrome coronavirus 2 (SARS-CoV-2) [sequence omitted]

SEQ ID NO: 18 - protein, 362 aa, Severe acute respiratory syndrome coronavirus 2 (SARS-CoV-2)
MLVMAPRTVLLLLSAALALTETWAGSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVMCRRKSSGGKGGSYSQAACSDSAQGSDVSLTA

SEQ ID NO: 19 - protein, 365 aa, Severe acute respiratory syndrome coronavirus 2 (SARS-CoV-2)
MRVMAPRALLLLLSGGLALTETWACSHSMRYFDTAVSRPGRGEPRFISVGYVDDTQFVRFDSDAASPRGEPRAPWVEQEGPEYWDRETQNYKRQAQADRVSLRNLRGYYNQSEDGSHTLQRMYGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCK

SEQ ID NO: 20 - protein, 275 aa, Severe acute respiratory syndrome coronavirus 2 (SARS-CoV-2)
GSHSMRYFFTSVSRPGRGEPRFIAVGYVDDTQFVRFDSDAASQKMEPRAPWIEQEGPEYWDQETRNMKAHSQTDRANLGTLRGYYNQSEDGSHTIQIMYGCDVGPDGRFLRGYRQDAYDGKDYIALNEDLRSWTAADMAAQITKRKWEAVHAAEQRRVYLEGRCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 21 - protein, 275 aa, Severe acute respiratory syndrome coronavirus 2 (SARS-CoV-2)
GSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWE

SEQ ID NO: 22 - protein, 275 aa, Severe acute respiratory syndrome coronavirus 2 (SARS-CoV-2)
CSHSMRYFDTAVSRPGRGEPRFISVGYVDDTQFVRFDSDAASPRGEPRAPWVEQEGPEYWDRETQNYKRQAQADRVSLRNLRGYYNQSEDGSHTLQRMYGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWE

SEQ ID NO: 23 - protein, 276 aa, Severe acute respiratory syndrome coronavirus 2 (SARS-CoV-2) [sequence omitted]

SEQ ID NO: 24 - protein, 274 aa, Severe acute respiratory syndrome coronavirus 2 (SARS-CoV-2)
GPHSLRYFVTAVSRPGLGEPRFIAVGYVDDTQFVRFDSDADNPRFEPRAPWMEQEGPEYWEEQTQRAKSDEQWFRVSLRTAQRYYNQSKGGSHTFQRMFGCDVGSDWRLLRGYQQFAYDGRDYIALNEDLKTWTAADTAALITRRKWEQAGDAEYYRAYLEGECVEWLRRYLELGNETLLRTDSPKAHVTYHPRSQVDVTLRCWALGFYPADITLTWQLNGEDLTQDMELVETRPAGDGTFQKWAAVVVPLGKEQNYTCHVHHKGLPEPLTLRW

SEQ ID NO: 25 - protein, 275 aa, Severe acute respiratory syndrome coronavirus 2 (SARS-CoV-2)
GSHSMRYFFTSVSRPGRGEPRFIAVGYVDDTQFVRFDSDAASQRMEPRAPWIEQEGPEYWDGETRKVKAHSQTHRVDLGTLRGAYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWE

SEQ ID NO: 26 - protein, 275 aa, Severe acute respiratory syndrome coronavirus 2 (SARS-CoV-2)
GSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWE

SEQ ID NO: 27 - protein, 276 aa, Severe acute respiratory syndrome coronavirus 2 (SARS-CoV-2)
GSHSMRYFFTSVSRPGRGEPRFIAVGYVDDTQFVRFDSDAASQRMEPRAPWIEQEGPEYWDGETRKVKAHSQTHRVDLGTLRGAYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWEP

SEQ ID NO: 28 - protein, 276 aa, Severe acute respiratory syndrome coronavirus 2 (SARS-CoV-2)
GSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGRCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEL

SEQ ID NO: 29 - protein, 276 aa, Severe acute respiratory syndrome coronavirus 2 (SARS-CoV-2)
GSHSMRYFSTSVSRPGRGEPRFIAVGYVDDTQFVRFDSDAASQRMEPRAPWIEQEGPEYWDEETGKVKAHSQTDRENLRIALRYYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEP

SEQ ID NO: 30 - protein, 276 aa, Severe acute respiratory syndrome coronavirus 2 (SARS-CoV-2)
GSHSMRYFTTSVSRPGRGEPRFIAVGYVDDTQFVRFDSDAASQRMEPRAPWIEQEGPEYWDRNTRNVKAHSQIDRVDLGTLRGYYNQSEAGSHTIQMMYGCDVGSDGRFLRGYQQDAYDGKDYIALNEDLRSWTAADMAAQITQRKWEAARVAEQLRAYLEGTCVEWLRRYLENGKETLQRTDPPKTHMTHHAVSDHEATLRCWALSFYPAEITLTWQRDGEDQTQDTELVETRPAGDGTFQKWASVVVPSGQEQRYTCHVQHEGLPKPLTLRWEP

SEQ ID NO: 31 - protein, 276 aa, Severe acute respiratory syndrome coronavirus 2 (SARS-CoV-2)
GSHSMRYFFTSVSRPGRGEPRFIAVGYVDDTQFVRFDSDAASQRMEPRAPWIEQEGPEYWDQETRNVKAQSQTDRVDLGTLRGYYNQSEAGSHTIQIMYGCDVGSDGRFLRGYRQDAYDGKDYIALNEDLRSWTAADMAAQITKRKWEAAHEAEQLRAYLDGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEL

SEQ ID NO: 32 - protein, 276 aa, Severe acute respiratory syndrome coronavirus 2 (SARS-CoV-2)
GSHSMRYFSTSVSRPGRGEPRFIAVGYVDDTQFVRFDSDAASQRMEPRAPWIEQEGPEYWDEETGKVKAHSQTDRENLRIALRYYNQSEAGSHTLQMMFGCDVGSDGRFLRGYHQYAYDGKDYIALKEDLRSWTAADMAAQITQRKWEAARVAEQLRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEP

SEQ ID NO: 33 - protein, 276 aa, Severe acute respiratory syndrome coronavirus 2 (SARS-CoV-2)
GSHSMRYFSTSVSRPGRGEPRFIAVGYVDDTQFVRFDSDAASQRMEPRAPWIEQEGPEYWDEETGKVKAQSQTDRENLRIALRYYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEP

SEQ ID NO: 34 - protein, 276 aa, Severe acute respiratory syndrome coronavirus 2 (SARS-CoV-2)
GSHSMRYFYTSVSRPGRGEPRFIAVGYVDDTQFVRFDSDAASQRMEPRAPWIEQEGPEYWDRNTRKVKAQSQTDRVDLGTLRGYYNQSEDGSHTIQRMYGCDVGPDGRFLRGYQQDAYDGKDYIALNEDLRSWTAADMAAQITQRKWETAHEAEQWRAYLEGTCVEWLRRYLENGKETLQRTDAPKTHMTHHAVSDHEATLRCWALSFYPAEITLTWQRDGEDQTQDTELVETRPAGDGTFQKWASVVVPSGQEQRYTCHVQHEGLPKPLTLRWEP

SEQ ID NO: 35 - protein, 276 aa, Severe acute respiratory syndrome coronavirus 2 (SARS-CoV-2)
  VARIANT (9)..(9): X1 is F, Y, S, or T
  VARIANT (44)..(44): X2 is K or R
  VARIANT (62)..(62): X3 is Q, G, E, or R
  VARIANT (63)..(63): X4 is N or E
  VARIANT (65)..(65): X5 is R or G
  VARIANT (66)..(66): X6 is N or K
  VARIANT (67)..(67): X7 is M or V
  VARIANT (70)..(70): X8 is H or Q
  VARIANT (73)..(73): X9 is T or I
  VARIANT (74)..(74): X10 is D or H
  VARIANT (76)..(76): X11 is A, V, or E
  VARIANT (77)..(77): X12 is N or D
  VARIANT (79)..(79): X13 is G or R
  VARIANT (80)..(80): X14 is T or I
  VARIANT (81)..(81): X15 is L or A
  VARIANT (82)..(82): X16 is R or L
  VARIANT (83)..(83): X17 is G or R
  VARIANT (90)..(90): X18 is A or D
  VARIANT (95)..(95): X19 is I, L, or V
  VARIANT (97)..(97): X20 is I, R or M
  VARIANT (99)..(99): X21 is F or Y
  VARIANT (105)..(105): X22 is S or P
  VARIANT (107)..(107): X23 is W or G
  VARIANT (114)..(114): X24 is R, H, or Q
  VARIANT (116)..(116): X25 is D or Y
  VARIANT (127)..(127): X26 is N or K
  VARIANT (142)..(142): X27 is T or I
  VARIANT (144)..(144): X28 is K or Q
  VARIANT (145)..(145): X29 is R or H
  VARIANT (149)..(149): X30 is A or T
  VARIANT (150)..(150): X31 is A or V
  VARIANT (151)..(151): X32 is H or R
  VARIANT (156)..(156): X33 is R, L, Q, or W
  VARIANT (158)..(158): X34 is V or A
  VARIANT (161)..(161): X35 is D or E
  VARIANT (163)..(163): X36 is R or T
  VARIANT (166)..(166): X37 is D or E
  VARIANT (167)..(167): X38 is W or G
  VARIANT (184)..(184): X39 is P or A
  VARIANT (193)..(193): X40 is P or A
  VARIANT (194)..(194): X41 is V or I
  VARIANT (207)..(207): X42 is S or G
  VARIANT (246)..(246): X43 is A or S
  VARIANT (253)..(253): X44 is Q or E
  VARIANT (276)..(276): X45 is P or L
GSHSMRYFXTSVSRPGRGEPRFIAVGYVDDTQFVRFDSDAASQXMEPRAPWIEQEGPEYWDXXTXXXKAXSQXXRXXLXXXXXYYNQSEXGSHTXQXMXGCDVGXDXRFLRGYXQXAYDGKDYIALXEDLRSWTAADMAAQXTXXKWEXXXEAEQXRXYLXGXCVXXLRRYLENGKETLQRTDXPKTHMTHHXXSDHEATLRCWALXFYPAEITLTWQRDGEDQTQDTELVETRPAGDGTFQKWAXVVVPSGXEQRYTCHVQHEGLPKPLTLRWEX

SEQ ID NO: 36 - protein, 276 aa, Severe acute respiratory syndrome coronavirus 2 (SARS-CoV-2) [sequence omitted]

SEQ ID NO: 37 - protein, 276 aa, Severe acute respiratory syndrome coronavirus 2 (SARS-CoV-2)
GSHSMRYFDTAMSRPGRGEPRFISVGYVDDTQFVRFDSDAASPREEPRAPWIEQEGPEYWDRNTQIFKTNTQTDRESLRNLRGYYNQSEAGSHTLQSMYGCDVGPDGRLLRGHNQYAYDGKDYIALNEDLRSWTAADTAAQITQRKWEAARVAEQDRAYLEGTCVEWLRRYLENGKDTLERADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 38 - protein, 276 aa, Severe acute respiratory syndrome coronavirus 2 (SARS-CoV-2)
GSHSMRYFYTAMSRPGRGEPRFIAVGYVDDTQFVRFDSDAASPRMAPRAPWIEQEGPEYWDRNTQISKTNTQTYRESLRNLRGYYNQSEAGSHIIQRMYGCDVGPDGRLLRGYDQSAYDGKDYIALNEDLSSWTAADTAAQITQRKWEAAREAEQLRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 39 - protein, 276 aa, Severe acute respiratory syndrome coronavirus 2 (SARS-CoV-2)
GSHSMRYFYTSVSRPGRGEPRFISVGYVDDTQFVRFDSDAASPREEPRAPWIEQEGPEYWDRNTQICKTNTQTYRENLRTALRYYNQSEAGSHTLQRMYGCDVGPDGRLLRGHNQFAYDGKDYIALNEDLSSWTAADTAAQITQRKWEAARVAEQLRTYLEGTCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 40 - protein, 276 aa, Severe acute respiratory syndrome coronavirus 2 (SARS-CoV-2)
GSHSMRYFHTAMSRPGRGEPRFITVGYVDDTLFVRFDSDATSPRKEPRAPWIEQEGPEYWDRETQISKTNTQTYRESLRNLRGYYNQSEAGSHTLQRMYGCDVGPDGRLLRGHNQYAYDGKDYIALNEDLRSWTAADTAAQISQRKLEAARVAEQLRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 41 - protein, 276 aa, Severe acute respiratory syndrome coronavirus 2 (SARS-CoV-2)
GSHSMRYFYTAMSRPGRGEPRFIAVGYVDDTQFVRFDSDAASPRMAPRAPWIEQEGPEYWDRETQKYKRQAQTDRVSLRNLRGYYNQSEAGSHTLQRMYGCDVGPDGRLLRGHDQSAYDGKDYIALNEDLSSWTAADTAAQITQRKWEAAREAEQWRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 42 - protein, 276 aa, Severe acute respiratory syndrome coronavirus 2 (SARS-CoV-2)
GSHSMRYFYTAMSRPGRGEPRFIAVGYVDDTQFVRFDSDAASPRTEPRAPWIEQEGPEYWDRNTQIFKTNTQTYRENLRIALRYYNQSEAGSHIIQRMYGCDLGPDGRLLRGHDQSAYDGKDYIALNEDLSSWTAADTAAQITQRKWEAARVAEQLRAYLEGLCVEWLRRYLENGKETLQRADPPKTHVTHHPVSDHEATLRCWALGFYPAEITLTWQRDGEDQTQDTELVETRPAGDRTFQKWAAVVVPSGEEQRYTCHVQHEGLPKPLTLRWEP

SEQ ID NO: 43 - protein, 276 aa, Severe acute respiratory syndrome coronavirus 2 (SARS-CoV-2)
  VARIANT (9)..(9): X1 is H, Y, or D
  VARIANT (11)..(11): X2 is A or S
  VARIANT (12)..(12): X3 is M or V
  VARIANT (24)..(24): X4 is A, S, or T
  VARIANT (32)..(32): X5 is Q or L
  VARIANT (41)..(41): X6 is A or T
  VARIANT (45)..(45): X7 is E, M K, or T
  VARIANT (46)..(46): X8 is A or T
  VARIANT (63)..(63): X9 is E or N
  VARIANT (66)..(66): X10 is I or K
  VARIANT (67)..(67): X11 is Y, F, S, or C
  VARIANT (70)..(70): X12 is N or Q
  VARIANT (71)..(71): X13 is A or T
  VARIANT (74)..(74): X14 is D or Y
  VARIANT (76)..(76): X15 is E or V
  VARIANT (77)..(77): X16 is S or N
  VARIANT (80)..(80): X17 is T, N, or I
  VARIANT (81)..(81): X18 is A or L
  VARIANT (82)..(82): X19 is L, or R
  VARIANT (83)..(83): X20 is R or G
  VARIANT (94)..(94): X21 is T or I
  VARIANT (95)..(95): X22 is L or I
  VARIANT (97)..(97): X23 is R or S
  VARIANT (131)..(131): X24 is R or S
  VARIANT (143)..(143): X25 is S or T
  VARIANT (147)..(147): X26 is L or W
  VARIANT (152)..(152): X27 is E OR V
  VARIANT (156)..(156): X28 is R, D, L or W
  VARIANT (158)..(158): X29 is A or T
  VARIANT (163)..(163): X30 is L, E or T
  VARIANT (177)..(177): X31 is E or D
  VARIANT (178)..(178): X32 is K or T
  VARIANT (180)..(180): X33 is E or Q
  VARIANT (194)..(194): X34 is I or V
GSHSMRYFXTXXSRPGRGEPRFIXVGYVDDTXFVRFDSDAXSPRXXPRAPWIEQEGPEYWDRXTQXXKTXXTQXYXXNLXXXXYYNQSEAGSHXXQXMYGCDLGPDGRLLRGHDQSAYDGKDYIALNEDLXSWTAADTAAQIXQRKXEAARXAEQXRXYLEGXCVEWLRRYLENGKXXLXRADPPKTHVTHHPXSDHEATLRCWALGFYPAEITLTWQRDGEDQTQDTELVETRPAGDRTFQKWAAVVVPSGEEQRYTCHVQHEGLPKPLTLRWEP

SEQ ID NO: 44 - protein, 276 aa, Severe acute respiratory syndrome coronavirus 2 (SARS-CoV-2)
CSHSMKYFFTSVSRPGRGEPRFISVGYVDDTQFVRFDSDAASPRGEPRAPWVEQEGPEYWDRETQKYKRQAQTDRVSLRNLRGYYNQSEAGSHTLQWMCGCDLGPDGRLLRGYDQYAYDGKDYIALNEDLRSWTAADTAAQITQRKWEAAREAEQRRAYLEGTCVEWLRRYLENGKETLQRAEHPKTHVTHHPVSDHEATLRCWALGFYPAEITLTWQWDGEDQTQDTELVETRPAGDGTFQKWAAVMVPSGEEQRYTCHVQHEGLPEPLTLRWEP

SEQ ID NO: 45 - protein, 276 aa, Severe acute respiratory syndrome coronavirus 2 (SARS-CoV-2)
GSHSMRYFYTAVSRPGRGEPHFIAVGYVDDTQFVRFDSDAASPRGEPRAPWVEQEGPEYWDRETQKYKRQAQTDRVSLRNLRGYYNQSEARSHIIQRMYGCDVGPDGRLLRGYDQYAYDGKDYIALNEDLRSWTAADTAAQITQRKWEAAREAEQLRAYLEGLCVEWLRRYLKNGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWEP

SEQ ID NO: 46 - protein, 276 aa, Severe acute respiratory syndrome coronavirus 2 (SARS-CoV-2)
GSHSMRYFYTAVSRPGRGEPHFIAVGYVDDTQFVRFDSDAASPRGEPRAPWVEQEGPEYWDRETQKYKRQAQTDRVSLRNLRGYYNQSEAGSHIIQRMYGCDVGPDGRLLRGYDQYAYDGKDYIALNEDLRSWTAADTAAQITQRKWEAAREAEQLRAYLEGLCVEWLRRYLKNGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWEP

SEQ ID NO: 47 - protein, 276 aa, Severe acute respiratory syndrome coronavirus 2 (SARS-CoV-2)
GSHSMRYFSTSVSWPGRGEPRFIAVGYVDDTQFVRFDSDAASPRGEPREPWVEQEGPEYWDRETQKYKRQAQADRVNLRKLRGYYNQSEDGSHTLQRMFGCDLGPDGRLLRGYNQFAYDGKDYIALNEDLRSWTAADTAAQITQRKWEAAREAEQRRAYLEGTCVEWLRRYLENGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWKP

SEQ ID NO: 48 - protein, 276 aa, Severe acute respiratory syndrome coronavirus 2 (SARS-CoV-2)
CSHSMRYFDTAVSRPGRGEPRFISVGYVDDTQFVRFDSDAASPRGEPRAPWVEQEGPEYWDRETQKYKRQAQADRVNLRKLRGYYNQSEDGSHTLQWMYGCDLGPDGRLLRGYDQSAYDGKDYIALNEDLRSWTAADTAAQITQRKWEAAREAEQWRAYLEGTCVEWLRRYLENGKETLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWEP

SEQ ID NO: 49 - protein, 276 aa, Severe acute respiratory syndrome coronavirus 2 (SARS-CoV-2)
CSHSMRYFDTAVSRPGRGEPRFISVGYVDDTQFVRFDSDAASPRGEPRAPWVEQEGPEYWDRETQNYKRQAQADRVSLRNLRGYYNQSEDGSHTLQRMYGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEP

SEQ ID NO: 50 - protein, 276 aa, Severe acute respiratory syndrome coronavirus 2 (SARS-CoV-2)
CSHSMRYFDTAVSRPGRGEPRFISVGYVDDTQFVRFDSDAASPRGEPRAPWVEQEGPEYWDRETQKYKRQAQADRVSLRNLRGYYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEP

SEQ ID NO: 51 - protein, 276 aa, Severe acute respiratory syndrome coronavirus 2 (SARS-CoV-2)
CSHSMRYFYTAVSRPGRGEPRFIAVGYVDDTQFVQFDSDAASPRGEPRAPWVEQEGPEYWDRETQKYKRQAQTDRVSLRNLRGYYNQSEAGSHTLQRMYGCDLGPDGRLLRGYNQFAYDGKDYIALNEDLRSWTAADTAAQITQRKWEAARTAEQLRAYLEGTCVEWLRRYLENGKKTLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWGP

SEQ ID NO: 52 - protein, 276 aa, Severe acute respiratory syndrome coronavirus 2 (SARS-CoV-2)
CSHSMRYFYTAVSRPGRGEPHFIAVGYVDDTQFVRFDSDAASPRGEPRAPWVEQEGPEYWDRETQNYKRQAQTDRVNLRKLRGYYNQSEAGSHIIQRMYGCDLGPDGRLLRGHDQLAYDGKDYIALNEDLRSWTAADTAAQITQRKWEAAREAEQLRAYLEGTCVEWLRRYLENGKETLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWEP

SEQ ID NO: 53 - protein, 276 aa, Severe acute respiratory syndrome coronavirus 2 (SARS-CoV-2)
  VARIANT (1)..(1): X1 is C or G
  VARIANT (6)..(6): X2 is R or K
  VARIANT (9)..(9): X3 is F, Y, S, or D
  VARIANT (14)..(14): X4 is R or W
  VARIANT (21)..(21): X5 is H or R
  VARIANT (24)..(24): X6 is A or S
  VARIANT (35)..(35): X7 is Q or R
  VARIANT (49)..(49): X8 is A or E
  VARIANT (66)..(66): X9 is N or K
  VARIANT (73)..(73): X10 is T or A
  VARIANT (77)..(77): X11 is S or N
  VARIANT (80)..(80): X12 is N or K
  VARIANT (90)..(90): X13 is A or D
  VARIANT (91)..(91): X14 is G or R
  VARIANT (94)..(94): X15 is T or I
  VARIANT (95)..(95): X16 is L or I
  VARIANT (97)..(97): X17 is W or R
  VARIANT (99)..(99): X18 is C, Y, F, or S
  VARIANT (103)..(103): X19 is L, or V
  VARIANT (113)..(113): X20 is Y or H
  VARIANT (114)..(114): X21 is D or N
  VARIANT (116)..(116): X22 is Y, F, S, or L
  VARIANT (147)..(147): X23 is L or W
  VARIANT (152)..(152): X24 is E, A, Or T
  VARIANT (156)..(156): X25 is R, L, or W
  VARIANT (163)..(163): X26 is L or T
  VARIANT (173)..(173): X27 is E OR K
  VARIANT (173)..(173): X27 is E or K
  VARIANT (177)..(177): X28 is E or K
  VARIANT (184)..(184): X29 is H or P
  VARIANT (194)..(194): X30 is R or V
  VARIANT (219)..(219): X31 is W or R
  VARIANT (248)..(248): X32 is V or M
  VARIANT (253)..(253): X33 is E or Q
  VARIANT (261)..(261): X34 is M or V
  VARIANT (267)..(267): X35 is P or Q
  VARIANT (273)..(273): X36 is R or S
  VARIANT (275)..(275): X37 is P or G
XSHSMXYFXTAVSXPGRGEPXFIXVGYVDDTQFVXFDSDAASPRGEPRXPWVEQEGPEYWDRETQXYKRQAQXDRVXLRXLRGYYNQSEXXSHXXQXMXGCDXGPDGRLLRGXXQXAYDGKDYIALNEDLRSWTAADTAAQITQRKXEAARXAEQXRAYLEGXCVEWLRRYLXNGKXTLQRAEXPKTHVTHHPXSDHEATLRCWALGFYPAEITLTWQXDGEDQTQDTELVETRPAGDGTFQKWAAVXVPSGXEQRYTCHXQHEGLXEPLTLXWXP

SEQ ID NO: 54 - protein, 284 aa, Severe acute respiratory syndrome coronavirus 2 (SARS-CoV-2)
  VARIANT (89)..(89): X1= K or E
  VARIANT (107)..(107): X2= R or G
  VARIANT (157)..(157): X3= R or G
  VARIANT (158)..(158): X4= A or V
  VARIANT (255)..(255): X5= Q or P
  VARIANT (267)..(267): X6= P or S
GSHSLKYFHTSVSRPGRGEPRFISVGYVDDTQFVRFDNDAASPRMVPRAPWMEQEGSEYWDRETRSARDTAQIFRVNLRTLRGYYNQSXAGSHTLQWMHGCELGPDXRFLRGYEQFAYDGKDYLTLNEDLRSWTAVDTAAQISEQKSNDASEAEHQXXYLEDTCVEWLHKYLEKGKETLLHLEPPKTHVTHHPISDHEATLRCWALGFYPAEITLTWQQDGEGHTQDTELVETRPAGDGTFQKWAAVVVPSGEEXRYTCHVQHEGLXEPVTLRWKPASQPTIPI

SEQ ID NO: 55 - protein, 284 aa, Severe acute respiratory syndrome coronavirus 2 (SARS-CoV-2)
  VARIANT (7)..(7): X1= Y or F
  VARIANT (50)..(50): X2= P or Q
  VARIANT (251)..(251): X3= S or P
  VARIANT (278)..(278): X4= P or L
GSHSLRXFSTAVSRPGRGEPRYIAVEYVDDTQFLRFDSDAAIPRMEPREXWVEQEGPQYWEWTTGYAKANAQTDRVALRNLLRRYNQSEAGSHTLQGMNGCDMGPDGRLLRGYHQHAYDGKDYISLNEDLRSWTAADTVAQITQRFYEAEEYAEEFRTYLEGECLELLRRYLENGKETLQRADPPKAHVAHHPISDHEATLRCWALGFYPAEITLTWQRDGEEQTQDTELVETRPAGDGTFQKWAAVVVPXGEEQRYTCHVQHEGLPQPLILRWEQSXQPTIPI

SEQ ID NO: 56 - protein, 284 aa, Severe acute respiratory syndrome coronavirus 2 (SARS-CoV-2)
  VARIANT (13)..(13): X1= S or F
  VARIANT (27)..(27): X2= Y or H
  VARIANT (31)..(31): X3= T, S, or M
  VARIANT (34)..(34): X4= L or V
  VARIANT (54)..(54): X5= Q or R
  VARIANT (81)..(81): X6= P or L
  VARIANT (100)..(100): X7= G or D
  VARIANT (104)..(104): X8= G or V
  VARIANT (105)..(105): X9= S or C
  VARIANT (110)..(110): X10= L or I
  VARIANT (159)..(159): X11= Y or H
  VARIANT (169)..(169): X12= H or R
  VARIANT (171)..(171): X13= Y or H
  VARIANT (178)..(178): X14= M or T
  VARIANT (185)..(185): X15= P or A
  VARIANT (219)..(219): X16= R, W, or Q
  VARIANT (258)..(258): X17= T or M
  VARIANT (275)..(275): X18= K or E
GSHSMRYFSAAVXRPGRGEPRFIAMGXVDDXQFXRFDSDSACPRMEPRAPWVEXEGPEYWEEETRNTKAHAQTDRMNLQTXRGYYNQSEASSHTLQWMIXCDLXXDGRLXRGYEQYAYDGKDYLALNEDLRSWTAADTAAQISKRKCEAANVAEQRRAXLEGTCVEWLXRXLENGKEXLQRADPXKTHVTHHPVFDYEATLRCWALGFYPAEIILTWQXDGEDQTQDVELVETRPAGDGTFQKWAAVVVPSGEEQRYXCHVQHEGLPEPLMLRWXQSSLPTIPI

SEQ ID NO: 57 - protein, 119 aa, Severe acute respiratory syndrome coronavirus 2 (SARS-CoV-2)
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 58 - protein, 119 aa, Severe acute respiratory syndrome coronavirus 2 (SARS-CoV-2); identical to SEQ ID NO: 57

SEQ ID NO: 59 - protein, 119 aa, Severe acute respiratory syndrome coronavirus 2 (SARS-CoV-2)
MSRSVALAVLALLSLSGLEAIQRTPKIQVYSRHPPENGKPNFLNCYVSGFHPSDIEVDLLKNGEKMGKVEHSDLSFSKDWSFYLLYYTEFTPNEKDEYACRVNHVTLSGPRTVKWDRDM

SEQ ID NO: 60 - protein, 118 aa, Severe acute respiratory syndrome coronavirus 2 (SARS-CoV-2)
MARFVALVLLGLLSLSGLDAIQRPPKIQVYSRHPPEDGKPNYLNCYVYGFHPPQIEIDLLKNGEKIKSEQSDLSFSKDWSFYLLSHAEFTPNSKDQYSCRVKHVTLEQPRIVKWDRDL

SEQ ID NO: 61 - protein, 119 aa, Severe acute respiratory syndrome coronavirus 2 (SARS-CoV-2)
MARSVTLVFLVLVSLTGLYAIQKTPQIQVYSRHPPENGKPNILNCYVTQFHPPHIEIQMLKNGKKIPKVEMSDMSFSKDWSFYILAHTEFTPTETDTYACRVKHASMAEPKTVYWDRDM

SEQ ID NO: 62 - protein, 6 aa, Severe acute respiratory syndrome coronavirus 2 (SARS-CoV-2)
  VARIANT (1)..(1): Position 1 (X1) is present or absent and, when present, can be any amino acid, usually L, M, V, S or T, more usually L, M, S or V, with the proviso that, when the sulfatase motif is at the N-terminus of the target polypeptide, X1 is present.
  VARIANT (2)..(2): Position 2 (Z1) is cysteine or serine.
  VARIANT (3)..(3): Position 3 (X2) can be any amino acid, though usually an aliphatic aa, a polar, uncharged aa, or a sulfur containing amino acid (e.g., other than an aromatic aa or a charged aa), usually S, T, A, V, G or C, more usually S, T, A, V or G.
  VARIANT (4)..(4): Position 4 (Z2) is either a proline or alanine residue (which can also be represented by “P/A”).
  VARIANT (5)..(5): Position 5 (X3) can be any amino acid, though usually an aliphatic aa, a polar, uncharged aa, or a sulfur containing amino acid (e.g., other than an aromatic amino acid or a charged amino acid), usually S, T, A, V, G or C, more usually S, T, A, V or G.
  VARIANT (6)..(6): Position 6 (Z3) is a basic amino acid (arginine, lysine, or histidine, usually lysine), or an aliphatic amino acid (alanine, glycine, leucine, valine, isoleucine, or proline, usually A, G, L, V, or I).
XXXXXX

SEQ ID NO: 63 - protein, 6 aa, Severe acute respiratory syndrome coronavirus 2 (SARS-CoV-2)
  VARIANT (1)..(1): Position 1 (X1) is present or absent and, when present, can be any aa, usually L, M, V, S or T, more usually L, M, S or V, with the proviso that, when the sulfatase motif is at the N-terminus of the target polypeptide, X1 is present.
  VARIANT (3)..(3): Position 3 (X2) can be any amino acid, though usually an aliphatic aa, a polar, uncharged aa, or a sulfur containing amino acid (e.g., other than an aromatic amino acid or a charged amino acid), usually S, T, A, V, G or C, more usually S, T, A, V or G.
  VARIANT (5)..(5): Position 5 (X3) can be any amino acid, though usually an aliphatic aa, a polar, uncharged amino acid, or a sulfur containing aa (e.g., other than an aromatic aa or a charged amino acid), usually S, T, A, V, G or C, more usually S, T, A, V or G.
  VARIANT (6)..(6): Position 6 (Z3) is a basic amino acid (arginine, lysine, or histidine, usually lysine), or an aliphatic aa (alanine, glycine, leucine, valine, isoleucine, or proline, usually A, G, L, V, or I).
XCXPXX

SEQ ID NO: 64 - protein, 5 aa, Severe acute respiratory syndrome coronavirus 2 (SARS-CoV-2)
  VARIANT (2)..(2): Position 2 (X1) is present or absent and, when present, can be any aa, usually L, M, V, S or T, more usually L, M, S or V, with the proviso that, when the sulfatase motif is at the N-terminus of the target polypeptide, X1 is present.
  VARIANT (4)..(4): Position 4 (X2) can be any aa, though usually an aliphatic aa, a polar, uncharged aa, or a sulfur containing aa (e.g., other than an aromatic aa or a charged aa), usually S, T, A, V, G or C, more usually S, T, A, V or G.
  VARIANT (5)..(5): Position 5 (Z3) is a basic amino acid (arginine, lysine, or histidine, usually lysine), or an aliphatic aa (alanine, glycine, leucine, valine, isoleucine, or proline, usually A, G, L, V, or I).
CXPXX

SEQ ID NO: 65 - protein, 5 aa, Severe acute respiratory syndrome coronavirus 2 (SARS-CoV-2)
  SITE (3)..(3): X5 can be any single amino acid.
  SITE (5)..(5): X can be a glycine or alanine.
LPXTX

SEQ ID NO: 66 - protein, 5 aa, Severe acute respiratory syndrome coronavirus 2 (SARS-CoV-2)
GGGGG

SEQ ID NO: 67 - protein, 4 aa, Severe acute respiratory syndrome coronavirus 2 (SARS-CoV-2)
GGGG

SEQ ID NO: 68 - protein, 5 aa, Severe acute respiratory syndrome coronavirus 2 (SARS-CoV-2)
AAAAA

SEQ ID NO: 69 - protein, 4 aa, Severe acute respiratory syndrome coronavirus 2 (SARS-CoV-2)
AAAA

SEQ ID NO: 70 - protein, 6 aa, Severe acute respiratory syndrome coronavirus 2 (SARS-CoV-2)
LPETGG

SEQ ID NO: 71 - protein, 6 aa, Severe acute respiratory syndrome coronavirus 2 (SARS-CoV-2)
LPETAA

SEQ ID NO: 72 - protein, 5 aa, Severe acute respiratory syndrome coronavirus 2 (SARS-CoV-2)
LLQGG

SEQ ID NO: 73 - protein, 4 aa, Severe acute respiratory syndrome coronavirus 2 (SARS-CoV-2)
LLQG

SEQ ID NO: 74 - protein, 6 aa, Severe acute respiratory syndrome coronavirus 2 (SARS-CoV-2)
LSLSQG

SEQ ID NO: 75 - protein, 6 aa, Severe acute respiratory syndrome coronavirus 2 (SARS-CoV-2)
LLQLQG

SEQ ID NO: 76 - protein, 276 aa, Severe acute respiratory syndrome coronavirus 2 (SARS-CoV-2)
  VARIANT (79)..(83): Residues 79-83 each can be any amino acid or any amino acid other than glycine in an embodiment it is the sequence GTLRG
  DISULFID (84)..(84): Residue 84 can form a disulfide bond with residue 139
  VARIANT (85)..(89): Residues 85-89 each can be any amino acid or any amino acid other than glycine, in an embodiment it is the sequence YNQSE
  VARIANT (134)..(138): Residues 134-138 each can be any amino acid or any amino acid other than glycine, in an embodiment it is the sequence TAADM
  DISULFID (139)..(139): Residue 139 can form a disulfide bond with residue 84
  VARIANT (140)..(144): Residues 140-144 each can be any amino acid or any amino acid other than glycine, in an embodiment it is the sequence AQITK
GSHSMRYFFTSVSRPGRGEPRFIAVGYVDDTQFVRFDSDAASQRMEPRAPWIEQEGPEYWDGETRKVKAHSQTHRVDLXXXXXCXXXXXAGSHTVQRMYGCDVGSDWRFLRGYHQYAYDGKDYIALKEDLRSWXXXXXCXXXXXHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEP

SEQ ID NO: 77 - protein, 99 aa, Severe acute respiratory syndrome coronavirus 2 (SARS-CoV-2)
IQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 78 - protein, 276 aa, Severe acute respiratory syndrome coronavirus 2 (SARS-CoV-2)
  VARIANT (79)..(83): Residues 79-83 each can be any amino acid or any amino acid other than glycine in an embodiment it is the sequence GTLRG
  DISULFID (84)..(84): Residue 84 can form a disulfide bond with residue 139
  VARIANT (85)..(89): Residues 85-89 each can be any amino acid or any amino acid other than glycine, in an embodiment it is the sequence YNQSE
  VARIANT (134)..(138): Residues 134-138 each can be any amino acid or any amino acid other than glycine, in an embodiment it is the sequence TAADM
  DISULFID (139)..(139): Residue 139 can form a disulfide bond with residue 84
  VARIANT (140)..(144): Residues 140-144 each can be any amino acid or any amino acid other than glycine, in an embodiment it is the sequence AQITK
  VARIANT (231)..(235): Residues 231-235 each can be any amino acid or any amino acid other than glycine, in an embodiment it is the sequence VETRP
  DISULFID (236)..(236): Residue 236 can form a disulfide bond with beta-2M-containing polypeptide
  VARIANT (237)..(241): Residues 237-241 each can be any amino acid or any amino acid other than glycine, in an embodiment it is the sequence GDGTF
GSHSMRYFFTSVSRPGRGEPRFIAVGYVDDTQFVRFDSDAASQRMEPRAPWIEQEGPEYWDGETRKVKAHSQTHRVDLXXXXXCXXXXXAGSHTVQRMYGCDVGSDWRFLRGYHQYAYDGKDYIALKEDLRSWXXXXXCXXXXXHKWEAAHVAEQLRAYLEGTCVEWLRRYLENGKETLQRTDAPKTHMTHHAVSDHEATLRCWALSFYPAEITLTWQRDGEDQTQDTELXXXXXCXXXXXQKWAAVVVPSGQEQRYTCHVQHEGLPKPLTLRWEP

SEQ ID NO: 79 - protein, 99 aa, Severe acute respiratory syndrome coronavirus 2 (SARS-CoV-2); identical to SEQ ID NO: 77

SEQ ID NO: 80 - protein, 5 aa, Severe acute respiratory syndrome coronavirus 2 (SARS-CoV-2)
  VARIANT (4)..(4): X is any amino acid other than proline.
VPGXG

SEQ ID NO: 81 - protein, 5 aa, Severe acute respiratory syndrome coronavirus 2 (SARS-CoV-2)
GSGGS

SEQ ID NO: 82 - protein, 4 aa, Severe acute respiratory syndrome coronavirus 2 (SARS-CoV-2)
GGGS

SEQ ID NO: 83 - protein, 4 aa, Severe acute respiratory syndrome coronavirus 2 (SARS-CoV-2)
GGSG

SEQ ID NO: 84 - protein, 5 aa, Severe acute respiratory syndrome coronavirus 2 (SARS-CoV-2)
GGSGG

SEQ ID NO: 85 - protein, 5 aa, Severe acute respiratory syndrome coronavirus 2 (SARS-CoV-2)
GSGSG

SEQ ID NO: 86 - protein, 5 aa, Severe acute respiratory syndrome coronavirus 2 (SARS-CoV-2)
GSGGG

SEQ ID NO: 87 - protein, 5 aa, Severe acute respiratory syndrome coronavirus 2 (SARS-CoV-2)
GGGSG

SEQ ID NO: 88 - protein, 5 aa, Severe acute respiratory syndrome coronavirus 2 (SARS-CoV-2)
GSSSG

SEQ ID NO: 89 - protein, 5 aa, Severe acute respiratory syndrome coronavirus 2 (SARS-CoV-2)
GSSSS

SEQ ID NO: 90 - protein, 5 aa, Severe acute respiratory syndrome coronavirus 2 (SARS-CoV-2)
GGGGS

SEQ ID NO: 91 - protein, 5 aa, Severe acute respiratory syndrome coronavirus 2 (SARS-CoV-2)
AAAGG

SEQ ID NO: 92 - protein, 5 aa, Severe acute respiratory syndrome coronavirus 2 (SARS-CoV-2)
GGGGS

SEQ ID NO: 93 - protein, 10 aa, Severe acute respiratory syndrome coronavirus 2 (SARS-CoV-2)
GCGGSGGGGS

SEQ ID NO: 94 - protein, 15 aa, Severe acute respiratory syndrome coronavirus 2 (SARS-CoV-2)
GCGASGGGGSGGGGS

SEQ ID NO: 95 - protein, 20 aa, Severe acute respiratory syndrome coronavirus 2 (SARS-CoV-2)
GCGGSGGGGSGGGGSGGGGS

SEQ ID NO: 96 - protein, 15 aa, Severe acute respiratory syndrome coronavirus 2 (SARS-CoV-2)
GCGGSGGGGSGGGGS

SEQ ID NO: 97 - protein, 245 aa, Severe acute respiratory syndrome coronavirus 2 (SARS-CoV-2)
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPGNILNVSIKICLTLSPST

SEQ ID NO: 98 - protein, 219 aa, Severe acute respiratory syndrome coronavirus 2 (SARS-CoV-2)
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPGNILNVSIKI

SEQ ID NO: 99 - protein, 124 aa, Severe acute respiratory syndrome coronavirus 2 (SARS-CoV-2)
AFTVTVPKDLYVVEYGSNMTIECKFPVEKQLDLAALIVYWEMEDKNIIQFVHGEEDLKTQHSSYRQRARLLKDQLSLGNAALQITDVKLQDAGVYRCMISYGGADYKRITVKVNAPYAAALHEH

SEQ ID NO: 100 - protein, 268 aa, Severe acute respiratory syndrome coronavirus 2 (SARS-CoV-2)
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 101 - protein, 208 aa, Severe acute respiratory syndrome coronavirus 2 (SARS-CoV-2) [sequence omitted]

SEQ ID NO: 102 - protein, 220 aa, Severe acute respiratory syndrome coronavirus 2 (SARS-CoV-2)
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 103 - protein, 123 aa, Severe acute respiratory syndrome coronavirus 2 (SARS-CoV-2)
MLRLLLALNLFPSIQVTGNKILVKQSPMLVAYDNAVNLSWKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 104 - protein, 101 aa, Severe acute respiratory syndrome coronavirus 2 (SARS-CoV-2)
MLRLLLALNLFPSIQVTGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 105 - protein, 224 aa, Severe acute respiratory syndrome coronavirus 2 (SARS-CoV-2)
APLKIQAYFNETADLPCQFANSQNQSLSELVVFWQDQENLVLNEVYLGKEKFDSVHSKYMNRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 106 - protein, 110 aa, Severe acute respiratory syndrome coronavirus 2 (SARS-CoV-2)
APLKIQAYFNETADLPCQFANSQNQSLSELVVFWQDQENLVLNEVYLGKEKFDSVHSKYMNRTSFDSDSWTLRLHNLQIKDKGLYQCIIHHKKPTGMIRIHQMNSELSVL

SEQ ID NO: 107 - protein, 254 aa, Severe acute respiratory syndrome coronavirus 2 (SARS-CoV-2)
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 108 - protein, 174 aa, Severe acute respiratory syndrome coronavirus 2 (SARS-CoV-2)
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 109 - protein, 175 aa, Severe acute respiratory syndrome coronavirus 2 (SARS-CoV-2)
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 110 - protein, 167 aa, Severe acute respiratory syndrome coronavirus 2 (SARS-CoV-2)
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPA

SEQ ID NO: 111 - protein, 255 aa, Severe acute respiratory syndrome coronavirus 2 (SARS-CoV-2)
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 112 - protein, 133 aa, Severe acute respiratory syndrome coronavirus 2 (SARS-CoV-2) [sequence omitted]

SEQ ID NO: 113 - protein, 251 aa, Severe acute respiratory syndrome coronavirus 2 (SARS-CoV-2)
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 114 - protein, 524 aa, Severe acute respiratory syndrome coronavirus 2 (SARS-CoV-2)
VNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 115 - protein, 347 aa, Severe acute respiratory syndrome coronavirus 2 (SARS-CoV-2)
LNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 116 - protein, 9 aa, Severe acute respiratory syndrome coronavirus 2 (SARS-CoV-2)
YPYDVPDYA

SEQ ID NO: 117 - protein, 10 aa, Severe acute respiratory syndrome coronavirus 2 (SARS-CoV-2)
EQKLISEEDL

SEQ ID NO: 118 - protein, 5 aa, Severe acute respiratory syndrome coronavirus 2 (SARS-CoV-2)
HHHHH

SEQ ID NO: 119 - protein, 6 aa, Severe acute respiratory syndrome coronavirus 2 (SARS-CoV-2)
HHHHHH

SEQ ID NO: 120 - protein, 10 aa, Severe acute respiratory syndrome coronavirus 2 (SARS-CoV-2)
EQKLISEEDL

SEQ ID NO: 121 - protein, 8 aa, Severe acute respiratory syndrome coronavirus 2 (SARS-CoV-2)
DYKDDDDK

SEQ ID NO: 122 - protein, 8 aa, Severe acute respiratory syndrome coronavirus 2 (SARS-CoV-2)
WSHPQFEK

SEQ ID NO: 123 - protein, 9 aa, Severe acute respiratory syndrome coronavirus 2 (SARS-CoV-2)
YPYDVPDYA

SEQ ID NO: 124 - protein, 5 aa, Severe acute respiratory syndrome coronavirus 2 (SARS-CoV-2)
RYIRS

SEQ ID NO: 125 - protein, 4 aa, Severe acute respiratory syndrome coronavirus 2 (SARS-CoV-2)
FHHT

SEQ ID NO: 126 - protein, 17 aa, Severe acute respiratory syndrome coronavirus 2 (SARS-CoV-2)
WEAAAREACCRECCARA

SEQ ID NO: 127 - protein, 276 aa, Severe acute respiratory syndrome coronavirus 2 (SARS-CoV-2)
GSHSMRYFYTAMSRPGRGEPRFIAVGYVDDTQFVRFDSDAASPRTEPRAPWIEQEGPEYWDRNTQIFKTNTQTYRESLRNLRGYYNQSEAGSHIIQRMYGCDLGPDGRLLRGHDQSAYDGKDYIALNEDLSSWTAADTAAQITQRKWEAARVAEQLRAYLEGLCVEWLRRYLENGKETLQRADPPKTHVTHHPVSDHEATLRCWALGFYPAEITLTWQRDGEDQTQDTELVETRPAGDRTFQKWAAVVVPSGEEQRYTCHVQHEGLPKPLTLRWEP

SEQ ID NO: 128 - protein, 276 aa, Severe acute respiratory syndrome coronavirus 2 (SARS-CoV-2)
GSHSMRYFYTAMSRPGRGEPRFITVGYVDDTLFVRFDSDATSPRKEPRAPWIEQEGPEYWDRETQISKTNTQTYRENLRTALRYYNQSEAGSHIIQRMYGCDVGPDGRLLRGYDQDAYDGKDYIALNEDLSSWTAADTAAQITQRKWEAARVAEQDRAYLEGLCVESLRRYLENGKETLQRADPPKTHVTHHPISDHEVTLRCWALGFYPAEITLTWQRDGEDQTQDTELVETRPAGDRTFQKWAAVVVPSGEEQRYTCHVQHEGLPKPLTLRWEP

SEQ ID NO: 129 - protein, 276 aa, Severe acute respiratory syndrome coronavirus 2 (SARS-CoV-2)
GSHSMRYFYTAMSRPGRGEPRFITVGYVDDTLFVRFDSDATSPRKEPRAPWIEQEGPEYWDRETQISKTNTQTYRENLRTALRYYNQSEAGSHIIQRMYGCDVGPDGRLLRGYDQDAYDGKDYIALNEDLSSWTAADTAAQITQRKWEAARVAEQLRAYLEGLCVESLRRYLENGKETLQRADPPKTHVTHHPISDHEVTLRCWALGFYPAEITLTWQRDGEDQTQDTELVETRPAGDRTFQKWAAVVVPSGEEQRYTCHVQHEGLPKPLTLRWEP

SEQ ID NO: 130 - protein, 276 aa, Severe acute respiratory syndrome coronavirus 2 (SARS-CoV-2)
GSHSMRYFYTAMSRPGRGEPRFIAVGYVDDTQFVRFDSDAASPRTEPRAPWIEQEGPEYWDGETRNMKASAQTYRENLRIALRYYNQSEAGSHIIQRMYGCDLGPDGRLLRGHDQSAYDGKDYIALNEDLSSWTAADTAAQITQRKWEAARVAEQLRAYLEGLCVEWLRRYLENGKETLQRADPPKTHVTHHPVSDHEATLRCWALGFYPAEITLTWQRDGEDQTQDTELVETRPAGDRTFQKWAAVVVPSGEEQRYTCHVQHEGLPKPLTLRWEP

SEQ ID NO: 131 - protein, 10 aa, Severe acute respiratory syndrome coronavirus 2 (SARS-CoV-2)
TKAYNVTQAF

SEQ ID NO: 132 - protein, 8 aa, Severe acute respiratory syndrome coronavirus 2 (SARS-CoV-2)
LEVLFQGP

SEQ ID NO: 133 - protein, 7 aa, Severe acute respiratory syndrome coronavirus 2 (SARS-CoV-2)
ENLYTQS

SEQ ID NO: 134 - protein, 5 aa, Severe acute respiratory syndrome coronavirus 2 (SARS-CoV-2)
DDDDK

SEQ ID NO: 135 - protein, 4 aa, Severe acute respiratory syndrome coronavirus 2 (SARS-CoV-2)
LVPR

SEQ ID NO: 136 - protein, 22 aa, Severe acute respiratory syndrome coronavirus 2 (SARS-CoV-2)
GSGATNFSLLKQAGDVEENPGP

SEQ ID NO: 137 - protein, 123 aa, Severe acute respiratory syndrome coronavirus 2 (SARS-CoV-2)
NLVPMVATVGGGASGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 138 - protein, 813 aa, Severe acute respiratory syndrome coronavirus 2 (SARS-CoV-2)
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGAYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 139 - protein, 99 aa, Severe acute respiratory syndrome coronavirus 2 (SARS-CoV-2)
  SITE (7)..(7): cysteine that acts as a chemical conjugation site
  SITE (44)..(44): cysteine that acts as a chemical conjugation site
IQRTPKIQVYSCHPAENGKSNFLNCYVSGFHPSDIEVDLLKNGCRIEKVEHSDLSFSKDWSFYLLYYTEFTPTEKDEYACRVNHVTLSQPKIVKWDRDM

SEQ ID NO: 140 - protein, 813 aa, Severe acute respiratory syndrome coronavirus 2 (SARS-CoV-2)
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 141 - protein, 135 aa, Severe acute respiratory syndrome coronavirus 2 (SARS-CoV-2)
MSRSVALAVLALLSLSGLEAGGGGSLCTPSRGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 142 - protein, 833 aa, Severe acute respiratory syndrome coronavirus 2 (SARS-CoV-2)
MYRMQLLSCIALSLALVTNSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 143 - protein, 20 aa, Severe acute respiratory syndrome coronavirus 2 (SARS-CoV-2)
MYRMQLLSCIALSLALVTNS

SEQ ID NO: 144 - protein, 507 aa, Severe acute respiratory syndrome coronavirus 2 (SARS-CoV-2)
GSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRFLRGYRQDAYDGKDYIALNEDLRSWTAADMCAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 145 - protein, 275 aa, Severe acute respiratory syndrome coronavirus 2 (SARS-CoV-2)
GSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRFLRGYRQDAYDGKDYIALNEDLRSWTAADMCAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWE

SEQ ID NO: 146 - protein, 813 aa, Severe acute respiratory syndrome coronavirus 2 (SARS-CoV-2)
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRFLRGYRQDAYDGKDYIALNEDLRSWTAADMCAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 147 - protein, 532 aa, Severe acute respiratory syndrome coronavirus 2 (SARS-CoV-2)
GSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRFLRGYRQDAYDGKDYIALNEDLRSWTAADMCAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEGGGGSGGGGSGGGGSGGGGSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 148 - protein, 838 aa, Severe acute respiratory syndrome coronavirus 2 (SARS-CoV-2)
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRFLRGYRQDAYDGKDYIALNEDLRSWTAADMCAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEGGGGSGGGGSGGGGSGGGGSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 149 - protein, 9 aa, Artificial Sequence
  Peptide of Coronavirus protein
GLMWLSYFV

SEQ ID NO: 150 - protein, 9 aa, Artificial Sequence
  Peptide of Coronavirus Protein
GLMWLSYFI

SEQ ID NO: 151 - protein, 10 aa, Artificial Sequence
  Peptide of Coronavirus Protein
TLACFVLAAV

SEQ ID NO: 152 - protein, 9 aa, Artificial Sequence
  Peptide of Coronavirus Protein
ALNTPKDHI

SEQ ID NO: 153 - protein, 9 aa, Artificial Sequence
  Peptide of Coronavirus Protein
GMSRIGMEV

SEQ ID NO: 154 - protein, 9 aa, Artificial Sequence
  Peptide of Coronavirus Protein
LALLLLDRL

SEQ ID NO: 155 - protein, 9 aa, Artificial Sequence
  Peptide of Coronavirus Protein
LLLDRLNQL

SEQ ID NO: 156 - protein, 9 aa, Artificial Sequence
  Peptide of Coronavirus Protein
LQLPQGTTL

SEQ ID NO: 157 - protein, 9 aa, Artificial Sequence
  Peptide of Coronavirus Protein
RLNQLESKV

SEQ ID NO: 158 - protein, 9 aa, Artificial Sequence
  Peptide of Coronavirus Protein
RLNQLESKM

SEQ ID NO: 159 - protein, 9 aa, Artificial Sequence
  Peptide of Coronavirus Protein
ALSGVFCGV

SEQ ID NO: 160 - protein, 9 aa, Artificial Sequence
  Peptide of Coronavirus Protein
SLPGVFCGV

SEQ ID NO: 161 - protein, 9 aa, Artificial Sequence
  Peptide of Coronavirus Protein
CLDAGINYV

SEQ ID NO: 162 - protein, 9 aa, Artificial Sequence
  Peptide of Coronavirus Protein
CLEASFNYL

SEQ ID NO: 163 - protein, 9 aa, Artificial Sequence
  Peptide of Coronavirus Protein
ILLLDQVLV

SEQ ID NO: 164 - protein, 9 aa, Artificial Sequence
  Peptide of Coronavirus Protein
ILLLDQALV

SEQ ID NO: 165 - protein, 9 aa, Artificial Sequence
  Peptide of Coronavirus Protein
LLCVLAALV

SEQ ID NO: 166 - protein, 9 aa, Artificial Sequence
  Peptide of Coronavirus Protein
SACVLAAEC

SEQ ID NO: 167 - protein, 9 aa, Artificial Sequence
  Peptide of Coronavirus Protein
SMWALVISV

SEQ ID NO: 168 - protein, 9 aa, Artificial Sequence
  Peptide of Coronavirus Protein
SMWALIISV

SEQ ID NO: 169 - protein, 9 aa, Artificial Sequence
  Peptide of Coronavirus Protein
TLMNVITLV

SEQ ID NO: 170 - protein, 9 aa, Artificial Sequence
  Peptide of Coronavirus Protein
TLMNVLTLV

SEQ ID NO: 171 - protein, 9 aa, Artificial Sequence
  Peptide of Coronavirus Protein
WLMWFIISI

SEQ ID NO: 172 - protein, 9 aa, Artificial Sequence
  Peptide of Coronavirus Protein
WLMWLIINL

SEQ ID NO: 173 - protein, 9 aa, Artificial Sequence
  Peptide of Coronavirus Protein
ALNTLVKQL

SEQ ID NO: 174 - protein, 9 aa, Artificial Sequence
  Peptide of Coronavirus Protein
FIAGLIAIV

SEQ ID NO: 175 - protein, 10 aa, Artificial Sequence
  Peptide of Coronavirus Protein
KLPDDFMGCV

SEQ ID NO: 176 - protein, 10 aa, Artificial Sequence
  Peptide of Coronavirus Protein
KLPDDFTGCV

SEQ ID NO: 177 - protein, 9 aa, Artificial Sequence
  Peptide of Coronavirus Protein
LITGRLQSL

SEQ ID NO: 178 - protein, 9 aa, Artificial Sequence
  Peptide of Coronavirus Protein
NLNESLIDL

SEQ ID NO: 179 - protein, 9 aa, Artificial Sequence
  Peptide of Coronavirus Protein
RLNEVAKNL

SEQ ID NO: 180 - protein, 9 aa, Artificial Sequence
  Peptide of Coronavirus Protein
SIVAYTMSL

SEQ ID NO: 181 - protein, 9 aa, Artificial Sequence
  Peptide of Coronavirus Protein
SIIAYTMSL

SEQ ID NO: 182 - protein, 9 aa, Artificial Sequence
  Peptide of Coronavirus Protein
VLNDILSRL

SEQ ID NO: 183 - protein, 9 aa, Artificial Sequence
  Peptide of Coronavirus Protein
QFKDNVILL

SEQ ID NO: 184 - protein, 9 aa, Artificial Sequence
  Peptide of Coronavirus Protein
NFKDQVILL

SEQ ID NO: 185 - protein, 10 aa, Artificial Sequence
  Peptide of Coronavirus Protein
GETALALLLL

SEQ ID NO: 186 - protein, 10 aa, Artificial Sequence
  Peptide of Coronavirus Protein
GDAALALLLL

SEQ ID NO: 187 - protein, 10 aa, Artificial Sequence
  Peptide of Coronavirus Protein
MEVTPSGTWL

SEQ ID NO: 188 - protein, 15 aa, Artificial Sequence
  Peptide of Coronavirus Protein
RFFTLGSITAQPVKI

SEQ ID NO: 189 - protein, 15 aa, Artificial Sequence
  Peptide of Coronavirus Protein
RIFTIGTVTLKQGEI

SEQ ID NO: 190 - protein, 9 aa, Artificial Sequence
  Peptide of Coronavirus Protein
SITAQPVKI

SEQ ID NO: 191 - protein, 9 aa, Artificial Sequence
  Peptide of Coronavirus Protein
TVTLKQGEI

SEQ ID NO: 192 - protein, 9 aa, Artificial Sequence
  Peptide of Coronavirus Protein
HLRMAGHSL

SEQ ID NO: 193 - protein, 9 aa, Artificial Sequence
  Peptide of Coronavirus Protein
HLRIAGHHL

SEQ ID NO: 194 - protein, 10 aa, Artificial Sequence
  Peptide of Coronavirus Protein
TKQYNVTQAF

SEQ ID NO: 195 - protein, 306 aa, Severe acute respiratory syndrome coronavirus 2 (SARS-CoV-2) [sequence omitted]

SEQ ID NO: 196 - protein, 75 aa, Severe acute respiratory syndrome coronavirus 2 (SARS-CoV-2)
MYSFVSEETGTLIVNSVLLFLAFVVFLLVTLAILTALRLCAYCCNIVNVSLVKPSFYVYSRVKNLNSSRVPDLLV

SEQ ID NO: 197 - protein, 601 aa, Severe acute respiratory syndrome coronavirus 2 (SARS-CoV-2)
AVGACVLCNSQTSLRCGACIRRPFLCCKCCYDHVISTSHKLVLSVNPYVCNAPGCDVTDVTQLYLGGMSYYCKSHKPPISFPLCANGQVFGLYKNTCVGSDNVTDFNAIATCDWTNAGDYILANTCTERLKLFAAETLKATEETFKLSYGIATVREVLSDRELHLSWEVGKPRPPLNRNYVFTGYRVTKNSKVQIGEYTFEKGDYGDAVVYRGTTTYKLNVGDYFVLTSHTVMPLSAPTLVPQEHYVRITGLYPTLNISDEFSSNVANYQKVGMQKYSTLQGPPGTGKSHFAIGLALYYPSARIVYTACSHAAVDALCEKALKYLPIDKCSRIIPARARVECFDKFKVNSTLEQYVFCTVNALPETTADIVVFDEISMATNYDLSVVNARLRAKHYVYIGDPAQLPAPRTLLTKGTLEPEYFNSVCRLMKTIGPDMFLGTCRRCPAEIVDTVSALVYDNKLKAHKDKSAQCFKMFYKGVITHDVSSAINRPQIGVVREFLTRNPAWRKAVFISPYNSQNAVASKILGLPTQTVDSSQGSEYDYVIFTQTTETAHSCNVNRFNVAITRAKVGILCIMSDRDLYDKLQFTSLEIPRRNVATLQ

SEQ ID NO: 198 - protein, 222 aa, Severe acute respiratory syndrome coronavirus 2 (SARS-CoV-2)
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 199 - protein, 180 aa, Severe acute respiratory syndrome coronavirus 2 (SARS-CoV-2)
MESLVPGFNEKTHVQLSLPVLQVRDVLVRGFGDSVEEVLSEARQHLKDGTCGLVEVEKGVLPQLEQPYVFIKRSDARTAPHGHVMVELVAELEGIQYGRSGETLGVLVPHVGEIPVAYRKVLLRKNGNKGAGGHSYGADLKSFDLGDELGTDPYEDFQENWNTKHSSGVTRELMRELNGG

SEQ ID NO: 200 - protein, 638 aa, Severe acute respiratory syndrome coronavirus 2 (SARS-CoV-2)
AYTRYVDNNFCGPDGYPLECIKDLLARAGKASCTLSEQLDFIDTKRGVYCCREHEHEIAWYTERSEKSYELQTPFEIKLAKKFDTFNGECPNFVFPLNSIIKTIQPRVEKKKLDGFMGRIRSVYPVASPNECNQMCLSTLMKCDHCGETSWQTGDFVKATCEFCGTENLTKEGATTCGYLPQNAVVKIYCPACHNSEVGPEHSLAEYHNESGLKTILRKGGRTIAFGGCVFSYVGCHNKCAYWVPRASANIGCNHTGVVGEGSEGLNDNLLEILQKEKVNINIVGDFKLNEEIAIILASFSASTSAFVETVKGLDYKAFKQIVESCGNFKVTKGKAKKGAWNIGEQKSILSPLYAFASEAARVVRSIFSRTLETAQNSVRVLQKAAITILDGISQYSLRLIDAMMFTSDLATNNLVVMAYITGGVVQLTSQWLTNIFGTVYEKLKPVLDWLEEKFKEGVEFLRDGWEIVKFISTCACEIVGGQIVTCAKEIKESVQTFFKLVNKFLALCADSIIIGGAKLKALNLGETFVTHSKGLYRKCVKSREETGLLMPLKAPKEIIFLEGETLPTEVLTEEVVLKTGDLQPLEQPTSEAVEAPLVGTPVCINGLMLLEIKDTEKYCALAPNMMVTNNTFTLKGG

SEQ ID NO: 201 - protein, 500 aa, Severe acute respiratory syndrome coronavirus 2 (SARS-CoV-2)
KIVNNWLKQLIKVTLVFLFVAAIFYLITPVHVMSKHTDFSSEIIGYKAIDGGVTRDIASTDTCFANKHADFDTWFSQRGGSYTNDKACPLIAAVITREVGFVVPGLPGTILRTTNGDFLHFLPRVFSAVGNICYTPSKLIEYTDFATSACVLAAECTIFKDASGKPVPYCYDTNVLEGSVAYESLRPDTRYVLMDGSIIQFPNTYLEGSVRVVTTFDSEYCRHGTCERSEAGVCVSTSGRWVLNNDYYRSLPGVFCGVDAVNLLTNMFTPLIQPIGALDISASIVAGGIVAIVVTCLAYYFMRFRRAFGEYSHVVAFNTLLFLMSFTVLCLTPVYSFLPGVYSVIYLYLTFYLTNDVSFLAHIQWMVMFTPLVPFWITIAYIICISTKHFYWFFSNYLKRRVVFNGVSFSTFEEAALCTFLLNKEMYLKLRSDVLLPLTQYNRYLALYNKYKYFSGAMDTTSYREAACCHLAKALNDFSNSGSDVLYQPPQTSITSAVLQ

SEQ ID NO: 202 - protein, 290 aa, Severe acute respiratory syndrome coronavirus 2 (SARS-CoV-2)
SAVKRTIKGTHHWLLLTILTSLLVLVQSTQWSLFFFLYENAFLPFAMGIIAMSAFAMMFVKHKHAFLCLFLLPSLATVAYFNMVYMPASWVMRIMTWLDMVDTSLSGFKLKDCVMYASAVVLLILMTARTVYDDGARRVWTLMNVLTLVYKVYYGNALDQAISMWALIISVTSNYSGVVTTVMFLARGIVFMCVEYCPIFFITGNTLQCIMLVYCFLGYFCTCYFGLFCLLNRYFRLTLGVYDYLVSTQEFRYMNSQGLLPPKNSIDAFKLNIKLLGVGGKPCIKVATVQ

SEQ ID NO: 203 - protein, 83 aa, Severe acute respiratory syndrome coronavirus 2 (SARS-CoV-2)
SKMSDVKCTSVVLLSVLQQLRVESSSKLWAQCVQLHNDILLAKDTTEAFEKMVSLLSVLLSMQGAVDINKLCEEMLDNRATLQ

SEQ ID NO: 204 - protein, 198 aa, Severe acute respiratory syndrome coronavirus 2 (SARS-CoV-2)
AIASEFSSLPSYAAFATAQEAYEQAVANGDSEVVLKKLKKSLNVAKSEFDRDAAMQRKLEKMADQAMTQMYKQARSEDKRAKVTSAMQTMLFTMLRKLDNDALNNIINNARDGCVPLNIIPLTTAAKLMVVIPDYNTYKNTCDGTTFTYASALWEIQQVVDADSKIVQLSEISMDNSPNLAWPLIVTALRANSAVKLQ

SEQ ID NO: 205 - protein, 113 aa, Severe acute respiratory syndrome coronavirus 2 (SARS-CoV-2)
NNELSPVALRQMSCAAGTTQTACTDDNALAYYNTTKGGRFVLALLSDLQDLKWARFPKSDGTGTIYTELEPPCRFVTDTPKGPKVKYLYFIKGLNNLNRGMVLGSLAATVRLQ

SEQ ID NO: 206 - protein, 139 aa, Severe acute respiratory syndrome coronavirus 2 (SARS-CoV-2)
AGNATEVPANSTVLSFCAFAVDAAKAYKDYLASGGQPITNCVKMLCTHTGTGQAITVTPEANMDQESFGGASCCLYCRCHIDHPNPKGFCDLKGKYVQIPTTCANDPVGFTLKNTVCTVCGMWKGYGCSCDQLREPMLQ

SEQ ID NO: 207 - protein, 527 aa, Severe acute respiratory syndrome coronavirus 2 (SARS-CoV-2)
AENVTGLFKDCSKVITGLHPTQAPTHLSVDTKFKTEGLCVDIPGIPKDMTYRRLISMMGFKMNYQVNGYPNMFITREEAIRHVRAWIGFDVEGCHATREAVGTNLPLQLGFSTGVNLVAVPTGYVDTPNNTDFSRVSAKPPPGDQFKHLIPLMYKGLPWNVVRIKIVQMLSDTLKNLSDRVVFVLWAHGFELTSMKYFVKIGPERTCCLCDRRATCFSTASDTYACWHHSIGFDYVYNPFMIDVQQWGFTGNLQSNHDLYCQVHGNAHVASCDAIMTRCLAVHECFVKRVDWTIEYPIIGDELKINAACRKVQHMVVKAALLADKFPVLHDIGNPKAIKCVPQADVEWKFYDAQPCSDKAYKIEELFYSYATHSDKFTDGVCLFWNCNVDRYPANSIVCRFDTRVLSNLNLPGCDGGSLYVNKHAFHTPAFDKSAFVNLKQLPFFYYSDSPCESHGKQVVSDIDYVPLKSATCITRCNLGGAVCRHHANEYRLYLDAYNMMISAGFSLWVYKQFDTYNLWNTFTRLQ

SEQ ID NO: 208 - protein, 346 aa, Severe acute respiratory syndrome coronavirus 2 (SARS-CoV-2)
SLENVAFNVVNKGHFDGQQGEVPVSIINNTVYTKVDGVDVELFENKTTLPVNVAFELWAKRNIKPVPEVKILNNLGVDIAANTVIWDYKRDAPAHISTIGVCSMTDIAKKPTETICAPLTVFFDGRVDGQVDLFRNARNGVLITEGSVKGLQPSVGPKQASLNGVTLIGEAVKTQFNYYKKVDGVVQQLPETYFTQSRNLQEFKPRSQMEIDFLELAMDEFIERYKLEGYAFEHIVYGDFSHSQLGGLHLLIGLAKRFKESPFELEDFIPMDSTVKNYFITDAQTGSSKCVCSVIDLLLDDFVEIIKSQDLSVVSKVVKVTIDYTEISFMLWCKDGHVETFYPKLQ

SEQ ID NO: 209 - protein, 298 aa, Severe acute respiratory syndrome coronavirus 2 (SARS-CoV-2)
SSQAWQPGVAMPNLYKMQRMLLEKCDLQNYGDSATLPKGIMMNVAKYTQLCQYLNTLTLAVPYNMRVIHFGAGSDKGVAPGTAVLRQWLPTGTLLVDSDLNDFVSDADSTLIGDCATVHTANKWDLIISDMYDPKTKNVTKENDSKEGFFTYICGFIQQKLALGGSVAIKITEHSWNADLYKLMGHFAWWTAFVTNVNASSSEAFLIGCNYLGKPREQIDGYVMHANYIFWRNTNPIQLSSYSLFDMSKFPLKLRGTAVMSLKEGQINDMILSLLSKGRLIIRENNRVVISSDVLVNN

SEQ ID NO: 210 - protein, 419 aa, Severe acute respiratory syndrome coronavirus 2 (SARS-CoV-2)
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 211 - protein, 275 aa, Severe acute respiratory syndrome coronavirus 2 (SARS-CoV-2)
MDLFMRIFTIGTVTLKQGEIKDATPSDFVRATATIPIQASLPFGWLIVGVALLAVFQSASKIITLKKRWQLALSKGVHFVCNLLLLFVTVYSHLLLVAAGLEAPFLYLYALVYFLQSINFVRIIMRLWLCWKCRSKNPLLYDANYFLCWHTNCYDYCIPYNSVTSSIVITSGDGTTSPISEHDYQIGGYTEKWESGVKDCVVLHSYFTSDYYQLYSTQLSTDTGVEHVTFFIYNKIVDEPEEHVQIHTIDGSSGVVNPVMEPIYDEPTTTTSVPL

SEQ ID NO: 212 - protein, 61 aa, Severe acute respiratory syndrome coronavirus 2 (SARS-CoV-2)
MFHLVDFQVTIAEILLIIMRTFKVSIWNLDYIINLIIKNLSKSLTENKYSQLDEEQPMEID

SEQ ID NO: 213 - protein, 121 aa, Severe acute respiratory syndrome coronavirus 2 (SARS-CoV-2)
MKIILFLALITLATCELYHYQECVRGTTVLLKEPCSSGTYEGNSPFHPLADNKFALTCFSTQFAFACPDGVKHVYQLRARSVSPKLFIRQEEVQELYSPIFLIVAAIVFITLCFTLKRKTE

SEQ ID NO: 214 - protein, 121 aa, Severe acute respiratory syndrome coronavirus 2 (SARS-CoV-2)
MKFLVFLGIITTVAAFHQECSLQSCTQHQPYVVDDPCPIHFYSKWYIRVGARKSAPLIELCVDEAGSKSPIQYIDIGNYTVSCLPFTINCQEPKLGSLVVRCSFYEDFLEYHDVRVVLDFI

SEQ ID NO: 215 - protein, 38 aa, Severe acute respiratory syndrome coronavirus 2 (SARS-CoV-2)
MGYINVFAFPFTIYSLLLCRMNSRNYIAQVDVVNFNLT

SEQ ID NO: 216 - protein, 1945 aa, Severe acute respiratory syndrome coronavirus 2 (SARS-CoV-2)
APTKVTFGDDTVIEVQGYKSVNITFELDERIDKVLNEKCSAYTVELGTEVNEFACVVADAVIKTLQPVSELLTPLGIDLDEWSMATYYLFDESGEFKLASHMYCSFYPPDEDEEEGDCEEEEFEPSTQYEYGTEDDYQGKPLEFGATSAALQPEEEQEEDWLDDDSQQTVGQQDGSEDNQTTTIQTIVEVQPQLEMELTPVVQTIEVNSFSGYLKLTDNVYIKNADIVEEAKKVKPTVVVNAANVYLKHGGGVAGALNKATNNAMQVESDDYIATNGPLKVGGSCVLSGHNLAKHCLHVVGPNVNKGEDIQLLKSAYENFNQHEVLLAPLLSAGIFGADPIHSLRVCVDTVRTNVYLAVFDKNLYDKLVSSFLEMKSEKQVEQKIAEIPKEEVKPFITESKPSVEQRKQDDKKIKACVEEVTTTLEETKFLTENLLLYIDINGNLHPDSATLVSDIDITFLKKDAPYIVGDVVQEGVLTAVVIPTKKAGGTTEMLAKALRKVPTDNYITTYPGQGLNGYTVEEAKTVLKKCKSAFYILPSIISNEKQEILGTVSWNLREMLAHAEETRKLMPVCVETKAIVSTIQRKYKGIKIQEGVVDYGARFYFYTSKTTVASLINTLNDLNETLVTMPLGYVTHGLNLEEAARYMRSLKVPATVSVSSPDAVTAYNGYLTSSSKTPEEHFIETISLAGSYKDWSYSGQSTQLGIEFLKRGDKSVYYTSNPTTFHLDGEVITFDNLKTLLSLREVRTIKVFTTVDNINLHTQVVDMSMTYGQQFGPTYLDGADVTKIKPHNSHEGKTFYVLPNDDTLRVEAFEYYHTTDPSFLGRYMSALNHTKKWKYPQVNGLTSIKWADNNCYLATALLTLQQIELKFNPPALQDAYYRARAGEAANFCALILAYCNKTVGELGDVRETMSYLFQHANLDSCKRVLNVVCKTCGQQQTTLKGVEAVMYMGTLSYEQFKKGVQIPCTCGKQATKYLVQQESPFVMMSAPPAQYELKHGTFTCASEYTGNYQCGHYKHITSKETLYCIDGALLTKSSEYKGPITDVFYKENSYTTTIKPVTYKLDGVVCTEIDPKLDNYYKKDNSYFTEQPIDLVPNQPYPNASFDNFKFVCDNIKFADDLNQLTGYKKPASRELKVTFFPDLNGDVVAIDYKHYTPSFKKGAKLLHKPIVWHVNNATNKATYKPNTWCIRCLWSTKPVETSNSFDVLKSEDAQGMDNLACEDLKPVSEEVVENPTIQKDVLECNVKTTEVVGDIILKPANNSLKITEEVGHTDLMAAYVDNSSLTIKKPNELSRVLGLKTLATHGLAAVNSVPWDTIANYAKPFLNKVVSTTTNIVTRCLNRVCTNYMPYFFTLLLQLCTFTRSTNSRIKASMPTTIAKNTVKSVGKFCLEASFNYLKSPNFSKLINIIIWFLLLSVCLGSLIYSTAALGVLMSNLGMPSYCTGYREGYLNSTNVTIATYCTGSIPCSVCLSGLDSLDTYPSLETIQITISSFKWDLTAFGLVAEWFLAYILFTRFFYVLGLAAIMQLFFSYFAVHFISNSWLMWLIINLVQMAPISAMVRMYIFFASFYYVWKSYVHVVDGCNSSTCMMCYKRNRATRVECTTIVNGVRRSFYVYANGGKGFCKLHNWNCVNCDTFCAGSTFISDEVARDLSLQFKRPINPTDQSSYIVDSVTVKNGSIHLYFDKAGQKTYERHSLSHFVNLDNLRANNTKGSLPINVIVFDGKSKCEESSAKSASVYYSQLMCQPILLLDQALVSDVGDSAEVAVKMFDAYVNTFSSTFNVPMEKLKTLVATAEAELAKNVSLDNVLSTFISAARQGFVDSDVETKDVVECLKLSHQSDIEVTGDSCNNYMLTYNKVENMTPRDLGACIDCSARHINAQVAKSHNIALIWNVKDFMSLSEQLRKQIRSAAKKNNLPFKLTCATTRQVVNVVTTKIALKGG

SEQ ID NO: 217 - protein, 932 aa, Severe acute respiratory syndrome coronavirus 2 (SARS-CoV-2)
SADAQSFLNRVCGVSAARLTPCGTGTSTDVVYRAFDIYNDKVAGFAKFLKTNCCRFQEKDEDDNLIDSYFVVKRHTFSNYQHEETIYNLLKDCPAVAKHDFFKFRIDGDMVPHISRQRLTKYTMADLVYALRHFDEGNCDTLKEILVTYNCCDDDYFNKKDWYDFVENPDILRVYANLGERVRQALLKTVQFCDAMRNAGIVGVLTLDNQDLNGNWYDFGDFIQTTPGSGVPVVDSYYSLLMPILTLTRALTAESHVDTDLTKPYIKWDLLKYDFTEERLKLFDRYFKYWDQTYHPNCVNCLDDRCILHCANFNVLFSTVFPPTSFGPLVRKIFVDGVPFVVSTGYHFRELGVVHNQDVNLHSSRLSFKELLVYAADPAMHAASGNLLLDKRTTCFSVAALTNNVAFQTVKPGNFNKDFYDFAVSKGFFKEGSSVELKHFFFAQDGNAAISDYDYYRYNLPTMCDIRQLLFVVEVVDKYFDCYDGGCINANQVIVNNLDKSAGFPFNKWGKARLYYDSMSYEDQDALFAYTKRNVIPTITQMNLKYAISAKNRARTVAGVSICSTMTNRQFHQKLLKSIAATRGATVVIGTSKFYGGWHNMLKTVYSDVENPHLMGWDYPKCDRAMPNMLRIMASLVLARKHTTCCSLSHRFYRLANECAQVLSEMVMCGGSLYVKPGGTSSGDATTAYANSVFNICQAVTANVNALLSTDGNKIADKYVRNLQHRLYECLYRNRDVDTDFVNEFYAYLRKHFSMMILSDDAVVCFNSTYASQGLVASIKNFKSVLYYQNNVFMSEAKCWTETDLTKGPHEFCSQHTMLVKQGDDYVYLPYPDPSRILGAGCFVDDIVKTDGTLMIERFVSLAIDAYPLTKHPNQEYADVFHLYLQYIRKLHDELTGHMLDMYSVMLTNDNTSRYWEPEFYEAMYTPHTVLQ

SEQ ID NO: 218 - protein, 1273 aa, Severe acute respiratory syndrome coronavirus 2 (SARS-CoV-2)
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 219 - protein, 10 aa, Artificial Sequence
  Peptide of Coronavirus Protein
TSEDMLNPNY

SEQ ID NO: 220 - protein, 14 aa, Artificial Sequence
  Peptide of Coronavirus Protein
TGVHAGTDLEGNFY

SEQ ID NO: 221 - protein, 9 aa, Artificial Sequence
  Peptide of Coronavirus Protein
TTDLEGNFY

SEQ ID NO: 222 - protein, 15 aa, Artificial Sequence
  Peptide of Coronavirus Protein.
DVTDVTQLYLGGMSY

SEQ ID NO: 223 - protein, 10 aa, Artificial Sequence
  Peptide of Coronavirus Protein.
ATEETFKLSY

SEQ ID NO: 224 - protein, 12 aa, Artificial Sequence
  Peptide of Coronavirus Protein.
ISDEFSSNVANY

SEQ ID NO: 225 - protein, 10 aa, Artificial Sequence
  Peptide of Coronavirus Protein
TVDSSQGSEY

SEQ ID NO: 226 - protein, 9 aa, Artificial Sequence
  Peptide of Coronavirus Protein
TVDSQGSEY

SEQ ID NO: 227 - protein, 14 aa, Artificial Sequence
  Peptide of Coronavirus Protein
ASQRVAGDSGFAAY

SEQ ID NO: 228 - protein, 9 aa, Artificial Sequence
  Peptide of Coronavirus Protein
ASDSGFAAY

SEQ ID NO: 229 - protein, 10 aa, Artificial Sequence
  Peptide of Coronavirus Protein
VAGDSGFAAY

SEQ ID NO: 230 - protein, 9 aa, Artificial Sequence
  Peptide of Coronavirus Protein
VADSGFAAY

SEQ ID NO: 231 - protein, 10 aa, Artificial Sequence
  Peptide of Coronavirus Protein
LAMDEFIERY

SEQ ID NO: 232 - protein, 9 aa, Artificial Sequence
  Peptide of Coronavirus Protein
LADEFIERY

SEQ ID NO: 233 - protein, 11 aa, Artificial Sequence
  Peptide of Coronavirus Protein
ITEHSWNADLY

SEQ ID NO: 234 - protein, 12 aa, Artificial Sequence
  Peptide of Coronavirus Protein
YVDNNFCGPDGY

SEQ ID NO: 235 - protein, 9 aa, Artificial Sequence
  Peptide of Coronavirus Protein
YVDNGPDGY

SEQ ID NO: 236 - protein, 9 aa, Artificial Sequence
  Peptide of Coronavirus Protein
FIDTKRGVY

SEQ ID NO: 237 - protein, 9 aa, Artificial Sequence
  Peptide of Coronavirus Protein
YTERSEKSY

SEQ ID NO: 238 - protein, 14 aa, Artificial Sequence
  Peptide of Coronavirus Protein
TSDLATNNLVVMAY

SEQ ID NO: 239 - protein, 9 aa, Artificial Sequence
  Peptide of Coronavirus Protein
TSDLATNAY

SEQ ID NO: 240 - protein, 11 aa, Artificial Sequence
  Peptide of Coronavirus Protein
HTDFSSEIIGY

SEQ ID NO: 241 - protein, 9 aa, Artificial Sequence
  Peptide of Coronavirus Protein
HTDFSSEIY

SEQ ID NO: 242 - protein, 9 aa, Artificial Sequence
  Peptide of Coronavirus Protein
FSAVGNICY

SEQ ID NO: 243 - protein, 10 aa, Artificial Sequence
  Peptide of Coronavirus Protein
FSNSGSDVLY

SEQ ID NO: 244 - protein, 9 aa, Artificial Sequence
  Peptide of Coronavirus Protein
FSNSGSDLY

SEQ ID NO: 245 - protein, 10 aa, Artificial Sequence
  Peptide of Coronavirus Protein
MADQAMTQMY

SEQ ID NO: 246 - protein, 14 aa, Artificial Sequence
  Peptide of Coronavirus Protein
NTYKNTCDGTTFTY

SEQ ID NO: 247 - protein, 9 aa, Artificial Sequence
  Peptide of Coronavirus Protein
NTDGTTFTY

SEQ ID NO: 248 - protein, 14 aa, Artificial Sequence
  Peptide of Coronavirus Protein
TTQTACTDDNALAY

SEQ ID NO: 249 - protein, 9 aa, Artificial Sequence
  Peptide of Coronavirus Protein
TTDDNALAY

SEQ ID NO: 250 - protein, 13 aa, Artificial Sequence
  Peptide of Coronavirus Protein
QTACTDDNALAYY

SEQ ID NO: 251 - protein, 9 aa, Artificial Sequence
  Peptide of Coronavirus Protein
QTDNALAYY

SEQ ID NO: 252 - protein, 10 aa, Artificial Sequence
  Peptide of Coronavirus Protein
CTDDNALAYY

SEQ ID NO: 253 - protein, 9 aa, Artificial Sequence
  Peptide of Coronavirus Protein
CTDDNALYY

SEQ ID NO: 254 - protein, 14 aa, Artificial Sequence
  Peptide of Coronavirus Protein
WARFPKSDGTGTIY

SEQ ID NO: 255 - protein, 14 aa, Artificial Sequence
  Peptide of Coronavirus Protein
VTDTPKGPKVKYLY

SEQ ID NO: 256 - protein, 9 aa, Artificial Sequence
  Peptide of Coronavirus Protein
VTDTPKYLY

SEQ ID NO: 257 - protein, 13 aa, Artificial Sequence
  Peptide of Coronavirus Protein
NTNSSPDDQIGYY

SEQ ID NO: 258 - protein, 10 aa, Artificial Sequence
  Peptide of Coronavirus Protein
YFTSDYYQLY

SEQ ID NO: 259 - protein, 14 aa, Artificial Sequence
  Peptide of Coronavirus Protein
STDTGVEHVTFFIY

SEQ ID NO: 260 - protein, 14 aa, Artificial Sequence
  Peptide of Coronavirus Protein
STDTGVEHVTFFIY

SEQ ID NO: 261 - protein, 11 aa, Artificial Sequence
  Peptide of Coronavirus Protein
VVDDPCPIHFY

SEQ ID NO: 262 - protein, 9 aa, Artificial Sequence
  Peptide of Coronavirus Protein
VVDDPIHFY

SEQ ID NO: 263 - protein, 8 aa, Artificial Sequence
  Peptide of Coronavirus Protein
LTENLLLY

SEQ ID NO: 264 - protein, 9 aa, Artificial Sequence
  Peptide of Coronavirus Protein
  UNSURE (6)..(6): Unsure
  UNSURE (6)..(6): Uncertain of the amino acid at this position, it may be absent.
LTENLXLLY

SEQ ID NO: 265 - protein, 14 aa, Artificial Sequence
  Peptide of Coronavirus Protein
ALRKVPTDNYITTY

SEQ ID NO: 266 - protein, 9 aa, Artificial Sequence
  Peptide of Coronavirus Protein
ATDNYITTY

SEQ ID NO: 267 - protein, 11 aa, Artificial Sequence
  Peptide of Coronavirus Protein
KVPTDNYITTY

SEQ ID NO: 268 - protein, 9 aa, Artificial Sequence
  Peptide of Coronavirus Protein
VVDYGARFY

SEQ ID NO: 269 - protein, 11 aa, Artificial Sequence
  Peptide of Coronavirus Protein
HTTDPSFLGRY

SEQ ID NO: 270 - protein, 8 aa, Artificial Sequence
  Peptide of Coronavirus Protein
TTDPSFLR

SEQ ID NO: 271 - protein, 10 aa, Artificial Sequence
  Peptide of Coronavirus Protein
TTDPSFLGRY

SEQ ID NO: 272 - protein, 11 aa, Artificial Sequence
  Peptide of Coronavirus Protein
ITDVFYKENSY

SEQ ID NO: 273 - protein, 9 aa, Artificial Sequence
  Peptide of Coronavirus Protein
ITDYKENSY

SEQ ID NO: 274 - protein, 14 aa, Artificial Sequence
  Peptide of Coronavirus Protein
FTEQPIDLVPNQPY

SEQ ID NO: 275 - protein, 9 aa, Artificial Sequence
  Peptide of Coronavirus Protein
FTEQPIDLY

SEQ ID NO: 276 - protein, 11 aa, Artificial Sequence
  Peptide of Coronavirus Protein
FADDLNQLTGY

SEQ ID NO: 277 - protein, 9 aa, Artificial Sequence
  Peptide of Coronavirus Protein
FADDLNQLY

SEQ ID NO: 278 - protein, 14 aa, Artificial Sequence
  Peptide of Coronavirus Protein
ITEEVGHTDLMAAY

SEQ ID NO: 279 - protein, 9 aa, Artificial Sequence
  Peptide of Coronavirus Protein
ITEEVGHAY

SEQ ID NO: 280 - protein, 12 aa, Artificial Sequence
  Peptide of Coronavirus Protein
STDVVYRAFDIY

SEQ ID NO: 281 - protein, 9 aa, Artificial Sequence
  Peptide of Coronavirus Protein
STDRAFDIY

SEQ ID NO: 282 - protein, 11 aa, Artificial Sequence
  Peptide of Coronavirus Protein
FVENPDILRVY

SEQ ID NO: 283 - protein, 13 aa, Artificial Sequence
  Peptide of Coronavirus Protein
FVDGVPFVVSTGY

SEQ ID NO: 284 - protein, 10 aa, Artificial Sequence
  Peptide of Coronavirus Protein
AISDYDYYRY

SEQ ID NO: 285 - protein, 9 aa, Artificial Sequence
  Peptide of Coronavirus Protein
ASDYDYYRY

SEQ ID NO: 286 - protein, 9 aa, Artificial Sequence
  Peptide of Coronavirus Protein
VVDKYFDCY

SEQ ID NO: 287 - protein, 14 aa, Artificial Sequence
  Peptide of Coronavirus Protein
YSDVENPHLMGWDY

SEQ ID NO: 288 - protein, 9 aa, Artificial Sequence
  Peptide of Coronavirus Protein
YSDVENPHY

SEQ ID NO: 289 - protein, 12 aa, Artificial Sequence
  Peptide of Coronavirus Protein
LSEMVMCGGSLY

SEQ ID NO: 290 - protein, 11 aa, Artificial Sequence
  Peptide of Coronavirus Protein
STDGNKIADKY

SEQ ID NO: 291 - protein, 9 aa, Artificial Sequence
  Peptide of Coronavirus Protein
STDGIADKY

SEQ ID NO: 292 - protein, 13 aa, Artificial Sequence
  Peptide of Coronavirus Protein
NRDVDTDFVNEFY

SEQ ID NO: 293 - protein, 9 aa, Artificial Sequence
  Peptide of Coronavirus Protein
NTDFVNEFY

SEQ ID NO: 294 - protein, 11 aa, Artificial Sequence
  Peptide of Coronavirus Protein
DTDFVNEFYAY

SEQ ID NO: 295 - protein, 13 aa, Artificial Sequence
  Peptide of Coronavirus Protein
LSDDAVVCFNSTY

SEQ ID NO: 296 - protein, 9 aa, Artificial Sequence
  Peptide of Coronavirus Protein
LSDDANSTY

SEQ ID NO: 297 - protein, 8 aa, Artificial Sequence
  Peptide of Coronavirus Protein
YADVFHLY

SEQ ID NO: 298 - protein, 9 aa, Artificial Sequence
  Peptide of Coronavirus Protein
  UNSURE (4)..(4): Uncertain of amino acid
  UNSURE (4)..(4): Uncertain of the amino acid at this position, it may be absent.
YADXVFHLY

SEQ ID NO: 299 - protein, 13 aa, Artificial Sequence
  Peptide of Coronavirus Protein
YSVMLTNDNTSRY

SEQ ID NO: 300 - protein, 9 aa, Artificial Sequence
  Peptide of Coronavirus Protein
YTNDNTSRY

SEQ ID NO: 301 - protein, 9 aa, Artificial Sequence
  Peptide of Coronavirus Protein
WTAGAAAYY

SEQ ID NO: 302 - protein, 9 aa, Artificial Sequence
  Peptide of Coronavirus Protein
TSNQVAVLY

SEQ ID NO: 303 - protein, 10 aa, Artificial Sequence
  Peptide of Coronavirus Protein
LADAGFIKQY

SEQ ID NO: 304 - protein, 9 aa, Artificial Sequence
  Peptide of Coronavirus Protein
LADAGFIQY

SEQ ID NO: 305 - protein, 14 aa, Artificial Sequence
  Peptide of Coronavirus Protein
VLPPLLTDEMIAQY

SEQ ID NO: 306 - protein, 9 aa, Artificial Sequence
  Peptide of Coronavirus Protein
VTDEMIAQY

SEQ ID NO: 307 - protein, 10 aa, Artificial Sequence
  Peptide of Coronavirus Protein
FLVQAGNVQL

SEQ ID NO: 308 - protein, 8 aa, Artificial Sequence
  Peptide of Coronavirus Protein
FLVAGNVQ

SEQ ID NO: 309 - protein, 9 aa, Artificial Sequence
  Peptide of Coronavirus Protein
FLNRFTTTL

SEQ ID NO: 310 - protein, 12 aa, Artificial Sequence
  Peptide of Coronavirus Protein
ALLEDEFTPFDV

SEQ ID NO: 311 - protein, 9 aa, Artificial Sequence
  Peptide of Coronavirus Protein
ALLEDEFTV

SEQ ID NO: 312 - protein, 9 aa, Artificial Sequence
  Peptide of Coronavirus Protein
SLVKPSFYV

SEQ ID NO: 313 - protein, 9 aa, Artificial Sequence
  Peptide of Coronavirus Protein
KLSYGIATV

SEQ ID NO: 314 - protein, 9 aa, Artificial Sequence
  Peptide of Coronavirus Protein
TLVPQEHYV

SEQ ID NO: 315 - protein, 9 aa, Artificial Sequence
  Peptide of Coronavirus Protein
KLLEQWNLV

SEQ ID NO: 316 - protein, 9 aa, Artificial Sequence
  Peptide of Coronavirus Protein
GLVEVEKGV

SEQ ID NO: 317 - protein, 9 aa, Artificial Sequence
  Peptide of Coronavirus Protein
VMVELVAEL

SEQ ID NO: 318 - protein, 9 aa, Artificial Sequence
  Peptide of Coronavirus Protein
TLGVLVPHV

SEQ ID NO: 319 - protein, 9 aa, Artificial Sequence
  Peptide of Coronavirus Protein
YLASGGQPI

SEQ ID NO: 320 - protein, 9 aa, Artificial Sequence
  Peptide of Coronavirus Protein
MLSDTLKNL

SEQ ID NO: 321 - protein, 9 aa, Artificial Sequence
  Peptide of Coronavirus Protein
NLSDRVVFV

SEQ ID NO: 322 - protein, 9 aa, Artificial Sequence
  Peptide of Coronavirus Protein
VLWAHGFEL

SEQ ID NO: 323 - protein, 9 aa, Artificial Sequence
  Peptide of Coronavirus Protein
LLADKFPVL

SEQ ID NO: 324 - protein, 9 aa, Artificial Sequence
  Peptide of Coronavirus Protein
YLDAYNMMI

SEQ ID NO: 325 - protein, 9 aa, Artificial Sequence
  Peptide of Coronavirus Protein
MMISAGFSL

SEQ ID NO: 326 - protein, 9 aa, Artificial Sequence
  Peptide of Coronavirus Protein
SLENVAFNV

SEQ ID NO: 327 - protein, 9 aa, Artificial Sequence
  Peptide of Coronavirus Protein
SQLGGLHLL

SEQ ID NO: 328 - protein, 10 aa, Artificial Sequence
  Peptide of Coronavirus Protein
LLLDDFVEII

SEQ ID NO: 329 - protein, 9 aa, Artificial Sequence
  Peptide of Coronavirus Protein
YLNTLTLAV

SEQ ID NO: 330 - protein, 9 aa, Artificial Sequence
  Peptide of Coronavirus Protein
TLIGDCATV

SEQ ID NO: 331 - protein, 12 aa, Artificial Sequence
  Peptide of Coronavirus Protein
SLFDMSKFPLKL

SEQ ID NO: 332 - protein, 9 aa, Artificial Sequence
  Peptide of Coronavirus Protein
SLFDFPLKL

SEQ ID NO: 333 - protein, 10 aa, Artificial Sequence
  Peptide of Coronavirus Protein
GLNDNLLEIL

SEQ ID NO: 334 - protein, 9 aa, Artificial Sequence
  Peptide of Coronavirus Protein
GLNDNLLEL

SEQ ID NO: 335 - protein, 9 aa, Artificial Sequence
  Peptide of Coronavirus Protein
KLNEEIAII

SEQ ID NO: 336 - protein, 9 aa, Artificial Sequence
  Peptide of Coronavirus Protein
RLIDAMMFT

SEQ ID NO: 337 - protein, 9 aa, Artificial Sequence
  Peptide of Coronavirus Protein
YITGGVVQL

SEQ ID NO: 338 - protein, 9 aa, Artificial Sequence
  Peptide of Coronavirus Protein
KLVNKFLAL

SEQ ID NO: 339 - protein, 9 aa, Artificial Sequence
  Peptide of Coronavirus Protein
ALNLGETFV

SEQ ID NO: 340 - protein, 9 aa, Artificial Sequence
  Peptide of Coronavirus Protein
YLITPVHVM

SEQ ID NO: 341 - protein, 9 aa, Artificial Sequence
  Peptide of Coronavirus Protein
FLPRVFSAV

SEQ ID NO: 342 - protein, 9 aa, Artificial Sequence
  Peptide of Coronavirus Protein
KLIEYTDFA

SEQ ID NO: 343 - protein, 9 aa, Artificial Sequence
  Peptide of Coronavirus Protein
IVAGGIVAI

SEQ ID NO: 344 - protein, 9 aa, Artificial Sequence
  Peptide of Coronavirus Protein
FLAHIQWMV

SEQ ID NO: 345 - protein, 9 aa, Artificial Sequence
  Peptide of Coronavirus Protein
FLLNKEMYL

SEQ ID NO: 346 - protein, 9 aa, Artificial Sequence
  Peptide of Coronavirus Protein
ILTSLLVLV

SEQ ID NO: 347 - protein, 9 aa, Artificial Sequence
  Peptide of Coronavirus Protein
WLDMVDTSL

SEQ ID NO: 348 - protein, 9 aa, Artificial Sequence
  Peptide of Coronavirus Protein
VLLSVLQQL

SEQ ID NO: 349 - protein, 9 aa, Artificial Sequence
  Peptide of Coronavirus Protein
KLWAQCVQL

SEQ ID NO: 350 - protein, 9 aa, Artificial Sequence
  Peptide of Coronavirus Protein
ALWEIQQVV

SEQ ID NO: 351 - protein, 9 aa, Artificial Sequence
  Peptide of Coronavirus Protein
ALLSDLQDL

SEQ ID NO: 352 - protein, 9 aa, Artificial Sequence
  Peptide of Coronavirus Protein
NVFAFPFTI

SEQ ID NO: 353 - protein, 9 aa, Artificial Sequence
  Peptide of Coronavirus Protein
ALSKGVHFV

SEQ ID NO: 354 - protein, 9 aa, Artificial Sequence
  Peptide of Coronavirus Protein
TVYSHLLLV

SEQ ID NO: 355 - protein, 9 aa, Artificial Sequence
  Peptide of Coronavirus Protein
YLYALVYFL

SEQ ID NO: 356 - protein, 9 aa, Artificial Sequence
  Peptide of Coronavirus Protein
LLYDANYFL

SEQ ID NO: 357 - protein, 9 aa, Artificial Sequence
  Peptide of Coronavirus Protein
HLVDFQVTI

SEQ ID NO: 358 - protein, 9 aa, Artificial Sequence
  Peptide of Coronavirus Protein
KLFIRQEEV

SEQ ID NO: 359 - protein, 11 aa, Artificial Sequence
  Peptide of Coronavirus Protein
YLFDESGEFKL

SEQ ID NO: 360 - protein, 11 aa, Artificial Sequence
  Peptide of Coronavirus Protein
YLKLTDNVYIK

SEQ ID NO: 361 - protein, 9 aa, Artificial Sequence
  Peptide of Coronavirus Protein
FLKKDAPYI

SEQ ID NO: 362 - protein, 9 aa, Artificial Sequence
  Peptide of Coronavirus Protein
TLNDLNETL

SEQ ID NO: 363 - protein, 10 aa, Artificial Sequence
  Peptide of Coronavirus Protein
YLDGADVTKI

SEQ ID NO: 364 - protein, 9 aa, Artificial Sequence
  Peptide of Coronavirus Protein
YLDGADVTI

SEQ ID NO: 365 - protein, 8 aa, Artificial Sequence
  Peptide of Coronavirus Protein
YLATALLT

SEQ ID NO: 366 - protein, 9 aa, Artificial Sequence
  Peptide of Coronavirus Protein
YLNSTNVTI

SEQ ID NO: 367 - protein, 11 aa, Artificial Sequence
  Peptide of Coronavirus Protein
QMAPISAMVRM

SEQ ID NO: 368 - protein, 9 aa, Artificial Sequence
  Peptide of Coronavirus Protein
YVWKSYVHV

SEQ ID NO: 369 - protein, 9 aa, Artificial Sequence
  Peptide of Coronavirus Protein
NLIDSYFVV

SEQ ID NO: 370 - protein, 10 aa, Artificial Sequence
  Peptide of Coronavirus Protein
YTMADLVYAL

SEQ ID NO: 371 - protein, 9 aa, Artificial Sequence
  Peptide of Coronavirus Protein
YMADLVYAL

SEQ ID NO: 372 - protein, 9 aa, Artificial Sequence
  Peptide of Coronavirus Protein
SLLMPILTL

SEQ ID NO: 373 - protein, 10 aa, Artificial Sequence
  Peptide of Coronavirus Protein
KIFVDGVPFV

SEQ ID NO: 374 - protein, 9 aa, Artificial Sequence
  Peptide of Coronavirus Protein
KIFDGVPFV

SEQ ID NO: 375 - protein, 9 aa, Artificial Sequence
  Peptide of Coronavirus Protein
RLANECAQV

SEQ ID NO: 376 - protein, 11 aa, Artificial Sequence
  Peptide of Coronavirus Protein
YLPYPDPSRIL

SEQ ID NO: 377 - protein, 9 aa, Artificial Sequence
  Peptide of Coronavirus Protein
LMIERFVSL

SEQ ID NO: 378 - protein, 9 aa, Artificial Sequence
  Peptide of Coronavirus Protein
MLDMYSVML

SEQ ID NO: 379 - protein, 9 aa, Artificial Sequence
  Peptide of Coronavirus Protein
YLQPRTFLL

SEQ ID NO: 380 - protein, 9 aa, Artificial Sequence
  Peptide of Coronavirus Protein
KIADYNYKL

SEQ ID NO: 381 - protein, 9 aa, Artificial Sequence
  Peptide of Coronavirus Protein
  UNSURE (4)..(4): Uncertain of the amino acid at this position, it may be absent.
AEHXVNNSY

SEQ ID NO: 382 - protein, 9 aa, Artificial Sequence
  Peptide of Coronavirus Protein
LLFNKVTLA

SEQ ID NO: 383 - protein, 9 aa, Artificial Sequence
  Peptide of Coronavirus Protein
RLDKVEAEV

SEQ ID NO: 384 - protein, 9 aa, Artificial Sequence
  Peptide of Coronavirus Protein
RLQSLQTYV

SEQ ID NO: 385 - protein, 9 aa, Artificial Sequence
  Peptide of Coronavirus Protein
HLMSFPQSA

SEQ ID NO: 386 - protein, 9 aa, Artificial Sequence
  Peptide of Coronavirus Protein
VSPTKLNDL

SEQ ID NO: 387 - protein, 9 aa, Artificial Sequence
  Peptide of Coronavirus Protein
KVGGNYNYL

SEQ ID NO: 388 - protein, 9 aa, Artificial Sequence
  Peptide of Coronavirus Protein
EGFNCYFPL

SEQ ID NO: 389 - protein, 9 aa, Artificial Sequence
  Peptide of Coronavirus Protein
FKNHTSPDV

SEQ ID NO: 390 - protein, 9 aa, Artificial Sequence
  Peptide of Coronavirus Protein
AMRPNFTIK

SEQ ID NO: 391 - protein, 9 aa, Artificial Sequence
  Peptide of Coronavirus Protein
KLFAAETLK

SEQ ID NO: 392 - protein, 10 aa, Artificial Sequence
  Peptide of Coronavirus Protein
YVFTGYRVTK

SEQ ID NO: 393 - protein, 10 aa, Artificial Sequence
  Peptide of Coronavirus Protein
VVYRGTTTYK

SEQ ID NO: 394 - protein, 9 aa, Artificial Sequence
  Peptide of Coronavirus Protein
ALKYLPIDK

SEQ ID NO: 395 - protein, 9 aa, Artificial Sequence
  Peptide of Coronavirus Protein
VVNARLRAK

SEQ ID NO: 396 - protein, 9 aa, Artificial Sequence
  Peptide of Coronavirus Protein
RIAGHHLGR

SEQ ID NO: 397 - protein, 9 aa, Artificial Sequence
  Peptide of Coronavirus Protein
SLVPGFNEK

SEQ ID NO: 398 - protein, 9 aa, Artificial Sequence
  Peptide of Coronavirus Protein
RLISMMGFK

SEQ ID NO: 399 - protein, 9 aa, Artificial Sequence
  Peptide of Coronavirus Protein
SMMGFKMNY

SEQ ID NO: 400 - protein, 9 aa, Artificial Sequence
  Peptide of Coronavirus Protein
VLHDIGNPK

SEQ ID NO: 401 - protein, 10 aa, Artificial Sequence
  Peptide of Coronavirus Protein
SIINNTVYTK

SEQ ID NO: 402 - protein, 9 aa, Artificial Sequence
  Peptide of Coronavirus Protein
GLQPSVGPK

SEQ ID NO: 403 - protein, 10 aa, Artificial Sequence
  Peptide of Coronavirus Protein
GVAMPNLYKM

SEQ ID NO: 404 - protein, 9 aa, Artificial Sequence
  Peptide of Coronavirus Protein
KMQRMLLEK

SEQ ID NO: 405 - protein, 9 aa, Artificial Sequence
  Peptide of Coronavirus Protein
ALGGSVAIK

SEQ ID NO: 406 - protein, 10 aa, Artificial Sequence
  Peptide of Coronavirus Protein
KTIQPRVEKK

SEQ ID NO: 407 - protein, 9 aa, Artificial Sequence
  Peptide of Coronavirus Protein
KVTKGKAKK

SEQ ID NO: 408 - protein, 9 aa, Artificial Sequence
  Peptide of Coronavirus Protein
NIFGTVYEK

SEQ ID NO: 409 - protein, 9 aa, Artificial Sequence
  Peptide of Coronavirus Protein
IIIGGAKLK

SEQ ID NO: 410 - protein, 9 aa, Artificial Sequence
  Peptide of Coronavirus Protein
VTNNTFTLK

SEQ ID NO: 411 - protein, 9 aa, Artificial Sequence
  Peptide of Coronavirus Protein
TLKGGAPTK

SEQ ID NO: 412 - protein, 9 aa, Artificial Sequence
  Peptide of Coronavirus Protein
TIFKDASGK

SEQ ID NO: 413 - protein, 9 aa, Artificial Sequence
  Peptide of Coronavirus Protein
ALCTFLLNK

SEQ ID NO: 414 - protein, 10 aa, Artificial Sequence
  Peptide of Coronavirus Protein
SMQGAVDINK

SEQ ID NO: 415 - protein, 9 aa, Artificial Sequence
  Peptide of Coronavirus Protein
SMQGAVINK

SEQ ID NO: 416 - protein, 10 aa, Artificial Sequence
  Peptide of Coronavirus Protein
KLKKSLNVAK

SEQ ID NO: 417 - protein, 9 aa, Artificial Sequence
  Peptide of Coronavirus Protein
KLKKSLVAK

SEQ ID NO: 418 - protein, 9 aa, Artificial Sequence
  Peptide of Coronavirus Protein
TMLFTMLRK

SEQ ID NO: 419 - protein, 10 aa, Artificial Sequence
  Peptide of Coronavirus Protein
VVIPDYNTYK

SEQ ID NO: 420 - protein, 9 aa, Artificial Sequence
  Peptide of Coronavirus Protein
ALRANSAVK

SEQ ID NO: 421 - protein, 9 aa, Artificial Sequence
  Peptide of Coronavirus Protein
ALAYYNTTK

SEQ ID NO: 422 - protein, 11 aa, Artificial Sequence
  Peptide of Coronavirus Protein
RIFTIGTVTLK

SEQ ID NO: 423 - protein, 9 aa, Artificial Sequence
  Peptide of Coronavirus Protein
SASKIITLK

SEQ ID NO: 424 - protein, 9 aa, Artificial Sequence
  Peptide of Coronavirus Protein
ASKIITLKK

SEQ ID NO: 425 - protein, 9 aa, Artificial Sequence
  Peptide of Coronavirus Protein
TVIEVQGYK

SEQ ID NO: 426 - protein, 9 aa, Artificial Sequence
  Peptide of Coronavirus Protein
RIDKVLNEK

SEQ ID NO: 427 - protein, 9 aa, Artificial Sequence
  Peptide of Coronavirus Protein
VLSGHNLAK

SEQ ID NO: 428 - protein, 9 aa, Artificial Sequence
  Peptide of Coronavirus Protein
HVVGPNVNK

SEQ ID NO: 429 - protein, 10 aa, Artificial Sequence
  Peptide of Coronavirus Protein
AVFDKNLYDK

SEQ ID NO: 430 - protein, 10 aa, Artificial Sequence
  Peptide of Coronavirus Protein
KLMPVCVETK

SEQ ID NO: 431 - protein, 9 aa, Artificial Sequence
  Peptide of Coronavirus Protein
KLMPVVETK

SEQ ID NO: 432 - protein, 9 aa, Artificial Sequence
  Peptide of Coronavirus Protein
AIVSTIQRK

SEQ ID NO: 433 - protein, 9 aa, Artificial Sequence
  Peptide of Coronavirus Protein
TISLAGSYK

SEQ ID NO: 434 - protein, 10 aa, Artificial Sequence
  Peptide of Coronavirus Protein
AVMYMGTLSY

SEQ ID NO: 435 - protein, 9 aa, Artificial Sequence
  Peptide of Coronavirus Protein
AVYMGTLSY

SEQ ID NO: 436 - protein, 9 aa, Artificial Sequence
  Peptide of Coronavirus Protein
VVENPTIQK

SEQ ID NO: 437 - protein, 10 aa, Artificial Sequence
  Peptide of Coronavirus Protein
KASMPTTIAK

SEQ ID NO: 438 - protein, 9 aa, Artificial Sequence
  Peptide of Coronavirus Protein
ATAEAELAK

SEQ ID NO: 439 - protein, 10 aa, Artificial Sequence
  Peptide of Coronavirus Protein
RQVVNVVTTK

SEQ ID NO: 440 - protein, 9 aa, Artificial Sequence
  Peptide of Coronavirus Protein
VVTTKIALK

SEQ ID NO: 441 - protein, 10 aa, Artificial Sequence
  Peptide of Coronavirus Protein
KVAGFAKFLK

SEQ ID NO: 442 - protein, 10 aa, Artificial Sequence
  Peptide of Coronavirus Protein
LLKDCPAVAK

SEQ ID NO: 443 - protein, 9 aa, Artificial Sequence
  Peptide of Coronavirus Protein
LLKDPAVAK

SEQ ID NO: 444 - protein, 9 aa, Artificial Sequence
  Peptide of Coronavirus Protein
AVAKHDFFK

SEQ ID NO: 445 - protein, 9 aa, Artificial Sequence
  Peptide of Coronavirus Protein
RVYANLGER

SEQ ID NO: 446 - protein, 9 aa, Artificial Sequence
  Peptide of Coronavirus Protein
KLFDRYFKY

SEQ ID NO: 447 - protein, 14 aa, Artificial Sequence
  Peptide of Coronavirus Protein
TVFPPTSFGPLVRK

SEQ ID NO: 448 - protein, 9 aa, Artificial Sequence
  Peptide of Coronavirus Protein
TSFGPLVRK

SEQ ID NO: 449 - protein, 9 aa, Artificial Sequence
  Peptide of Coronavirus Protein
TVKPGNFNK

SEQ ID NO: 450 - protein, 9 aa, Artificial Sequence
  Peptide of Coronavirus Protein
KSAGFPFNK

SEQ ID NO: 451 - protein, 9 aa, Artificial Sequence
  Peptide of Coronavirus Protein
RLYYDSMSY

SEQ ID NO: 452 - protein, 12 aa, Artificial Sequence
  Peptide of Coronavirus Protein
MTNRQFHQKLLK

SEQ ID NO: 453 - protein, 9 aa, Artificial Sequence
  Peptide of Coronavirus Protein
ATVVIGTSK

SEQ ID NO: 454 - protein, 9 aa, Artificial Sequence
  Peptide of Coronavirus Protein
LVASIKNFK

SEQ ID NO: 455 - protein, 9 aa, Artificial Sequence
  Peptide of Coronavirus Protein
AIDAYPLTK

SEQ ID NO: 456 - protein, 9 aa, Artificial Sequence
  Peptide of Coronavirus Protein
KVFRSSVLH

SEQ ID NO: 457 - protein, 9 aa, Artificial Sequence
  Peptide of Coronavirus Protein
GVYFASTEK

SEQ ID NO: 458 - protein, 9 aa, Artificial Sequence
  Peptide of Coronavirus Protein
GVYYHKNNK

SEQ ID NO: 459 - protein, 9 aa, Artificial Sequence
  Peptide of Coronavirus Protein
TLKSFTVEK

SEQ ID NO: 460 - protein, 10 aa, Artificial Sequence
  Peptide of Coronavirus Protein
RQIAPGQTGK

SEQ ID NO: 461 - protein, 9 aa, Artificial Sequence
  Peptide of Coronavirus Protein
RIAPGQTGK

SEQ ID NO: 462 - protein, 9 aa, Artificial Sequence
  Peptide of Coronavirus Protein
RLFRKSNLK

SEQ ID NO: 463 - protein, 10 aa, Artificial Sequence
  Peptide of Coronavirus Protein
KQIYKTPPIK

SEQ ID NO: 464 - protein, 9 aa, Artificial Sequence
  Peptide of Coronavirus Protein
KIYKTPPIK

SEQ ID NO: 465 - protein, 9 aa, Artificial Sequence
  Peptide of Coronavirus Protein
TLADAGFIK

SEQ ID NO: 466 - protein, 9 aa, Artificial Sequence
  Peptide of Coronavirus Protein
ASANLAATK

SEQ ID NO: 467 - protein, 9 aa, Artificial Sequence
  Peptide of Coronavirus Protein
VTYVPAQEK

SEQ ID NO: 468 - protein, 10 aa, Artificial Sequence
  Peptide of Coronavirus Protein
AVLDMCASLK

SEQ ID NO: 469 - protein, 9 aa, Artificial Sequence
  Peptide of Coronavirus Protein
AVLDMASLK

SEQ ID NO: 470 - protein, 9 aa, Artificial Sequence
  Peptide of Coronavirus Protein
VTFQSAVKR

SEQ ID NO: 471 - protein, 9 aa, Artificial Sequence
  Peptide of Coronavirus Protein
SSNVANYQK

SEQ ID NO: 472 - protein, 11 aa, Artificial Sequence
  Peptide of Coronavirus Protein
STLQGPPGTGK

SEQ ID NO: 473 - protein, 9 aa, Artificial Sequence
  Peptide of Coronavirus Protein
GTITVEELK

SEQ ID NO: 474 - protein, 10 aa, Artificial Sequence
  Peptide of Coronavirus Protein
ATSRTLSYYK

SEQ ID NO: 475 - protein, 9 aa, Artificial Sequence
  Peptide of Coronavirus Protein
ASCDAIMTR

SEQ ID NO: 476 - protein, 9 aa, Artificial Sequence
  Peptide of Coronavirus Protein
KTQFNYYKK

SEQ ID NO: 477 - protein, 9 aa, Artificial Sequence
  Peptide of Coronavirus Protein
HSWNADLYK

SEQ ID NO: 478 - protein, 9 aa, Artificial Sequence
  Peptide of Coronavirus Protein
SSYSLFDMS

SEQ ID NO: 479 - protein, 10 aa, Artificial Sequence
  Peptide of Coronavirus Protein
STSAFVETVK

SEQ ID NO: 480 - protein, 9 aa, Artificial Sequence
  Peptide of Coronavirus Protein
STSAFVTVK

SEQ ID NO: 481 - protein, 9 aa, Artificial Sequence
  Peptide of Coronavirus Protein
RVVRSIFSR

SEQ ID NO: 482 - protein, 9 aa, Artificial Sequence
  Peptide of Coronavirus Protein
GAMDTTSYR

SEQ ID NO: 483 - protein, 10 aa, Artificial Sequence
  Peptide of Coronavirus Protein
SAVLQSGFRK

SEQ ID NO: 484 - protein, 9 aa, Artificial Sequence
  Peptide of Coronavirus Protein
SAFAMMFVK

SEQ ID NO: 485 - protein, 12 aa, Artificial Sequence
  Peptide of Coronavirus Protein
AVANGDSEVVLK

SEQ ID NO: 486 - protein, 9 aa, Artificial Sequence
  Peptide of Coronavirus Protein
AVANGVVLK

SEQ ID NO: 487 - protein, 9 aa, Artificial Sequence
  Peptide of Coronavirus Protein
QTMLFTMLR

SEQ ID NO: 488 - protein, 9 aa, Artificial Sequence
  Peptide of Coronavirus Protein
ASAFFGMSR

SEQ ID NO: 489 - protein, 11 aa, Artificial Sequence
  Peptide of Coronavirus Protein
AYKTFPPTEPK

SEQ ID NO: 490 - protein, 10 aa, Artificial Sequence
  Peptide of Coronavirus Protein
VVNAANVYLK

SEQ ID NO: 491 - protein, 9 aa, Artificial Sequence
  Peptide of Coronavirus Protein
VVNAANVYK

SEQ ID NO: 492 - protein, 11 aa, Artificial Sequence
  Peptide of Coronavirus Protein
LVSDIDITFLK

SEQ ID NO: 493 - protein, 9 aa, Artificial Sequence
  Peptide of Coronavirus Protein
LTAVVIPTK

SEQ ID NO: 494 - protein, 10 aa, Artificial Sequence
  Peptide of Coronavirus Protein
STQLGIEFLK

SEQ ID NO: 495 - protein, 9 aa, Artificial Sequence
  Peptide of Coronavirus Protein
STLGIEFLK

SEQ ID NO: 496 - protein, 9 aa, Artificial Sequence
  Peptide of Coronavirus Protein
GTLSYEQFK

SEQ ID NO: 497 - protein, 12 aa, Artificial Sequence
  Peptide of Coronavirus Protein
SYTTTIKPVTYK

SEQ ID NO: 498 - protein, 9 aa, Artificial Sequence
  Peptide of Coronavirus Protein
TSNSFDVLK

SEQ ID NO: 499 - protein, 9 aa, Artificial Sequence
  Peptide of Coronavirus Protein
TTIAKNTVK

SEQ ID NO: 500 - protein, 13 aa, Artificial Sequence
  Peptide of Coronavirus Protein
ASFNYLKSPNFSK

SEQ ID NO: 501 - protein, 12 aa, Artificial Sequence
  Peptide of Coronavirus Protein
SSTFNVPMEKLK

SEQ ID NO: 502 - protein, 9 aa, Artificial Sequence
  Peptide of Coronavirus Protein
GTSTDVVYR

SEQ ID NO: 503 - protein, 9 aa, Artificial Sequence
  Peptide of Coronavirus Protein
RAFDIYNDK

SEQ ID NO: 504 - protein, 9 aa, Artificial Sequence
  Peptide of Coronavirus Protein
NSASFSTFK

SEQ ID NO: 505 - protein, 10 aa, Artificial Sequence
  Peptide of Coronavirus Protein
TEILPVSMTK

SEQ ID NO: 506 - protein, 9 aa, Artificial Sequence
  Peptide of Coronavirus Protein
TILPVSMTK

SEQ ID NO: 507 - protein, 9 aa, Artificial Sequence
  Peptide of Coronavirus Protein
SSTASALGK

SEQ ID NO: 508 - protein, 9 aa, Artificial Sequence
  Peptide of Coronavirus Protein
GTHWFVTQR

SEQ ID NO: 509 - protein, 9 aa, Artificial Sequence
  Peptide of Coronavirus Protein
AEVQIDRLI

SEQ ID NO: 510 - protein, 14 aa, Artificial Sequence
  Peptide of Coronavirus Protein
TVYDPLQPELDSFK

SEQ ID NO: 511 - protein, 12 aa, Artificial Sequence
  Peptide of Coronavirus Protein
KYKFVRIQPGQT

SEQ ID NO: 512 - protein, 13 aa, Artificial Sequence
  Peptide of Coronavirus Protein
SYYCKSHKPPISF

SEQ ID NO: 513 - protein, 11 aa, Artificial Sequence
  Peptide of Coronavirus Protein
TYKLNVGDYFV

SEQ ID NO: 514 - protein, 9 aa, Artificial Sequence
  Peptide of Coronavirus Protein
VYIGDPAQL

SEQ ID NO: 515 - protein, 10 aa, Artificial Sequence
  Peptide of Coronavirus Protein
SYFIASFRLF

SEQ ID NO: 516 - protein, 9 aa, Artificial Sequence
  Peptide of Coronavirus Protein
SYGADLKSF

SEQ ID NO: 517 - protein, 9 aa, Artificial Sequence
  Peptide of Coronavirus Protein
VFVLWAHGF

SEQ ID NO: 518 - protein, 9 aa, Artificial Sequence
  Peptide of Coronavirus Protein
SYATHSDKF

SEQ ID NO: 519 - protein, 9 aa, Artificial Sequence
  Peptide of Coronavirus Protein
KFTDGVCLF

SEQ ID NO: 520 - protein, 9 aa, Artificial Sequence
  Peptide of Coronavirus Protein
LYLDAYNMM

SEQ ID NO: 521 - protein, 11 aa, Artificial Sequence
  Peptide of Coronavirus Protein
VYKQFDTYNLW

SEQ ID NO: 522 - protein, 14 aa, Artificial Sequence
  Peptide of Coronavirus Protein
VFFDGRVDGQVDLF

SEQ ID NO: 523 - protein, 9 aa, Artificial Sequence
  Peptide of Coronavirus Protein
VFFDGRVLF

SEQ ID NO: 524 - protein, 9 aa, Artificial Sequence
  Peptide of Coronavirus Protein
RYKLEGYAF

SEQ ID NO: 525 - protein, 9 aa, Artificial Sequence
  Peptide of Coronavirus Protein
TFFKLVNKF

SEQ ID NO: 526 - protein, 9 aa, Artificial Sequence
  Peptide of Coronavirus Protein
VFLFVAAIF

SEQ ID NO: 527 - protein, 13 aa, Artificial Sequence
  Peptide of Coronavirus Protein
TYLEGSVRVVTTF

SEQ ID NO: 528 - protein, 9 aa, Artificial Sequence
  Peptide of Coronavirus Protein
YYRSLPGVF

SEQ ID NO: 529 - protein, 10 aa, Artificial Sequence
  Peptide of Coronavirus Protein
GVYSVIYLYL

SEQ ID NO: 530 - protein, 11 aa, Artificial Sequence
  Peptide of Coronavirus Protein
TFYLTNDVSFL

SEQ ID NO: 531 - protein, 9 aa, Artificial Sequence
  Peptide of Coronavirus Protein
MFTPLVPFW

SEQ ID NO: 532 - protein, 10 aa, Artificial Sequence
  Peptide of Coronavirus Protein
VFNGVSFSTF

SEQ ID NO: 533 - protein, 9 aa, Artificial Sequence
  Peptide of Coronavirus Protein
LYQPPQTSI

SEQ ID NO: 534 - protein, 14 aa, Artificial Sequence
  Peptide of Coronavirus Protein
VYMPASWVMRIMTW

SEQ ID NO: 535 - protein, 9 aa, Artificial Sequence
  Peptide of Coronavirus Protein
MYASAVVLL

SEQ ID NO: 536 - protein, 9 aa, Artificial Sequence
  Peptide of Coronavirus Protein
MFLARGIVF

SEQ ID NO: 537 - protein, 9 aa, Artificial Sequence
  Peptide of Coronavirus Protein
IFFITGNTL

SEQ ID NO: 538 - protein, 10 aa, Artificial Sequence
  Peptide of Coronavirus Protein
YDYLVSTQEF

SEQ ID NO: 539 - protein, 9 aa, Artificial Sequence
  Peptide of Coronavirus Protein
TYASALWEI

SEQ ID NO: 540 - protein, 9 aa, Artificial Sequence
  Peptide of Coronavirus Protein
YFIKGLNNL

SEQ ID NO: 541 - protein, 10 aa, Artificial Sequence
  Peptide of Coronavirus Protein
GYINVFAFPF

SEQ ID NO: 542 - protein, 9 aa, Artificial Sequence
  Peptide of Coronavirus Protein
LYLYALVYF

SEQ ID NO: 543 - protein, 9 aa, Artificial Sequence
  Peptide of Coronavirus Protein
VYFLQSINF

SEQ ID NO: 544 - protein, 9 aa, Artificial Sequence
  Peptide of Coronavirus Protein
YYQLYSTQL

SEQ ID NO: 545 - protein, 10 aa, Artificial Sequence
  Peptide of Coronavirus Protein
QYIDIGNYTV

SEQ ID NO: 546 - protein, 10 aa, Artificial Sequence
  Peptide of Coronavirus Protein
QGYKSVNITF

SEQ ID NO: 547 - protein, 9 aa, Artificial Sequence
  Peptide of Coronavirus Protein
DYQGKPLEF

SEQ ID NO: 548 - protein, 9 aa, Artificial Sequence
  Peptide of Coronavirus Protein
LYDKLVSSF

SEQ ID NO: 549 - protein, 9 aa, Artificial Sequence
  Peptide of Coronavirus Protein
LYIDINGNL

SEQ ID NO: 550 - protein, 10 aa, Artificial Sequence
  Peptide of Coronavirus Protein
VYYTSNPTTF

SEQ ID NO: 551 - protein, 10 aa, Artificial Sequence
  Peptide of Coronavirus Protein
EYYHTTDPSF

SEQ ID NO: 552 - protein, 9 aa, Artificial Sequence
  Peptide of Coronavirus Protein
SYLFQHANL

SEQ ID NO: 553 - protein, 10 aa, Artificial Sequence
  Peptide of Coronavirus Protein
NYYKKDNSYF

SEQ ID NO: 554 - protein, 9 aa, Artificial Sequence
  Peptide of Coronavirus Protein
DYKHYTPSF

SEQ ID NO: 555 - protein, 9 aa, Artificial Sequence
  Peptide of Coronavirus Protein
NYMPYFFTL

SEQ ID NO: 556 - protein, 11 aa, Artificial Sequence
  Peptide of Coronavirus Protein
AYILFTRFFYV

SEQ ID NO: 557 - protein, 9 aa, Artificial Sequence
  Peptide of Coronavirus Protein
FFASFYYVW

SEQ ID NO: 558 - protein, 9 aa, Artificial Sequence
  Peptide of Coronavirus Protein
VYANGGKGF

SEQ ID NO: 559 - protein, 10 aa, Artificial Sequence
  Peptide of Coronavirus Protein
KMFDAYVNTF

SEQ ID NO: 560 - protein, 10 aa, Artificial Sequence
  Peptide of Coronavirus Protein
AYVNTFSSTF

SEQ ID NO: 561 - protein, 12 aa, Artificial Sequence
  Peptide of Coronavirus Protein
IYNDKVAGFAKF

SEQ ID NO: 562 - protein, 10 aa, Artificial Sequence
  Peptide of Coronavirus Protein
SYFVVKRHTF

SEQ ID NO: 563 - protein, 9 aa, Artificial Sequence
  Peptide of Coronavirus Protein
NFNKDFYDF

SEQ ID NO: 564 - protein, 9 aa, Artificial Sequence
  Peptide of Coronavirus Protein
AYANSVFNI

SEQ ID NO: 565 - protein, 10 aa, Artificial Sequence
  Peptide of Coronavirus Protein
HLYLQYIRKL

SEQ ID NO: 566 - protein, 9 aa, Artificial Sequence
  Peptide of Coronavirus Protein
PFFSNVTWF

SEQ ID NO: 567 - protein, 9 aa, Artificial Sequence
  Peptide of Coronavirus Protein
RFDNPVLPF

SEQ ID NO: 568 - protein, 10 aa, Artificial Sequence
  Peptide of Coronavirus Protein
VYSSANNCTF

SEQ ID NO: 569 - protein, 9 aa, Artificial Sequence
  Peptide of Coronavirus Protein
EYVSQPFLM

SEQ ID NO: 570 - protein, 11 aa, Artificial Sequence
  Peptide of Coronavirus Protein
YYVGYLQPRTF

SEQ ID NO: 571 - protein, 10 aa, Artificial Sequence
  Peptide of Coronavirus Protein
LYNSASFSTF

SEQ ID NO: 572 - protein, 9 aa, Artificial Sequence
  Peptide of Coronavirus Protein
NYNYLYRLF

SEQ ID NO: 573 - protein, 9 aa, Artificial Sequence
  Peptide of Coronavirus Protein
YFPLQSYGF

SEQ ID NO: 574 - protein, 9 aa, Artificial Sequence
  Peptide of Coronavirus Protein
PYRVVVLSF

SEQ ID NO: 575 - protein, 9 aa, Artificial Sequence
  Peptide of Coronavirus Protein
VYSTGSNVF

SEQ ID NO: 576 - protein, 10 aa, Artificial Sequence
  Peptide of Coronavirus Protein
IYKTPPIKDF

SEQ ID NO: 577 - protein, 10 aa, Artificial Sequence
  Peptide of Coronavirus Protein
TYVPAQEKNF

SEQ ID NO: 578 - protein, 9 aa, Artificial Sequence
  Peptide of Coronavirus Protein
QYIKWPWYI

SEQ ID NO: 579 - protein, 9 aa, Artificial Sequence
  Peptide of Coronavirus Protein
DYVYNPFMI

SEQ ID NO: 580 - protein, 8 aa, Artificial Sequence
  Peptide of Coronavirus Protein
TYNLWNTF

SEQ ID NO: 581 - protein, 9 aa, Artificial Sequence
  Peptide of Coronavirus Protein
  UNSURE (3)..(3): Uncertain of the amino acid at this position it may be absent.
TYXNLWNTF

SEQ ID NO: 582 - protein, 9 aa, Artificial Sequence
  Peptide of Coronavirus Protein
NYLKRRVVF

SEQ ID NO: 583 - protein, 11 aa, Artificial Sequence
  Peptide of Coronavirus Protein
NYSGVVTTVMF

SEQ ID NO: 584 - protein, 9 aa, Artificial Sequence
  Peptide of Coronavirus Protein
NYSGVVTTF

SEQ ID NO: 585 - protein, 9 aa, Artificial Sequence
  Peptide of Coronavirus Protein
AFPFTIYSL

SEQ ID NO: 586 - protein, 9 aa, Artificial Sequence
  Peptide of Coronavirus Protein
PYNSVTSSI

SEQ ID NO: 587 - protein, 12 aa, Artificial Sequence
  Peptide of Coronavirus Protein
YYRARAGEAANF

SEQ ID NO: 588 - protein, 9 aa, Artificial Sequence
  Peptide of Coronavirus Protein
YYAGEAANF

SEQ ID NO: 589 - protein, 11 aa, Artificial Sequence
  Peptide of Coronavirus Protein
MYMGTLSYEQF

SEQ ID NO: 590 - protein, 8 aa, Artificial Sequence
  Peptide of Coronavirus Protein
MYMGSYEQ

SEQ ID NO: 591 - protein, 12 aa, Artificial Sequence
  Peptide of Coronavirus Protein
PYPNASFDNFKF

SEQ ID NO: 592 - protein, 9 aa, Artificial Sequence
  Peptide of Coronavirus Protein
TYKPNTWCI

SEQ ID NO: 593 - protein, 9 aa, Artificial Sequence
  Peptide of Coronavirus Protein
FYGGWHNML

SEQ ID NO: 594 - protein, 9 aa, Artificial Sequence
  Peptide of Coronavirus Protein
YYHKNNKSW

SEQ ID NO: 595 - protein, 11 aa, Artificial Sequence
  Peptide of Coronavirus Protein
RFPNITNLCPF

SEQ ID NO: 596 - protein, 9 aa, Artificial Sequence
  Peptide of Coronavirus Protein
RFPNITNLF

SEQ ID NO: 597 - protein, 13 aa, Artificial Sequence
  Peptide of Coronavirus Protein
AYSNNSIAIPTNF

SEQ ID NO: 598 - protein, 9 aa, Artificial Sequence
  Peptide of Coronavirus Protein
AYIAIPTNF

SEQ ID NO: 599 - protein, 9 aa, Artificial Sequence
  Peptide of Coronavirus Protein
QPGQTFSVL

SEQ ID NO: 600 - protein, 10 aa, Artificial Sequence
  Peptide of Coronavirus Protein
SPSGVYQCAM

SEQ ID NO: 601 - protein, 9 aa, Artificial Sequence
  Peptide of Coronavirus Protein
SPSGVYQAM

SEQ ID NO: 602 - protein, 10 aa, Artificial Sequence
  Peptide of Coronavirus Protein
RPNFTIKGSF

SEQ ID NO: 603 - protein, 9 aa, Artificial Sequence
  Peptide of Coronavirus Protein
RPNTIKGSF

SEQ ID NO: 604 - protein, 9 aa, Artificial Sequence
  Peptide of Coronavirus Protein
TPHTVLQAV

SEQ ID NO: 605 - protein, 12 aa, Artificial Sequence
  Peptide of Coronavirus Protein
KPRPPLNRNYVF

SEQ ID NO: 606 - protein, 9 aa, Artificial Sequence
  Peptide of Coronavirus Protein
KPRPPLNVF

SEQ ID NO: 607 - protein, 8 aa, Artificial Sequence
  Peptide of Coronavirus Protein
MPLSAPTL

SEQ ID NO: 608 - protein, 9 aa, Artificial Sequence
  Peptide of Coronavirus Protein
  UNSURE (7)..(7): Uncertain of the amino acid at this position it may be absent.
MPLSAPXTL

SEQ ID NO: 609 - protein, 10 aa, Artificial Sequence
  Peptide of Coronavirus Protein
IPARARVECF

SEQ ID NO: 610 - protein, 9 aa, Artificial Sequence
  Peptide of Coronavirus Protein
IPARARVEF

SEQ ID NO: 611 - protein, 11 aa, Artificial Sequence
  Peptide of Coronavirus Protein
APRTLLTKGTL

SEQ ID NO: 612 - protein, 10 aa, Artificial Sequence
  Peptide of Coronavirus Protein
RPQIGVVREF

SEQ ID NO: 613 - protein, 9 aa, Artificial Sequence
  Peptide of Coronavirus Protein
RPQIGVVEF

SEQ ID NO: 614 - protein, 10 aa, Artificial Sequence
  Peptide of Coronavirus Protein
RNPAWRKAVF

SEQ ID NO: 615 - protein, 14 aa, Artificial Sequence
  Peptide of Coronavirus Protein
SPYNSQNAVASKIL

SEQ ID NO: 616 - protein, 9 aa, Artificial Sequence
  Peptide of Coronavirus Protein
SPYNSQNAL

SEQ ID NO: 617 - protein, 9 aa, Artificial Sequence
  Peptide of Coronavirus Protein
IPRRNVATL

SEQ ID NO: 618 - protein, 8 aa, Artificial Sequence
  Peptide of Coronavirus Protein
VPLHGTIL

SEQ ID NO: 619 - protein, 9 aa, Artificial Sequence
  Peptide of Coronavirus Protein
  Unsure (4)..(4): Uncertain of the amino acid at this position it may be absent.
VPLXHGTIL

SEQ ID NO: 620 - protein, 9 aa, Artificial Sequence
  Peptide of Coronavirus Protein
RTAPHGHVM

SEQ ID NO: 621 - protein, 10 aa, Artificial Sequence
  Peptide of Coronavirus Protein
APHGHVMVEL

SEQ ID NO: 622 - protein, 9 aa, Artificial Sequence
  Peptide of Coronavirus Protein
APHGHVMVL

SEQ ID NO: 623 - protein, 10 aa, Artificial Sequence
  Peptide of Coronavirus Protein
VPANSTVLSF

SEQ ID NO: 624 - protein, 9 aa, Artificial Sequence
  Peptide of Coronavirus Protein
VPANSTVSF

SEQ ID NO: 625 - protein, 10 aa, Artificial Sequence
  Peptide of Coronavirus Protein
HPNPKGFCDL

SEQ ID NO: 626 - protein, 9 aa, Artificial Sequence
  Peptide of Coronavirus Protein
HPNPKGFDL

SEQ ID NO: 627 - protein, 9 aa, Artificial Sequence
  Peptide of Coronavirus Protein
HPTQAPTHL

SEQ ID NO: 628 - protein, 9 aa, Artificial Sequence
  Peptide of Coronavirus Protein
TPAFDKSAF

SEQ ID NO: 629 - protein, 9 aa, Artificial Sequence
  Peptide of Coronavirus Protein
LPVNVAFEL

SEQ ID NO: 630 - protein, 9 aa, Artificial Sequence
  Peptide of Coronavirus Protein
KPVPEVKIL

SEQ ID NO: 631 - protein, 8 aa, Artificial Sequence
  Peptide of Coronavirus Protein
APAHISTI

SEQ ID NO: 632 - protein, 9 aa, Artificial Sequence
  Peptide of Coronavirus Protein
  Unsure (4)..(4): Uncertain of the amino acid at this position it may be absent.
APAXHISTI

SEQ ID NO: 633 - protein, 12 aa, Artificial Sequence
  Peptide of Coronavirus Protein
GPKQASLNGVTL

SEQ ID NO: 634 - protein, 9 aa, Artificial Sequence
  Peptide of Coronavirus Protein
GPKQAGVTL

SEQ ID NO: 635 - protein, 8 aa, Artificial Sequence
  Peptide of Coronavirus Protein
KPRSQMEI

SEQ ID NO: 636 - protein, 9 aa, Artificial Sequence
  Peptide of Coronavirus Protein
  unsure (4)..(4): Uncertain of the amino acid at this position it may be absent.
KPRXSQMEI

SEQ ID NO: 637 - protein, 9 aa, Artificial Sequence
  Peptide of Coronavirus Protein
LPKGIMMNV

SEQ ID NO: 638 - protein, 10 aa, Artificial Sequence
  Peptide of Coronavirus Protein
VPYNMRVIHF

SEQ ID NO: 639 - protein, 11 aa, Artificial Sequence
  Peptide of Coronavirus Protein
KPREQIDGYVM

SEQ ID NO: 640 - protein, 9 aa, Artificial Sequence
  Peptide of Coronavirus Protein
QPRVEKKKL

SEQ ID NO: 641 - protein, 9 aa, Artificial Sequence
  Peptide of Coronavirus Protein
MPLKAPKEI

SEQ ID NO: 642 - protein, 8 aa, Artificial Sequence
  Peptide of Coronavirus Protein
APKEIIFL

SEQ ID NO: 643 - protein, 9 aa, Artificial Sequence
  Peptide of Coronavirus Protein
  Unsure (8)..(8): Uncertain of the amino acid at this position it may be absent.
APKEIIFXL

SEQ ID NO: 644 - protein, 10 aa, Artificial Sequence
  Peptide of Coronavirus Protein
VVPGLPGTIL

SEQ ID NO: 645 - protein, 9 aa, Artificial Sequence
  Peptide of Coronavirus Protein
VPGLPGTIL

SEQ ID NO: 646 - protein, 10 aa, Artificial Sequence
  Peptide of Coronavirus Protein
VPYCYDTNVL

SEQ ID NO: 647 - protein, 9 aa, Artificial Sequence
  Peptide of Coronavirus Protein
VPYYDTNVL

SEQ ID NO: 648 - protein, 9 aa, Artificial Sequence
  Peptide of Coronavirus Protein
RPDTRYVLM

SEQ ID NO: 649 - protein, 10 aa, Artificial Sequence
  Peptide of Coronavirus Protein
TPLIQPIGAL

SEQ ID NO: 650 - protein, 9 aa, Artificial Sequence
  Peptide of Coronavirus Protein
TPIQPIGAL

SEQ ID NO: 651 - protein, 10 aa, Artificial Sequence
  Peptide of Coronavirus Protein
LPFAMGIIAM

SEQ ID NO: 652 - protein, 9 aa, Artificial Sequence
  Peptide of Coronavirus Protein
IPLTTAAKL

SEQ ID NO: 653 - protein, 9 aa, Artificial Sequence
  Peptide of Coronavirus Protein
SPNLAWPLI

SEQ ID NO: 654 - protein, 8 aa, Artificial Sequence
  Peptide of Coronavirus Protein
SPVALRQM

SEQ ID NO: 655 - protein, 9 aa, Artificial Sequence
  Peptide of Coronavirus Protein
  unsure (7)..(7): Uncertain of the amino acid at this position it may be absent.
SPVALRXQM

SEQ ID NO: 656 - protein, 10 aa, Artificial Sequence
  Peptide of Coronavirus Protein
TPKGPKVKYL

SEQ ID NO: 657 - protein, 10 aa, Artificial Sequence
  Peptide of Coronavirus Protein
KFPRGQGVPI

SEQ ID NO: 658 - protein, 9 aa, Artificial Sequence
  Peptide of Coronavirus Protein
KPRGQGVPI

SEQ ID NO: 659 - protein, 9 aa, Artificial Sequence
  Peptide of Coronavirus Protein
SPRWYFYYL

SEQ ID NO: 660 - protein, 9 aa, Artificial Sequence
  Peptide of Coronavirus Protein
IPIQASLPF

SEQ ID NO: 661 - protein, 9 aa, Artificial Sequence
  Peptide of Coronavirus Protein
APFLYLYAL

SEQ ID NO: 662 - protein, 13 aa, Artificial Sequence
  Peptide of Coronavirus Protein
SPFHPLADNKFAL

SEQ ID NO: 663 - protein, 9 aa, Artificial Sequence
  Peptide of Coronavirus Protein
SPFHPLAAL

SEQ ID NO: 664 - protein, 9 aa, Artificial Sequence
  Peptide of Coronavirus Protein
RARSVSPKL

SEQ ID NO: 665 - protein, 11 aa, Artificial Sequence
  Peptide of Coronavirus Protein
KPLEFGATSAA

SEQ ID NO: 666 - protein, 9 aa, Artificial Sequence
  Peptide of Coronavirus Protein
APLLSAGIF

SEQ ID NO: 667 - protein, 9 aa, Artificial Sequence
  Peptide of Coronavirus Protein
APYIVGDVV

SEQ ID NO: 668 - protein, 10 aa, Artificial Sequence
  Peptide of Coronavirus Protein
YPQVNGLTSI

SEQ ID NO: 669 - protein, 9 aa, Artificial Sequence
  Peptide of Coronavirus Protein
YPQNGLTSI

SEQ ID NO: 670 - protein, 11 aa, Artificial Sequence
  Peptide of Coronavirus Protein
KPASRELKVTF

SEQ ID NO: 671 - protein, 9 aa, Artificial Sequence
  Peptide of Coronavirus Protein
KPRELKVTF

SEQ ID NO: 672 - protein, 10 aa, Artificial Sequence
  Peptide of Coronavirus Protein
TPSFKKGAKL

SEQ ID NO: 673 - protein, 9 aa, Artificial Sequence
  Peptide of Coronavirus Protein
TPFKKGAKL

SEQ ID NO: 674 - protein, 9 aa, Artificial Sequence
  Peptide of Coronavirus Protein
KPANNSLKI

SEQ ID NO: 675 - protein, 10 aa, Artificial Sequence
  Peptide of Coronavirus Protein
KKPNELSRVL

SEQ ID NO: 676 - protein, 9 aa, Artificial Sequence
  Peptide of Coronavirus Protein
KPNELSRVL

SEQ ID NO: 677 - protein, 9 aa, Artificial Sequence
  Peptide of Coronavirus Protein
MPYFFTLLL

SEQ ID NO: 678 - protein, 9 aa, Artificial Sequence
  Peptide of Coronavirus Protein
TPRDLGACI

SEQ ID NO: 679 - protein, 9 aa, Artificial Sequence
  Peptide of Coronavirus Protein
VAKSHNIAL

SEQ ID NO: 680 - protein, 10 aa, Artificial Sequence
  Peptide of Coronavirus Protein
MVPHISRQRL

SEQ ID NO: 681 - protein, 10 aa, Artificial Sequence
  Peptide of Coronavirus Protein
MPILTLTRAL

SEQ ID NO: 682 - protein, 9 aa, Artificial Sequence
  Peptide of Coronavirus Protein
MPILTTRAL

SEQ ID NO: 683 - protein, 9 aa, Artificial Sequence
  Peptide of Coronavirus Protein
KPYIKWDLL

SEQ ID NO: 684 - protein, 9 aa, Artificial Sequence
  Peptide of Coronavirus Protein
IPTITQMNL

SEQ ID NO: 685 - protein, 9 aa, Artificial Sequence
  Peptide of Coronavirus Protein
LPFNDGVYF

SEQ ID NO: 686 - protein, 9 aa, Artificial Sequence
  Peptide of Coronavirus Protein
TPINLVRDL

SEQ ID NO: 687 - protein, 8 aa, Artificial Sequence
  Peptide of Coronavirus Protein
LPQGFSAL

SEQ ID NO: 688 - protein, 9 aa, Artificial Sequence
  Peptide of Coronavirus Protein
  Unsure (4)..(4): Uncertain of the amino acid at this position it may be absent.
LPQXGFSAL

SEQ ID NO: 689 - protein, 11 aa, Artificial Sequence
  Peptide of Coronavirus Protein
KPFERDISTEI

SEQ ID NO: 690 - protein, 8 aa, Artificial Sequence
  Peptide of Coronavirus Protein
QPYRVVVL

SEQ ID NO: 691 - protein, 9 aa, Artificial Sequence
  Peptide of Coronavirus Protein
GPKKSTNLV

SEQ ID NO: 692 - protein, 9 aa, Artificial Sequence
  Peptide of Coronavirus Protein
SPRRARSVA

SEQ ID NO: 693 - protein, 9 aa, Artificial Sequence
  Peptide of Coronavirus Protein
IPTNFTISV

SEQ ID NO: 694 - protein, 8 aa, Artificial Sequence
  Peptide of Coronavirus Protein
APHGVVFL

SEQ ID NO: 695 - protein, 9 aa, Artificial Sequence
  Peptide of Coronavirus Protein
  Unsure (4)..(4): Uncertain of the amnio acid at this position it may be absent.
APHXGVVFL

SEQ ID NO: 696 - protein, 10 aa, Artificial Sequence
  Peptide of Coronavirus Protein
SEPVLKGVKL

SEQ ID NO: 697 - protein, 9 aa, Artificial Sequence
  Peptide of Coronavirus Protein
QLRVIGHSM

SEQ ID NO: 698 - protein, 9 aa, Artificial Sequence
  Peptide of Coronavirus Protein
TPKYKFVRI

SEQ ID NO: 699 - protein, 9 aa, Artificial Sequence
  Peptide of Coronavirus Protein
YVYSRVKNL

SEQ ID NO: 700 - protein, 8 aa, Artificial Sequence
  Peptide of Coronavirus Protein
VGKPRPPL

SEQ ID NO: 701 - protein, 9 aa, Artificial Sequence
  Peptide of Coronavirus Protein
  Unsure (7)..(7): Uncertain of the amino acid at this position, it may be absent
VGKPRPXPL

SEQ ID NO: 702 - protein, 8 aa, Artificial Sequence
  Peptide of Coronavirus Protein
VGMQKYST

SEQ ID NO: 703 - protein, 9 aa, Artificial Sequence
  Peptide of Coronavirus Protein
YDKLQFTSL

SEQ ID NO: 704 - protein, 9 aa, Artificial Sequence
  Peptide of Coronavirus Protein
RLFARTRSM

SEQ ID NO: 705 - protein, 9 aa, Artificial Sequence
  Peptide of Coronavirus Protein
DLKGKYVQI

SEQ ID NO: 706 - protein, 9 aa, Artificial Sequence
  Peptide of Coronavirus Protein
MTYRRLISM

SEQ ID NO: 707 - protein, 9 aa, Artificial Sequence
  Peptide of Coronavirus Protein
VVRIKIVQM

SEQ ID NO: 708 - protein, 8 aa, Artificial Sequence
  Peptide of Coronavirus Protein
HMVVKAAL

SEQ ID NO: 709 - protein, 9 aa, Artificial Sequence
  Peptide of Coronavirus Protein
  Unsure (7)..(7): Uncertain of  the amino acid at this position, it may be absent.
HMVVKAXAL

SEQ ID NO: 710 - protein, 9 aa, Artificial Sequence
  Peptide of Coronavirus Protein
SLYVNKHAF

SEQ ID NO: 711 - protein, 9 aa, Artificial Sequence
  Peptide of Coronavirus Protein
HANEYRLYL

SEQ ID NO: 712 - protein, 9 aa, Artificial Sequence
  Peptide of Coronavirus Protein
NTFTRLQSL

SEQ ID NO: 713 - protein, 9 aa, Artificial Sequence
  Peptide of Coronavirus Protein
SVGPKQASL

SEQ ID NO: 714 - protein, 8 aa, Artificial Sequence
  Peptide of Coronavirus Protein
NVAKYTQL

SEQ ID NO: 715 - protein, 9 aa, Artificial Sequence
  Peptide of Coronavirus Protein
  Unsure (4)..(4): Uncertain of the amino acid at this position, it may be absent.
NVAXKYTQL

SEQ ID NO: 716 - protein, 8 aa, Artificial Sequence
  Peptide of Coronavirus Protein
FIQQKLAL

SEQ ID NO: 717 - protein, 9 aa, Artificial Sequence
  Peptide of Coronavirus Protein
  Unsure (7)..(7): Uncertain of the amino acid at this position, it may be absent.
FIQQKLXAL

SEQ ID NO: 718 - protein, 9 aa, Artificial Sequence
  Peptide of Coronavirus Protein
GAKLKALNL

SEQ ID NO: 719 - protein, 9 aa, Artificial Sequence
  Peptide of Coronavirus Protein
HVMSKHTDF

SEQ ID NO: 720 - protein, 8 aa, Artificial Sequence
  Peptide of Coronavirus Protein
FMRFRRAF

SEQ ID NO: 721 - protein, 9 aa, Artificial Sequence
  Peptide of Coronavirus Protein
  Unsure (7)..(7): Uncertain of the amino acid at this position, it may be absent.
FMRFRRXAF

SEQ ID NO: 722 - protein, 9 aa, Artificial Sequence
  Peptide of Coronavirus Protein
YLKLRSDVL

SEQ ID NO: 723 - protein, 9 aa, Artificial Sequence
  Peptide of Coronavirus Protein
TQYNRYLAL

SEQ ID NO: 724 - protein, 9 aa, Artificial Sequence
  Peptide of Coronavirus Protein
FVKHKHAFL

SEQ ID NO: 725 - protein, 9 aa, Artificial Sequence
  Peptide of Coronavirus Protein
DGARRVWTL

SEQ ID NO: 726 - protein, 9 aa, Artificial Sequence
  Peptide of Coronavirus Protein
EMLDNRATL

SEQ ID NO: 727 - protein, 8 aa, Artificial Sequence
  Peptide of Coronavirus Protein
VLKKLKKS

SEQ ID NO: 728 - protein, 9 aa, Artificial Sequence
  Peptide of Coronavirus Protein
GPKVKYLYF

SEQ ID NO: 729 - protein, 9 aa, Artificial Sequence
  Peptide of Coronavirus Protein
NNLNRGMVL

SEQ ID NO: 730 - protein, 9 aa, Artificial Sequence
  Peptide of Coronavirus Protein
ITLKKRWQL

SEQ ID NO: 731 - protein, 9 aa, Artificial Sequence
  Peptide of Coronavirus Protein
IIKNLSKSL

SEQ ID NO: 732 - protein, 9 aa, Artificial Sequence
  Peptide of Coronavirus Protein
DGVKHVYQL

SEQ ID NO: 733 - protein, 8 aa, Artificial Sequence
  Peptide of Coronavirus Protein
NLKTLLSL

SEQ ID NO: 734 - protein, 9 aa, Artificial Sequence
  Peptide of Coronavirus Protein
  Unsure (7)..(7): Uncertain of the amino acid at this position, it may be absent.
NLKTLLXSL

SEQ ID NO: 735 - protein, 9 aa, Artificial Sequence
  Peptide of Coronavirus Protein
TIKPVTYKL

SEQ ID NO: 736 - protein, 9 aa, Artificial Sequence
  Peptide of Coronavirus Protein
ILKPANNSL

SEQ ID NO: 737 - protein, 8 aa, Artificial Sequence
  Peptide of Coronavirus Protein
TIKKPNEL

SEQ ID NO: 738 - protein, 9 aa, Artificial Sequence
  Peptide of Coronavirus Protein
  Unsure (4)..(4): Uncertain of the amino acid at this position, it may be absent.
TIKXKPNEL

SEQ ID NO: 739 - protein, 8 aa, Artificial Sequence
  Peptide of Coronavirus Protein
NSRIKASM

SEQ ID NO: 740 - protein, 9 aa, Artificial Sequence
  Peptide of Coronavirus Protein
  Unsure (7)..(7): Uncertain of the amino acid at this position, it may be absent.
NSRIKAXSM

SEQ ID NO: 741 - protein, 8 aa, Artificial Sequence
  Peptide of Coronavirus Protein
KTYERHSL

SEQ ID NO: 742 - protein, 8 aa, Artificial Sequence
  Peptide of Coronavirus Protein
KTYERHSL

SEQ ID NO: 743 - protein, 10 aa, Artificial Sequence
  Peptide of Coronavirus Protein
NVPMEKLKTL

SEQ ID NO: 744 - protein, 8 aa, Artificial Sequence
  Peptide of Coronavirus Protein
NVKDFMSL

SEQ ID NO: 745 - protein, 9 aa, Artificial Sequence
  Peptide of Coronavirus Protein
  UNSURE (5)..(5): Uncertain of the amino acid at this position, it may be absent.
NVKDXFMSL

SEQ ID NO: 746 - protein, 8 aa, Artificial Sequence
  Peptide of Coronavirus Protein
SAAKKNNL

SEQ ID NO: 747 - protein, 9 aa, Artificial Sequence
  Peptide of Coronavirus Protein
  UNSURE (4)..(4): Uncertain of the amino acid at this position, it may be absent.
SAAXKKNNL

SEQ ID NO: 748 - protein, 9 aa, Artificial Sequence
  Peptide of Coronavirus Protein
FAYTKRNVI

SEQ ID NO: 749 - protein, 9 aa, Artificial Sequence
  Peptide of Coronavirus Protein
SAKNRARTV

SEQ ID NO: 750 - protein, 9 aa, Artificial Sequence
  Peptide of Coronavirus Protein
IAATRGATV

SEQ ID NO: 751 - protein, 9 aa, Artificial Sequence
  Peptide of Coronavirus Protein
SLSHRFYRL

SEQ ID NO: 752 - protein, 8 aa, Artificial Sequence
  Peptide of Coronavirus Protein
YAYLRKHF

SEQ ID NO: 753 - protein, 9 aa, Artificial Sequence
  Peptide of Coronavirus Protein
  UNSURE (8)..(8): Uncertain of the amino acid at this position, it may be absent.
YAYLRKHXF

SEQ ID NO: 754 - protein, 9 aa, Artificial Sequence
  Peptide of Coronavirus Protein
YLRKHFSMM

SEQ ID NO: 755 - protein, 9 aa, Artificial Sequence
  Peptide of Coronavirus Protein
SIKNFKSVL

SEQ ID NO: 756 - protein, 8 aa, Artificial Sequence
  Peptide of Coronavirus Protein
NLTTRTQL

SEQ ID NO: 757 - protein, 9 aa, Artificial Sequence
  Peptide of Coronavirus Protein
  UNSURE (7)..(7): Uncertain of the amino acid at this position, it may be absent.
NLTTRTXQL

SEQ ID NO: 758 - protein, 9 aa, Artificial Sequence
  Peptide of Coronavirus Protein
TLDSKTQSL

SEQ ID NO: 759 - protein, 9 aa, Artificial Sequence
  Peptide of Coronavirus Protein
INITRFQTL

SEQ ID NO: 760 - protein, 9 aa, Artificial Sequence
  Peptide of Coronavirus Protein
LLALHRSYL

SEQ ID NO: 761 - protein, 8 aa, Artificial Sequence
  Peptide of Coronavirus Protein
YLQPRTFL

SEQ ID NO: 762 - protein, 9 aa, Artificial Sequence
  Peptide of Coronavirus Protein
  UNSURE (7)..(7): Uncertain of the amino acid at this position, it may be absent.
YLQPRTXFL

SEQ ID NO: 763 - protein, 9 aa, Artificial Sequence
  Peptide of Coronavirus Protein
TANPKTPKY

SEQ ID NO: 764 - protein, 10 aa, Artificial Sequence
  Peptide of Coronavirus Protein
FPLCANGQVF

SEQ ID NO: 765 - protein, 9 aa, Artificial Sequence
  Peptide of Coronavirus Protein
FPLANGQVF

SEQ ID NO: 766 - protein, 10 aa, Artificial Sequence
  Peptide of Coronavirus Protein
YPTLNISDEF

SEQ ID NO: 767 - protein, 9 aa, Artificial Sequence
  Peptide of Coronavirus Protein
YPLNISDEF

SEQ ID NO: 768 - protein, 9 aa, Artificial Sequence
  Peptide of Coronavirus Protein
FAIGLALYY

SEQ ID NO: 769 - protein, 8 aa, Artificial Sequence
  Peptide of Coronavirus Protein
YPSARIVY

SEQ ID NO: 770 - protein, 9 aa, Artificial Sequence
  Peptide of Coronavirus Protein
  UNSURE (4)..(4): Uncertain of the amino acid at this position, it may be absent.
YPSXARIVY

SEQ ID NO: 771 - protein, 9 aa, Artificial Sequence
  Peptide of Coronavirus Protein
FAYANRNRF

SEQ ID NO: 772 - protein, 9 aa, Artificial Sequence
  Peptide of Coronavirus Protein
YANRNRFLY

SEQ ID NO: 773 - protein, 11 aa, Artificial Sequence
  Peptide of Coronavirus Protein
VPHVGEIPVAY

SEQ ID NO: 774 - protein, 9 aa, Artificial Sequence
  Peptide of Coronavirus Protein
HVGEIPVAY

SEQ ID NO: 775 - protein, 10 aa, Artificial Sequence
  Peptide of Coronavirus Protein
FAVDAAKAYK

SEQ ID NO: 776 - protein, 9 aa, Artificial Sequence
  Peptide of Coronavirus Protein
EANMDQESF

SEQ ID NO: 777 - protein, 10 aa, Artificial Sequence
  Peptide of Coronavirus Protein
IPGIPKDMTY

SEQ ID NO: 778 - protein, 9 aa, Artificial Sequence
  Peptide of Coronavirus Protein
IPIPKDMTY

SEQ ID NO: 779 - protein, 9 aa, Artificial Sequence
  Peptide of Coronavirus Protein
HSIGFDYVY

SEQ ID NO: 780 - protein, 10 aa, Artificial Sequence
  Peptide of Coronavirus Protein
YPANSIVCRF

SEQ ID NO: 781 - protein, 9 aa, Artificial Sequence
  Peptide of Coronavirus Protein
YPANSIVRF

SEQ ID NO: 782 - protein, 9 aa, Artificial Sequence
  Peptide of Coronavirus Protein
EAVKTQFNY

SEQ ID NO: 783 - protein, 8 aa, Artificial Sequence
  Peptide of Coronavirus Protein
YAFEHIVY

SEQ ID NO: 784 - protein, 9 aa, Artificial Sequence
  Peptide of Coronavirus Protein
  UNSURE (6)..(6): Uncertain of the amino acid at this position, it may be absent.
YAFEHXIVY

SEQ ID NO: 785 - protein, 10 aa, Artificial Sequence
  Peptide of Coronavirus Protein
IPMDSTVKNY

SEQ ID NO: 786 - protein, 9 aa, Artificial Sequence
  Peptide of Coronavirus Protein
IPMDSTVKY

SEQ ID NO: 787 - protein, 9 aa, Artificial Sequence
  Peptide of Coronavirus Protein
QAWQPGVAM

SEQ ID NO: 788 - protein, 9 aa, Artificial Sequence
  Peptide of Coronavirus Protein
TILDGISQY

SEQ ID NO: 789 - protein, 9 aa, Artificial Sequence
  Peptide of Coronavirus Protein
DASGKPVPY

SEQ ID NO: 790 - protein, 9 aa, Artificial Sequence
  Peptide of Coronavirus Protein
NVLEGSVAY

SEQ ID NO: 791 - protein, 9 aa, Artificial Sequence
  Peptide of Coronavirus Protein
SIIQFPNTY

SEQ ID NO: 792 - protein, 10 aa, Artificial Sequence
  Peptide of Coronavirus Protein
FLPGVYSVIY

SEQ ID NO: 793 - protein, 9 aa, Artificial Sequence
  Peptide of Coronavirus Protein
VPFWITIAY

SEQ ID NO: 794 - protein, 9 aa, Artificial Sequence
  Peptide of Coronavirus Protein
LPLTQYNRY

SEQ ID NO: 795 - protein, 11 aa, Artificial Sequence
  Peptide of Coronavirus Protein
FLLPSLATVAY

SEQ ID NO: 796 - protein, 9 aa, Artificial Sequence
  Peptide of Coronavirus Protein
NALDQAISM

SEQ ID NO: 797 - protein, 9 aa, Artificial Sequence
  Peptide of Coronavirus Protein
LAKDTTEAF

SEQ ID NO: 798 - protein, 9 aa, Artificial Sequence
  Peptide of Coronavirus Protein
LPNNTASWF

SEQ ID NO: 799 - protein, 9 aa, Artificial Sequence
  Peptide of Coronavirus Protein
KAYNVTQAF

SEQ ID NO: 800 - protein, 9 aa, Artificial Sequence
  Peptide of Coronavirus Protein
TPSGTWLTY

SEQ ID NO: 801 - protein, 9 aa, Artificial Sequence
  Peptide of Coronavirus Protein
LPAADLDDF

SEQ ID NO: 802 - protein, 9 aa, Artificial Sequence
  Peptide of Coronavirus Protein
NPLLYDANY

SEQ ID NO: 803 - protein, 13 aa, Artificial Sequence
  Peptide of Coronavirus Protein
FAFACPDGVKHVY

SEQ ID NO: 804 - protein, 9 aa, Artificial Sequence
  Peptide of Coronavirus Protein
FPDGVKHVY

SEQ ID NO: 805 - protein, 9 aa, Artificial Sequence
  Peptide of Coronavirus Protein
YPGQGLNGY

SEQ ID NO: 806 - protein, 11 aa, Artificial Sequence
  Peptide of Coronavirus Protein
SPDAVTAYNGY

SEQ ID NO: 807 - protein, 9 aa, Artificial Sequence
  Peptide of Coronavirus Protein
  UNSURE (7)..(7): Uncertain of the amino acid at this position, it may be absent.
SPDAVTXAY

SEQ ID NO: 808 - protein, 8 aa, Artificial Sequence
  Peptide of Coronavirus Protein
SPDAVTAY

SEQ ID NO: 809 - protein, 9 aa, Artificial Sequence
  Peptide of Coronavirus Protein
QVVDMSMTY

SEQ ID NO: 810 - protein, 14 aa, Artificial Sequence
  Peptide of Coronavirus Protein
LPNDDTLRVEAFEY

SEQ ID NO: 811 - protein, 9 aa, Artificial Sequence
  Peptide of Coronavirus Protein
LPNDDTLEY

SEQ ID NO: 812 - protein, 9 aa, Artificial Sequence
  Peptide of Coronavirus Protein
NPPALQDAY

SEQ ID NO: 813 - protein, 10 aa, Artificial Sequence
  Peptide of Coronavirus Protein
TKPVETSNSF

SEQ ID NO: 814 - protein, 9 aa, Artificial Sequence
  Peptide of Coronavirus Protein
VPWDTIANY

SEQ ID NO: 815 - protein, 9 aa, Artificial Sequence
  Peptide of Coronavirus Protein
YPSLETIQI

SEQ ID NO: 816 - protein, 9 aa, Artificial Sequence
  Peptide of Coronavirus Protein
VARDLSLQF

SEQ ID NO: 817 - protein, 9 aa, Artificial Sequence
  Peptide of Coronavirus Protein
SAKSASVYY

SEQ ID NO: 818 - protein, 9 aa, Artificial Sequence
  Peptide of Coronavirus Protein
VPFVVSTGY

SEQ ID NO: 819 - protein, 9 aa, Artificial Sequence
  Peptide of Coronavirus Protein
NAAISDYDY

SEQ ID NO: 820 - protein, 9 aa, Artificial Sequence
  Peptide of Coronavirus Protein
FVVEVVDKY

SEQ ID NO: 821 - protein, 9 aa, Artificial Sequence
  Peptide of Coronavirus Protein
NPHLMGWDY

SEQ ID NO: 822 - protein, 9 aa, Artificial Sequence
  Peptide of Coronavirus Protein
FVSLAIDAY

SEQ ID NO: 823 - protein, 11 aa, Artificial Sequence
  Peptide of Coronavirus Protein
YPLTKHPNQEY

SEQ ID NO: 824 - protein, 9 aa, Artificial Sequence
  Peptide of Coronavirus Protein
YPLTPNQEY

SEQ ID NO: 825 - protein, 13 aa, Artificial Sequence
  Peptide of Coronavirus Protein
HPNQEYADVFHLY

SEQ ID NO: 826 - protein, 9 aa, Artificial Sequence
  Peptide of Coronavirus Protein
LPPAYTNSF

SEQ ID NO: 827 - protein, 9 aa, Artificial Sequence
  Peptide of Coronavirus Protein
LPFFSNVTW

SEQ ID NO: 828 - protein, 9 aa, Artificial Sequence
  Peptide of Coronavirus Protein
FVFKNIDGY

SEQ ID NO: 829 - protein, 10 aa, Artificial Sequence
  Peptide of Coronavirus Protein
LPIGINITRF

SEQ ID NO: 830 - protein, 9 aa, Artificial Sequence
  Peptide of Coronavirus Protein
LPIGINITF

SEQ ID NO: 831 - protein, 9 aa, Artificial Sequence
  Peptide of Coronavirus Protein
QPTESIVRF

SEQ ID NO: 832 - protein, 9 aa, Artificial Sequence
  Peptide of Coronavirus Protein
NATRFASVY

SEQ ID NO: 833 - protein, 11 aa, Artificial Sequence
  Peptide of Coronavirus Protein
IPIGAGICASY

SEQ ID NO: 834 - protein, 9 aa, Artificial Sequence
  Peptide of Coronavirus Protein
IPIGAGISY

SEQ ID NO: 835 - protein, 9 aa, Artificial Sequence
  Peptide of Coronavirus Protein
VASQSIIAY

SEQ ID NO: 836 - protein, 9 aa, Artificial Sequence
  Peptide of Coronavirus Protein
LGAENSVAY

SEQ ID NO: 837 - protein, 9 aa, Artificial Sequence
  Peptide of Coronavirus Protein
IPFAMQMAY

SEQ ID NO: 838 - protein, 11 aa, Artificial Sequence
  Peptide of Coronavirus Protein
FPQSAPHGVVF

SEQ ID NO: 839 - protein, 9 aa, Artificial Sequence
  Peptide of Coronavirus Protein
FPAPHGVVF

SEQ ID NO: 840 - protein, 11 aa, Artificial Sequence
  Peptide of Coronavirus Protein
SDRELHLSWEV

SEQ ID NO: 841 - protein, 10 aa, Artificial Sequence
  Peptide of Coronavirus Protein
SELVIGAVIL

SEQ ID NO: 842 - protein, 9 aa, Artificial Sequence
  Peptide of Coronavirus Protein
SEVIGAVIL

SEQ ID NO: 843 - protein, 11 aa, Artificial Sequence
  Peptide of Coronavirus Protein
KEITVATSRTL

SEQ ID NO: 844 - protein, 9 aa, Artificial Sequence
  Peptide of Coronavirus Protein
KEITVATTL

SEQ ID NO: 845 - protein, 9 aa, Artificial Sequence
  Peptide of Coronavirus Protein
VEKGVLPQL

SEQ ID NO: 846 - protein, 12 aa, Artificial Sequence
  Peptide of Coronavirus Protein
GEIPVAYRKVLL

SEQ ID NO: 847 - protein, 10 aa, Artificial Sequence
  Peptide of Coronavirus Protein
TEVPANSTVL

SEQ ID NO: 848 - protein, 9 aa, Artificial Sequence
  Peptide of Coronavirus Protein
TEVPANTVL

SEQ ID NO: 849 - protein, 10 aa, Artificial Sequence
  Peptide of Coronavirus Protein
REAVGTNLPL

SEQ ID NO: 850 - protein, 9 aa, Artificial Sequence
  Peptide of Coronavirus Protein
REAVGTNLL

SEQ ID NO: 851 - protein, 10 aa, Artificial Sequence
  Peptide of Coronavirus Protein
IEYPIIGDEL

SEQ ID NO: 852 - protein, 9 aa, Artificial Sequence
  Peptide of Coronavirus Protein
IEYPIGDEL

SEQ ID NO: 853 - protein, 10 aa, Artificial Sequence
  Peptide of Coronavirus Protein
VELFENKTTL

SEQ ID NO: 854 - protein, 9 aa, Artificial Sequence
  Peptide of Coronavirus Protein
QEFKPRSQM

SEQ ID NO: 855 - protein, 10 aa, Artificial Sequence
  Peptide of Coronavirus Protein
MEIDFLELAM

SEQ ID NO: 856 - protein, 9 aa, Artificial Sequence
  Peptide of Coronavirus Protein
MEIDFELAM

SEQ ID NO: 857 - protein, 9 aa, Artificial Sequence
  Peptide of Coronavirus Protein
FELEDFIPM

SEQ ID NO: 858 - protein, 9 aa, Artificial Sequence
  Peptide of Coronavirus Protein
VEIIKSQDL

SEQ ID NO: 859 - protein, 11 aa, Artificial Sequence
  Peptide of Coronavirus Protein
YELQTPFEIKL

SEQ ID NO: 860 - protein, 9 aa, Artificial Sequence
  Peptide of Coronavirus Protein
YELQTPIKL

SEQ ID NO: 861 - protein, 13 aa, Artificial Sequence
  Peptide of Coronavirus Protein
ACHNSEVGPEHSL

SEQ ID NO: 862 - protein, 9 aa, Artificial Sequence
  Peptide of Coronavirus Protein
AEVGPEHSL

SEQ ID NO: 863 - protein, 9 aa, Artificial Sequence
  Peptide of Coronavirus Protein
AEYHNESGL

SEQ ID NO: 864 - protein, 9 aa, Artificial Sequence
  Peptide of Coronavirus Protein
NESGLKTIL

SEQ ID NO: 865 - protein, 9 aa, Artificial Sequence
  Peptide of Coronavirus Protein
SEGLNDNLL

SEQ ID NO: 866 - protein, 10 aa, Artificial Sequence
  Peptide of Coronavirus Protein
GEQKSILSPL

SEQ ID NO: 867 - protein, 9 aa, Artificial Sequence
  Peptide of Coronavirus Protein
GEQKSILSL

SEQ ID NO: 868 - protein, 10 aa, Artificial Sequence
  Peptide of Coronavirus Protein
KEIKESVQTF

SEQ ID NO: 869 - protein, 11 aa, Artificial Sequence
  Peptide of Coronavirus Protein
KEIIFLEGETL

SEQ ID NO: 870 - protein, 9 aa, Artificial Sequence
  Peptide of Coronavirus Protein
KEFLEGETL

SEQ ID NO: 871 - protein, 9 aa, Artificial Sequence
  Peptide of Coronavirus Protein
GETLPTEVL

SEQ ID NO: 872 - protein, 11 aa, Artificial Sequence
  Peptide of Coronavirus Protein
FSSEIIGYKAI

SEQ ID NO: 873 - protein, 10 aa, Artificial Sequence
  Peptide of Coronavirus Protein
REVGFVVPGL

SEQ ID NO: 874 - protein, 9 aa, Artificial Sequence
  Peptide of Coronavirus Protein
REVGFVVGL

SEQ ID NO: 875 - protein, 13 aa, Artificial Sequence
  Peptide of Coronavirus Protein
GEYSHVVAFNTLL

SEQ ID NO: 876 - protein, 12 aa, Artificial Sequence
  Peptide of Coronavirus Protein
FLYENAFLPFAM

SEQ ID NO: 877 - protein, 10 aa, Artificial Sequence
  Peptide of Coronavirus Protein
TEAFEKMVSL

SEQ ID NO: 878 - protein, 10 aa, Artificial Sequence
  Peptide of Coronavirus Protein
KSEFDRDAAM

SEQ ID NO: 879 - protein, 11 aa, Artificial Sequence
  Peptide of Coronavirus Protein
SEISMDNSPNL

SEQ ID NO: 880 - protein, 10 aa, Artificial Sequence
  Peptide of Coronavirus Protein
QECVRGTTVL

SEQ ID NO: 881 - protein, 9 aa, Artificial Sequence
  Peptide of Coronavirus Protein
QEVRGTTVL

SEQ ID NO: 882 - protein, 10 aa, Artificial Sequence
  Peptide of Coronavirus Protein
YEGNSPFHPL

SEQ ID NO: 883 - protein, 9 aa, Artificial Sequence
  Peptide of Coronavirus Protein
YENSPFHPL

SEQ ID NO: 884 - protein, 9 aa, Artificial Sequence
  Peptide of Coronavirus Protein
QELYSPIFL

SEQ ID NO: 885 - protein, 14 aa, Artificial Sequence
  Peptide of Coronavirus Protein
YEDFLEYHDVRVVL

SEQ ID NO: 886 - protein, 9 aa, Artificial Sequence
  Peptide of Coronavirus Protein
IEVQGYKSV

SEQ ID NO: 887 - protein, 11 aa, Artificial Sequence
  Peptide of Coronavirus Protein
FELDERIDKVL

SEQ ID NO: 888 - protein, 9 aa, Artificial Sequence
  Peptide of Coronavirus Protein
SELLTPLGI

SEQ ID NO: 889 - protein, 9 aa, Artificial Sequence
  Peptide of Coronavirus Protein
SEDNQTTTI

SEQ ID NO: 890 - protein, 9 aa, Artificial Sequence
  Peptide of Coronavirus Protein
VEVQPQLEM

SEQ ID NO: 891 - protein, 12 aa, Artificial Sequence
  Peptide of Coronavirus Protein
LEMELTPVVQTI

SEQ ID NO: 892 - protein, 9 aa, Artificial Sequence
  Peptide of Coronavirus Protein
LEMELTPVV

SEQ ID NO: 893 - protein, 10 aa, Artificial Sequence
  Peptide of Coronavirus Protein
YENFNQHEVL

SEQ ID NO: 894 - protein, 9 aa, Artificial Sequence
  Peptide of Coronavirus Protein
YEFNQHEVL

SEQ ID NO: 895 - protein, 9 aa, Artificial Sequence
  Peptide of Coronavirus Protein
HEVLLAPLL

SEQ ID NO: 896 - protein, 9 aa, Artificial Sequence
  Peptide of Coronavirus Protein
NETLVTMPL

SEQ ID NO: 897 - protein, 9 aa, Artificial Sequence
  Peptide of Coronavirus Protein
GEVITFDNL

SEQ ID NO: 898 - protein, 11 aa, Artificial Sequence
  Peptide of Coronavirus Protein
SLREVRTIKVF

SEQ ID NO: 899 - protein, 9 aa, Artificial Sequence
  Peptide of Coronavirus Protein
HEGKTFYVL

SEQ ID NO: 900 - protein, 10 aa, Artificial Sequence
  Peptide of Coronavirus Protein
IELKFNPPAL

SEQ ID NO: 901 - protein, 9 aa, Artificial Sequence
  Peptide of Coronavirus Protein
GEAANFCAL

SEQ ID NO: 902 - protein, 10 aa, Artificial Sequence
  Peptide of Coronavirus Protein
YEQFKKGVQI

SEQ ID NO: 903 - protein, 9 aa, Artificial Sequence
  Peptide of Coronavirus Protein
YEFKKGVQI

SEQ ID NO: 904 - protein, 12 aa, Artificial Sequence
  Peptide of Coronavirus Protein
SEYKGPITDVFY

SEQ ID NO: 905 - protein, 9 aa, Artificial Sequence
  Peptide of Coronavirus Protein
KENSYTTTI

SEQ ID NO: 906 - protein, 9 aa, Artificial Sequence
  Peptide of Coronavirus Protein
TEVVGDIIL

SEQ ID NO: 907 - protein, 9 aa, Artificial Sequence
  Peptide of Coronavirus Protein
NELSRVLGL

SEQ ID NO: 908 - protein, 10 aa, Artificial Sequence
  Peptide of Coronavirus Protein
AEWFLAYILF

SEQ ID NO: 909 - protein, 11 aa, Artificial Sequence
  Peptide of Coronavirus Protein
AEAELAKNVSL

SEQ ID NO: 910 - protein, 13 aa, Artificial Sequence
  Peptide of Coronavirus Protein
AELAKNVSLDNVL

SEQ ID NO: 911 - protein, 9 aa, Artificial Sequence
  Peptide of Coronavirus Protein
VENMTPRDL

SEQ ID NO: 912 - protein, 9 aa, Artificial Sequence
  Peptide of Coronavirus Protein
HEETIYNLL

SEQ ID NO: 913 - protein, 9 aa, Artificial Sequence
  Peptide of Coronavirus Protein
AESHVDTDL

SEQ ID NO: 914 - protein, 10 aa, Artificial Sequence
  Peptide of Coronavirus Protein
HEFCSQHTML

SEQ ID NO: 915 - protein, 9 aa, Artificial Sequence
  Peptide of Coronavirus Protein
HEFSQHTML

SEQ ID NO: 916 - protein, 11 aa, Artificial Sequence
  Peptide of Coronavirus Protein
YEAMYTPHTVL

SEQ ID NO: 917 - protein, 9 aa, Artificial Sequence
  Peptide of Coronavirus Protein
YEYTPHTVL

SEQ ID NO: 918 - protein, 9 aa, Artificial Sequence
  Peptide of Coronavirus Protein
FEYVSQPFL

SEQ ID NO: 919 - protein, 9 aa, Artificial Sequence
  Peptide of Coronavirus Protein
GEVFNATRF

SEQ ID NO: 920 - protein, 10 aa, Artificial Sequence
  Peptide of Coronavirus Protein
VEAEVQIDRL

SEQ ID NO: 921 - protein, 9 aa, Artificial Sequence
  Peptide of Coronavirus Protein
VEAEVQIRL

SEQ ID NO: 922 - protein, 9 aa, Artificial Sequence
  Peptide of Coronavirus Protein
AEVQIDRLI

SEQ ID NO: 923 - protein, 9 aa, Artificial Sequence
  Peptide of Coronavirus Protein
AEIRASANL

SEQ ID NO: 924 - protein, 9 aa, Artificial Sequence
  Peptide of Coronavirus Protein
KEIDRLNEV

SEQ ID NO: 925 - protein, 13 aa, Artificial Sequence
  Peptide of Coronavirus Protein
REVLSDRELHLSW

SEQ ID NO: 926 - protein, 10 aa, Artificial Sequence
  Peptide of Coronavirus Protein
GEYTFEKGDY

SEQ ID NO: 927 - protein, 9 aa, Artificial Sequence
  Peptide of Coronavirus Protein
DEISMATNY

SEQ ID NO: 928 - protein, 10 aa, Artificial Sequence
  Peptide of Coronavirus Protein
REFLTRNPAW

SEQ ID NO: 929 - protein, 10 aa, Artificial Sequence
  Peptide of Coronavirus Protein
EELKKLLEQW

SEQ ID NO: 930 - protein, 8 aa, Artificial Sequence
  Peptide of Coronavirus Protein
AELEGIQY

SEQ ID NO: 931 - protein, 8 aa, Artificial Sequence
  Peptide of Coronavirus Protein
AENVTGLF

SEQ ID NO: 932 - protein, 9 aa, Artificial Sequence
  Peptide of Coronavirus Protein
  UNSURE (7)..(7): Uncertain of the amino acid at this position, it may be absent.
AENVTGXLF

SEQ ID NO: 933 - protein, 11 aa, Artificial Sequence
  Peptide of Coronavirus Protein
REEAIRHVRAW

SEQ ID NO: 934 - protein, 9 aa, Artificial Sequence
  Peptide of Coronavirus Protein
FELTSMKYF

SEQ ID NO: 935 - protein, 10 aa, Artificial Sequence
  Peptide of Coronavirus Protein
GEAVKTQFNY

SEQ ID NO: 936 - protein, 8 aa, Artificial Sequence
  Peptide of Coronavirus Protein
TEISFMLW

SEQ ID NO: 937 - protein, 12 aa, Artificial Sequence
  Peptide of Coronavirus Protein
KENDSKEGFFTY

SEQ ID NO: 938 - protein, 10 aa, Artificial Sequence
  Peptide of Coronavirus Protein
REHEHEIAWY

SEQ ID NO: 939 - protein, 9 aa, Artificial Sequence
  Peptide of Coronavirus Protein
FEIKLAKKF

SEQ ID NO: 940 - protein, 12 aa, Artificial Sequence
  Peptide of Coronavirus Protein
SEVGPEHSLAEY

SEQ ID NO: 941 - protein, 10 aa, Artificial Sequence
  Peptide of Coronavirus Protein
EEIAIILASF

SEQ ID NO: 942 - protein, 10 aa, Artificial Sequence
  Peptide of Coronavirus Protein
YEKLKPVLDW

SEQ ID NO: 943 - protein, 10 aa, Artificial Sequence
  Peptide of Coronavirus Protein
EEKFKEGVEF

SEQ ID NO: 944 - protein, 9 aa, Artificial Sequence
  Peptide of Coronavirus Protein
LEIKDTEKY

SEQ ID NO: 945 - protein, 13 aa, Artificial Sequence
  Peptide of Coronavirus Protein
SEAGVCVSTSGRW

SEQ ID NO: 946 - protein, 9 aa, Artificial Sequence
  Peptide of Coronavirus Protein
SEAGVSGRW

SEQ ID NO: 947 - protein, 10 aa, Artificial Sequence
  Peptide of Coronavirus Protein
ASEFSSLPSY

SEQ ID NO: 948 - protein, 9 aa, Artificial Sequence
  Peptide of Coronavirus Protein
MDNSPNLAW

SEQ ID NO: 949 - protein, 9 aa, Artificial Sequence
  Peptide of Coronavirus Protein
TELEPPCRF

SEQ ID NO: 950 - protein, 13 aa, Artificial Sequence
  Peptide of Coronavirus Protein
QELIRQGTDYKHW

SEQ ID NO: 951 - protein, 9 aa, Artificial Sequence
  Peptide of Coronavirus Protein
QELTDYKHW

SEQ ID NO: 952 - protein, 13 aa, Artificial Sequence
  Peptide of Coronavirus Protein
SRIGMEVTPSGTW

SEQ ID NO: 953 - protein, 9 aa, Artificial Sequence
  Peptide of Coronavirus Protein
SEVTPSGTW

SEQ ID NO: 954 - protein, 11 aa, Artificial Sequence
  Peptide of Coronavirus Protein
GMEVTPSGTWL

SEQ ID NO: 955 - protein, 10 aa, Artificial Sequence
  Peptide of Coronavirus Protein
GLEAPFLYLY

SEQ ID NO: 956 - protein, 14 aa, Artificial Sequence
  Peptide of Coronavirus Protein
SEHDYQIGGYTEKW

SEQ ID NO: 957 - protein, 9 aa, Artificial Sequence
  Peptide of Coronavirus Protein
EEHVQIHTI

SEQ ID NO: 958 - protein, 9 aa, Artificial Sequence
  Peptide of Coronavirus Protein
DEWSMATYY

SEQ ID NO: 959 - protein, 10 aa, Artificial Sequence
  Peptide of Coronavirus Protein
GEFKLASHMY

SEQ ID NO: 960 - protein, 14 aa, Artificial Sequence
  Peptide of Coronavirus Protein
GDCEEEEFEPSTQY

SEQ ID NO: 961 - protein, 12 aa, Artificial Sequence
  Peptide of Coronavirus Protein
EEEFEPSTQYEY

SEQ ID NO: 962 - protein, 9 aa, Artificial Sequence
  Peptide of Coronavirus Protein
IEVNSFSGY

SEQ ID NO: 963 - protein, 9 aa, Artificial Sequence
  Peptide of Coronavirus Protein
SEKQVEQKI

SEQ ID NO: 964 - protein, 11 aa, Artificial Sequence
  Peptide of Coronavirus Protein
AEIPKEEVKPF

SEQ ID NO: 965 - protein, 13 aa, Artificial Sequence
  Peptide of Coronavirus Protein
SNEKQEILGTVSW

SEQ ID NO: 966 - protein, 11 aa, Artificial Sequence
  Peptide of Coronavirus Protein
QEGVVDYGARF

SEQ ID NO: 967 - protein, 12 aa, Artificial Sequence
  Peptide of Coronavirus Protein
GELGDVRETMSY

SEQ ID NO: 968 - protein, 12 aa, Artificial Sequence
  Peptide of Coronavirus Protein
SEYTGNYQCGHY

SEQ ID NO: 969 - protein, 11 aa, Artificial Sequence
  Peptide of Coronavirus Protein
TEIDPKLDNYY

SEQ ID NO: 970 - protein, 11 aa, Artificial Sequence
  Peptide of Coronavirus Protein
AEVAVKMFDAY

SEQ ID NO: 971 - protein, 14 aa, Artificial Sequence
  Peptide of Coronavirus Protein
TVYSDVENPHLMGW

SEQ ID NO: 972 - protein, 9 aa, Artificial Sequence
  Peptide of Coronavirus Protein
TENPHLMGW

SEQ ID NO: 973 - protein, 11 aa, Artificial Sequence
  Peptide of Coronavirus Protein
SDVENPHLMGW

SEQ ID NO: 974 - protein, 10 aa, Artificial Sequence
  Peptide of Coronavirus Protein
TEKSNIIRGW

SEQ ID NO: 975 - protein, 10 aa, Artificial Sequence
  Peptide of Coronavirus Protein
SETKCTLKSF

SEQ ID NO: 976 - protein, 11 aa, Artificial Sequence
  Peptide of Coronavirus Protein
QEVFAQVKQIY

SEQ ID NO: 977 - protein, 9 aa, Artificial Sequence
  Peptide of Coronavirus Protein
ADAGFIKQY

SEQ ID NO: 978 - protein, 12 aa, Artificial Sequence
  Peptide of Coronavirus Protein
REGVFVSNGTHW

SEQ ID NO: 979 - protein, 12 aa, Artificial Sequence
  Peptide of Coronavirus Protein
QELGKYEQYIKW

SEQ ID NO: 980 - protein, 9 aa, Artificial Sequence
  Peptide of Coronavirus Protein
YEQYIKWPW

SEQ ID NO: 981 - protein, 12 aa, Artificial Sequence
  Peptide of Coronavirus Protein
AEIVDTVSALVY

SEQ ID NO: 982 - protein, 11 aa, Artificial Sequence
  Peptide of Coronavirus Protein
RELNGGAYTRY

SEQ ID NO: 983 - protein, 9 aa, Artificial Sequence
  Peptide of Coronavirus Protein
RELNGGARY

SEQ ID NO: 984 - protein, 11 aa, Artificial Sequence
  Peptide of Coronavirus Protein
SYEDQDALFAY

SEQ ID NO: 985 - protein, 8 aa, Artificial Sequence
  Peptide of Coronavirus Protein
AEHVNNSY

(19) United States
(12) Patent Application Publication
(10) Pub. No.: US 2023/0117521 A1
(43) Pub. Date: Apr. 20, 2023

(54) T-CELL MODULATORY MULTIMERIC POLYPEPTIDES WITH CONJUGATION SITES AND METHODS OF USE THEREOF

(71) Applicant: Cue Biopharma, Inc., Boston, MA (US)

(72) Inventors: Ronald D. SEIDEL, III, Natick, MA (US); Anish SURI, Boston, MA (US); John F. ROSS, Boston, MA (US); Chee Meng LOW, Boston, MA (US)

(21) Appl. No.: 17/914,734

(22) PCT Filed: Mar. 25, 2021
(86) PCT No.: PCT/US2021/024201

§ 371 Date: Sep. 26, 2022

Related U.S. Application Data
(60) Provisional application No. 62/994,790, filed on Mar. 25, 2020.

(51) Int. Cl. C07K 14/74; C07K 14/005; C07K 14/55

(52) U.S. Cl. CPC C07K 14/70539; C07K 14/005; C07K 14/55; A61K 2039/605

(57) ABSTRACT

The present disclosure provides T-cell modulatory multimeric polypeptides (T-Cell-MMPs) conjugated to a coronavirus epitope and comprising at least one immunomodulatory polypeptide (“MOD”) that may be selected to exhibit reduced binding affinity to a cognate co-immunomodulatory polypeptide (“Co-MOD”). By presenting the coronavirus epitope and MOD to a T-cell, the T-Cell-MMP-coronavirus epitope conjugates are useful for modulating the activity (e.g., increasing proliferation or cytotoxic activity) of T-cells specific to the coronavirus peptide in an epitope selective/specific manner, and accordingly, for treating individuals with a coronavirus infection.

### CROSS-REFERENCE TO RELATED APPLICATIONS

This application claims the benefit of U.S. Provisional Patent Application No. 62/994,790, filed Mar. 25, 2020.

### INCORPORATION OF SEQUENCE LISTING

This application contains a sequence listing submitted electronically via EFS-web, which serves as both the paper copy and the computer readable form (CRF) and consists of a file entitled “123640-8020WO00_seqlist.txt”, which was created on Mar. 25, 2021, which is 539,333 bytes in size, and which is herein incorporated by reference in its entirety.

### INTRODUCTION

Coronaviruses including MERS-CoV, Bat-SL-CoV, SARS-CoV, and SARS-CoV-2 (COVID-19) have been associated with a number of disease outbreaks having high lethality rates (e.g., Severe Acute Respiratory Syndrome or “SARS” and Middle East Respiratory Syndrome or “MERS”). The ability to induce an adaptive immune response to such viruses involves the engagement of the T-cell receptor (TCR), present on the surface of a T-cell, with a small peptide antigen of the virus that is non-covalently presented on the surface of an antigen presenting cell (APC) by a major histocompatibility complex (MHC; also referred to in humans as a human leukocyte antigen (HLA) complex). This engagement represents the immune system's targeting mechanism and is a requisite molecular interaction for T-cell modulation (activation or inhibition) and effector function. Following epitope-specific cell targeting, the targeted T-cells are activated through engagement of costimulatory proteins found on the APC with counterpart costimulatory proteins on the T-cells. Both signals—epitope/TCR binding and engagement of APC costimulatory proteins with T-cell costimulatory proteins—are required to drive T-cell specificity and activation or inhibition. The TCR is specific for a given epitope; however, the costimulatory protein is not epitope specific and instead is generally expressed on all T-cells or on large T-cell subsets.

### SUMMARY

The present disclosure provides T-cell modulatory multimeric polypeptides (a “T-Cell-MMP” or multiple “T-Cell-MMPs”) that in one embodiment comprise a portion of an MHC receptor and at least one immunomodulatory polypeptide (also referred to herein as a “MOD polypeptide” or, simply, a “MOD”). Any one or more of the MODs present in the T-Cell-MMP may be wild-type (“wt.”) or a variant that exhibits reduced binding affinity to its cellular (e.g., T-cell surface) binding partner/receptor (generally referred to as a “Co-MOD”). The unconjugated T-Cell-MMPs comprise at least one chemical conjugation site at which a molecule comprising a target epitope (e.g., a peptide, glycopeptide, or non-peptide such as a carbohydrate) from a coronavirus may be covalently bound for presentation to a cell bearing a T-cell receptor. T-Cell-MMPs comprising a chemical conjugation site for linking an epitope are useful for rapidly preparing T-Cell-MMP-epitope conjugates that can modulate the activity of T-cells specific to the epitope presented and, accordingly, for modulating an immune response in an individual involving those T-cells. The T-Cell-MMPs described herein are suitable for production in cell expression systems where most, substantially all (e.g., greater than 85% or 90% of the T-Cell-MMP), or all of the expressed protein is in a soluble non-aggregated state (e.g., in the form of dimers) that is suitably stable at 37° C. for production in tissue culture and use at least up to that temperature. Most, substantially all (e.g., greater than 85% or 90% of the T-Cell-MMP), or all of the expressed protein remains in a soluble non-aggregated state even after conjugation to epitope peptides and is similarly stable. The T-Cell-MMPs and their epitope conjugates may additionally comprise sites for the conjugation of payloads such as antiviral agents for co-delivery with a specific target epitope. As such, T-Cell-MMP-epitope conjugates may be considered a means by which to deliver MODs (e.g., IL-2, 4-1BBL, FasL, TGF-β, CD70, CD80, CD86, OX40L, ICOS-L, ICAM, JAG1, or fragments thereof, or altered (mutated) variants thereof) and/or payloads (e.g., labels or antivirals) to cells in an epitope specific manner.

In embodiments described herein the T-Cell-MMPs may comprise modifications that assist in the stabilization of the T-Cell-MMP during intracellular trafficking and/or following secretion by cells expressing the multimeric polypeptide even in the absence of an associated epitope peptide. In embodiments described herein the T-Cell-MMPs may include modifications that link the carboxyl end of the MHC-I α₁ helix and the amino end of the MHC-I α₂₋₁ helix. Such modifications include the insertion of cysteine residues that result in the formation of disulfide linkages linking the indicated regions of those helices. For example, the insertion of cysteine residues at amino acid (aa) 84 (Y84C substitution) and 139 (A139C substitution) of MHC-I, or the equivalent positions relative to the sequences forming the helices, may form a disulfide linkage that helps stabilize the T-Cell-MMP. See, e.g., Z. Hein et al. (2014), Journal of Cell Science 127:2885-2897.

### BRIEF DESCRIPTION OF THE DRAWINGS

FIG. 1 depicts preferential activation of an epitope-specific T-cell to an epitope non-specific T-cell by an embodiment of a T-Cell-MMP of the present disclosure bearing an epitope attached by chemical coupling (denoted by “CC”) to a β-2 microglobulin (β2M) polypeptide sequence.

FIGS. 2A-2G provide amino acid sequences of immunoglobulin Fc polypeptides (including SEQ ID NOs. 1-13).

FIGS. 3A, 3B and 3C provide amino acid sequences of human leukocyte antigen (HLA) Class I heavy chain polypeptides. Signal sequences, aas 1-24, are bolded and underlined. FIG. 3A entry: 3A.1 is the HLA-A heavy chain (HLA-A*01:01:01:01 or A*0101) (NCBI accession NP_001229687.1), SEQ ID NO:14; entry 3A.2 is HLA-A*1101, SEQ ID NO:15; entry 3A.3 is HLA-A*2402, SEQ ID NO:16, and entry 3A.4 is HLA-A*3303, SEQ ID NO:17. FIG. 3B provides the sequence for HLA-B*07:02:01 (HLA-B*0702) (NCBI GenBank Accession NP_005505.2 (see, also, GenBank Accession SEQ ID NO:18). FIG. 3C provides the sequence for HLA-C*0701 (GenBank Accession NP_001229971.1) (HLA-C*07:01:01:01 or HLA-Cw*070101), (HLA-Cw*07) (see GenBank Accession CA078194.1), SEQ ID NO:19.

FIG. 3D provides an alignment of eleven mature MHC Class I heavy chain peptide sequences without all, or substantially all, of their leader, transmembrane and intracellular domain regions. The aligned sequences include human HLA-A*0101, SEQ ID NO:20 (see also SEQ ID NO:14); HLA-B*0702, SEQ ID NO:21; HLA-C, SEQ ID NO:22; HLA-A*0201, SEQ ID NO:23; a mouse H2K protein sequence, SEQ ID NO:24; three variants of HLA-A (var.2, var. 2C [having Y84C and A139C substitutions], and var.2CP), SEQ ID NOs:25-27; 3 human HLA-A molecules (HLA-A*1101 (HLA-A11), SEQ ID NO:28; HLA-A*2402 (HLA-A24), SEQ ID NO:29; and HLA-A*3303 (HLA-A33), SEQ ID NO:30). HLA-A*0201 is a variant of HLA-A. The Y84A and A236C variant of HLA-A is marked as HLA-A (var. 2). The seventh HLA-A sequence. marked as HLA-A (var. 2C), shows HLA-A substituted with C residues at positions 84, 139 and 236, and the eighth sequence adds one additional proline to the C-terminus of the preceding sequence. The ninth through the eleventh sequences are from HLA-A11 (HLA-A*1101); HLA-A24 (HLA-A*2402); and HLA-A33 (HLA-A*3303), respectively, which are prevalent in certain Asian populations. Indicated in the alignment are the locations (84 and 139 of the mature proteins) where cysteine residues may be inserted in place of the aa at that position for the formation of a disulfide bond to stabilize the MHC-H-β2M complex in the absence of a bound epitope peptide. Also shown in the alignment is position 236 (of the mature polypeptide), which may be replaced by a cysteine residue that can form an interchain disulfide bond with β2M (e.g., at aa 12 of the mature polypeptide). An arrow appears above each of those locations and the residues are bolded. The boxes flanking residues 84, 139 and 236 show the groups of five aas on either side of those six sets of five residues, denoted aa cluster 1, aa cluster 2, aa cluster 3, aa cluster 4, aa cluster 5, and aa cluster 6 (shown in the figure as aac 1 through aac 6, respectively), that may be replaced by 1 to 5 aas selected independently from (i) any naturally occurring aa or (ii) any naturally occurring aa except proline or glycine.

FIGS. 3E-3G provide alignments of the an sequences of mature HLA-A, -B, and -C class I heavy chains, respectively. The sequences are provided for a portion of the mature proteins (without all or substantially all of their leader sequences, transmembrane domains or intracellular domains). As described in FIG. 3D, the positions of aa residues 84, 139, and 236 and their flanking residues (aac 1 to aac 6) that may be replaced by 1 to 5 aas selected independently from (i) any naturally occurring aa or (ii) any naturally occurring aa except proline or glycine are also shown. A consensus sequence is also provided for each group of HLA alleles provided in the figures showing the variable aa positions as “X” residues sequentially numbered and the locations of aas 84, 139 and 236 double underlined.

FIG. 3H provides a consensus sequence for each of HLA-E, -F, and -G with the variable an positions indicated as “X” residues sequentially numbered and the locations of aas 84, 139 and 236 double underlined.

FIG. 3I provides an alignment of the consensus an sequences for HLA-A, -B, -C, -E, -F, and -G, which are given in FIGS. 3E to 3H (SEQ ID NOs: 35, 43, and 53-56). The alignment shows the correspondence of aas between the different sequences. Variable residues in each sequence are listed as “X” with the sequential numbering removed. The permissible aas at each variable residue can be determined by reference to FIGS. 3E-3H. As indicated in FIG. 3D, the locations of aas 84, 139 and 236 with their flanking five-aa clusters that may be replaced by 1 to 5 aas selected independently from (i) any naturally occurring aa or (ii) any naturally occurring aa except proline or glycine are also shown.

FIG. 4 provides a multiple aa sequence alignment of β2M precursors (i.e., including the leader sequence) from Homo sapiens (NP_004039.1; SEQ ID NO:57), Pan troglodytes (NP_001009066.1; SEQ ID NO:58), Macaca mulatta (NP_001040602.1; SEQ ID NO:59), Bos Taurus (NP_776318.1; SEQ ID NO:60) and Mus musculus (NP_033865.2; SEQ ID NO:61). Underlined aas 1-20 are the signal peptide (sometime referred to as a leader sequence).

FIG. 5 provides six T-Cell-MMP embodiments (structures) marked as A through F. In each case the T-CeIl-MMPs comprise: a first polypeptide having an N-terminus and C-terminus and which comprises a first major histocompatibility complex (MHC) polypeptide (MHC-1); and a second polypeptide having an N-terminus and C-terminus and a second MHC polypeptide (MHC-2), and optionally comprising an immunoglobulin (Fc) polypeptide or a non-Ig polypeptide scaffold. In the embodiments shown the first and second polypeptides are shown linked by a disulfide bond; however, the T-Cell-MMPs do not require a disulfide linkage or any other covalent linkage between the first and second polypeptides. The T-Cell-MMPs may also comprise independently selected linker sequences indicated by the dashed line (- - -). The first polypeptide, the second polypeptide, or both the first and second polypeptides of the T-Cell-MMP comprise at least one chemical conjugation site. Some potential locations for the first polypeptide chemical conjugation sites (CC-1) and second polypeptide chemical conjugation sites (CC-2) are shown by arrows. Locations for one or more MODs that are selected independently (e.g., a sequence comprising one, two, three or more MODs connected in sequence with optional aa linkers between the MODs) are shown by “MOD” in the stippled box. The MODs may be variant MODs as described within this disclosure. In A the MOD(s) are located at the C-terminus of the first polypeptide, in B the MOD(s) are located at the N-terminus of the second polypeptide, in C the MOD(s) are located at the C-terminus of the second polypeptide, in D the MODs are located at the C-terminus of the first peptide and the N-terminus of the second peptide, in E the MOD(s) are added with the epitope peptide, and in F the MOD(s) are between the MHC-2 and Fc peptide. Where more than one MOD is present they may be the same (e.g., two IL-2 MODs) or different, and may be placed adjacent to each other.

FIG. 6 provides twelve embodiments of T-Cell-MMP-epitope conjugates, marked as A through L, that parallel the embodiments in FIG. 5. As in FIG. 5, the first polypeptide has an N-terminus and C-terminus with the first MHC polypeptide given as comprising a β-2-microglobulin polypeptide (β2M capable of interacting with the MHC Class I heavy chain (MHC-H) and presenting the epitope to a T-Cell receptor. The second polypeptide has an N-terminus and C-terminus and an MHC-H polypeptide, and optionally comprises an immunoglobulin (Fc) polypeptide or a non-Ig polypeptide scaffold. The optional disulfide bond joining the first and second polypeptides of the T-Cell-MMP-epitope conjugates is shown connecting the β2M peptide sequence and MHC-H peptide sequence in A to F, and the independently selected optional linker sequences, indicated by the dashed line (- - -), are not required. In G to L, the complexes in A to F are repeated; however, a disulfide bond joining the first and second polypeptides is shown joining the MHC-H peptide sequence to a linker sequence interposed between the epitope and β2M peptide sequence (e.g., a bond from a Cys residue at position 84 of an MHC-H chain sequence as indicated in FIG. 3 to the interposed linker). The first polypeptide, the second polypeptide, or both the first and second polypeptides of the T-Cell-MMP may also comprise one or more chemical conjugation sites in addition to the site employed for the conjugation of the epitope. The potential locations for such sites (CC-1 and CC-2) are shown by arrows. The one or more immunomodulatory polypeptides (either MODs or variant MODs) are as described in FIG. 5. The MODs (e.g., tandem IL-2 polypeptides) may be placed on the N-terminus of the MHC-H polypeptide (Position 1 as in B and H), between the MHC-H and Fc (Position 2 as in F and L), on the C-terminus of the MHC-H (Position 3 as in C and I); N-terminal to the peptide (Position 4 as in E and K); or C-terminal to the β2M (Position 5 as in A and G).

FIG. 7 provides examples of two dimers formed from T-Cell-MMPs. The dimer labeled “A” is the result of dimerizing two of the T-Cell-MMPs labeled “A” in FIG. 6. The dimer labeled “B” is the result of dimerizing two of the T-Cell-MMPs labeled “B” in FIG. 6. The embodiment as shown includes one or more disulfide bonds between the polypeptides, each of which is optional. In addition, only a subset of CC-2 sites in the Fc region or the attached optional linker are shown.

FIG. 8 shows some schematics of epitopes having a maleimide group appended for conjugation to a free nucleophile (e.g., cysteine) present in a T-Cell-MMP to form an epitope conjugate. In “a” the maleimide group is attached by an optional linker (e.g., a peptide linker sequence) to the epitope. In “b” through “e,” the linker is a glycine serine polypeptide (GGGGS) repeated n times, where n is 1-5 when present, and n is 0 when the linker is absent. In “c”-“e” the attachment of a maleimide group is through a lysine (K), such as through the epsilon amino group of the lysine. In “d” and “e” the maleimide group is linked to the peptide through an alkyl amide formed with the epsilon amino group of a lysine residue, where m is 1-7.

FIG. 9 shows in part A a map of a T-Cell-MMP with the first polypeptide having a sulfatase motif (aas 26-31 bolded) between two linker sequences as the location for developing a chemical conjugation site (an fGly residue) through the action of an FGE enzyme. At B, FIG. 9 shows a second polypeptide of a T-Cell-MMP having tandem IL-2 MODs attached to the amino end of a human MHC Class I HLA-A heavy chain polypeptide followed by a human IgG1 Fc polypeptide. Linkers are bolded, italicized and underlined.

FIG. 10A to FIG. 10 D show a series of HLA A*1101 heavy chain constructs having, from N-terminus to C-terminus, a human IL-2 signal sequence, shown in underline and bold. The signal (leader) sequence is followed by a MOD, which is indicated as a human IL-2 or an “optional peptide linker-immunomodulatory polypeptide-optional peptide linker.” Where the MOD is not specified, it may be any desired MOD. The remainder of the sequence is HLA A*1101 H chain sequence with three cysteine substitutions (Y84C; A139C; A236C); a linker; and a hIgG1 Fc with two aa substitutions (L234A; L235A). The asterisks indicate stops to the sequences.

FIG. 11A to FIG. 11J provide as sequences of COVID-19 (SARS-CoV-2) proteins/polypeptides, including some derived from the polyprotein of open reading frame 1ab (orf 1ab) set forth in NCBI Reference Sequence: YP_009724389.1

FIG. 12 shows a comparison of two immunomodulatory proteins each having a first polypeptide (comprising a β2M polypeptide sequence) and a second polypeptide (comprising an MHC-H chain α1-α3 segments and an IgFc). The immunomodulatory proteins appear as dimers comprising two copies of both the first and second polypeptides that are associated by a disulfide bond between the β2M and MHC-H sequences and disulfide bonds between the Fc regions. Structure A is a control immunomodulatory protein that has a 9 as cytomegalovirus (CMV) epitope at the N-terminus of a β2M polypeptide sequence of the first polypeptide. Structure B is a T-Cell-MMP of the present disclosure having a chemical conjugation site indicated with an “*” in the β2M polypeptide sequence; in this instance the chemical conjugation site is within a linker (not shown) at the N-terminus of a β2M polypeptide sequence. An SDS PAGE gel of the expressed and purified proteins under non-reducing and reducing conditions is shown at C with molecular weight markers (left lane), the non-reduced samples conjugated to MART1 and CMV peptides are in the 2nd and 3rd lanes from the left, reduced samples conjugated to MART1 and CMV peptides are in the 4th and 5th lanes from the left. The first polypeptides are labeled as “light chain” and the second polypeptides are labeled as “heavy chain.” See Example 2 for more details.

FIG. 13 shows size exclusion chromatography of T-Cell-MMPs conjugated to CMV (CMV+ T-Cell-MMP) and MART-1 (MART+ T-Cell-MMP) polypeptides plotted in mAU (milli-absorbance units) vs time in minutes. See example 3.

FIG. 14 shows the response of Ficoll-Paque® samples of leukocytes from CMV responsive donors simulated with various concentrations of MOD constructs and control treatments measured as the number of CMV or MART-1 responsive CD8+ T-cells. See Example 4 for details.

FIG. 15 provides a table of predicted Coronavirus epitopes (SARS-CoV and SARS-CoV-2), indicating for each their respective HLA restriction(s); adapted from Grifoni et al., Cell Host & Microbe 27: 1-10 (2020).

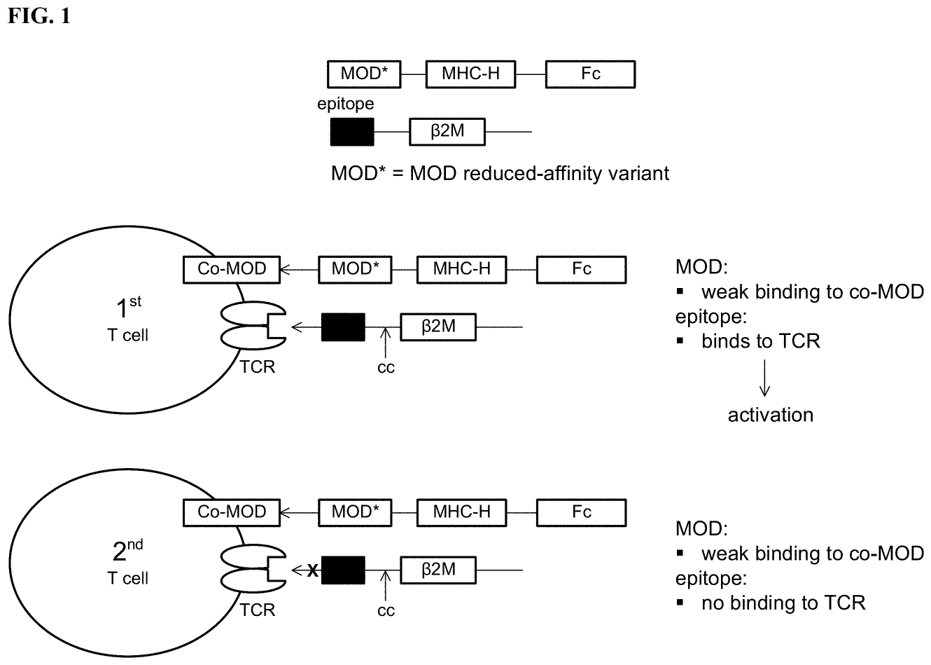

### DEFINITIONS

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

A polynucleotide or polypeptide has a certain percent “sequence identity” to another polynucleotide or polypeptide, meaning that, when aligned, that percentage of bases or aas is the same, and in the same relative position, when comparing the two sequences. Sequence identity can be determined in a number of different ways. To determine sequence identity, sequences can be aligned using various convenient methods and computer programs (e.g., BLAST, T-COFFEE, MUSCLE, MAFFT, etc.) available over the world wide web at sites including ncbi.nlm.nili.gov/BLAST, ebi.ac.uk/fools/msa/tcoffee/, ebi.ac.uk/Tools/msa/muscle/, and mafft.cbrc.jp/alignment/software/. See, e.g., Altschul et al. (1990), J. Mol. Biol. 215:403-10. Unless stated otherwise, sequence alignments are prepared using BLAST.

The terms “amino acid” and “amino acids” are abbreviated as “aa” and “aas,” respectively; however, as may be understood from the context, in some cases singular usage implies the plural. Naturally occurring aa or naturally occurring aas, unless stated otherwise, means: L (Leu, leucine), A (Ala, alanine), G (Gly, glycine), S (Ser, serine), V (Val, valine), F (Phe, phenylalanine), Y (Tyr, tyrosine), H (His, histidine), R (Arg, arginine), N (Asn, asparagine), E (Glu, glutamic acid), D (Asp, asparagine), C (Cys, cysteine), Q (Gln, glutamine), I (Ile, isoleucine), M (Met, methionine), P (Pro, proline), T (Thr, threonine), K (Lys, lysine), and W (Trp, tryptophan); all of the L-configuration. Both selenocysteine and hydroxyproline are naturally occurring aas that are specifically referred to in any instance where they are intended to be encompassed.

Non-natural aas are any aa other than the naturally occurring aas recited above, selenocysteine, and hydroxyproline.

“Chemical conjugation” as used herein means formation of a covalent bond. “Chemical conjugation site” as used herein means a location in a polypeptide at which a covalent bond can be formed, including any contextual elements (e.g., surrounding aa sequences) that are required or assist in the formation of a covalent bond to the polypeptide. Accordingly, a site comprising a group of aas that direct enzymatic modification, and ultimately covalent bond formation at an as within the group, may also be referred to as a chemical conjugation site. In some instances, as will be clear from the context, the term chemical conjugation site may be used to refer to a location where covalent bond formation or chemical modification has already occurred.

The term “conservative aa substitution” refers to the interchangeability in proteins of aa residues having similar side chains. For example, a group of aas having aliphatic side chains consists of glycine, alanine, valine, leucine, and isoleucine; a group of aas having aliphatic-hydroxyl side chains consists of serine and threonine; a group of aas having amide containing side chains consists of asparagine and glutamine; a group of aas having aromatic side chains consists of phenylalanine, tyrosine, and tryptophan; a group of aas having basic side chains consists of lysine, arginine, and histidine; a group of aas having acidic side chains consists of glutamate and aspartate; and a group of aas having sulfur containing side chains consists of cysteine and methionine. Exemplary conservative aa substitution groups are: valine-leucine-isoleucine, phenylalanine-tyrosine, lysine-arginine, alanine-valine-glycine, and asparagine-glutamine.

“T-cell” includes all types of immune cells expressing CD3, including T-helper cells (CD4⁺ cells), cytotoxic T-cells (CD8⁺ cells), T-regulatory cells (Treg) including CD8+T reg cells, and NK-T-cells.

Unless stated otherwise, as used herein, the terms “first major histocompatibility complex (MHC) polypeptide” or “first MHC polypeptide”, and the terms “second MHC polypeptide”, “MHC heavy chain”, and “MHC-H”, refer to MHC Class I receptor elements.

An immunomodulatory domain or “MOD” (also termed a co-immunomodulatory or co-stimulatory polypeptide), as the term is used herein, includes a polypeptide on an APC (e.g., a dendritic cell, a B cell, and the like), or a portion of the polypeptide on an APC, that specifically binds a “Co-MOD”(also termed a cognate co-immunomodulatory polypeptide or a cognate co-stimulatory polypeptide) on a T-cell, thereby providing a signal which, in addition to the primary signal provided by, for instance, binding of a TCR/CD3 complex with an MHC polypeptide loaded with peptide, mediates a T-cell response including, but not limited to, proliferation, activation, differentiation, and the like. MODs include, but are not limited to, CD7, B7-1 (CD80), B7-2 (CD86), PD-L1, PD-L2, 4-1BBL, OX40L, Fas ligand (FasL), inducible costimulatory ligand (ICOS-L), intercellular adhesion molecule (ICAM), transforming growth factor beta (TGF-β), CD30L, CD40, CD70, CD83, HVEM, lymphotoxin beta receptor, 3/TR6, ILT3, ILT4, HVEM, an agonist or antibody that binds the Toll ligand receptor, and a ligand that specifically binds with B7-H3. A MOD also encompasses, inter alia, an antibody (or an antigen binding portion thereof, such as a Fab) that specifically binds with a Co MOD present on a T-cell, such as, but not limited to, CD27, CD28, 4-1BB, OX40, CD30, CD40, PD-1, ICOS, lymphocyte function-associated antigen-1 (LFA-1), CD2, LIGHT, NKG2C, B7-H3, and a ligand that specifically binds to CD83.

An immunomodulatory domain of a T-Cell-MMP is a polypeptide of the T-Cell-MMP or part thereof that acts as a MOD.

“Heterologous,” as used herein, means a nucleotide or polypeptide that is not found in the native nucleic acid or protein, respectively.

“Recombinant,” as used herein, means that a particular nucleic acid (DNA or RNA) is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments, or from a series of synthetic oligonucleotides, to provide a synthetic nucleic acid which is capable of being expressed from a recombinant transcriptional unit contained in a cell or in a cell-free transcription and translation system.

The terms “recombinant expression vector” and “DNA construct” are used interchangeably herein to refer to a DNA molecule comprising a vector and at least one insert. Recombinant expression vectors are usually generated for the purpose of expressing and/or propagating the insert(s), or for the construction of other recombinant nucleotide sequences. The insert(s) may or may not be operably linked to a promoter sequence and may or may not be operably linked to DNA regulatory sequences.

As used herein, the term “affinity” refers to the equilibrium constant for the reversible binding of two agents (e.g., an antibody and an antigen) and is expressed as a dissociation constant (K_{D}). Affinity can be expressed as a fold increase (e.g., at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least q100-fold greater, or at least 1,000-fold greater, or more) relative to the affinity of a reference molecular interaction such as an antibody for a target aa sequence. As used herein, the term “avidity” refers to the resistance of a complex of two or more agents to dissociation after dilution. The terms “immunoreactive” and “preferentially binds” are used interchangeably herein with respect to antibodies and/or antigen-binding fragments.

“Binding” as used herein (e.g., with reference to binding of a molecule such as a T-cell-MMP comprising one or more MODs or its epitope conjugate to one or more polypeptides (e.g., a T-cell receptor and a Co-MOD on a T-cell) refers to a non-covalent interaction(s) between the molecules. Non-covalent binding refers to a direct association between two molecules due to, for example, electrostatic, hydrophobic, ionic, and/or hydrogen-bond interactions, including interactions such as salt bridges and water bridges. Non-covalent binding interactions are generally characterized by a dissociation constant (K_{D}) of less than 10⁻⁶ M, less than 10⁻⁷ M, less than 10⁻⁸ M, less than 10⁻⁹ M, less than 10⁻¹⁰ M, less than 10⁻¹¹ M, or less than 10⁻¹² M. “Affinity” refers to the strength of non-covalent binding, increased binding affinity being correlated with a lower K_{D}. “Specific binding” generally refers to, e.g., binding between a ligand molecule and its binding site or “receptor” with an affinity of at least about 10⁻⁷ M or greater (e.g., less than 5×10⁻⁷ M, less than 10⁻⁸ M, less than 5×10⁻⁸ M, less than 10⁻⁹ M, less than 10⁻¹⁰ M, less than 10⁻¹¹ M, or less than 10⁻¹¹ M and greater affinity, or in a range from 10⁻⁷ to 10⁻⁹ or from 10⁻⁹ to 10⁻¹²). In some contexts, e.g., binding between a TCR and a peptide/MHC complex, “specific binding” can be in the range of from 1 μM to 100 μM, or from 100 μM to 1 mM. “Covalent binding” as used herein means the formation of one or more covalent chemical bonds between two different molecules

The terms “treatment,” “treating” and the like are used herein to generally mean obtaining a desired pharmacologic and/or physiologic effect. The effect may be prophylactic in terms of completely or partially preventing a disease or symptom thereof, and/or may be therapeutic in terms of a partial or complete cure for a disease and/or adverse effect attributable to the disease. “Treatment” as used herein covers any treatment of a disease or symptom in a mammal and includes: (a) preventing the disease or symptom from occurring in a subject which may be predisposed to acquiring the disease or symptom but has not yet been diagnosed as having it; (b) inhibiting the disease or symptom, i.e., arresting its development; and/or (c) relieving the disease, i.e., causing regression of the disease. The therapeutic agent may be administered before, during and/or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

The terms “individual,” “subject,” “host,” and “patient” are used interchangeably herein and refer to any mammalian subject for whom diagnosis, treatment, or therapy is desired. Mammals include, e.g., humans, non-human primates, rodents (e.g., rats; mice), lagomorphs (e.g., rabbits), ungulates (e.g., cows, sheep, pigs, horses, goats, and the like), canines, felines, etc.

Before the present technology is further described, it is to be understood that this disclosure is not limited to the particular embodiments described, as such may, of course, vary. It is also to be understood that the terminology used herein is for the purpose of describing particular embodiments only, and is not intended to be limiting.

Where a range of values is provided, it is understood that the range includes each intervening value, to the tenth of the lower limit, unless the context clearly dictates otherwise, between the upper and lower limit of that range and any other stated or intervening value in that stated range, is encompassed within the disclosure. The upper and lower limits of smaller ranges may independently be included in the smaller ranges, and are also encompassed within the disclosure, subject to any specifically excluded limit in the stated range. Where a range includes upper and/or lower limits, ranges excluding either or both of those limits are also included in the disclosure.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, the preferred methods and materials are now described. All publications mentioned herein are incorporated herein by reference to disclose and describe the methods and/or materials in connection with which the publications are cited.

It must be noted that, as used herein and in the appended claims, the singular forms “a,” “an,” and “the” include plural referents unless the context clearly dictates otherwise. Thus, for example, reference to “multimeric T-cell modulatory polypeptide” includes a plurality of such polypeptides and reference to “the immunomodulatory polypeptide” or “the MOD” includes reference to one or more immunomodulatory polypeptides and equivalents thereof known to those skilled in the art, and so forth. It is further noted that the claims may be drafted to include or exclude any optional element. As such, this statement is intended to serve as antecedent basis for use of such exclusive terminology as “solely,” “only” and the like in connection with the recitation of claim elements or use of a “negative” limitation excluding any recited claim element.

It is appreciated that certain features of the technology, which are, for clarity, described in the context of separate embodiments, may also be provided in combination in a single embodiment. Conversely, various features of the technology, which are, for brevity, described in the context of a single embodiment, may also be provided separately or in any suitable sub-combination. All combinations of the embodiments pertaining to the technology are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

The publications discussed herein are provided solely for their disclosure prior to the filing date of the present application. Nothing herein is to be construed as an admission that the technology presented herein is not entitled to antedate such publication by virtue of prior invention. Further, the dates of publication provided may be different from the actual publication dates which may need to be independently confirmed.

### DETAILED DESCRIPTION

### I. T-Cell Modulatory Multimeric Polypeptides (T-Cell-MMPS) with Chemical Conjugation Sites for Epitope Binding

The present disclosure provides T-Cell-MMP-epitope conjugates that present a coronavirus epitope. Such T-Cell-MMP-epitope conjugates are useful for modulating the activity of T cells to, for example, modulate an immune response to a coronavirus in an individual.

The present disclosure also provides methods of T-Cell MMP preparation and use in modulating an immune response to a coronavirus in vitro, ex vivo, or in vivo in an individual that may be a human or non-human test subject or patient. The T-Cell-MMPs may comprise one or more independently selected wild-type and/or variant MOD polypeptides that exhibit reduced binding affinity to their Co-MODs and chemical conjugation sites for coupling epitopes and payloads. Included in this disclosure are T-Cell-MMPs that are heterodimeric, comprising two types of polypeptides (a first polypeptide and a second polypeptide), wherein at least one of those polypeptides comprises a chemical conjugation site for the attachment (e.g., covalent attachment) of payloads such as therapeutic agents and/or materials (e.g., fluorescent labels) that can bind a TCR. Also included in this disclosure are T-Cell-MMPs which have been chemically conjugated to an epitope and/or a payload (e.g., a therapeutic agent). Depending on the type of MOD(s) present in the T-Cell-MMP-epitope conjugate, when an epitope specific to a target T cell's TCR is present, the target T-cell can respond by undergoing activation including, for example, clonal expansion (e.g., when activating MODs such as IL-2, 4-1BBL and/or CD80 are incorporated into the T-Cell-MMP). Alternatively, the T-cell may undergo inhibition that down regulates T-cell activity when MODs such as FASL and/or PD-L1 are incorporated into the T-Cell-MMPs. Because MODs are not specific to any epitope, activation or inhibition of T-cells can be biased toward epitope-specific interactions by incorporating variant MODs having reduced affinity for their Co-MOD into the T-Cell-MMPs such that the binding of a T-Cell-MMP to a T-cell is strongly affected by, or even dominated by, the MHC-epitope-TCR interaction.

A T-Cell-MMP-epitope conjugate may be considered to function as a surrogate APC, and mimic the adaptive immune response. The T-Cell-MMP-epitope conjugate does so by engaging a TCR present on the surface of a T-cell with a covalently bound epitope presented in the T-Cell-MMP-epitope conjugate complex. This engagement provides the T-Cell-MMP-epitope conjugate with the ability to achieve epitope-specific cell targeting. In embodiments described herein, T-Cell-MMP-epitope conjugates also possess at least one MOD that engages a counterpart costimulatory protein (Co-MOD) on the T-cell. Both signals—epitope/MHC binding to a TCR and MOD binding to a Co-MOD—then drive both the desired T-cell specificity and either inhibition or activation/proliferation.

T-Cell-MMPs having chemical conjugation sites find use as a platform into which different epitopes and/or payloads may be inserted to prepare materials for therapeutic, diagnostic and research applications, and in this case, specifically coronavirus epitopes. Such T-Cell-MMPs comprising a chemical conjugation site permit the rapid preparation of diagnostics and therapeutics as they permit the coronavirus epitope containing material (e.g., a peptide) to be rapidly inserted into the T-Cell-MMP and tested for activation or inhibition of T-cells bearing TCRs specific to the epitope.

In an embodiment, a chemical conjugation site of such a T-Cell-MMP may be utilized to attach a payload such as an antiviral agent or enzyme to the T-Cell-MMP.

In an embodiment, where variant MODs that stimulate T-cell proliferation and an epitope are incorporated into a T-Cell-MMP, contacting the T-cells with at least one concentration of the T-Cell-MMP induces at least a twofold (e.g., at least a 2, 3, 4, 5, 10, 20, 30, 50, 75, or 100 fold) difference in the activation of T-cells (as measured by T-cell proliferation or ZAP-70 activity, see e.g., Wang, et al., Cold Spring Harbor perspectives in biology 2.5 (2010): a002279) having a TCR specific to the epitope, as compared to T-cells contacted with the same concentration of the T-Cell-MMP that do not have a TCR specific to the epitope.

In an embodiment where variant MODs that inhibit T-cell activation and a coronavirus epitope are incorporated into a T-Cell-MMP, contacting the T-cells with at least one concentration of the T-Cell-MMP prevents activation of T-cells in an epitope specific manner as measured by T-cell proliferation.

The specificity of T-Cell-MMP-epitope conjugates depends on the relative contributions of the epitope and MODs to the binding. Where the MODs dominate the T-Cell-MMPs in the binding interactions, the specificity of the T-Cell-MMP epitope conjugates will be reduced relative to T-Cell-MMP complexes where the epitope dominates the binding interactions by contributing more to the overall binding energy than the MODs. The greater the contribution of binding energy between an epitope and a TCR specific to the epitope, the greater the specificity of the T-Cell-MMP will be for the T-cell bearing that type of TCR. Where an epitope has strong affinity for its TCR, the use of variant MODs with reduced affinity for their Co-MODs will favor epitope selective interactions of the T-Cell-MMP-epitope conjugates, and also facilitate selective delivery of any payload that may be conjugated to the T-Cell-MMP-epitope conjugate.

The present disclosure provides T-Cell-MMP epitope conjugates presenting coronavirus epitopes that are useful for modulating the activity of T-cells in an epitope specific manner and, accordingly, for modulating an immune response to coronaviruses (e.g., SARS-CoV or SARS-CoV-2) in an individual. Such T-Cell-MMPs may comprise a MOD that exhibits reduced binding affinity to a Co-MOD.

A. T-Cell-MMPs and T-Cell-MMP Epitope Conjugates

The T-Cell-MMP frameworks described herein comprise at least one chemical conjugation site on either the first polypeptide chain or the second polypeptide chain.

In an embodiment, the present disclosure provides a T-Cell-MMP comprising a heterodimer comprising: a) a first polypeptide comprising: a first MHC polypeptide; b) a second polypeptide comprising a second MHC polypeptide; c) at least one of first or second polypeptides comprises a chemical conjugation site, and d) at least one MOD, where the first and/or the second polypeptide comprises the at least one MOD (e.g., one, two, three, or more). Optionally, the first or the second polypeptide comprises an Ig Fc polypeptide or a non-Ig scaffold. One or more of the MODs, which are selected independently, may be a variant MOD that exhibits reduced affinity to a Co-MOD compared to the affinity of a corresponding wild-type MOD for the Co-MOD. The disclosure also provides T-Cell-MMPs in which an epitope (e.g., a peptide bearing an epitope) is covalently bound (directly or indirectly) to the chemical conjugation site forming a T-Cell-MMP-epitope conjugate. In such an embodiment, the epitope (e.g., epitope peptide) present in a T-Cell-MMP-epitope conjugate of the present disclosure may bind to a T-cell receptor (TCR) on a T-cell with an affinity of at least 100 micro molar (M) (e.g., at least 10 μM, at least 1 μM, at least 100 nM, at least 10 nM, or at least 1 nM). A T-Cell-MMP-epitope conjugate may bind to a first T-cell with an affinity that is at least 25% higher than the affinity with which the T-Cell-MMP-epitope conjugate binds to a second T-cell, where the first T-cell expresses on its surface the Co-MOD and a TCR that binds the epitope with an affinity of at least 100 μM, and where the second T-cell expresses on its surface the Co-MOD but does not express on its surface a TCR that binds the epitope with an affinity of at least 100 μM (e.g., at least 10 μM, at least 1 μM, at least 100 nM, at least 10 nM, or at least 1 nM).

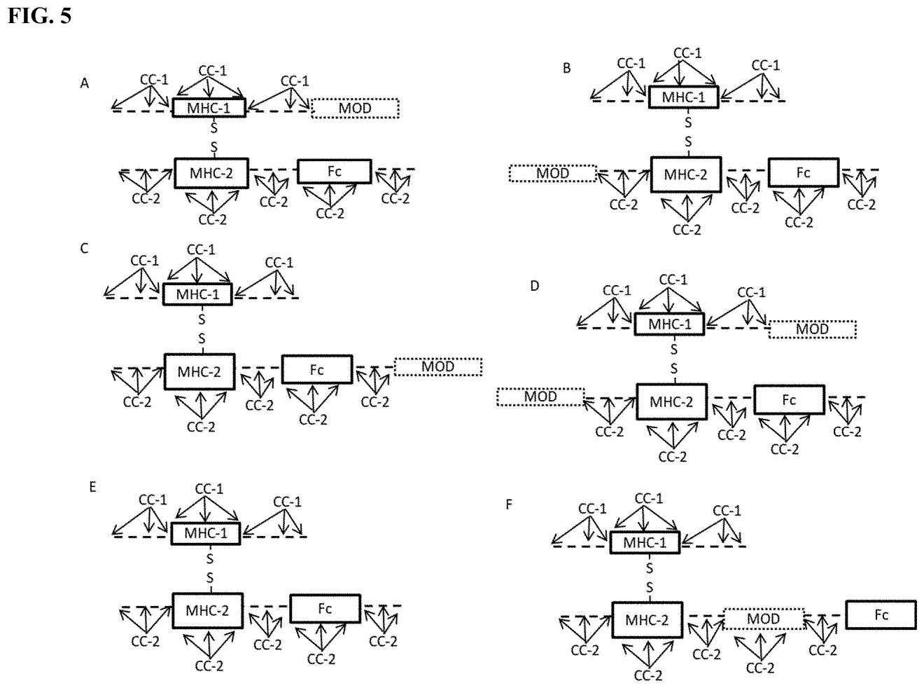

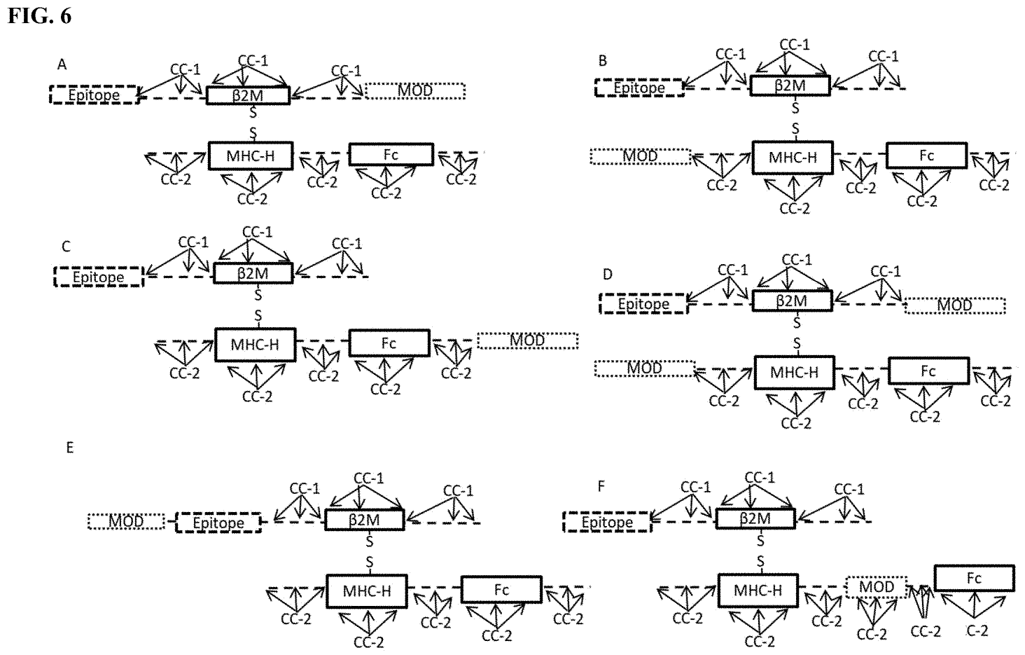

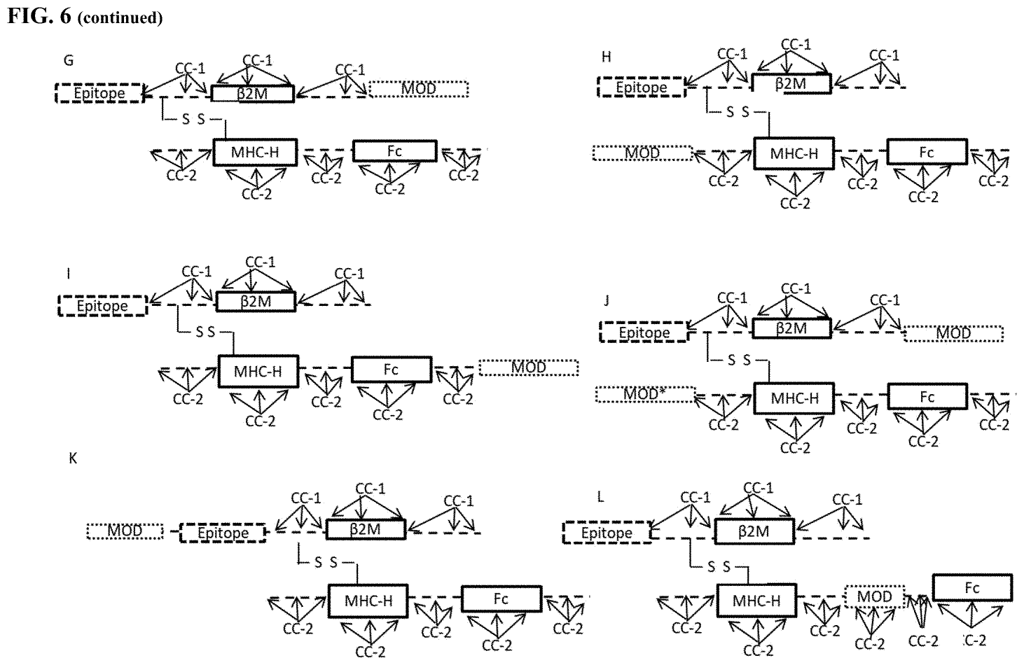

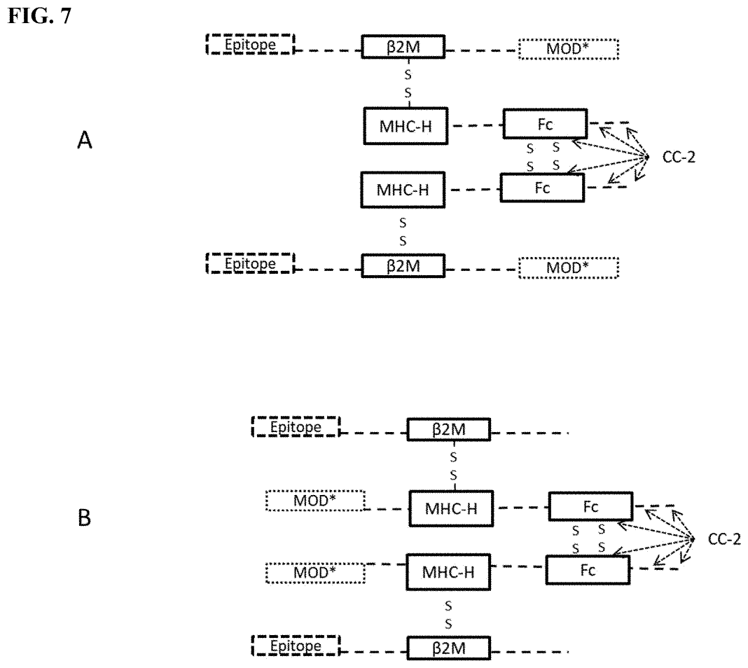

In an embodiment, the present disclosure provides a heterodimeric T-Cell-MMP (which may form higher level multimers, dimers, trimers, etc. of the heterodimers) comprising:
a) a first polypeptide comprising a first MHC polypeptide;
b) a second polypeptide comprising, in order from N-terminus to C-terminus: i) a second MHC polypeptide and ii) an optional immunoglobulin (Ig) Fc polypeptide scaffold or a non-Ig polypeptide scaffold;
c) one or more first polypeptide chemical conjugation sites attached to or within the first polypeptide, and/or one or more second polypeptide chemical conjugation sites attached to or within the second polypeptide; and
d) one or more immunomodulatory polypeptides (MODs), wherein at least one of the one or more MODs is
   A) at the C-terminus of the first polypeptide (see, e.g., A in FIG. 5 or 6),
   B) at the N-terminus of the second polypeptide (see, e.g., B in FIG. 5 or 6),
   C) at the C-terminus of the second polypeptide (see, e.g., C in FIG. 5 or 6), or
   D) at the C-terminus of the first polypeptide and at the N-terminus of the second polypeptide (see, e.g., D in FIG. 5 or 6);

wherein each of the one or more MODs is an independently selected wild-type or variant MOD.

Such T-Cell-MMP frameworks act as a platform on which coronavirus epitopes (e.g., peptide, phosphopeptide, or glycopeptide epitopes such as those from a spike glycoprotein, nucleoprotein, membrane protein, replicase protein, non-structural protein (nsp) and the like) can be covalently attached through a linkage to one of the first or second chemical conjugation sites bound to at least one of the first and second MHC polypeptides forming a T-Cell-MMP-epitope conjugate. This permits facile introduction of different epitopes into the framework for presentation in the context of the T-Cell-MMP to a T-cell receptor (TCR) on a T-cell. Payload (e.g., antivirals) can similarly be attached to a T-Cell-MMP by covalent attachment to one of the first or second chemical conjugation sites (e.g., a site not employed for attachment of an epitope).

Where an immunoglobulin (Ig) Fc polypeptide or a non-Ig polypeptide scaffold that can multimerize is employed, the T-Cell-MMPs may multimerize. The complexes may be in the form of dimers (see, e.g., FIG. 7), trimers, tetramers, or pentamers. Compositions comprising multimers of T-Cell-MMPs may also comprise monomers and, accordingly. may comprise monomers, dimers, trimers, tetramers, pentamers, or combinations of any thereof (e.g., a mixture of monomers and dimers).

In an embodiment, the MODs are independently selected wild-type MODs and/or variant MODs present in a T-Cell-MMP or its epitope conjugate. In an embodiment, the MODs are one or more wt MODs and/or variant MODs capable of stimulating epitope-specific T-cell activation/proliferation (e.g., IL-2, 4-1BBL and/or CD80). In another embodiment, the MODs are one or more wt MODs and/or variant MODs capable of inhibiting T-cell activation/proliferation or otherwise suppressing cytotoxic T cells. (e.g., FAS-L and/or PD-L1). When used in conjunction with a T-Cell-MMP bearing a suitable epitope, such activating or inhibitory MODs are capable of epitope-specific T-cell action, particularly where the MODs are variant MODs and the MHC-epitope-TCR interaction is sufficiently strong to dominate the interaction of the T-Cell-MMP with the T-cells.

1 Locations of the First and Second Chemical Conjugation Sites in T-Cell-MMPs

Prior to being subject to chemical conjugation reactions that incorporate an epitope (e.g., an epitope containing peptide) and/or payload, the unconjugated T-Cell-MMPs described herein comprise at least one chemical conjugation site. Where the T-Cell-MMPs comprise more than one chemical conjugation site, there may be two or more conjugation sites on the first polypeptide (first polypeptide chemical conjugation sites), two or more conjugation sites on the second polypeptide (second polypeptide chemical conjugation sites), or at least one first polypeptide chemical conjugation site and at least one second polypeptide chemical conjugation site. In each instance where more than one chemical conjugation site is present in a T-Cell-MMP molecule, the sites are independently selected and may employ the same or different chemistries, amino acid sequences, or chemical groups for conjugation. Some examples of the locations for first polypeptide chemical conjugation sites (indicated as CC-1) and second polypeptide chemical conjugation sites (indicated as CC-1) are shown in FIGS. 5-7.

In embodiments, the first polypeptide of the T-Cell-MMPs comprise: a first MHC polypeptide without a linker on its N-terminus and C-terminus; a first MHC polypeptide bearing a linker on its N-terminus; a first MHC polypeptide bearing a linker on its C-terminus, or a first MHC polypeptide bearing a linker on its N-terminus and C-terminus. At least one of the one or more first polypeptide chemical conjugation sites is: a) attached to (e.g., at the N- or C-terminus), or within, the sequence of the first MHC polypeptide when the first MHC polypeptide is without a linker on its N- and C-termini; b) attached to, or within, the sequence of the first MHC polypeptide, where the first MHC polypeptide comprises a linker on its N- and C-termini; c) attached to, or within, the sequence of a linker on the N-terminus of the first MHC polypeptide; and/or d) attached to, or within, the sequence of a linker on the C-terminus of the first MHC polypeptide. Additional first polypeptide chemical conjugation sites of a T-Cell-MMP may be present at (attached to or within) any location on the first polypeptide (e.g., more than one enzyme modification sequence serving as a site for chemical conjugation), including the first MHC polypeptide, or in any linker attached to the MHC peptide. In such embodiments, the first MHC polypeptide may comprise a β2M polypeptide sequence as described below.

In embodiments, the second polypeptide of the T-Cell-MMPs comprise: a second MHC polypeptide without a linker on its N-terminus and C-terminus; a second MHC polypeptide bearing a linker on its N-terminus; a second MHC polypeptide bearing a linker on its C-terminus, or a second MHC polypeptide bearing a linker on its N-terminus and C-terminus. At least one of the one or more second polypeptide chemical conjugation sites is: a) attached to (e.g., at the N- or C-terminus), or within, the sequence of the second MHC polypeptide when the second MHC polypeptide is without a linker on its N- and C-termini; b) attached to, or within, the sequence of the second MHC polypeptide where the second MHC polypeptide comprises a linker on its N- and C-termini; c) attached to, or within, the sequence of the linker on the N-terminus of the second MHC polypeptide; and/or d) attached to, or within, the sequence of the linker on the C-terminus of the second MHC polypeptide. In addition, when the second polypeptide contains an immunoglobulin (Fc) polypeptide aa sequence or a non-Ig polypeptide scaffold, along with an additional linker attached thereto, the second polypeptide chemical conjugation sites may be attached to or within the second MHC polypeptide, the immunoglobulin polypeptide, the polypeptide scaffold, or the attached linker. Additional second polypeptide chemical conjugation sites of a T-Cell-MMP may be present at (attached to or within) any location on the second polypeptide (e.g., more than one enzyme modification sequence serving as a site for chemical conjugation), including the second MHC polypeptide, or in any linker attached to it. In such embodiments, the second MHC polypeptide may comprise an MHC heavy chain (MHC-H) polypeptide sequence as described below.

In an embodiment, the first and second MHC polypeptides may be selected to be Class I MHC polypeptides, with the first MHC polypeptide comprising a β2M polypeptide sequence and the second polypeptide comprising an MHC heavy chain sequence, wherein there is at least one chemical conjugation site on the first or second polypeptide. In an embodiment, at least one of the one or more first chemical conjugation sites in the T-Cell-MMP may be attached to (including at the N- or C-terminus) or within either the β2M polypeptide or the linker attached to its N-terminus or C-terminus. In an embodiment, at least one of the one or more second polypeptide chemical conjugation sites in the T-Cell-MMP may be attached to (including at the N- or C-terminus) or within: the MHC-H polypeptide; a linker attached to the N-terminus or C-terminus of the MHC-H polypeptide; or, when present, attached to or within an immunoglobulin (Fc) polypeptide (or a non-Ig polypeptide scaffold) or a linker attached thereto. In another embodiment of such a Class I MHC polypeptide construct, both the first and second polypeptides comprise at least one chemical conjugation site.

The β2M polypeptide sequence of a T-Cell-MMP may have at least 85% aa sequence identity (e.g., at least 90%, 95%, 98% or 99% identity, or even 100% identity) to one of the aa sequences set forth in FIG. 4. The β2M polypeptide may comprise an aa sequence having at least 85% aa sequence identity (e.g., at least 90%, 95%, 98% or 99% identity, or even 100% identity) to at least 20, 30, 40, 50, 80, 90, 100, or 110 contiguous aas of an aa sequence set forth in FIG. 4. The chemical conjugation sequences/sites can be attached to the β2M polypeptide (e.g., at the N- and/or C-termini or to linkers attached thereto) or within the β2M polypeptide.

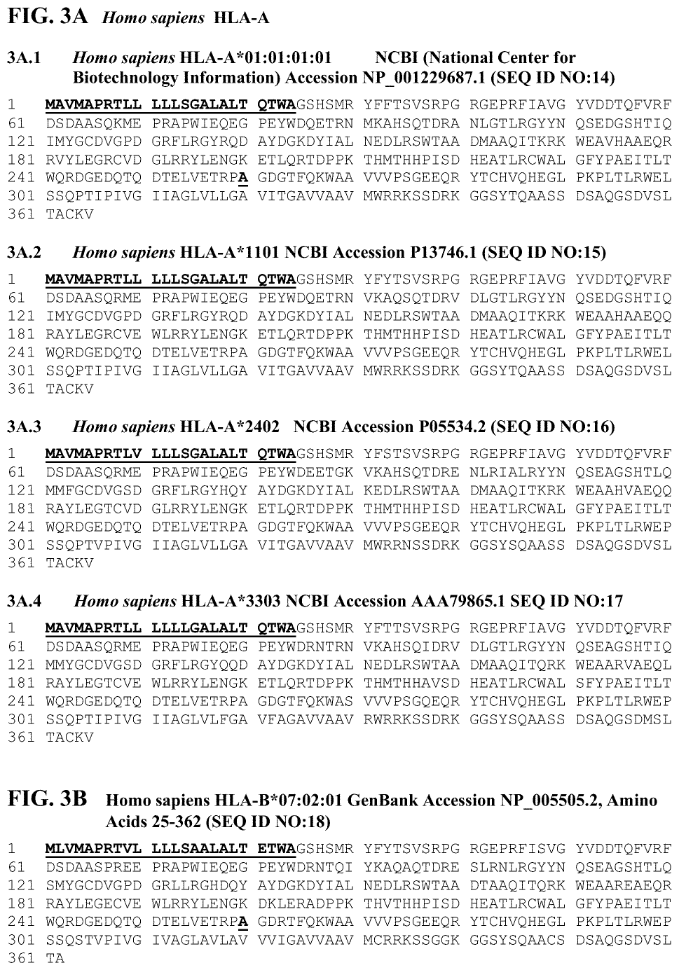

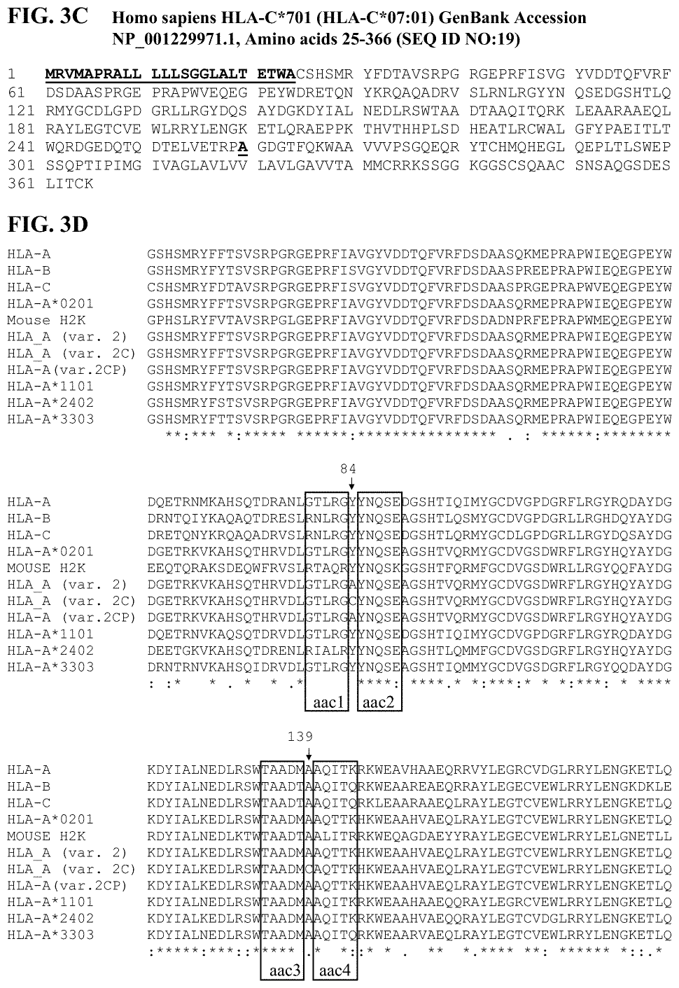

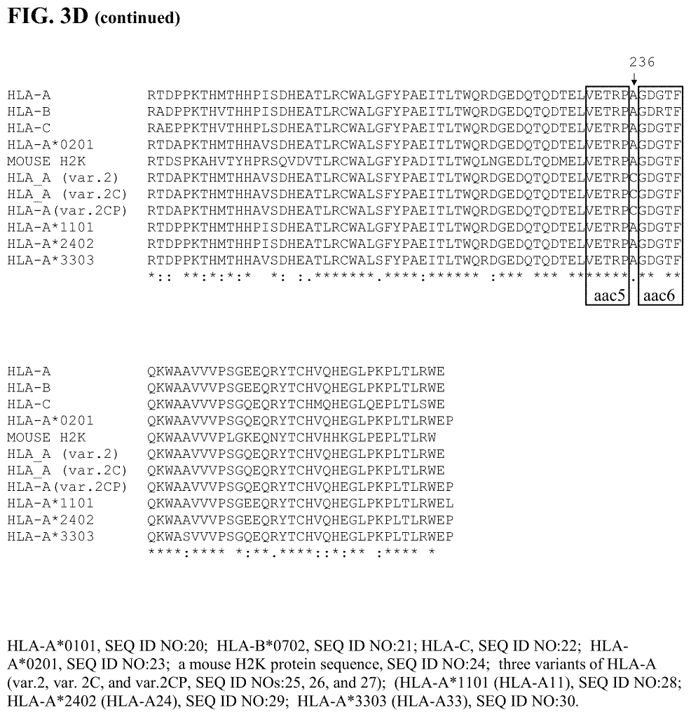

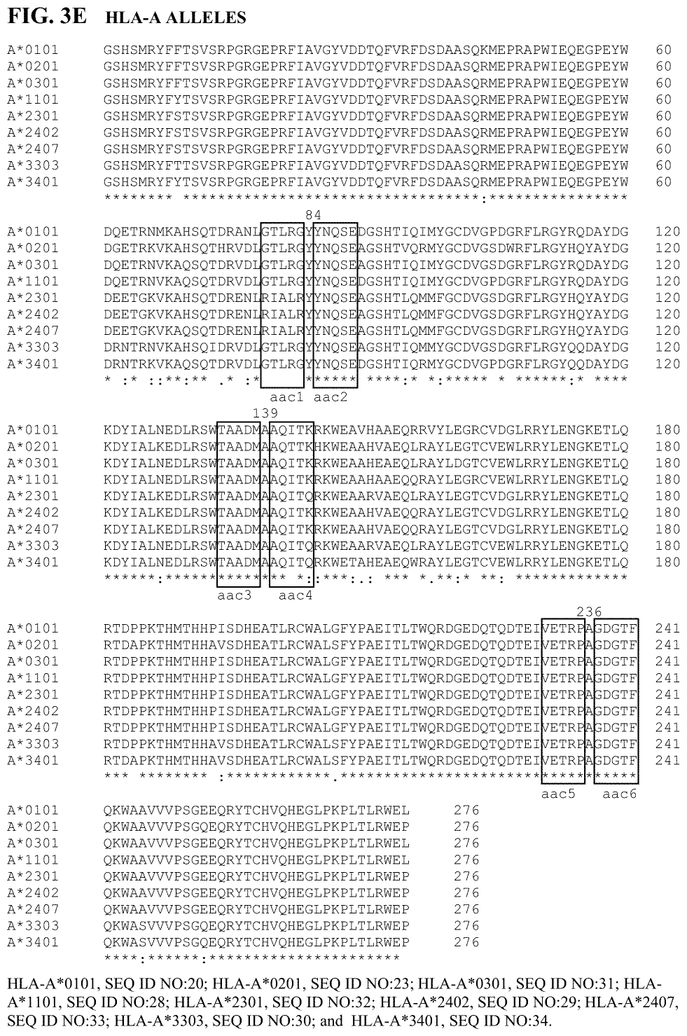

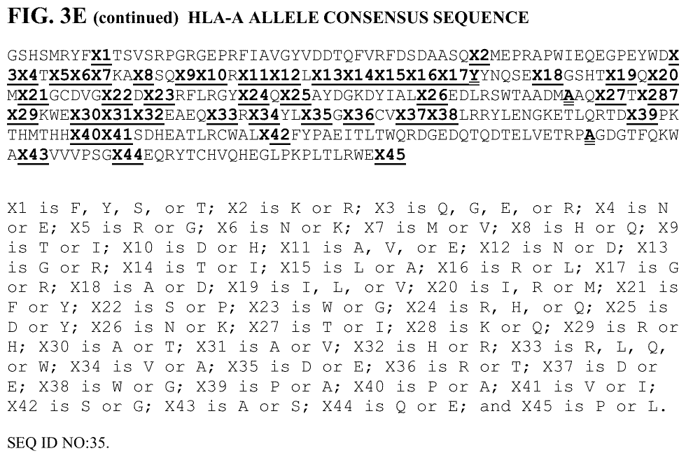

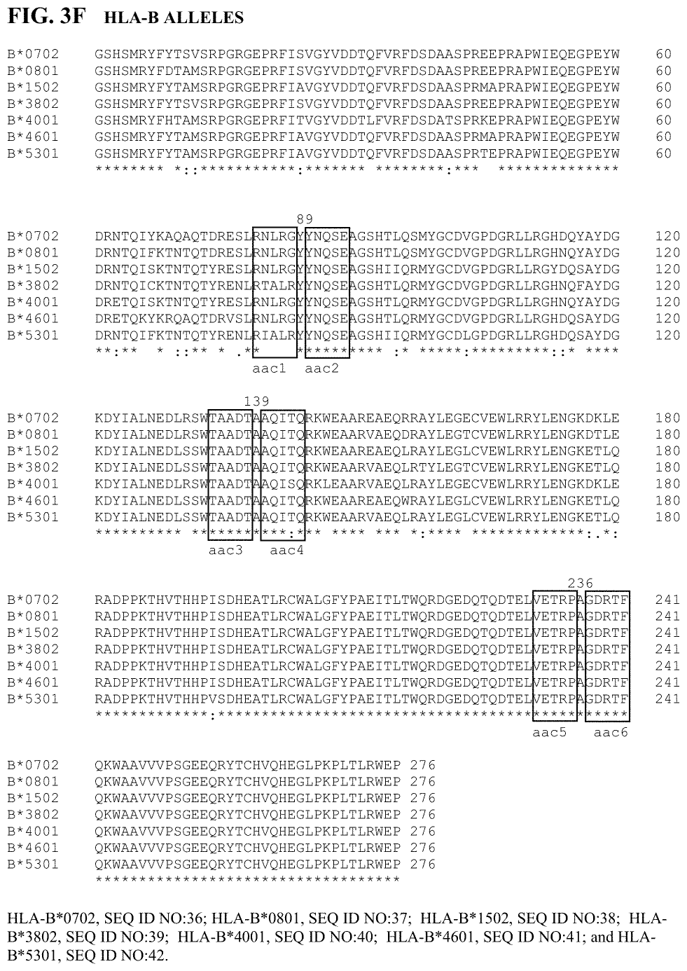

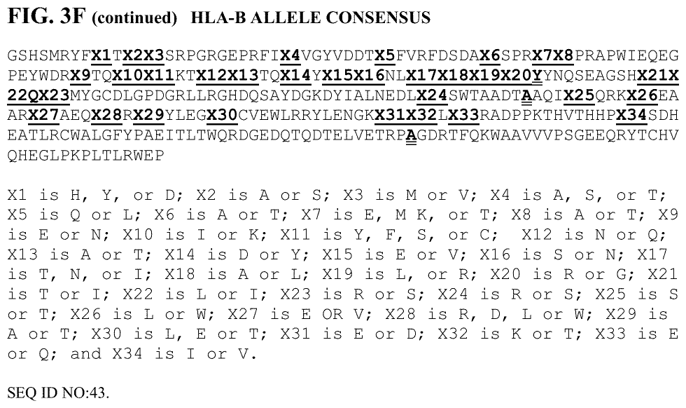

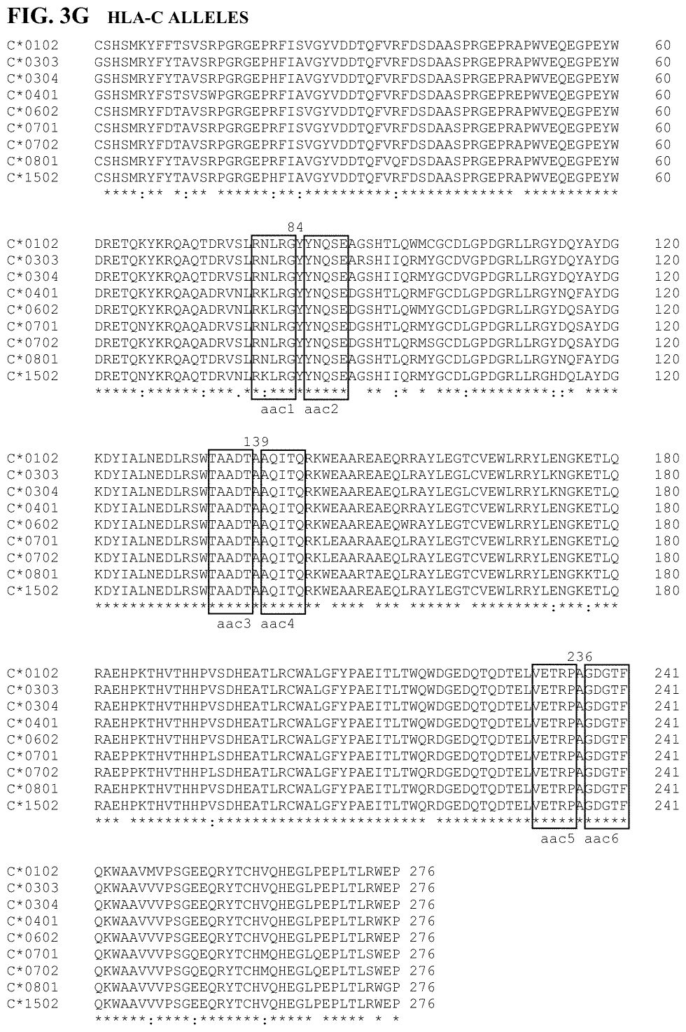

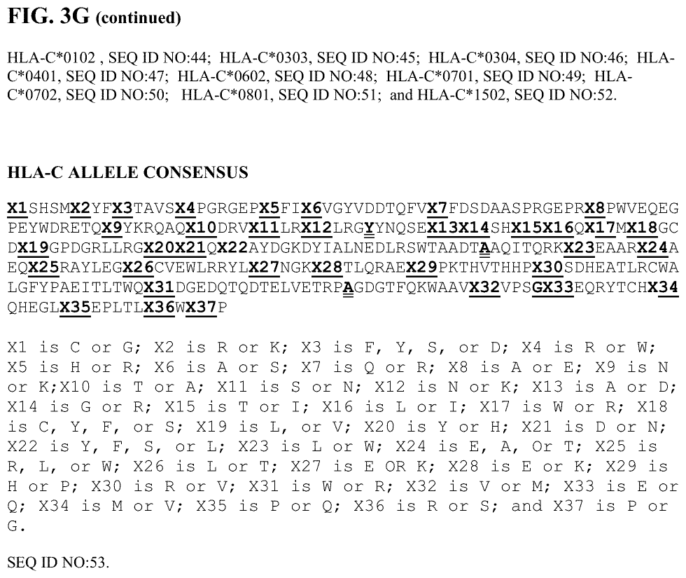

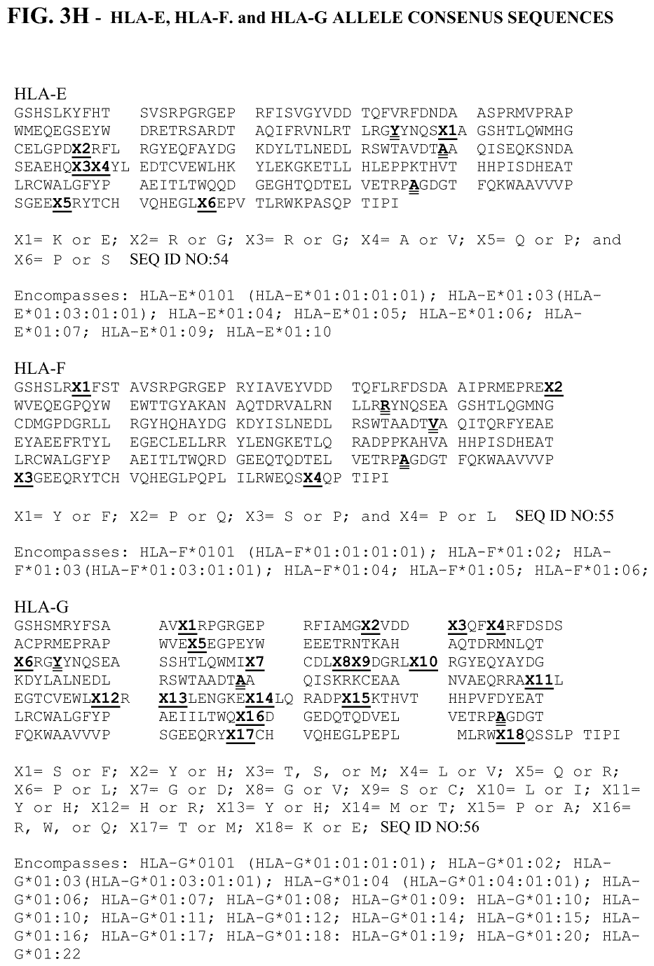

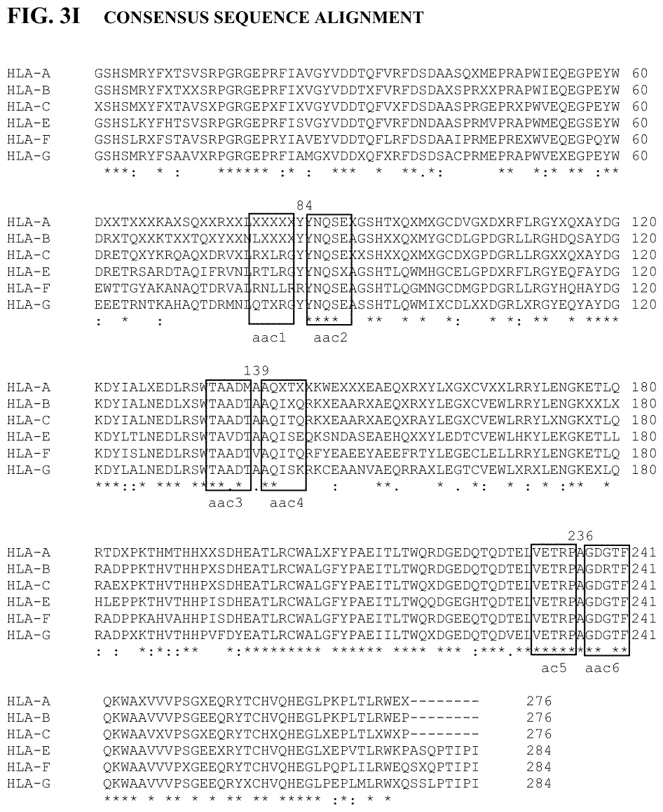

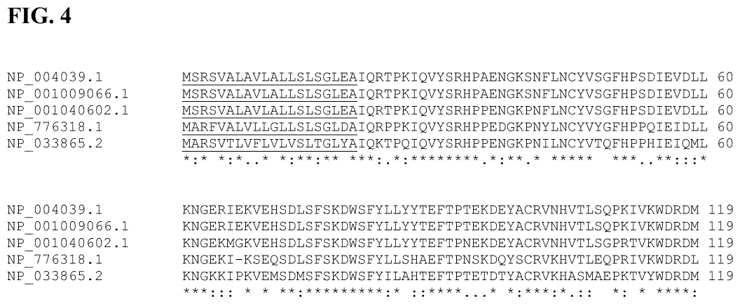

The MHC-H polypeptide of a T-Cell-MMP may be an HLA-A, -B, -C, -E, -F, or -G heavy chain polypeptide sequence. In an embodiment, the MHC-H polypeptide may comprise an aa sequence having at least 85% aa sequence identity (e.g., at least 90%, 95%, 98% or 99% identity, or even 100% identity) to the aa sequence set forth in one of FIGS. 3A-3H. The MHC Class I heavy chain polypeptides may comprise an aa sequence having at least 85% aa sequence identity (e.g., at least 90%, 95%, 98% or 99% identity, or even 100% identity) to at least 20, 30, 40, 50, 80, 100, 150, 200, 250, 260, 270, 300, or 330 contiguous aas with identity to a portion of an aa sequence set forth in FIGS. 3A-3H. The chemical conjugation sequences can be attached (e.g., at the N- and/or C-termini or linkers attached thereto) or within the MHC-H polypeptides.

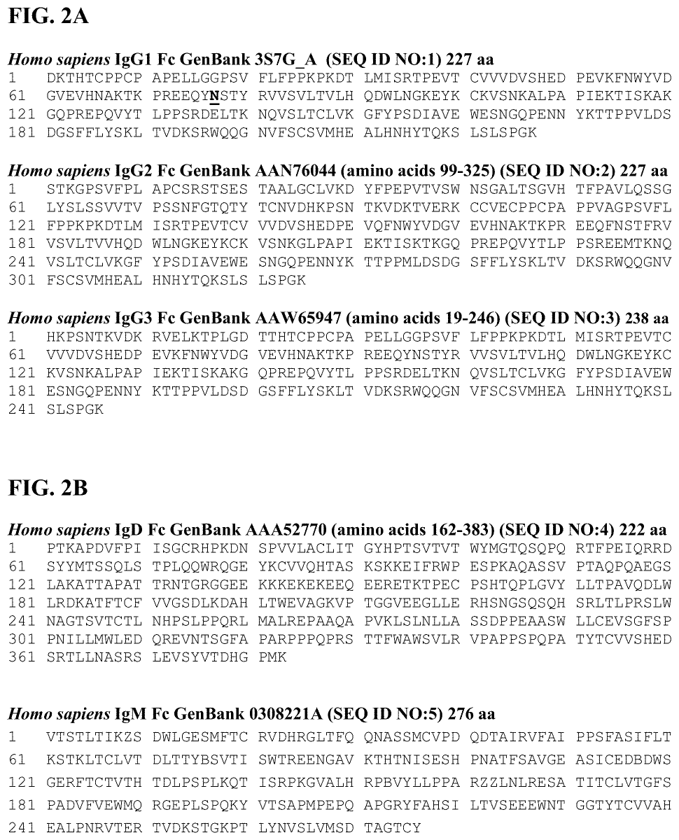

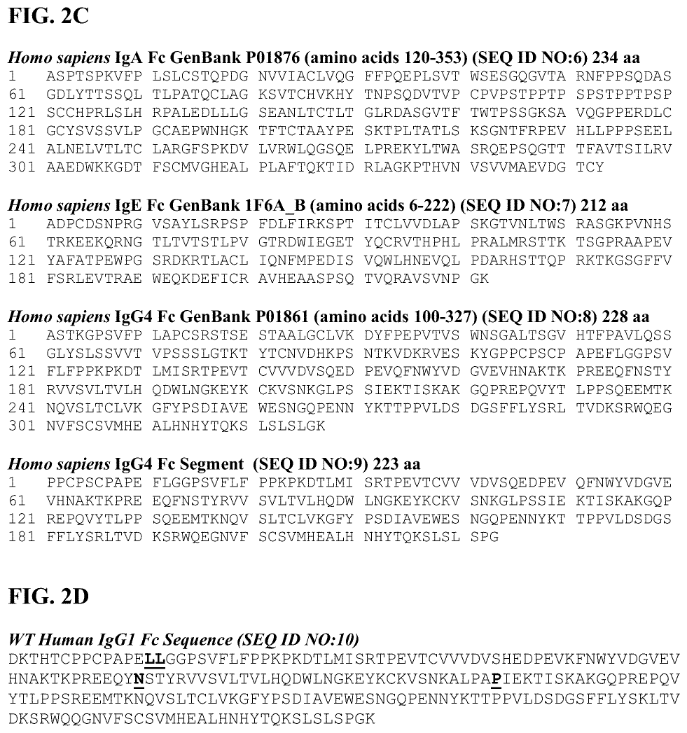

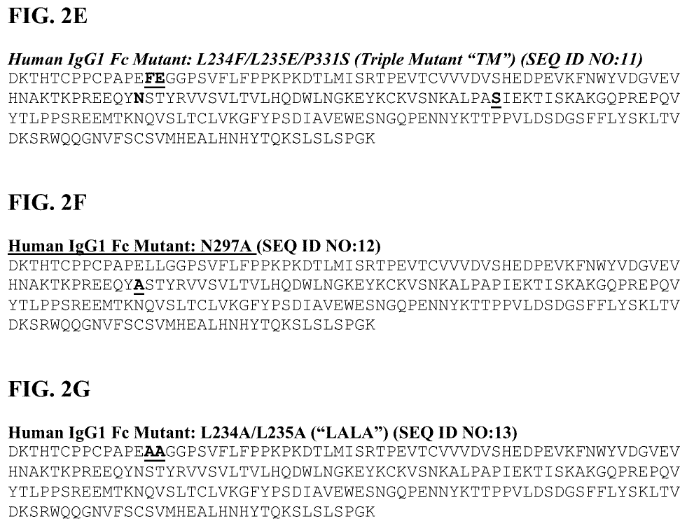

The second polypeptide of the T-Cell-MMP may comprise an Ig Fc polypeptide sequence that can act as part of a molecule scaffold providing structure and the ability to multimerize to the T-Cell-MMP (or its epitope conjugate) and, in addition, potential locations for chemical conjugation. In some embodiments the Ig Fc polypeptide is an IgG1 Fc polypeptide, an IgG2 Fc polypeptide, an IgG3 Fc polypeptide, an IgG4 Fc polypeptide, an IgA Fc polypeptide, or an IgM Fc polypeptide. In such embodiments the Ig Fc polypeptide may comprise an aa sequence that has at least 85%, 90%, 95%, 98, or 99%, or even 100%, aa sequence identity to an aa sequence depicted in one of FIGS. 2A-2G. Ig Fc polypeptides may comprise a sequence having at least 20, 30, 40, 50, 60, 80, 100, 120, 140, 160, 180, 200, or 220 contiguous aas with identity to a portion of an aa sequence in any of FIGS. 2A-2G. In an embodiment where the second polypeptide comprises an IgG1 Fc polypeptide, the polypeptide may also comprise one or more aa substitutions selected from N297A, L234A, L235A, L234F, L235E, and P331S. In one such embodiment, the IgG1 Fc polypeptide comprises L234A and L235A substitutions either alone or in combination with a second polypeptide chemical conjugation site. The chemical conjugation sites can be located/attached at the N- and/or C-termini or to linkers attached thereto, or within the Ig Fc polypeptides.

2 Chemical Conjugation Sites of Unconjugated T-CeIl-MMPs

The first and second polypeptide chemical conjugation sites of the T-Cell-MMPs may be any suitable site that can be modified upon treatment with a reagent and/or catalyst such as an enzyme that permits the formation of a covalent linkage to either one or both of the T-Cell-MMP polypeptides. In an embodiment, there is only one chemical conjugation site that has been introduced into either the first or second polypeptide of a T-Cell-MMP. In an embodiment, each first and second polypeptide chemical conjugations sites are selected to be either the sane or different types of chemical conjugation sites, thereby permitting the same or different molecules to be selectively conjugated to each of the polypeptides. In another embodiment, each first and second polypeptide chemical conjugation site is selected such that they are different types of conjugation site on the respective polypeptides, permitting different molecules to be selectively conjugated to each of the polypeptides. In other embodiments, such as where both an epitope molecule and one or more payload molecules are to be incorporated into a T-Cell-MMP, more than one copy of a first and/or a second polypeptide chemical conjugation may be introduced into the T-Cell-MMP. For example, a T-Cell-MMP may have one first polypeptide chemical conjugation site (e.g., for conjugating an epitope) and multiple second polypeptide chemical conjugation sites for delivering molecules of payload (or vice versa).

In embodiments, the first and second chemical conjugation sites may be selected independently from:
a) peptide sequence attached to or within the first or second polypeptide that acts as an enzyme modification sequence (e.g., sulfatase, sortase, and/or transglutaminase sequences);
b) non-natural aas and/or selenocysteines attached to or within the first or second polypeptide;
c) engineered aa chemical conjugation sites;
d) carbohydrate or oligosaccharide moieties attached to the first or second polypeptide; and
e) IgG nucleotide binding sites attached to or within the first or second polypeptide.

a. Sulfatase Motifs

In those embodiments where enzymatic modification is chosen as the means of chemical conjugation, at least one of the one or more first and second chemical conjugation sites may comprise a sulfatase motif. Sulfatase motifs are usually 5 or 6 aas in length, and are described, for example, in U.S. Pat. No. 9,540,438 and U.S. Pat. Pub. No. 2017/0166639 A1, which are incorporated by reference. Insertion of the motif results in the formation of a protein or polypeptide that is sometimes referred to as aldehyde tagged or having an aldehyde tag. The motif may be acted on by formylglycine generating enzyme(s) (“FGE” or “FGEs”) to convert a cysteine or serine in the motif to a formylglycine residue (“fGly” although sometimes denoted “FGly”), which is an aldehyde containing aa that may be utilized for selective (e.g., site specific) chemical conjugation reactions. Accordingly, as used herein, “aldehyde tag” or “aldehyde tagged” polypeptides refer to an aa sequence comprising an unconverted sulfatase motif, as well as to an aa sequence comprising a sulfatase motif in which the cysteine or the serine residue of the motif has been converted to fGly by action of an FGE. In addition, where a sulfatase motif is provided in the context of an aa sequence, both the as sequence (e.g., polypeptide) containing the unconverted motif as well as its fGly containing counterpart are disclosed. Once incorporated into a polypeptide (e.g., of a T-Cell-MMP), a fGly residue may be reacted with molecules (e.g., epitope peptides) comprising a variety of reactive groups including, but not limited to, thiosemicarbazide, aminooxy, hydrazide, and hydrazino groups to form a conjugate (e.g., a T-Cell-MMP-epitope conjugate) having a covalent bond between the peptide and the molecule via the fGly residue. Sulfatase motifs may be used to incorporate not only epitopes (e.g., epitope presenting peptides), but also to incorporate payloads (e.g., in the formation of conjugates with drugs and diagnostic molecules such as labels).

In embodiments, the sulfatase motif is at least 5 or 6 as residues, but can be, for example, from 5 to 16 (e.g., 6-16, 5-14, 6-14, 5-12, 6-12, 5-10, 6-10, 5-8, or 6-8) aas in length. The sulfatase motif may be limited to a length less than 16, 14, 12, 10, or 8 as residues.

In an embodiment, the sulfatase motif contains the sequence shown in Formula (I):

X1Z1X2Z2X3Z3(SEQ ID NO:62), where
Z1 is cysteine or serine;
Z2 is either a proline or alanine residue (which can also be represented by “P/A”);
Z3 is a basic aa (arginine, lysine, or histidine, usually lysine), or an aliphatic aa (alanine, glycine, leucine, valine, isoleucine, or proline, usually A, G, L, V. or I);
X1 is present or absent and, when present, can be any aa, though usually an aliphatic aa, a sulfur-containing aa, or a polar uncharged an (e.g., other than am aromatic an or a charged aa), usually L, M, V, S or T, more usually L, M, S or V, with the proviso that, when the sulfatase motif is at the N-terminus of the target polypeptide, X1 is present; and
X2 and X3 independently can be any aa, though usually an aliphatic aa, a polar, uncharged aa, or a sulfur containing an (e.g., other than an aromatic an or a charged aa), usually S, T, A, V, G or C, more usually S, T, A, V or G.

As indicated above, a sulfatase motif of an aldehyde tag is at least 5 or 6 aa residues, but can be, for example, from 5 to 16 aas in length. The motif can contain additional residues at one or both of the N- and C-termini, such that the aldehyde tag includes both a sulfatase motif and an “auxiliary motif.” In an embodiment, the sulfatase motif includes a C-terminal auxiliary motif (i.e., following the Z3 position of the motif).

A variety of FGEs may be employed for the conversion (oxidation) of cysteine or serine in a sulfatase motif to fGly. As used herein, the term formylglycine generating enzyme, or FGE, refers to fGly-generating enzymes that catalyze the conversion of a cysteine or serine of a sulfatase motif to fGly. As discussed in U.S. Pat. No. 9,540,438, the literature often uses the term formylglycine-generating enzymes for those enzymes that convert a cysteine of the motif to fGly, whereas enzymes that convert a serine in a sulfatase motif to fGly are referred to as Ats-B-like.

Sulfatase motifs of Formula (I) amenable to conversion by a prokaryotic FGE often contain a cysteine or serine at Z1 and a proline at Z2 that may be modified either by the “SUMP I-type” FGE or the “AtsB-type” FGE, respectively. Prokaryotic FGE enzymes that may be employed include the enzymes from Clostridium perfringens (a cysteine type enzyme), Klebsiella pneumoniae (a Serine-type enzyme) or the FGE of Mycobacterium tuberculosis. Where peptides containing a sulfatase motif are being prepared for conversion into fGly-containing peptides by a eukaryotic FGE, for example by expression and conversion of the peptide in a eukaryotic cell or cell free system using a eukaryotic FGE, sulfatase motifs amenable to conversion by a eukaryotic FGE may advantageously be employed.

Host cells for production of polypeptides with unconverted sulfatase motifs, or where the cell expresses a suitable FGE for converting fGly-containing polypeptide sequences, include those of a prokaryotic and eukaryotic organism. Non-limiting examples include Escherichia coli strains, Bacillus spp. (e.g., B. subtilis, and the like), yeast or fungi (e.g., S. cerevisiae, Pichia spp., and the like). Examples of other host cells, including those derived from a higher organism such as insects and vertebrates, particularly mammals, include, but are not limited to, CHO cells, HEK cells, and the like (e.g., American Type Culture Collection (ATCC) No. CCL-2), CHO cells (e.g., ATCC Nos. CRL %18 and CRL90%), CHO DG44 cells, CHO-Ki cells (ATCC CCL-61), 293 cells (e.g., ATCC No. CRL-1573), Vero cells, NIH 3T3 cells (e.g., ATCC No. CRL-1658), Hnh-7 cells, BHK cells (e.g., ATCC No. CCLlO), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, and the like.

Sulfatase motifs may be incorporated into any desired location on the first or second polypeptide of the T-Cell-MMP (or its epitope conjugate). In an embodiment, a sulfatase motif may be added at or near the terminus of any element in the first or second polypeptide of the T-Cell-MMP (or its epitope conjugate), including the first and/or second MHC polypeptides (e.g., MHC-H and/or β2M polypeptides), the scaffold or Ig Fc, and the linkers adjoining those elements. Accordingly, the sulfatase motif may be linked to an aa in the N-terminal region of β2M (with or without a linker).

In an embodiment a sulfatase motif is incorporated into, or attached to (e.g., via a peptide linker), a T-Cell-MMP (or its epitope conjugate) in a first or second polypeptide that has a β2M polypeptide with a sequence having at least 85% (e.g., at least 90%, 95%, 98% or 99%, or even 100%) aa sequence identity to a sequence shown in FIG. 4 (e.g., any of the full length sequences shown in FIG. 4, or the sequence of any of the mature β2M polypeptides starting at aa 21 and ending at their C-terminus). For the purposes of this embodiment, sequence identity of the β2M polypeptide is determined relative to the corresponding portion of a β2M polypeptide in FIG. 4 without consideration of the added sulfatase motif and any linker sequences present.

U.S. Pat. No. 9,540,438 discusses the incorporation of sulfatase motifs into the various immunoglobulin sequences, including Fc region polypeptides, and is herein incorporated by reference for its teachings on sulfatase motifs and modification of Fc polypeptides and other polypeptides. That patent is also incorporated by reference for its guidance on FGE enzymes, and their use in forming fGly residues, as well as the chemistry related to the coupling of molecules such as epitopes and payloads to fGly residues.

The incorporation of a sulfatase motif may be accomplished by incorporating a nucleic acid sequence encoding the motif at the desired location in a nucleic acid encoding the first and/or second polypeptide of the T-Cell-MMP. As discussed below, the nucleic acid sequence may be placed under the control of a transcriptional regulatory sequence(s) (a promoter) and provided with regulatory elements that direct its expression. The expressed protein may be treated with one or more FGEs after expression and partial or complete purification. Alternatively, expression of the nucleic acid in cells that express a FGE that recognizes the sulfatase motif results in the conversion of the cysteine or serine of the motif to fGly, which is sometimes called oxoalanine.

In view of the foregoing, this disclosure provides for T-Cell-MMPs comprising one or more fGly residues incorporated into the sequence of the first or second polypeptide chain as discussed above. The fGly residues may, for example, be in the context of the sequence X1(fGly)X2Z2X3Z3, where: fGly is the formylglycine residue; and Z2, Z3, X1, X2 and X3 are as defined in Formula (I) above.

Epitopes and/or payloads may be conjugated either directly or indirectly to the reactive formyl glycine of the sulfatase motif directly or through a peptide or chemical linker. After chemical conjugation the T-Cell-MMPs comprise one or more fGly′ residues incorporated into the sequence of the first or second polypeptide chain in the context of the sequence X1(fGly′)X2Z2X3Z3, where the fGly′ residue is formylglycine that has undergone a chemical reaction and now has a covalently attached moiety (e.g., epitope or payload).

A number of chemistries and commercially available reagents can be utilized to conjugate a molecule (e.g., an epitope or payload) to a fGly residue, including, but not limited to, the use of thiosemicarbazide, aminooxy, hydrazide, or hydrazino derivatives of the molecules to be coupled at a fGly-containing chemical conjugation site. For example, epitopes (e.g., epitope peptides) and/or payloads bearing thiosemicarbazide, aminooxy, hydrazide, hydrazino or hydrazinyl functional groups (e.g., attached directly to an aa of a peptide or via a linker such as a PEG) can be reacted with fGly-containing first or second polypeptides of the T-Cell-MMP to form a covalently linked epitope. Similarly, payloads such as drugs or therapeutics can be incorporated using, for example, biotin hydrazide as a linking agent.

An epitope (e.g., an epitope presenting peptide, phosphopeptide, lipopeptide, or glycopeptide) such as an epitope having a length from about 4 aa to about 20 aa (e.g., 4 aa, 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, or 20 aa) in length and/or one or more payloads may be conjugated to a fGly containing polypeptide.

The disclosure provides for methods of preparing T-Cell-MMP-epitope conjugates and/or T-Cell-MMP-payload conjugates comprising:
a) incorporating a sequence encoding a sulfatase motif including a serine or cysteine (e.g., a sulfatase motif of Formula (I) or (II) such as X1CX2PX3Z3 (SEQ ID NO:63); CX1PX2Z3 (SEQ ID NO:64) discussed above) into a nucleic acid encoding a first polypeptide and/or second polypeptide of a T-Cell-MMP;
b) expressing the sulfatase motif-containing first polypeptide and/or second polypeptide in a cell that
   i) expresses a FGE and converts the serine or cysteine of the sulfatase motif to a fGly and partially or completely purifying the fGly-containing first polypeptide and/or second polypeptide separately or as the T-Cell-MMP, or
   ii) does not express a FGE that converts a serine or cysteine of the sulfatase motif to a fGly, purifying or partially purifying the T-Cell-MMP containing the fGly residue and contacting the purified or partially purified T-Cell-MMP with a FGE that converts the serine or cysteine of the sulfatase motif into a fGly residue; and
c) contacting the fGly-containing first and/or second polypeptides separately, or as part of a T-Cell-MMP, with an epitope and/or payload that has been functionalized with a group that forms a covalent bond between the aldehyde of the fGly and epitope and/or payload;

thereby forming a T-Cell-MMP-epitope conjugate and/or T-Cell-MMP payload conjugate.

In such methods the epitope (epitope containing molecule) and/or payload may be functionalized by any suitable function group that reacts selectively with an aldehyde group. Such groups may, for example, be selected from the group consisting of thiosemicarbazide, aminooxy, hydrazide, and hydrazino. In an embodiment a sulfatase motif is incorporated into a first or second polypeptide comprising a β2M aa sequence with at least 85% (e.g., at least 90%, 95%, 98% or 99%, or even 100%) sequence identity to at least 60, 70, 80 or 90 contiguous aas of a β2M sequence shown in FIG. 4 (e.g., with identity calculated without including or before the addition of the sulfatase motif sequence). For example, the sulfatase motif may be placed between the signal sequence and the sequence of the mature peptide, or at the N-terminus of the mature peptide, and the motif may be separated from the β2M sequence(s) by peptide linkers.

In an embodiment, the method of preparing a T-Cell-MMP-epitope conjugate and/or T-Cell-MMP payload conjugate, a sulfatase motif is incorporated into a polypeptide comprising a sequence having at least 85% (e.g., at least 90%, 95%, 98% or 99%, or even 100%) as sequence identity to at least 150, 175, 200, or 225 contiguous aas of a sequence shown in FIG. 3 (e.g., 3A-3I, with sequence identity calculated without including the addition of the sulfatase motif sequence). In one such embodiment, the sulfatase motifs may be utilized as sites for the conjugation of, for example, epitopes and/or payloads either directly or indirectly through a peptide or chemical linker.

b. Sortase A Enzyme Sites

Epitopes (e.g., peptides comprising the sequence of an epitope) and payloads may be attached at the N- and/or C-termini of the first and/or second polypeptides of a T-Cell-MMP by incorporating sites for Sortase A conjugation at those locations.

Sortase A recognizes a C-terminal pentapeptide sequence LP(X5)TG/A (SEQ ID NO:65, with X5 being any single amino acid, and G/A being a glycine or alanine), and creates an amide bond between the threonine within the sequence and glycine or alanine in the N-terminus of the conjugation partner.

For attachment of epitopes or payloads to the C-terminus of the first or second polypeptide of the T-Cell-MMP, an LP(X5)TG/A is engineered into the carboxy terminal portion of the desired polypeptide(s). An exposed stretch of glycines or alanines (e.g., (G)₃₋₅ (SEQ ID NOs:66 and 67 when using Sortase A from Staphylococcus aureus or alanines (A)₃₋₅, SEQ ID NOs:68 and 69 when using Sortase A from Streptococcus pyogenes) is engineered into the N-terminus of a peptide that comprises an epitope (or a linker attached thereto), a peptide payload (or a linker attached thereto), or a peptide covalently attached to a non-peptide epitope or payload.

For attachment of epitopes or payloads to the amino terminus of the first or second polypeptide of the T-Cell-MMP, an aa sequence comprising an exposed stretch of glycines (e.g., (G)_{2, 3, 4, or 5}) or alanines (e.g., (A)_{2, 3, 4, or 5}) is engineered to appear at the N-terminus of the desired polypeptide(s), and a LP(X5)TG/A is engineered into the carboxy terminal portion of a peptide that comprises an epitope (or a linker attached thereto), a peptide payload (or a linker attached thereto), or a peptide covalently attached to a non-peptide epitope or payload.

Combining Sortase A with the amino and carboxy engineered peptides results in a cleavage between the Thr and Gly/Ala residues in the LP(X5)TG/A sequence, forming a thioester intermediate with the carboxy labeled peptide. Nucleophilic attack by the N-terminal modified polypeptide results in the formation of a covalently coupled complex of the form: carboxy-modified polypeptide-LP(X5)T*G/A-amino-modified polypeptide, where the “*” represents the bond formed between the threonine of the LP(X5)TG/A motif and the glycine or alanine of the N-terminal modified peptide.

In place of LP(X5)TG/A, a LPETGG (SEQ ID NO:70) peptide may be used for S. aureus Sortase A coupling, or a LPETAA (SEQ ID NO:71) peptide may be used for S. pyogenes Sortase A coupling. The conjugation reaction is still between the threonine and the amino terminal oligoglycine or oligoalanine peptide to yield a carboxy-modified polypeptide-LP(X5)T*G/A-amino-modified polypeptide, where the “*” represents the bond formed between the threonine and the glycine or alanine of the N-terminal modified peptide.

In an embodiment, a A₂₋₅ or a G₂₋₅ motif is incorporated into a polypeptide comprising a sequence having at least 85% (e.g., at least 90%, 95%, 98% or 99%, or even 100%) aa sequence identity to at least 60, 70, 80 or 90 contiguous aas of a sequence shown in FIG. 4 (e.g., either the entire sequences shown in FIG. 4, or the sequence of the mature polypeptides starting at aa 21 and ending at their C-terminus), with sequence identity assessed without consideration of the added A₂₋₅ or G₂₋₅ motif and any linker sequences present.

In an embodiment, an A₂₋₅ or a G₂₋₅ motif is incorporated into a polypeptide comprising a β2M sequence having 1 to 15 (e.g., 1, 2, 3, 4, 5, 6, 7, 8, 9, 10, 11, 12, 13, 14, or 15) aa deletions, insertions and/or changes compared with a sequence shown in FIG. 4 (e.g., any of the full length sequences shown in FIG. 4, or any of the mature polypeptide sequences starting at aa 21 and ending at their C-terminus), with aa deletions, insertions and/or changes assessed without consideration of the added A₂₋₅ or G₂₋₅ motif and any linker sequences present. In one such embodiment an A₂₋₅ or a G₂₋₅ motif may either replace and/or be inserted between any of the amino terminal 15 (e.g., 1-5, 5-10 or 10-15) aas of a mature β2M sequence, such as those shown in FIG. 4.

c. Transglutaminase Enzyme Sites

Transglutaminases (mTGs) catalyze the formation of a covalent bond between the amide group on the side chain of a glutamine residue and a primary amine donor (e.g., a primary alkyl amine, such as is found on the side chain of a lysine residue in a polypeptide). Transglutaminases may be employed to conjugate epitopes and payloads to T-Cell-MMPs, either directly through a free amine or indirectly via a linker comprising a free primary amine. As such, glutamine residues present in or added to the first and/or second polypeptides of the T-Cell-MMP in the context of a transglutaminase site may be considered as chemical conjugation sites when they can be accessed by enzymes such as Streptoverticillium mobaraense transglutaminase. That enzyme (EC 2.3.2.13) is a stable, calcium-independent enzyme catalyzing the γ-acyl transfer of glutamine to the ε-amino group of lysine. Glutamine residues appearing in a sequence are, however, not always accessible for enzymatic modification. The limited accessibility can be advantageous as it limits the number of locations where modification may occur. For example, bacterial mTGs are generally unable to modify glutamine residues in native IgG1s; however, Schibli and co-workers (Jeger, S., et al. Angew Chem (Int Engl). 2010; 49:99957 and Dennler P, et al. Bioconjug Chem. 2014; 25(3):569-78) found that deglycosylating IgG1s at N297 rendered glutamine residue Q295 accessible and permitted enzymatic ligation to create an antibody drug conjugate. Further, by producing a N297 to Q297 IgG1 mutant, they introduce two sites for enzymatic labeling by transglutaminase. Modification at N297 also offer the potential to reduce the interaction of the IgG Fc reaction with complement C1q protein.

Where a first and/or second polypeptide of the T-Cell-MMP does not contain a glutamine that may be employed as a chemical conjugation site (e.g., it is not accessible to a transglutaminase or not placed in the desired location), a glutamine residue may be added to a sequence to form a transglutaminase site, or a sequence comprising a transglutaminase (sometimes referred to as a “glutamine tag” or a “Q-tag”), may be incorporated into the polypeptide. The added glutamine or Q-tag may act as a first polypeptide chemical conjugation site or a second polypeptide chemical conjugation site. US Pat. Pub. No. 2017/0043033 A1 describes the incorporation of glutamine residues and Q-tags and the use of transglutaminase for modifying polypeptides and is incorporated herein for those teachings.

Incorporation of glutamine residues and Q-tags may be accomplished chemically where the peptide is synthesized, or by modifying a nucleic acid that encodes the polypeptide and expressing the modified nucleic acid in a cell or cell free system. In embodiments, the glutamine-containing Q-tag comprises an aa sequence selected from the group consisting of LQG, LLQGG (SEQ ID NO:72), LLQG (SEQ ID NO:73), LSLSQG (SEQ ID NO:74), and LLQLQG (SEQ ID NO:75) (numerous others are available).

Glutamine residues and Q-tags may be incorporated into any desired location of a T-Cell-MMP. In an embodiment, a glutamine residue or Q-tag may be added in (e.g., at or near the terminus) of any T-Cell-MMP element, including the MHC-H or β2M polypeptide sequences or any linker sequence joining them (the L3 linker). Glutamine residues and Q-tags may also be added to the scaffold polypeptide (e.g., the Ig Fc) or any of the linkers present in the T-Cell-MMP (e.g., L1 to 13).

A glutamine residue or Q-tag may be incorporated into, or attached to (e.g., via a peptide linker) a β2M polypeptide in a T-Cell-MMP with a sequence having at least 85% (e.g., at least 90%, 95%, 98% or 99%, or even 100%) aa sequence identity to at least 50 (e.g., at least 60, 70 80 90, 96, 97, or 98 or all) contiguous aas of a mature β2M polypeptide sequence shown in FIG. 4 (e.g., the sequences shown in FIG. 4 starting at aa 21 and ending at their C-terminus). The mature human β2M polypeptide sequence in FIG. 4, may be selected for incorporation of the glutamine residue or Q-tag. Sequence identity to the β2M polypeptides is determined relative to the corresponding portion of a β2M polypeptide in FIG. 4 without consideration of the added glutamine residue, Q-tag, or any linker or other sequences present.

In an embodiment, a glutamine residue or Q-tag may be incorporated into a β2M polypeptide sequence having 1 to 15 (e.g., 1, 2, 3, 4, 5, 6, 7, 8, 9, 10, 11, 12, 13, 14, or 15) aa deletions, insertions and/or changes compared with a sequence shown in FIG. 4 (either an entire sequence shown in FIG. 4, or the sequence of a mature polypeptides starting at aa 21 and ending at its C-terminus). Changes are assessed without consideration of the aas of the glutamine residue, Q-tag and any linker sequences present. In one such embodiment a glutamine residue or Q-tag may be place and/or be inserted within aas 1 aas 1-15, 15-35, 35-55, 40-50, or 50-70 of a mature β2M sequence, such as those shown in FIG. 4. In one embodiment, glutamine residue or Q-tag may be located between aas 35-55 (e.g., 40 to 50) of the human mature β2M polypeptide sequence of FIG. 4 and having 0 to 15 aa substitutions.

A glutamine residue or Q-tag may be incorporated into, or attached to (e.g., via a peptide linker) an MHC Class I heavy chain polypeptide sequence having at least 85% (e.g., at least 90%, 95%, 98% or 99%, or even 100%) aa sequence identity to at least 150, 175, 200, or 225 contiguous aas of an MHC-H sequence shown in FIGS. 3A to 3I before the addition of the glutamine residue or Q-tag.

In an embodiment, the added glutamine residue or Q-tag is attached to the N- or C-terminus of a T-Cell-MMP or, if present, attached to or within a linker located at the N- or C-terminus of the T-Cell-MMP.

Payloads and epitopes that contain, or have been modified to contain, a primary amine group may be used as the amine donor in a transglutaminase catalyzed reaction forming a covalent bond between a glutamine residue (e.g., a glutamine residue in a Q-tag) and the epitope or payload.

Where an epitope or payload does not comprise a suitable primary amine to permit it to act as the amine donor, the epitope or payload may be chemically modified to incorporate an amine group (e.g., modified to incorporate a primary amine by linkage to a lysine, aminocaproic acid, cadaverine etc.). Where an epitope or payload comprises a peptide and requires a primary amine to act as the amine donor, a lysine or another primary amine that a transglutaminase can act on may be incorporated into the peptide. Other amine containing compounds that may provide a primary amine group and that may be incorporated into, or at the end of, an alpha amino acid chain include, but are not limited to, homolysine, 2,7-diaminoheptanoic acid, and aminoheptanoic acid. Alternatively, the epitope or payload may be attached to a peptide or non-peptide linker that comprises a suitable amine group. Examples of suitable non-peptide linkers include an alkyl linker and a PEG (polyethylene glycol) linker.

Transglutaminase can be obtained from a variety of sources, including enzymes from: mammalian liver (e.g., guinea pig liver); fungi (e.g., Oomycetes, Actinomycetes, Saccharomyces, Candida, Cryptococcus, Monascus, or Rhizopus transglutaminases); myxomycetes (e.g., Physarum polycephalum transglutaminase); and/or bacteria including a variety of Streptoverticillium, Streptomyces, Actinomadura sp., Bacillus, and the like.

As discussed above for other first polypeptide chemical conjugation sites and second polypeptide chemical conjugation sites, a glutamine or Q-tag may be incorporated into any desired location on the first or second polypeptide of the T-Cell-MMP. In an embodiment, a glutamine or Q-tag may be added at or near the terminus of any element in the first or second polypeptide of the T-Cell-MMP, including the first and second MHC polypeptides (e.g., MHC-H and β2M polypeptides), the scaffold or Ig Fc, and the linkers adjoining those elements.

In one embodiment, where the first polypeptide of the T-Cell-MMP comprises a β2M polypeptide sequence, the first polypeptide contains a glutamine or Q-tag at the N-terminus of the polypeptide, or at the N-terminus of a polypeptide linker attached to the first polypeptide (e.g., the linker is attached to the N-terminus of the first polypeptide).

In an embodiment a Q-tag motif is incorporated into a polypeptide comprising a β2M sequence having at least 85% (e.g., at least 90%, 95%, 98% or 99%, or even 100%) aa sequence identity to at least 60, 70, 80 or 90 contiguous aas of a sequence shown in FIG. 4 (e.g., any of the full-length sequences shown in FIG. 4, or the sequence of any of the mature β2M polypeptide starting at aa 21 and ending at their C-terminus), with identity assessed without consideration of the added Q-tag motif and any linker sequences present.

In an embodiment a Q-tag motif is incorporated into a sequence having 1 to 15 (e.g., 1, 2, 3, 4, 5, 6, 7, 8, 9, 10, 11, 12, 13, 14, or 15) aa deletions, insertions and/or changes compared with a sequence shown in FIG. 4 (either the entire sequences shown in FIG. 4, or the sequence of the mature polypeptides starting at aa 21 and ending at their C-terminus). Changes are assessed without consideration of the aas of the Q-tag motif and any linker sequences present. In one such embodiment a Q-tag motif may replace and/or be inserted between any of the amino terminal 15 (e.g., 1-5, 5-10, or 10-15) aas of a mature β2M sequence, such as those shown in FIG. 4.

Q-tags may be created by modifying the an sequence around any one, two, or three of the glutamine residues appearing in a β2M and/or MHC-H chain sequence appearing in a T-Cell-MMP and used as a chemical conjugation site for addition of an epitope or payload. Similarly, Q-tags may be incorporated into the IgFc region as second polypeptide chemical conjugation sites and used for the conjugation of, for example, epitopes and/or payloads either directly or indirectly through a peptide or chemical linker bearing primary amine.

d. Selenocysteine and Non-Natural Amino Acids as Chemical Conjugation Sites

One strategy for providing site-specific chemical conjugation sites in the first and/or second polypeptides of a T-Cell-MMP employs the insertion of aas with reactivity distinct from the other aas present in the polypeptide. Such aas include, but are not limited to, the non-natural aas, acetylphenylalanine (p-acetyl-L-phenylalanine, pAcPhe), parazido phenylalanine, and propynyl-tyrosine, and the naturally occurring aa, selenocysteine (Sec).

Thanos et a). in US Pat. Publication No. 20140051836 A1 discuss some other non-natural aas including O-methyl-L-tyrosine, L-3-(2-naphthyl)alanine, a 3-methyl-phenylalanine, an O-4-allyl-L-tyrosine, a 4-propyl-L-tyrosine, a tri-O-acetyl-GlcNAcβ-serine, L-Dopa, a fluorinated phenylalanine, an isopropyl-L-phenylalanine, a p-acyl-L-phenylalanine, a p-benzoyl-L-phenylalanine, L-phosphoserine, a phosphonoserine, a phosphonotyrosine, a p-iodo-phenylalanine, a p-bromophenylalanine, a p-amino-L-phenylalanine, an isopropyl-L-phenylalanine, and a p-propargyloxy-phenylalanine. Other non-natural aas include reactive groups including amino, carboxy, acetyl, hydrazino, hydrazido, semicarbazido, sulfanyl, azido and alkynyl. See, e.g., US Pat. Publication No. 20140046030 A1.

In addition to directly synthesizing polypeptides in the laboratory, two methods utilizing stop codons have been developed to incorporate non-natural aas into proteins and polypeptides utilizing transcription-translation systems. The first incorporates selenocysteine (Sec) by pairing the opal stop codon, UGA, with a Sec insertion sequence. The second incorporates non-natural aas into a polypeptide generally through the use of amber, ochre, or opal stop codons. The use of other types of codons such as a unique codon, a rare codon, an unnatural codon, a five-base codon, and a four-base codon, and the use of nonsense and frameshift suppression have also been reported. See, e.g., US Pat. Publication No. 20140046030 A1 and Rodriguez et al., PNAS 103(23)8650-8655 (2006). By way of example, the non-natural amino acid acetylphenylalanine may be incorporated at an amber codon using a tRNA/aminoacyl tRNA synthetase pair in an in vivo or cell free transcription-translation system.

Incorporation of both selenocysteine and non-natural aas requires engineering the necessary stop codon(s) into the nucleic acid coding sequence of the first and/or second polypeptide of the T-Cell-MMP at the desired location(s), after which the coding sequence is used to express the first or second polypeptide strand of the T-Cell-MMP in an in vivo or cell free transcription-translation system.

In vivo systems generally rely on engineered cell-lines to incorporate non-natural aas that act as bio-orthogonal chemical conjugation sites into polypeptides and proteins. See, e.g., International Published Application No. 2002/085923 entitled “In vivo incorporation of unnatural amino acids.” In vivo non-natural aa incorporation relies on a tRNA and an aminoacyl tRNA synthetase (aaRS) pair that is orthogonal to all the endogenous tRNAs and synthetases in the host cell. The non-natural aa of choice is supplemented to the media during cell culture or fermentation, making cell-permeability and stability important considerations.

Various cell-free synthesis systems provided with the charged tRNA may also be utilized to incorporate non-natural aas. Such systems include those described in US Pat. Publication No. 20160115487A1; Gubens et al., RNA. 2010 August; 16(8): 1660-1672; Kim, D. M. and Swartz, J. R. Biotechnol. Bioeng. 66:180-8 (1999); Kim, D. M. and Swartz, J. R. Biotechnol. Prog. 16:385-90 (2000); Kim, D. M. and Swartz, J. R. Biotechnol. Bioeng. 74:309-16 (2001); Swartz et al, Methods Mol. Biol. 267:169-82 (2004); Kim, D. M. and Swartz, J. R. Biotechnol. Bioeng. 85:122-29 (2004); Jewett, M. C. and Swartz, J. R., Biotechnol. Bioeng. 86:19-26 (2004); Yin, G. and Swartz, J. R., Biotechnol. Bioeng. 86:188-95 (2004); Jewett, M. C. and Swartz, J. R., Biotechnol. Bioeng. 87:465-72 (2004); Voloshin, A. M. and Swartz, J. R., Biotechnol. Bioeng. 91:516-21 (2005).

Once incorporated into the first or second polypeptide of the T-Cell-MMP, epitopes and/or payload bearing groups reactive with the incorporated selenocysteine or non-natural aa are brought into contact with the T-Cell-MMP under suitable conditions to form a covalent bond. By way of example, the keto group of the pAcPhe is reactive towards alkoxy-amines, via oxime coupling, and can be conjugated directly to alkoxyamine containing epitopes and/or payloads or indirectly to epitopes and payloads via an alkoxyamine containing linker. Selenocysteine reacts with, for example, primary alkyl iodides (e.g., iodoacetamide which can be used as a linker), maleimides, and methylsulfone phenyloxadiazole groups. Accordingly, epitopes and/or payloads bearing those groups or bound to linkers bearing those groups can be covalently bound to polypeptide chains bearing selenocysteines.

As discussed above for other first polypeptide chemical conjugation sites and second polypeptide chemical conjugation sites, selenocysteines and/or non-natural aas may be incorporated into any desired location in the first or second polypeptide of the T-Cell-MMP. In an embodiment, selenocysteines and/or non-natural aas may be added at or near the terminus of any element in the first or second polypeptide of the T-Cell-MMP, including the first and second MHC polypeptides (e.g., MHC-H and β2M polypeptides), the scaffold or Ig Fc, and the linkers adjoining those elements. In embodiments selenocysteines and/or non-natural aas may be incorporated into a β2M, class I MHC heavy chain, and/or a Fc Ig polypeptide. In an embodiment, selenocysteines and/or non-natural aas may be incorporated into the first polypeptide near or at the amino terminal end of the first MHC polypeptide (e.g., the β2M polypeptide) or a linker attached to it. For example, where the first polypeptide comprises a β2M sequence, selenocysteines and/or non-natural aas may be incorporated at or near the N-terminus of a β2M sequence (e.g., within about the first 15 aas of the mature β2M aa sequence), permitting the chemical conjugation of, for example, an epitope either directly or through a linker. By way of example, the sequences of β2M as shown in FIG. 4 begin with a 20 aa leader sequence, and the mature polypeptide begins with the initial sequence IQRTP(K/Q)IQVYS . . . and continues through the remainder of the polypeptide (see SEQ ID NOs:57-61).

In an embodiment selenocysteines and/or non-natural aas are incorporated into a polypeptide comprising a β2M sequence having at least 85% (e.g., at least 90%, 95%, 98% or 99%, or even 100%) aa sequence identity to a β2M sequence shown in FIG. 4 (e.g., any of the full length sequences shown in FIG. 4, or the sequence of any of the mature β2M polypeptides starting at as 21 and ending at their C-terminus), with sequence identity assessed without consideration of the added selenocysteines and/or non-natural aas and any linker sequences present.

In an embodiment selenocysteines and/or non-natural aas are incorporated into a polypeptide comprising a β2M sequence having 1 to 15 (e.g., 1, 2, 3, 4, 5, 6, 7, 8, 9, 10, 11, 12, 13, 14, or 15) aa deletions, insertions and/or changes compared with a β2M sequence shown in FIG. 4 (e.g., any of the full-length sequences shown in FIG. 4, or the sequence of any of the mature β2M polypeptides starting at as 21 and ending at their C-terminus). Changes are assessed without consideration of the aas of the selenocysteines and/or non-natural aas and any linker sequences present. In one such embodiment a selenocysteine and/or non-natural aa may replace and/or be inserted between any of the amino terminal 15 aas of a mature β2M sequence, such as those shown in FIG. 4.

In other embodiments, selenocysteines and/or non-natural aas may be incorporated into polypeptides comprising an MHC-H chain or IgFc polypeptide sequences (including linkers attached thereto) as chemical conjugation sites. In one such embodiment they may be utilized as sites for the conjugation of, for example, epitopes and/or payloads conjugated to the T-Cell-MMP either directly or indirectly through a peptide or chemical linker.

e. Amino Acid Chemical Conjugation Sites

Any of the variety of functionalities (e.g., —SH, —NH₃, —OH, —COOH and the like) present in the side chains of naturally occurring amino acids, or at the termini of polypeptides, can be used as chemical conjugation sites. This includes the side chains of lysine and cysteine, which are readily modifiable by reagents including N-hydroxysuccinimide and maleimide functionalities, respectively. The main disadvantages of utilizing such amino acid residues is the potential variability and heterogeneity of the products. For example, an IgG has over 80 lysines, with over 20 at solvent-accessible sites. See, e.g., McComb and Owen, AAPS J. 117(2): 339-351. Cysteines tend to be less widely distributed; they tend to be engaged in disulfide bonds, and may be inaccessible (e.g., not accessible by solvent or to molecules used to modify the cysteines, and not located where it is desirable to place a chemical conjugation site. It is, however, possible to selectively modify T-Cell-MMP polypeptides to provide naturally occurring and, as discussed above, non-naturally occurring amino acids at the desired locations for placement of a chemical conjugation site. Modification may take the form of direct chemical synthesis of the polypeptides (e.g., by coupling appropriately blocked amino acids) and/or by modifying the sequence of a nucleic acid encoding the polypeptide followed expression in a cell or cell-free system. Accordingly, this disclosure includes and provides for the preparation of the T-Cell-MMP polypeptides by transcription/translation systems capable of incorporating a non-natural aa or natural aa (including selenocysteine) to be used as a chemical conjugation site for epitope or payload conjugation.

The disclosure includes and provides for the preparation of all or part of the first and/or second polypeptide of a T-Cell-MMP by transcription/translation systems and joining to an C- or N-terminus of the translated portion of the first and/or second polypeptide a polypeptide bearing a non-natural aa or natural aa (including selenocysteine) prepared by, for example, chemical synthesis to be used, for example, as a chemical conjugation site (e.g., for epitopes). The polypeptide, which may include a linker, may be joined by any suitable method including the use of a sortase as described above for peptide epitopes. In an embodiment, the polypeptide may comprise a sequence of 2, 3, 4, or 5 alanines or glycines that may serve for sortase conjugation and/or as part of a linker sequence.

A naturally occurring aa (e.g., a cysteine) to be used as a chemical conjugation site may be provided at any desired location of a T-Cell-MMP. In an embodiment, a the naturally occurring aa may be provided (e.g., at or near the terminus) of any T-Cell-MMP element, including the MHC-H or β2M polypeptide sequences or any linker sequence joining them (the any of the L1, L2 and/or L3 linkers). Naturally occurring aa(s) may also be provided in the scaffold polypeptide (e.g., the Ig Fc) or any of the linkers present in the T-Cell-MMP.

A naturally occurring aa (e.g., a cysteine) may also be provided, e.g., via protein engineering, in, or attached to (e.g., via a peptide linker), a β2M polypeptide in a T-Cell-MMP with a sequence having at least 85% (e.g., at least 90%, 95%, 98% or 99%, or even 100%) aa sequence identity to at least 50 (e.g., at least 60, 70 80 90, 96, 97, or 98 or all) contiguous aas of a mature β2M polypeptide sequence shown in FIG. 4 (e.g., the sequences shown in FIG. 4 starting at an 21 and ending at their C-terminus). The mature human β2M polypeptide sequence in FIG. 4, may be selected for incorporation of the naturally occurring aa. Sequence identity to the β2M polypeptides is determined relative to the corresponding portion of a β2M polypeptide in FIG. 4 without consideration of the added naturally occurring aa, any linker, or any other sequences present.

In an embodiment, a naturally occurring aa (e.g., a cysteine) may be provided, e.g., via protein engineering in a β2M polypeptide sequence having 1 to 15 (e.g., 1, 2, 3, 4, 5, 6, 7, 8, 9, 10, 11, 12, 13, 14, or 15) an deletions, insertions and/or changes compared with a sequence shown in FIG. 4 (either an entire sequence shown in FIG. 4, or the sequence of a mature polypeptides starting at an 21 and ending at its C-terminus). Changes are assessed without consideration of the aas of the naturally occurring aa sequences, any linker, or other sequences present. In one such embodiment a naturally occurring aa (e.g., a cysteine) may be engineered (e.g., using the techniques of molecular biology) within aas 1-15, 15-35, 35-55, 40-50, or 50-70 of a mature β2M sequence, such as those shown in FIG. 4. In one embodiment, a naturally occurring as (e.g., a cysteine) may be provided in the sequence between aas 35-55 (e.g., between 40 and 50. between 42 and 48, between 43 and 45, or at as 44) of the human mature β2M polypeptide sequence of FIG. 4 and having 0 to 15 as additional as substitutions.

A naturally occurring as (e.g., a cysteine) may be provided in, or attached to (e.g., via a peptide linker) an MHC Class I heavy chain polypeptide sequence having at least 85% (e.g., at least 90%, 95%, 98% or 99%, or even 100%) as sequence identity to at least 150, 175, 200, or 225 contiguous aas of an MHC-H sequence shown in FIGS. 3A to 3I before the addition of the naturally occurring aa.

In an embodiment, the naturally occurring as (e.g., a cysteine) may be attached to the N- or C-terminus of a T-Cell-MMP, or attached to or within a linker, if present, located at the N- or C-terminus of the T-Cell-MMP

In one embodiment, a first or second polypeptide of a T-Cell-MMP contains at least one naturally occurring aa (e.g., a cysteine) to be used as a chemical conjugation site provided, e.g., via protein engineering, in a β2M sequence as shown in FIG. 4, an Ig Fc sequence as shown in any of FIGS. 2A-G, or an MHC Class I heavy chain polypeptide as shown in FIGS. 3A-3I. In an embodiment, at least one naturally occurring aa to be used as a chemical conjugation site is provided in a polypeptide having at least 85% (e.g., at least 90%, 95%, 98% or 99%, or even 100%) as sequence identity to at least 50 (e.g., at least 60, 70 80 90, 96, 97, or 98 or all) contiguous aas of a mature β2M sequence as shown in FIG. 4, an Ig Fc sequence as shown in FIGS. 2A to 2G, or at least 85% (e.g., at least 90%, 95%, 98% or 99%, or even 100%) aa sequence identity to at least 150, 175, 200, or 225 contiguous aas of an MHC Class I heavy chain polypeptide as shown in any of FIGS. 3A-3I. At least one naturally occurring as (e.g., a cysteine) may be provided as a chemical conjugation site in a T-Cell-MMP first or second polypeptide comprising a β2M as sequence having at least 90% (e.g., at least 93%, 95%, 98% or 99%, or even 100%) as sequence identity with at least the amino terminal 10, 20, 30, 40, 50, 60 or 70 aas of a mature β2M sequence as shown in FIG. 4. At least one naturally occurring as (e.g., a cysteine) may be provided as a chemical conjugation site in a T-Cell-MMP first or second polypeptide comprising an Ig Fc sequence (e.g., as shown in any of FIGS. 2A-2G). At least one naturally occurring as (e.g., a cysteine) may be provided as a chemical conjugation site in a T-Cell-MMP first or second polypeptide comprising an MHC Class I heavy chain polypeptide sequence having at least 85% (e.g., at least 90%, 95%, 98% or 99%, or even 100%) aa sequence identity to at least 150, 175, 200, or 225 contiguous aas of an MHC H polypeptide sequence provided in any of FIGS. 3A to 3I. In another embodiment, at least one naturally occurring as to be used as a chemical conjugation site is provided in a first or second polypeptide comprising at least 30, 40, 50, 60, 70, 80, 90, or 100 contiguous aas having 100% as sequence identity to an MHC Class I heavy chain sequence as shown in any of FIGS. 3A to 3I or a mature β2M sequence as shown in FIG. 4.

In any of the embodiments mentioned above where a naturally occurring aa is provided, e.g., via protein engineering, in a polypeptide, the aa may be selected from the group consisting of arginine, lysine, cysteine, serine, threonine, glutamic acid, glutamine, aspartic acid, and asparagine. Alternatively, the aa provided as a conjugation site is selected from the group consisting of lysine, cysteine, serine, threonine, and glutamine. The aa provided as a conjugation site may also be selected from the group consisting of lysine, glutamine, and cysteine. In one instance, the provided aa is cysteine. In another instance, the provided aa is lysine. In still another instance, the provided aa is glutamine.

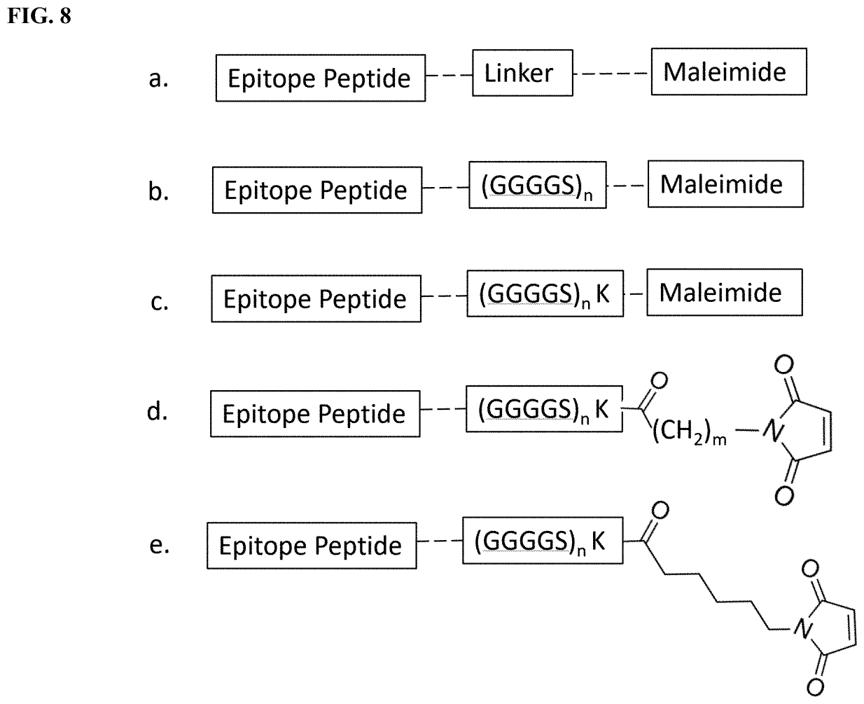

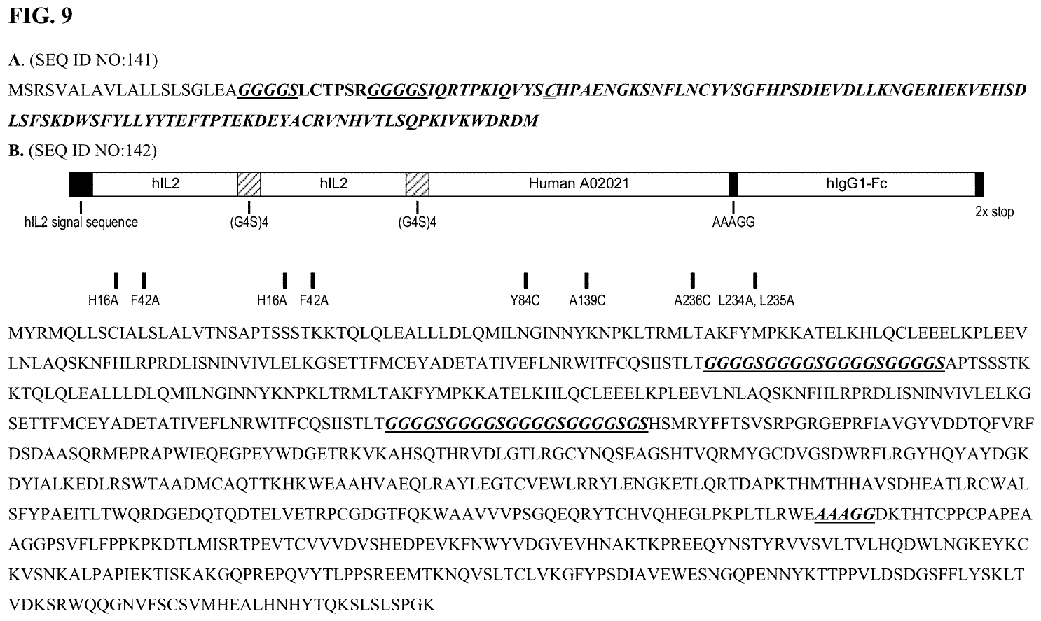

Any method known in the art may be used to couple payloads or epitopes to amino acids provided in the first or second polypeptides of the T-Cell-MMP. By way of example, maleimides may be utilized to couple to sulfhydryls, N-hydroxysuccinimide may be utilized to couple to amine groups, acid anhydrides or chlorides may be used to couple to alcohols or amines, and dehydrating agents may be used to couple alcohols or amines to carboxylic acid groups. Accordingly, using such chemistry an epitope or payload may be coupled directly, or indirectly through a linker (e.g., a homo- or hetero-bifunctional crosslinker), to a location on a first and/or second polypeptide. A number of bifunctional crosslinkers may be utilized, including, but not limited to, those described for linking a payload to a T-Cell-MMP described herein below. An epitope peptide (or a peptide-containing payload) including a maleimide group attached by way of a homo- or hetero-bifunctional linker (see, e.g., FIG. 8) or a maleimide amino acid can be conjugated to a sulfhydryl of a chemical conjugation site (e.g., a cysteine residue) that is naturally occurring or provided in a T-Cell-MMP.

Maleimido amino acids can be incorporated directly into peptides (e.g., epitope peptides) using a Diels-Alder/retro-Diels-Alder protecting scheme as part of a solid phase peptide synthesis. See, e.g., Koehler, Kenneth Christopher (2012), “Development and Implementation of Clickable Amino Acids,” Chemical & Biological Engineering Graduate Theses & Dissertations, 31, https://scholar.colorado.edu/chbe_gradetds/31.

A maleimide group may also be appended to an epitope peptide using a homo- or hetero-bifunctional linker (sometimes referred to as a crosslinker) that attaches a maleimide directly (or indirectly, e.g., through an intervening linker that may comprise additional aas bound to the peptide presenting the epitope) to the epitope peptide. For example, a heterobifunctional N-hydroxysuccinimide-maleimide crosslinker can attach maleimide to an amine group of, a peptide lysine. Some specific cross linkers include molecules with a maleimide functionality and either a N-hydroxysuccinimide ester (NHS) or N-succinimidyl group that can attach a maleimide to an amine (e.g., an epsilon amino group of lysine). Examples of such crosslinkers include, but are not limited to, NHS-PEG4-maleimide, γ-maleimide butyric acid N-succinimidyl ester (GMBS); s-maleimidocaproic acid N-hydroxysuccinimide ester (EMCS); m-maleimide benzoyl-N-hydroxysuccinimide ester (MBS); and N-(α-maleimidoacetoxy)-succinimide ester (AMAS), which offer different lengths and properties for peptide immobilization. Other amine reactive crosslinkers that incorporate a maleimide group include N-succinimidyl 4-(2-pyridyldithio)butanoate (SPDB). Additional crosslinkers (bifunctional agents) are recited below. In an embodiment the epitopes coupled to the T-Cell-MMP have a maleimido alkyl carboxylic acid coupled to the peptide by an optional linker (see, e.g., FIG. 8), for example by an amide formed with the epsilon amino group of a lysine. The maleimido carboxylic acid can be, for example, a maleimido ethanoic, propanoic, butanoic, pentanoic, hexanoic, heptanoic, or octanoic acid.

An epitope peptide may be coupled to a naturally occurring cysteine or a cysteine provided in (e.g., engineered into), for example, the binding pocket of a T-Cell-MMP through a bifunctional linker comprising a maleimide or a maleimide amino acid incorporated into the peptide thereby forming a T-Cell-MMP epitope conjugate. An epitope peptide may be conjugated (e.g., by one or two maleimide amino acids or at least one maleimide containing bifunctional linker) to an MHC heavy chain having cysteine residues at any one or more locations within or adjacent to the MHC-H binding pocket. By way of example, a peptide epitope comprising maleimido amino acids or bearing a maleimide group as part of a crosslinker attached to the peptide may be covalently attached at 1 or 2 aas (e.g., cysteines) at MHC-H positions 2, 5, 7, 59, 84, 116, 139, 167, 168, 170, and/or 171 (e.g., Y7C, Y59C, Y84C, Y116C, A139C, W167C, L168C, R170C, and Y171C substitutions) with the numbering as in FIGS. 3D-3I. A peptide epitope may also be conjugated (e.g., by one or two maleimide amino acids or at least one maleimide containing bifunctional linker) to an MHC heavy chain having cysteine residues at any one or more (e.g., 1 or 2) aa positions selected from positions 7 and/or 116, (e.g., Y7C and Y116C substitutions) with the numbering as in FIGS. 3D-3H. Cysteine substitution at positions 116 (e.g., Y116C) and/or 167 (e.g., W167C), with the numbering as in FIGS. 3D-3H, may be used separately or in combination to anchor epitopes (e.g., peptide epitopes) with one or two bonds for by maleimide groups (e.g., at one or both of the ends of the epitope containing peptide).

Peptide epitopes may also be coupled to a naturally occurring cysteine present or provided in (e.g., engineered into) a β2M polypeptide sequence having at least 85% (e.g., at least 90%, 95% 97% or 100%) sequence identity to at least 60 contiguous amino acids (e.g., at least 70, 80, 90 or all contiguous aas) of a mature β2M polypeptide sequence set forth in FIG. 4. Some solvent accessible positions of mature β2M polypeptides that may be substituted by a cysteine to create a chemical conjugation site include: 2, 14, 16, 34, 36, 44, 45, 47, 48, 50, 58, 74, 77, 85, 88, 89, 91, 94, and 98 (Gln 2, Pro 14, Glu 16, Asp 34, Glu 36, Glu 44, Arg 45, Glu 47, Arg 48, Glu 50, Lys 58, Glu 74, Glu 77, Val 85, Ser 88, Gln 89, Lys 91, Lys 94, and Asp 98) of the mature peptide from NP_004039.1, or their corresponding amino acids in other β2M sequences (see the sequence alignment in FIG. 4). For example, epitopes may be conjugated to cysteines at positions 2, 44, 50, 77, 85, 88, 91, or 98 of the mature β2M polypeptides (aas 22, 64, 70, 97, 85, 108, 111, or 118 of the mature β2M sequences as shown in FIG. 4). Accordingly, the β2M sequences of a T-Cell-MMP may contain cysteine chemical conjugation site provided (e.g., by protein engineering) in the mature β2M sequence selected from Q2C, E44C, E50C, E77C, V85V, S88C, K91C, and D98C. The cysteine chemical conjugation sites in β2M sequences may also be combined with MHC-H Y84C and A139C substitutions made to stabilize the MHC H by forming an intrachain disulfide bond between MHC-H sequences. In one instance, the cysteine chemical conjugation site provided in the mature β2M is located at E44 (an E44C substitution). In another instance, the cysteine chemical conjugation site provided in the mature β2M is located at E44 (an E44C substitution) and the β2M sequences also be comprises MHC-H Y84C and A139C substitutions that form an intrachain disulfide bond.

Where conjugation of an epitope and/or, payload is to be conducted through a cysteine chemical conjugation site present in an unconjugated T-Cell-MMP (e.g., using a maleimide modified epitope or payload) a variety of process conditions may affect the conjugation efficiency and the quality (e.g., the amount/fraction of unaggregated dimer T-Cell-MMP epitope conjugate resulting from the reaction) resulting from the conjugation reaction. Conjugation process conditions that may be individually optimized including, but not limited to, (i) prior to conjugation unblocking of cysteine sulfhydryls (e.g., potential blocking groups may be present and removed), (ii) the ratio of the T-Cell-MMP to the epitope or payload, reaction pH, (iii) the buffer employed, (iv) additives present in the reaction, (v) the reaction temperature, and (vi) the reaction time.

Prior to conjugation T-Cell-MMPs may be treated with a disulfide reducing agent such as dithiothreitol (DTT), mercaptoethanol, or tris(2-carboxyethyl)phosphine (TCEP) to reduce and free cysteines sulfhydryls that may be blocked. Treatment may be conducted using relatively low amounts of reducing agent, for example from about 0.5 to 2.0 reducing equivalents per cysteine conjugation site for relatively short periods, and the cysteine chemical conjugation site of the unconjugated T-Cell MP may be available as a reactive nucleophile for conjugation from about 10 minutes to about 1 hour, or from about 1 hour to 5 hours.

The ratio of the unconjugated T-Cell-MMP to the epitope or payload being conjugated may be varied from about 1:2 to about 1:100, such as from about 1:2 to about 1:3, from about 1:3 to about 1:10, from about 1:10 to about 1:20, from about 1:20 to about 1:40, or from about 1:40 to about 1:100. The use of sequential additions of the reactive epitope or payload may be made to drive the coupling reaction to completion (e.g., multiple does of maleimide or N-hydroxy succinimide modified epitopes may be added to react with the T-Cell-MMP).

As previously indicated, conjugation reaction may be affected by the buffer, its pH, and additives that may be present. For maleimide coupling to reactive cysteines present in a T-Cell-MMP the reactions are typically carried out from about pH 6.5 to about pH 8.0 (e.g., from about pH 6.5 to about pH 7.0, from about pH 7.0 to about pH 7.5, from about pH 7.5 to about pH 8.0, or from about pH 8.0 to about pH 8.5. Any suitable buffer not containing active nucleophiles (e.g., reactive thiols) and preferably degassed to avoid reoxidation of the sulfhydryl may be employed for the reaction. Some suitable traditional buffers include phosphate buffered saline (PBS), Tris-HCl, and (4-(2-hydroxyethyl)-1-piperazineethanesulfonic acid) HEPES. As an alternative to traditional buffers, maleimide conjugation reactions may be conducted in buffers/reaction mixtures comprising amino acids such as arginine, glycine, lysine, or histidine. The use of high concentrations of amino acids, e.g., from about 0.1 M (molar) to about 1.5 M (e.g., from about 0.1 to about 0.25, from about 0.25 to about 0.5 from about 0.3 to about 0.6, from about 0.4 to about 0.7, from about 0.5 to about 0.75, from about 0.75 to about 1.0, from about 1.0 to about 1.25 M, or from about 1.25 to about 1.5 M may stabilize the unconjugated and/or unconjugated T-Cell-MMP.

Additives useful for maleimide and other conjugation reactions include, but are not limited to: protease inhibitors; metal chelator (e.g., EDTA) that can block unwanted side reactions and inhibit metal dependent proteases if they are present, detergents; detergents (e.g., polysorbate 80 sold as TWEEN 80®, or nonylphenoxypolyethoxyethanol sold under the names NP40 and Tergitol™ NP); and polyols such a sucrose or glycerol that can add to protein stability.

Conjugation of T-Cell-MMPs with epitopes and/or payloads, and particularly conjugation at cysteines using maleimide chemistry, can be conducted over a range of temperatures, such as 0° to 40° C. For example, conjugation reactions, including cysteine-maleimide reactions, can be conducted from about 0° to about 10° C., from about 10° to about 20° C., from about 20° to about 30° C., from about 25° to about 37° C., or from about 30° to about 40° C. (e.g., at about 20° C., at about 30° C. or at about 37° C.).

Where a pair of sulfhydryl groups are present, they may be employed simultaneously for chemical conjugation to a T-Cell-MMP. In such an embodiment, an unconjugated T-Cell-MMP that has a disulfide bond, or that has two cysteines (or selenocysteines) provided at locations proximate to each other, may be utilized as a chemical conjugation site by incorporation of bis-thiol linkers. Bis-thiol linkers, described by Godwin and co-workers, avoid the instability associated with reducing a disulfide bond by forming a bridging group in its place and at the same time permit the incorporation of another molecule, which can be an epitope or payload. See, e.g., Badescu G, et al., (2014), Bioconjug Chem., 25(6):1124-36, entitled Bridging disulfides for stable and defined antibody drug conjugates, describing the use of bis-sulfone reagents, which incorporate a hydrophilic linker (e.g., PEG (polyethylene glycol) linker).

Generally, stoichiometric or near stoichiometric amounts of dithiol reducing agents (e.g., dithiothreitol) are employed to reduce the disulfide bond and allow the bis-thiol linker to react with both cysteine and/or selenocysteine residues. Where multiple disulfide bonds are present, the use of stoichiometric or near stoichiometric amounts of reducing agents may allow for selective modification at one site. See, e.g., Brocchini, et al., Adv. Drug. Delivery Rev. (2008) 60:3-12. Where a T-Cell-MMP or dimerized T-Cell-MMP does not comprise a pair of cysteines and/or selenocysteines (e.g., a selenocysteine and a cysteine), they may be provided in the polypeptide (by introducing one or both of the cysteines or selenocysteines) to provide a pair of residues that can interact with a bis-thiol linker. The cysteines and/or selenocysteines should be located such that a bis-thiol linker can bridge them (e.g., at a location where two cysteines could form a disulfide bond). Any combination of cysteines and selenocysteines may be employed (i.e. two cysteines, two selenocysteines, or a selenocysteine and a cysteine). The cysteines and/or selenocysteines may both be present on a T-Cell-MMP. Alternatively, in a dimerized T-Cell-MMP the first cysteine and/or selenocysteine is present in the first T-Cell-MMP of the dimer and a second cysteine and/or selenocysteine is present in the second T-Cell-MMP of the dimer, with the bis-thiol linker acting as a covalent bridge between the dimerized T-Cell-MMPs.

In an embodiment, a pair of cysteine and/or selenocysteine residues is incorporated into a β2M sequence of a T-Cell-MMP having at least 85% (e.g., at least 90%, 95%, 98% or 99%, or even 100%) aa sequence identity to at least 50 (e.g., at least 60, 70 80 90, 96, 97, or 98 or all) contiguous aas of a mature β2M polypeptide sequence shown in FIG. 4 before the addition of the pair of cysteines and/or selenocysteines, and/or into an L2 or L3 peptide linker attached to one of those sequences. In one such embodiment the pair of cysteines and/or selenocysteines may be utilized as a bis-thiol linker coupling site for the conjugation of an epitope and/or payload through a peptide or chemical linker attached to the bis-thiol group.

In another embodiment, a pair of cysteines and/or selenocysteines is incorporated into an MHC-H polypeptide sequence of a T-Cell-MMP as a chemical conjugation site. In an embodiment, a pair of cysteines and/or selenocysteines is incorporated into a polypeptide comprising a sequence having at least 85% (e.g., at least 90%, 95%, 98% or 99%, or even 100%) aa sequence identity to a sequence having at least 150, 175, 200, or 225 contiguous aas of an MHC-H sequence shown in any of FIGS. 3A-3I before the addition of a pair of cysteines or selenocysteines, or into a peptide linker attached to one of those sequences. In one such embodiment the pair of cysteines and/or selenocysteines may be utilized as a bis-thiol linker coupling site for the conjugation of an epitope and/or payload through a peptide or chemical linker attached to the bis-thiol linker. Where the MHC-H sequence includes a Y84C and A139C substitutions, the bis-thiol linker may be used to form a covalent bridge between those sites for the covalent coupling of an epitope (e.g., a peptide epitope).

In another embodiment, a pair of cysteines and/or selenocysteines is incorporated into an Ig Fc sequence of a T-Cell-MMP to provide a chemical conjugation site. In an embodiment a pair of cysteines and/or selenocysteines is incorporated into a polypeptide comprising an Ig Fc sequence having at least 85% (e.g., at least 90%, 95%, 98% or 99%, or even 100%) an sequence identity to a sequence shown in any of the Fc sequences of FIGS. 2A-2G before the addition of the pair of cysteines or selenocysteines. In one such embodiment the pair of cysteines and/or selenocysteines is utilized as a bis-thiol linker coupling site for the conjugation of an epitope and/or payload through a peptide or chemical linker attached to the bis-thiol group. The bis-thiol linker may be used to form a covalent bridge between scaffold polypeptides of a dimerized T-Cell-MMP. In such a case the cysteines of the lower hinge region that form interchain disulfide bonds, if present in the Ig Fc scaffold polypeptide sequence, may be used to insert the bis-thiol linker.

f. Other Chemical Conjugation Sites

(i) Carbohydrate Chemical Conjugation Sites

Many proteins prepared by cellular expression contain added carbohydrates (e.g., oligosaccharides of the type added to antibodies expressed in mammalian cells). Accordingly, where first and/or second polypeptides of a T-Cell-MMP are prepared by cellular expression, carbohydrates may be present and available as selective chemical conjugation sites in, for example, glycol-conjugation reactions. McCombs and Owen, AAPS Journal, (2015) 17(2): 339-351, and references cited therein, describe the use of carbohydrate residues for glycol-conjugation of molecules to antibodies.

The addition and modification of carbohydrate residues may also be conducted ex vivo, through the use of chemicals that alter the carbohydrates (e.g., periodate, which introduces aldehyde groups), or by the action of enzymes (e.g., fucosyltransferases) that can incorporate chemically reactive carbohydrates or carbohydrate analogs for use as chemical conjugation sites.

In an embodiment, the incorporation of an IgFc scaffold with known glycosylation sites may be used to introduce site specific chemical conjugation sites.

This disclosure includes and provides for T-Cell-MMPs and their epitope conjugates having carbohydrates as chemical conjugation (e.g., glycol-conjugation) sites. The disclosure also includes and provides for the use of such molecules in forming conjugates with epitopes and with other molecules such as drugs and diagnostic agents, and the use of those molecules in methods of medical treatment and diagnosis.

(ii) Nucleotide Binding Sites

Nucleotide binding sites offer site-specific functionalization through the use of a UV-reactive moiety that can covalently link to the binding site. Bilgicer et al., Bioconjug Chem. 2014; 25(7):1198-202, reported the use of an indole-3-butyric acid (IBA) moiety that can be covalently linked to an IgG at a nucleotide binding site. By incorporation of the sequences required to form a nucleotide binding site, chemical conjugates of T-Cell-MMP with suitably modified epitopes and/or other molecules (e.g., drugs or diagnostic agents) bearing a reactive nucleotide may be employed to prepare T-Cell-MMP-epitope conjugates. The epitope or payload may be coupled to the nucleotide binding site through the reactive entity (e.g., an IBA moiety) either directly or indirectly though an interposed linker.

This disclosure includes and provides for T-Cell-MMPs having nucleotide binding sites as chemical conjugation sites and the use of such molecules in forming conjugates with epitopes and with other molecules such as drugs and diagnostic agents, and the use of the conjugates in methods of treatment and diagnosis.

3 Binding and Properties of T-Cell-MMPs, Epitopes and MODs

The present disclosure provides T-Cell-MMP-epitope conjugates. In one embodiment the disclosure provides for a T-Cell-MMP-epitope conjugate comprising: a) a first polypeptide; and b) a second polypeptide, wherein the first and second polypeptides of the multimeric polypeptide comprise an epitope (e.g., a coronavirus epitope such as a SARS-CoV or SARS-CoV-2 spike, nucleoprotein, membrane protein, replicase protein, 3a protein, or the like); a first MHC polypeptide; a second MHC polypeptide; and optionally an immunoglobulin (Ig) Fc polypeptide or a non-Ig scaffold. In another embodiment, the present disclosure also provides a T-Cell-MMP-epitope conjugate comprising: a) a first polypeptide comprising, in order from N-terminus to C-terminus: i) an epitope (e.g., a coronavirus epitope); and ii) a first MHC polypeptide; and b) a second polypeptide comprising, in order from N-terminus to C-terminus: i) a second MHC polypeptide; and ii) optionally an Ig Fc polypeptide or a non-Ig scaffold. In addition to those components recited above, at least one of the first and second polypeptides of the T-Cell-MMP-epitope conjugates of the present disclosure comprise one or more (e.g., at least one or at least two) MODs. The one or more MODs are located: A) at the C-terminus of the first polypeptide; B) at the N-terminus of the second polypeptide; C) at the C-terminus of the second polypeptide; D) at the C-terminus of the first polypeptide and at the N-terminus of the second polypeptide; and/or E) between the MHC polypeptide and an Ig Fc polypeptide of the second polypeptide. In an embodiment, at least one (e.g., at least two, or at least three) of the one or more MODs is a variant MOD that exhibits reduced affinity to a Co-MOD compared to the affinity of a corresponding wild-type MOD for the Co-MOD.

In an embodiment, the epitope present in a T-Cell-MMP-epitope conjugate of the present disclosure (see, e.g., FIG. 6) binds to a T-cell receptor (TCR) on a T-cell with an affinity of at least 100 μM (e.g., at least 10 LM, at least 1 μM, at least 100 nM, at least 10 nM or at least 1 nM). In an embodiment, a T-Cell-MMP-epitope conjugate of the present disclosure binds to a first T-cell with an affinity that is at least 25% higher than the affinity with which the T-Cell-MMP-epitope conjugate binds to a second T-cell, where the first T-cell expresses on its surface the Co-MOD and a TCR that binds the epitope with an affinity of at least 100 μM, and where the second T-cell expresses on its surface the Co-MOD but does not express on its surface a TCR that binds the epitope with an affinity of at least 100 μM (e.g., at least 10 μM, at least 1 μM, at least 100 nM, at least 10 nM, or at least 1 nM). In some cases, the peptide presenting an epitope present in a T-Cell-MMP-epitope conjugate of the present disclosure presents a coronavirus epitope (such as a SARS-CoV or SARS-CoV-2 spike, nucleoprotein, membrane protein, replicase protein, 3a protein, or the like).

In some cases, the epitope present in a T-Cell-MMP-epitope conjugate of the present disclosure binds to a TCR on a T-cell with an affinity of from about 10⁻⁴ M to about 10⁻⁵ M, from about 10⁻⁵ M to about 10⁻⁶ M, from about 10⁻⁶ M to about 10⁻⁷ M, from about 10⁻⁷ M to about 10⁻⁸ M or from about 10⁻⁸ M to about 10⁻⁹ M. Expressed another way, in some cases, the epitope present in a T-Cell-MMP-epitope conjugate of the present disclosure binds to a TCR on a T-cell with an affinity of from about 0.1 μM to about 1 μM, from about 1 μM to about 10 μM, from about 10 M to about 25 μM, from about 25 μM to about 50 μM, from about 50 μM to about 100 μM.

In an embodiment, a variant MOD present in a T-Cell-MMP-epitope conjugate of the present disclosure binds to its Co-MOD with an affinity that is at least 10% less, at least 15% less, at least 20% less, at least 25% less, at least 30% less, at least 40% less, at least 50% less, at least 60% less, at least 70% less, at least 80% less, at least 90% less, or more than 95% less, than the affinity of a corresponding wild-type MOD for the Co-MOD.

In some cases, a variant MOD present in a T-Cell-MMP-epitope conjugate of the present disclosure has a binding affinity for a Co-MOD that is from 1 nM to 100 nM, or from 100 nM to 100 μM. For example, in some cases, a variant MOD present in a T-Cell-MMP-epitope conjugate of the present disclosure has a binding affinity for a Co-MOD that is from about 1 nM to about 10 nM, from about 10 nM to about 50 nM, from about 50 nM to about 100 nM, from about 100 nM to about 200 nM, from about 200 nM to about 300 nM, from about 300 nM to about 500 nM, from about 500 nM to about 700 nM, from about 700 nM to about 1 μM, from about 1 μM to about 5 μM, from about 5 μM to about 10 μM, from about 10 μM to about 20 μM, from about 20 μM to about 50 μM, from about 50 μM to about 75 μM, or from about 75 μM to about 100 μM. In some cases, a variant MOD present in a T-Cell-MMP-epitope conjugate of the present disclosure has a binding affinity for a Co-MOD that is from about 1 nM to about 5 nM, from about 5 nM to about 10 nM, from about 10 nM to about 50 nM, or from about 50 nM to about 100 nM.

The combination of the reduced affinity of the MOD for its Co-MOD and the affinity of the epitope for a TCR provides for enhanced selectivity of a T-Cell-MMP-epitope conjugate of the present disclosure, while still allowing for activity of the MOD. For example, a T-Cell-MMP-epitope conjugate of the present disclosure binds selectively to a first T-cell that displays both: i) a TCR specific for the epitope present in the T-Cell-MMP-epitope conjugate; and ii) a Co-MOD that binds to the MOD present in the T-Cell-MMP-epitope conjugate, compared to binding to a second T-cell that displays: i) a TCR specific for an epitope other than the epitope present in the T-Cell-MMP-epitope conjugate; and ii) a Co-MOD that binds to the MOD present in the T-Cell-MMP-epitope conjugate. For example, a T-Cell-MMP-epitope conjugate of the present disclosure binds to the first T-cell with an affinity that is at least 10%, at least 15%, at least 20%, at least 25%, at least 30%, at least 40%, at least 50%, at least 60%, at least 70%, at least 80%, at least 90%, at least 200% (2-fold), at least 250% (2.5-fold), at least 500% (5-fold), at least 1,000% (10-fold), at least 1,500% (15-fold), at least 2,000% (20-fold), at least 2,500% (25-fold), at least 5,000% (50-fold), at least 10,000% (100-fold), or more than 100-fold, higher than the affinity to which it binds the second T-cell.

In some cases, a T-Cell-MMP-epitope conjugate of the present disclosure, when administered to an individual in need thereof, induces both an epitope-specific T-cell response and an epitope non-specific T-cell response. In other words, in some cases, the T-Cell-MMP-epitope conjugate of the present disclosure, when administered to an individual in need thereof, induces an epitope-specific T-cell response by modulating the activity of a first T-cell that displays both: i) a TCR specific for the epitope present in the T-Cell-MMP-epitope conjugate; and ii) a Co-MOD that binds to the MOD present in the T-Cell-MMP-epitope conjugate. The T-Cell-MMP-epitope conjugate also induces an epitope non-specific T-cell response by modulating the activity of a second T-cell that displays: i) a TCR specific for an epitope other than the epitope present in the T-Cell-MMP-epitope conjugate; and ii) a Co-MOD that binds to the MOD present in the T-Cell-MMP-epitope conjugate. The ratio of the epitope-specific T-cell response to the epitope-non-specific T-cell response is at least 2:1, at least 5:1, at least 10:1, at least 15:1, at least 20:1, at least 25:1, at least 50:1, or at least 100:1. The ratio of the epitope-specific T-cell response to the epitope-non-specific T-cell response is from about 2:1 to about 5:1, from about 5:1 to about 10:1, from about 10:1 to about 15:1, from about 15:1 to about 20:1, from about 20:1 to about 25:1, from about 25:1 to about 50:1, from about 50:1 to about 100:1, or more than 100:1. “Modulating the activity” of a T-cell can include, but is not limited to, one or more of: i) activating a cytotoxic (e.g., CD8⁺) T-cell to proliferate; ii) inducing cytotoxic activity of a cytotoxic (e.g., CD8⁺) T-cell; iii) inducing production and/or release of a cytotoxin (e.g., a perforin, a granzyme, a granulysin) by a cytotoxic (e.g., CD8+) T cell; and/or iv) increasing the number of epitope-specific T cells. As one example, the ratio of the increase of the number of epitope-specific T cells to the increase in the number of epitope non-specific T cells can be readily determined by known methods.

The combination of the reduced affinity of the MOD for its Co-MOD and the affinity of the epitope for a TCR provides for enhanced selectivity of a T-Cell-MMP-epitope conjugate of the present disclosure. Thus, for example, a T-Cell-MMP-epitope conjugate of the present disclosure binds with higher avidity to a first T-cell that displays both: i) a TCR specific for the epitope present in the T-Cell-MMP-epitope conjugate; and ii) a Co-MOD that binds to the MOD present in the T-Cell-MMP-epitope conjugate, compared to the avidity with which it binds to a second T-cell that displays: i) a TCR specific for an epitope other than the epitope present in the T-Cell-MMP-epitope conjugate; and ii) a Co-MOD that binds to the MOD present in the T-Cell-MMP-epitope conjugate.

a. Determining Binding Affinity

Binding affinity between a MOD and its Co-MOD can be determined by bio-layer interferometry (BLI) using purified MOD and purified Co-MOD. Binding affinity between a T-Cell-MMP-epitope conjugate and its Co-MOD(s) can be determined by BLI using purified T-Cell-MMP-epitope conjugate and the Co-MOD. BLI methods are well known to those skilled in the art. See, e.g., Lad et al. (2015) J. Biomol. Screen., 20(4):498-507; and Shah and Duncan (2014) J. Vis. Exp. 18:e51383. The specific and relative binding affinities described in this disclosure between a Co-MOD and a MOD, or between a Co-MOD and a T-Cell-MMP (or its epitope conjugate), can be determined using the procedures described or assessment of TMMP molecules in PCT/US2018/049756 published as WO 2019/051091. See e.g., paragraphs [0052] to [0064].

b. Dimerized Multimeric T-Cell Modulatory Polypeptides

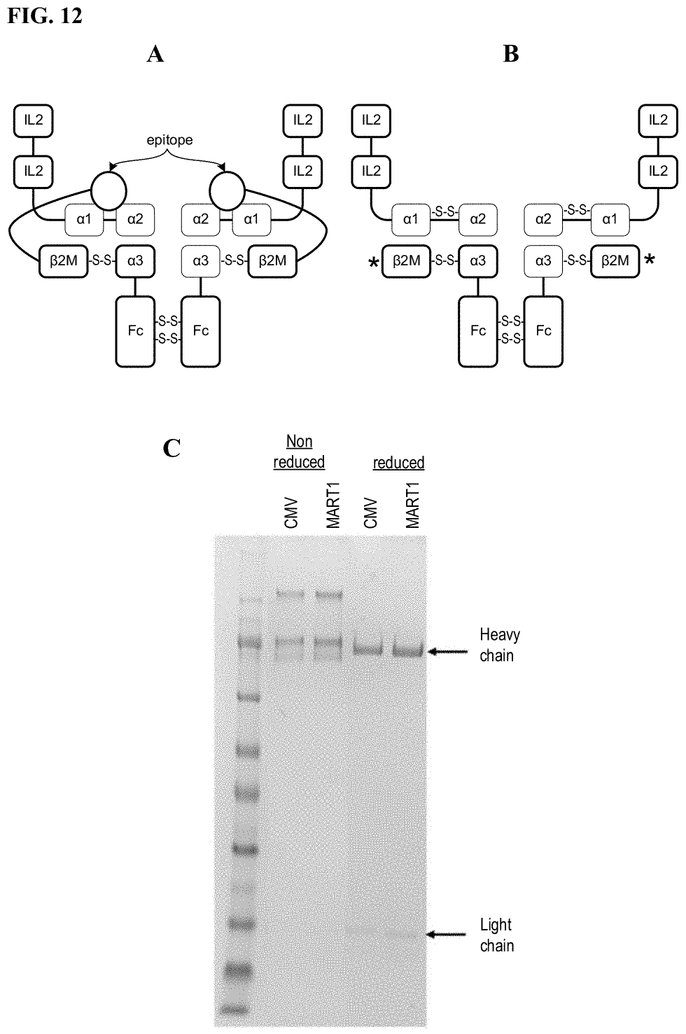

T-Cell-MMPs and T-Cell-MMP-epitope conjugates of the present disclosure can be in the form of dimers, i.e., the present disclosure provides a multimeric polypeptide comprising a dimer of a multimeric T-Cell-MMP of the present disclosure. An example of a dimerized T-Cell-MMP is shown in FIG. 12 structure B, and examples of dimerized T-Cell-MMP-epitope conjugates are shown in FIG. 7. Thus, the present disclosure provides a multimeric T-Cell-MMP comprising: A) a first heterodimer comprising: a) a first polypeptide comprising: i) a peptide epitope; and ii) a first MHC polypeptide; and b) a second polypeptide comprising a second MHC polypeptide, wherein the first heterodimer comprises one or more MODs; and B) a second heterodimer comprising: a) a first polypeptide comprising: i) a peptide epitope; and ii) a first MHC polypeptide; and b) a second polypeptide comprising a second MHC polypeptide, wherein the second heterodimer comprises one or more MODs, and wherein the first heterodimer and the second heterodimer are covalently linked to one another. In some cases, the two heterodimers that form the dimerized T-Cell-MMPs are identical to one another in as sequence. In some cases, the first heterodimer and the second heterodimer are covalently linked to one another via a C-terminal region of the second polypeptide of the first heterodimer and a C-terminal region of the second polypeptide of the second heterodimer (see, e.g., the disulfide bonds between the IgFc regions (“Fc”) in FIG. 7 and FIG. 12 structure B). In some cases, the first heterodimer and the second heterodimer are covalently linked to one another via an aa in the C-terminal region of the second polypeptide of the first heterodimer and an aa in the C-terminal region of the second polypeptide of the second heterodimer; for example, in some cases, the C-terminal aa of the second polypeptide of the first heterodimer and the C-terminal aa of the second polypeptide of the second heterodimer are linked to one another, either directly or via a linker. The linker can be a peptide linker. The peptide linker can have a length of from 1 aa to 200 aa (e.g., from 1 aa to 5 aa, from 5 aa to 10 aa, from 10 aa to 25 aa, from 25 aa to 50 aa, from 50 aa to 100 aa, from 100 aa to 150 aa, or from 150 aa to 200 aa).

The first MHC polypeptides of the first and second heterodimers may be β2M polypeptides, and the second MHC polypeptides of the first and second heterodimers may be MHC Class I heavy chain polypeptides. In some cases, the MOD of the first heterodimer and the MOD of the second heterodimer comprise the same an sequence. In some cases, the MOD of the first heterodimer and the MOD of the second heterodimer are variant MODs that comprise from 1 to 10 an substitutions compared to a corresponding parental wt. MOD, wherein from 1 to 10 an substitutions result in reduced affinity binding of the variant MOD to a Co-MOD. In some cases, the MOD(s) of the first heterodimer and the MOD(s) of the second heterodimer are each independently selected from the group consisting of IL-2, 4-1BBL, PD-L1, CD70, CD80, CD86, ICOS-L, OX-40L, FasL, JAG1(CD339), TGF-β, ICAM, and variant MODs thereof (e.g., variant MODs having 1 to 10 an substitutions compared to a corresponding parental wt. MOD). Examples of suitable MHC polypeptides, MODs, and peptide epitopes are described below.

In some cases, the peptide epitope of the first heterodimer and the peptide epitope of the second heterodimer comprise the same amino acid sequence.

In addition to dimers, the T-Cell-MMPs and T-Cell-MMP-epitope conjugates of the present disclosure may form higher order complexes including trimers, tetramers, or pentamers. Compositions comprising multimers of T-Cell-MMPs may also comprise lower order complexes such as monomers and, accordingly, may comprise monomers, dimers, trimers, tetramers, pentamers, or combinations of any thereof (e.g., a mixture of monomers and dimers).

4 MHC Polypeptides of T-Cell-MMPs

As noted above, T-Cell-MMPs and T-Cell-MMP-epitope conjugates include MHC polypeptides. For the purposes of the instant disclosure, the term “major histocompatibility complex (MHC) polypeptides” is meant to include MHC Class I polypeptides of various species, including human MHC (also referred to as human leukocyte antigen (HLA)) polypeptides, rodent (e.g., mouse, rat, etc.) MHC polypeptides, and MHC polypeptides of other mammalian species (e.g., lagomorphs, non-human primates, canines, felines, ungulates (e.g., equines, bovines, ovines, caprines, etc.), and the like. The term “MHC polypeptide” is meant to include Class I MHC polypeptides (e.g., β-2 microglobulin and MHC Class I heavy chain and/or portions thereof). In some cases, the first MHC polypeptide is an MHC Class I β2M (β2M) polypeptide, and the second MHC polypeptide is an MHC Class I heavy chain (MHC-H). In an embodiment, both the β2M and MHC-H chain sequences in a T-Cell-MMP (or its epitope conjugate) are of human origin. Unless expressly stated otherwise, the T-Cell-MMPs and the T-Cell-MMP-epitope conjugates described herein are not intended to include membrane anchoring domains (transmembrane regions) of an MHC Class I heavy chain, or a part of that molecule sufficient to anchor the resulting T-Cell-MMP, or a peptide thereof, to a cell (e.g., eukaryotic cell such as a mammalian cell) in which it is expressed. In some cases, the MHC Class I heavy chain present in T-Cell-MMPs and T-Cell-MMP-epitope conjugates does not include a signal peptide, a transmembrane domain, or an intracellular domain (cytoplasmic tail) associated with a native MHC Class I heavy chain. Thus, e.g., in some cases, the MHC Class I heavy chain present in a T-Cell-MMP of the present disclosure includes only the α1, α2, and α3 domains of an MHC Class I heavy chain. In some cases, the MHC Class I heavy chain present in a T-Cell-MMP of the present disclosure has a length of from about 270 amino acids (aa) to about 290 aa. In some cases, the MHC Class I heavy chain present in a T-Cell-MMP of the present disclosure has a length of 270 aa, 271 aa, 272 aa, 273 aa, 274 aa, 275 aa, 276 aa, 277 aa, 278 aa, 279 aa, 280 aa, 281 aa, 282 aa, 283 aa, 284 aa, 285 aa, 286 aa, 287 aa, 288 aa, 289 aa, or 290 aa.

In some cases, an MHC polypeptide of a T-Cell-MMP, or a T-Cell-MMP-epitope conjugate is a human MHC polypeptide, where human MHC polypeptides are also referred to as “human leukocyte antigen” (“HLA”) polypeptides, more specifically, a Class I HLA polypeptide, e.g., a β2M polypeptide, or a Class I HLA heavy chain polypeptide. Class I HLA heavy chain polypeptides that can be included in T-Cell-MMPs or their epitope conjugates include HLA-A, -B, -C, -E, -F, and/or -G heavy chain polypeptides. In an embodiment, the Class I HLA heavy chain polypeptides of T-Cell-MMPs or their epitope conjugates comprise polypeptides having a sequence having at least 75%, at least 80%, at least 85%, at least 90%, at least 95%, at least 98%, at least 99%, or 100% aa sequence identity to all or part (e.g., 50, 75, 100, 150, 200, 225, 250, or 260 contiguous aas) of the as sequence of any of the human HLA heavy chain polypeptides depicted in FIGS. 3A to 3I. For example, they may comprise 1-30, 1-5, 5-10, 10-15, 15-20, 20-25 or 25-30 as insertions, deletions, and/or substitutions (in addition to those locations indicated as being variable in the heavy chain consensus sequences of FIGS. 3E to 3I).

As an example, an MHC Class I heavy chain polypeptide of a multimeric polypeptide can comprise an aa sequence having at least 75%, at least 80%, at least 85%, at least 90%, at least 95%, at least 98%, at least 99%, or 100% as sequence identity to aas 25-300 (lacking all, or substantially all, of the leader, transmembrane and cytoplasmic sequences) or 25-365 (lacking the leader) of the human HLA-A heavy chain polypeptides depicted in FIGS. 3A, 3B and/or 3C.

a. MHC Class I Heavy Chains

Class I human MHC polypeptides may be drawn from the classical HLS alleles (HLA-A, B, and C), or the non-classical HLA alleles (e.g., HLA-E, F and G). The following are non-limiting examples of MHC-H alleles and variants of those alleles that may be incorporated into T-Cell-MMPs and their epitope conjugates.

(I) HLA-A Heavy Chains

The HLA-A heavy chain peptide sequences, or portions thereof, that may be incorporated into a T-Cell-MMP or its epitope conjugate include, but are not limited to, the alleles: A*0101, A*0201, A*0301, A*1101, A*2301, A*2402, A*2407, A*3303, and A*3401, which are aligned without all, or substantially all, of the leader, transmembrane and cytoplasmic sequences in FIG. 3E. Any of those alleles may comprise a substitution at one or more of positions 84, 139 and/or 236 (as shown in FIG. 3E) selected from: a tyrosine to alanine at position 84 (Y84A); a tyrosine to cysteine at position 84 (Y84C); an alanine to cysteine at position 139 (A139C); and an alanine to cysteine substitution at position 236 (A236C). In addition, an HLA-A sequence having at least 75% (e.g., at least 80%, at least 85%, at least 90%, at least 95%, at least 98%, at least 99% or 100%) as sequence identity to all or part (e.g., 50, 75, 100, 150, 200, 225, 250, or 260 contiguous aas) of the sequence of those HLA-A alleles may also be incorporated into a T-Cell-MMP (e.g., it may comprise 1-30, 1-5, 5-10, 10-15, 15-20, 20-25, or 25-30 as insertions, deletions, and/or substitutions). The HLA-A heavy chain polypeptide sequence of a T-Cell-MMP may comprise the Y84C and A139C substitutions.

(a) HLA-A*0101 (HLA-A*01:01:01:01)

An MHC Class I heavy chain polypeptide of a T-Cell-MMP or a T-Cell-MMP-epitope conjugate may comprise aa sequence of HLA-A*01:01:01:01 (HLA-A in FIG. 3D (SEQ ID NO:20) or FIG. 3E), or a sequence having at least 75% (at least 80%, at least 85%, at least 90%, at least 95%, at least 98%, at least 99% or 100%) as sequence identity to all or part (e.g., 50, 75, 100, 150, 200, 225, 250, or 260 contiguous aas) of that sequence (e.g., it may comprise 1-30, 1-5, 5-10, 10-15, 15-20, 20-25, or 25-30 as insertions, deletions, and/or substitutions). In an embodiment, where the HLA-A heavy chain polypeptide of a T-Cell-MMP or its epitope conjugate has less than 100% identity to the sequence labeled HLA-A in FIG. 3D, it may comprise a substitution at one or more of positions 84, 139 and/or 236 selected from: a tyrosine to alanine at position 84 (Y84A); a tyrosine to cysteine at position 84 (Y84C); an alanine to cysteine at position 139 (A139C); and an alanine to cysteine at position 236 (A236C). The HLA-A heavy chain polypeptide sequence of a T-Cell-MMP or its epitope conjugate may comprise the Y84C and A139C substitutions.

(b) HLA-A*0201 (HLA-A*02:01)

An MHC Class I heavy chain polypeptide of a T-Cell-MMP or a T-Cell-MMP-epitope conjugate may comprise an aa sequence of HLA-A*0201 (SEQ ID NO:23) provided in FIG. 3D or FIG. 3E, or a sequence having at least 75% (e.g., at least 80%, at least 85%, at least 90%, at least 95%, at least 98%, at least 99% or 100%) as sequence identity to all or part (e.g., 50, 75, 100, 150, 200, 225, 250, or 260 contiguous aas) of that sequence (e.g., it may comprise 1-30, 1-5, 5-10, 10-15, 15-20, 20-25, or 25-30 as insertions, deletions, and/or substitutions). In an embodiment, where the HLA-A*0201 heavy chain polypeptide of a T-Cell-MMP or its epitope conjugate has less than 100% identity to the sequence labeled HLA-A*0201 in FIG. 3D or 3E, it may comprise a substitution at one or more of positions 84, 139 and/or 236 selected from: a tyrosine to alanine at position 84 (Y84A); a tyrosine to cysteine at position 84 (Y84C); an alanine to cysteine at position 139 (A139C); and an alanine to cysteine at position 236 (A236C). In an embodiment, the HLA-A*0201 heavy chain polypeptide of a T-Cell-MMP or its epitope conjugate comprises the Y84A and A236C substitutions. The HLA-A*0201 heavy chain polypeptide sequence of a T-Cell-MMP may comprise the Y84C and A139C substitutions. In an embodiment, the HLA-A*0201 heavy chain polypeptide of a T-Cell-MMP or its epitope conjugate comprises the Y84C, A139C and A236C substitutions.

(c) HLA-A*1101 (HLA-A*11:01)

An MHC Class I heavy chain polypeptide of a T-Cell-MMP or a T-Cell-MMP-epitope conjugate may comprise an aa sequence of HLA-A*1101 (SEQ ID NO:28) provided in FIG. 3D or 3E, or a sequence having at least 75% (e.g., at least 80%, at least 85%, at least 90%, at least 95%, at least 98%, at least 99% or 100%) aa sequence identity to all or part (e.g., 50, 75, 100, 150, 200, 225, 250, or 260 contiguous aas) of that sequence (e.g., it may comprise 1-30, 1-5, 5-10, 10-15, 15-20, 20-25, or 25-30 aa insertions, deletions, and/or substitutions). The HLA-A*1101 heavy chain allele may be prominent in Asian populations, including populations of individuals of Asian descent.

In an embodiment, where the HLA-A*1101 heavy chain polypeptide of a T-Cell-MMP or its epitope conjugate has less than 100% identity to the sequence labeled HLA-A*1101 in FIG. 3D or 3E, it may comprise a substitution at one or more of positions 84, 139 and/or 236 selected from: a tyrosine to alanine at position 84 (Y84A); a tyrosine to cysteine at position 84 (Y84C); an alanine to cysteine at position 139 (A139C); and an alanine to cysteine at position 236 (A236C). In an embodiment, the HLA-A*1101 heavy chain polypeptide of a T-Cell-MMP or its epitope conjugate comprises the Y84A and A236C substitutions. The HLA-A*1101 heavy chain polypeptide of a T-Cell-MMP or its epitope conjugate may comprise the Y84C and A139C substitutions. In an embodiment, the HLA-A*1101 heavy chain polypeptide of a T-Cell-MMP or its epitope conjugate comprises the Y84C, A139C and A236C substitutions.

(d) HLA-A*2402 (HLA-A*24:02)

An MHC Class I heavy chain polypeptide of a T-Cell-MMP or a T-Cell-MMP-epitope conjugate may comprise an aa sequence of HLA-A*2402 (SEQ ID NO:29) provided in FIG. 3D or 3E, or a sequence having at least 75% (e.g., at least 80%, at least 85%, at least 90%, at least 95%, at least 98%, at least 99% or 100%) an sequence identity to all or part (e.g., 50, 75, 100, 150, 200, 225, 250, or 260 contiguous aas) of that sequence (e.g., it may comprise 1-30, 1-5, 5-10, 10-15, 15-20, 20-25, or 25-30 an insertions, deletions, and/or substitutions). The HLA-A*2402 heavy chain allele may be prominent in Asian populations, including populations of individuals of Asian descent.

In an embodiment, where the HLA-A*2402 heavy chain polypeptide of a T-Cell-MMP or its epitope conjugate has less than 100% identity to the sequence labeled HLA-A*2402 in FIG. 3D or 3E, it may comprise a substitution at one or more of positions 84, 139 and/or 236 selected from: a tyrosine to alanine at position 84 (Y84A); a tyrosine to cysteine at position 84 (Y84C); an alanine to cysteine at position 139 (A139C); and an alanine to cysteine at position 236 (A236C). The HLA-A*2402 heavy chain polypeptide of a T-Cell-MMP or its epitope conjugate may comprise the Y84A and A236C substitutions. The HLA-A*2402 heavy chain polypeptide of a T-Cell-MMP or its epitope conjugate may comprise the Y84C and A139C substitutions. In an embodiment, the HLA-A*2402 heavy chain polypeptide of a T-Cell-MMP or its epitope conjugate comprises the Y84C, A139C and A236C substitutions.

(e) HLA-A*3303 (HLA-A*33:03)

An MHC Class I heavy chain polypeptide of a T-Cell-MMP or a T-Cell-MMP-epitope conjugate may comprise an aa sequence of HLA-A*3303 (SEQ ID NO:30) provided in FIG. 3D or 3E, or a sequence having at least 75% (e.g., at least 80%, at least 85%, at least 90%, at least 95%, at least 98%, or at least 99%) or 100% aa sequence identity to all or part (e.g., 50, 75, 100, 150, 200, 225, 250, or 260 contiguous aas) of that sequence (e.g., it may comprise 1-25, 1-5, 5-10, 10-15, 15-20, 20-25, or 25-30 aa insertions, deletions, and/or substitutions). The HLA-A*3303 heavy chain allele may be prominent in Asian populations, including populations of individuals of Asian descent.

In an embodiment, where the HLA-A*3303 heavy chain polypeptide of a T-Cell-MMP or its epitope conjugate has less than 100% identity to the sequence labeled HLA-A*3303 in FIG. 3D, it may comprise a substitution at one or more of positions 84, 139 and/or 236 selected from: a tyrosine to alanine at position 84 (Y84A); a tyrosine to cysteine at position 84 (Y84C); an alanine to cysteine at position 139 (A139C); and an alanine to cysteine at position 236 (A236C). The HLA-A*3303 heavy chain polypeptide of a T-Cell-MMP or its epitope conjugate may comprise the Y84A and A236C substitutions. The HLA-A*3303 heavy chain polypeptide of a T-Cell-MMP or its epitope conjugate may comprise the Y84C and A139C substitutions. The HLA-A*3303 heavy chain polypeptide of a T-Cell-MMP or its epitope conjugate may comprise the Y84C, A139C and A236C substitutions.

(ii) HLA-B Heavy Chains.

The HLA-B heavy chain peptide sequences, or portions thereof, that may be incorporated into a T-Cell-MMP or its epitope conjugate include, but are not limited to, the alleles: B*0702, B*0801, B*1502, B*3802, B*4001, B*4601, and B*5301, which are aligned without all, or substantially all, of the leader, transmembrane and cytoplasmic sequences in FIG. 3F. Any of those alleles may comprise a substitution at one or more of positions 84, 139 and/or 236 (as shown in FIG. 3F) selected from: a tyrosine to alanine at position 84 (Y84A); a tyrosine to cysteine at position 84 (Y84C); an alanine to cysteine at position 139 (A139C); and an alanine to cysteine at position 236 (A236C). In addition, an HLA-B sequence having at least 75% (e.g., at least 80%, at least 85%, at least 90%, at least 95%, at least 98%, at least 99%) or 100% aa sequence identity to all or part (e.g., 50, 75, 100, 150, 200, 225, 250, or 260 contiguous aas) of the sequence of those HLA-B alleles may also be incorporated into a T-Cell-MMP (e.g., it may comprise 1-25, 1-5, 5-10, 10-15, 15-20, 20-25, or 25-30 aa insertions, deletions, and/or substitutions). The HLA-B heavy chain polypeptide sequence of a T-Cell-MMP or its epitope conjugate may comprise either Y84A and A236C substitutions or Y84C and A139C substitutions.

(a) HLA-B*0702 (HLA-B*07:02)

An MHC Class I heavy chain polypeptide of a T-Cell-MMP or a T-Cell-MMP-epitope conjugate may comprise an aa sequence of HLA-B*0702 (SEQ ID NO:21) in FIG. 3D (labeled HLA-B in FIG. 3D), HLA-B*03501, HLA-B*4402, HLA-B*4403, HLA-B*5801 or a sequence having at least 75% (e.g., at least 80%, at least 85%, at least 90%, at least 95%, at least 98%, at least 99%) or 100% aa sequence identity to all or part (e.g., 50, 75, 100, 150, 200, 225, 250, or 260 contiguous aas) of any of those sequences (e.g., it may comprise 1-25, 1-5, 5-10, 10-15, 15-20, 20-25, or 25-30 as insertions, deletions, and/or substitutions). In an embodiment, where the HLA-B heavy chain polypeptide of a T-Cell-MMP or its epitope conjugate has less than 100% identity to the sequence labeled HLA-B in FIG. 3D, it may comprise a substitution at one or more of positions 84, 139 and/or 236 selected from: a tyrosine to alanine at position 84 (Y84A); a tyrosine to cysteine at position 84 (Y84C); an alanine to cysteine at position 139 (A139C); and an alanine to cysteine at position 236 (A236C). The HLA-B heavy chain polypeptide of a T-Cell-MMP or its epitope conjugate may comprise the Y84A and A236C substitutions. The HLA-B heavy chain polypeptide of a T-Cell-MMP or its epitope conjugate may comprise the Y84C and A139C substitutions. The HLA-B heavy chain polypeptide of a T-Cell-MMP or its epitope conjugate may comprise the Y84C, A139C and A236C substitutions.

(b) HLA-B*3501 (HLA-B*35:01)

An MHC Class I heavy chain polypeptide of a T-Cell-MMP or a T-Cell-MMP-epitope conjugate may comprise an aa sequence of HLA-B*3501: GSHSMRYFYTAMSRPGRGEPRFIAVGYVDDTQ-FVRFDSDAASPRTEPRAPWIEQEGPEYWDRNTQIFKTNTQTYRESLRNLRGYYNQSEAGSHIIQR MYGCDLGPDGRLLRGHDQSAYDGKDYIALNEDLSSWTAADTAAQITQRKWEAARVAEQLRAY LEGLCVEWLRRYLENGKETLQRADPPKTHVTHHPVSDHEATLRCWALGFYPAEITLTWQRDGE DQTQDTELVETRPAGDRTFQKWAAVVVPSGEEQRYTCHVQHEGLPKPLTLRWEP (shown lacking its signal sequence and transmembrane/intracellular regions SEQ ID NO:127), or a sequence having at least 75% (e.g., at least 80%, at least 85%, at least 90%, at least 95%, at least 98%, at least 99%) or 100% aa sequence identity to all or part (e.g., 50, 75, 100, 150, 200, 225, 250, or 260 contiguous aas) of that sequence (e.g., it may comprise 1-25, 1-5, 5-10, 10-15, 15-20, 20-25, or 25-30 aa insertions, deletions, and/or substitutions). In an embodiment, the sequence may comprise a substitution at one or more of positions 84, 139 and/or 236 selected from: a tyrosine to alanine at position 84 (Y84A); a tyrosine to cysteine at position 84 (Y84C); an alanine to cysteine at position 139 (A139C); and an alanine to cysteine at position 236 (A236C). The HLA-B*3501 heavy chain polypeptide of a T-Cell-MMP or its epitope conjugate may comprise the Y84A and A236C substitutions. The HLA-B*3501 heavy chain polypeptide of a T-Cell-MMP or its epitope conjugate may comprise the Y84C and A139C substitutions. The HLA-B*3501 heavy chain polypeptide of a T-Cell-MMP or its epitope conjugate may comprise the Y84C, A139C and A236C substitutions.

(c) HLA-B*4402 (HLA-B*44:02)

An MHC Class I heavy chain polypeptide of a T-Cell-MMP or a T-Cell-MMP-epitope conjugate may comprise an aa sequence of HLA-B*4402: GSHSMRYFYTAMSRPGRGEPRFITVGYVDDTL-FVRFDSDATSPRKEPRAPWIEQEGPEYWDRETQISKTNTQTYRENLRTALRYYNQSEAGSHIIQR MYGCDVGPDGRLLRGYDQDAYDGKDYIALNEDLSSWTAADTAAQITQRKWEAARVAEQDRA YLEGLCVESLRRYLENGKETLQRADPPKTHVTHHPISDHEVTLRCWALGFYPAEITLTWQRDGE DQTQDTELVETRPAGDRTFQKWAAVVVPSGEEQRYTCHVQHEGLPKPLTLRWEP (shown lacking its signal sequence and transmembrane/intracellular regions SEQ ID NO:128), or a sequence having at least 75% (e.g., at least 80%, at least 85%, at least 90%, at least 95%, at least 98%, at least 99%) or 100% aa sequence identity to all or part (e.g., 50, 75, 100, 150, 200, 225, 250, or 260 contiguous aas) of that sequence (e.g., it may comprise 1-25, 1-5, 5-10, 10-15, 15-20, 20-25, or 25-30 an insertions, deletions, and/or substitutions). In an embodiment, the sequence may comprise a substitution at one or more of positions 84, 139 and/or 236 selected from: a tyrosine to alanine at position 84 (Y84A); a tyrosine to cysteine at position 84 (Y84C); an alanine to cysteine at position 139 (A139C); and an alanine to cysteine at position 236 (A236C). The HLA-B*4402 heavy chain polypeptide of a T-Cell-MMP or its epitope conjugate may comprise the Y84A and A236C substitutions. The HLA-B*4402 heavy chain polypeptide of a T-Cell-MMP or its epitope conjugate may comprise the Y84C and A139C substitutions. The HLA-B*4402 heavy chain polypeptide of a T-Cell-MMP or its epitope conjugate may comprise the Y84C, A139C and A236C substitutions.

(d) HLA-B*4403 (HLA-B*44:03)

An MHC Class I heavy chain polypeptide of a T-Cell-MMP or a T-Cell-MMP-epitope conjugate may comprise an aa sequence of HLA-B*4403: GSHSMRYFYTAMSRPGRGEPRFITVGYVDDT-LFVRFDSDATSPRKEPRAPWIEQEGPEYWDRETQISKTNTQTYRENLRTALRYYNQSEAGSHIIQR MYGCDVGPDGRLLRGYDQDAYDGKDYIALNEDLSSWTAADTAAQITQRKWEAARVAEQLRA YLEGLCVESLRRYLENGKETLQRADPPKTHVTHHPISDHEVTLRCWALGFYPAEITLTWQRDGE DQTQDTELVETRPAGDRTFQKWAAVVVPSGEEQRYTCHVQHEGLPKPLTLRWEP (shown lacking its signal sequence and transmembrane/intracellular regions SEQ ID NO:129), or a sequence having at least 75% (e.g., at least 80%, at least 85%, at least 90%, at least 95%, at least 98%, at least 99%) or 100% aa sequence identity to all or part (e.g., 50, 75, 100, 150, 200, 225, 250, or 260 contiguous aas) of that sequence (e.g., it may comprise 1-25, 1-5, 5-10, 10-15, 15-20, 20-25, or 25-30 an insertions, deletions, and/or substitutions). In an embodiment, the sequence may comprise a substitution at one or more of positions 84, 139 and/or 236 selected from: a tyrosine to alanine at position 84 (Y84A); a tyrosine to cysteine at position 84 (Y84C); an alanine to cysteine at position 139 (A139C); and an alanine to cysteine at position 236 (A236C). The HLA-B*4403 heavy chain polypeptide of a T-Cell-MMP or its epitope conjugate may comprise the Y84A and A236C substitutions. The HLA-B*4403 heavy chain polypeptide of a T-Cell-MMP or its epitope conjugate may comprise the Y84C and A139C substitutions. The HLA-B*4403 heavy chain polypeptide of a T-Cell-MMP or its epitope conjugate may comprise the Y84C, A139C and A236C substitutions.

(e) HLA-B*5801 (HLA-B*58:01)

An MHC Class I heavy chain polypeptide of a T-Cell-MMP or a T-Cell-MMP-epitope conjugate may comprise an aa sequence of HLA-B*4403: GSHSMRYFYTAMSRPGRGEPRFIAVGYVDDT-QFVRFDSDAASPRTEPRAPWIEQEGPEYWDGETRNMKASAQTYRENLRIALRYYNQSEAGSHIIQ RMYGCDLGPDGRLLRGHDQSAYDGKDYIALNEDLSSWTAADTAAQTTQRKWEAARVAEQLRA YLEGLCVEWLRRYLENGKETLQRADPPKTHVTHHPVSDHEATLRCWALGFYPAEITLTWQRDG EDQTQDTELVETRPAGDRTFQKWAAVVVPSGEEQRYTCHVQHEGLPKPLTLRWEP (shown lacking its signal sequence and transmembrane/intracellular regions SEQ ID NO:129), or a sequence having at least 75% (e.g., at least 80%. at least 85%, at least 90%, at least 95%, at least 98%, at least 99%) or 100% aa sequence identity to all or part (e.g., 50, 75, 100, 150, 200, 225, 250, or 260 contiguous aas) of that sequence (e.g., it may comprise 1-25, 1-5, 5-10, 10-15, 15-20, 20-25, or 25-30 an insertions, deletions, and/or substitutions). In an embodiment, the sequence may comprise a substitution at one or more of positions 84, 139 and/or 236 selected from: a tyrosine to alanine at position 84 (Y84A); a tyrosine to cysteine at position 84 (Y84C); an alanine to cysteine at position 139 (A139C); and an alanine to cysteine at position 236 (A236C). The HLA-B*5801 heavy chain polypeptide of a T-Cell-MMP or its epitope conjugate may comprise the Y84A and A236C substitutions. The HLA-B*5801 heavy chain polypeptide of a T-Cell-MMP or its epitope conjugate may comprise the Y84C and A139C substitutions. The HLA-B*5801 heavy chain polypeptide of a T-Cell-MMP or its epitope conjugate may comprise the Y84C, A139C and A236C substitutions.

(iii) HLA-C heavy chains

The HLA-C heavy chain peptide sequences, or portions thereof, that may be incorporated into a T-Cell-MMP or its epitope conjugate include, but are not limited to, the alleles: C*0102, C*0303, C*0304, C*0401, C*0602, C*0701, C*0702, C*0801, and C*1502, which are aligned without all, or substantially all, of the leader, transmembrane and cytoplasmic sequences in FIG. 3G. Any of those alleles may comprise a substitution at one or more of positions 84, 139 and/or 236 (as shown in FIG. 3G) selected from: a tyrosine to alanine at position 84 (Y84A); a tyrosine to cysteine at position 84 (Y84C); an alanine to cysteine at position 139 (A139C); and an alanine to cysteine at position 236 (A236C). In addition, an HLA-C sequence having at least 75% (e.g., at least 80%, at least 85%, at least 90%, at least 95%, at least 98%, at least 99%) or 100% aa sequence identity to all or part (e.g., 50, 75, 100, 150, 200, 225, 250, or 260 contiguous aas) of the sequence of those HLA-C alleles may also be incorporated into a T-Cell-MMP (e.g., it may comprise 1-25, 1-5, 5-10, 10-15, 15-20, 20-25, or 25-30 aa insertions, deletions, and/or substitutions). The HLA-C heavy chain polypeptide sequence of a T-Cell-MMP or its epitope conjugate may comprise either Y84A and A139C substitutions, or Y84C and A139C substitutions.

(a) HLA-C*701 (HLA-C*07:01) and HLA-C*702 (HLA-C*07:02)

An MHC Class I heavy chain polypeptide of a T-Cell-MMP or a T-Cell-MMP-epitope conjugate may comprise an aa sequence of HLA-C*701 (SEQ ID NO:49 in FIG. 3C) or HLA-C*702 (SEQ ID NO:50) in FIG. 3G (labeled HLA-C in FIG. 3D), or a sequence having at least 75% (e.g., at least 80%, at least 85%, at least 90%, at least 95%, at least 98%, at least 99%) or 100% aa sequence identity to all or part (e.g., 50, 75, 100, 150, 200, 225, 250, or 260 contiguous aas) of one of those sequences (e.g., it may comprise 1-25, 1-5, 5-10, 10-15, 15-20, 20-25, or 25-30 aa insertions, deletions, and/or substitutions relative to those sequences). In an embodiment, where the HLA-C heavy chain polypeptide of a T-Cell-MMP or its epitope conjugate has less than 100% identity to the sequence labeled HLA-C in FIG. 3D, it may comprise a substitution at one or more of positions 84, 139 and/or 236 selected from: a tyrosine to alanine at position 84 (Y84A); a tyrosine to cysteine at position 84 (Y84C); an alanine to cysteine at position 139 (A139C); and an alanine to cysteine at position 236 (A236C). The HLA-C heavy chain polypeptide of a T-Cell-MMP or its epitope conjugate may comprise the Y84A and A236C substitutions. The HLA-C heavy chain polypeptide of a T-Cell-MMP or its epitope conjugate may comprise the Y84C and A139C substitutions. The HLA-C heavy chain polypeptide of a T-Cell-MMP or its epitope conjugate may comprise the Y84C, A139C and A236C substitutions.

(iv) Non-Classical HLA-E, F and G Heavy Chains

The non-classical HLA heavy chain peptide sequences, or portions thereof, that may be incorporated into a T-Cell-MMP or its epitope conjugate include, but are not limited to, those of the HLA-E, F, and/or G alleles. Sequences for those alleles, (and the HLA-A, B and C alleles) may be found on the world wide web at, for example, hla.alleles.org/nomenclature/index.html, the European Bioinformatics Institute (www.ebi.ac.uk), which is part of the European Molecular Biology Laboratory (EMBL), and the National Center for Bioecology Information (www.ncbi.nlm.nih.gov).

Some suitable HLA-E alleles include, but are not limited to, HLA-E*0101 (HLA-E*01:01:01:01), HLA-E*01:03 (HLA-E*01:03:01:01), HLA-E*01:04, HLA-E*01:05, HLA-E*01:06, HLA-E*01:07, HLA-E*01:09, and HLA-E*01:10. Some suitable HLA-F alleles include, but are not limited to, HLA-F*0101 (HLA-F*01:01:01:01), HLA-F*01:02, HLA-F*01:03 (HLA-F*01:03:01:01), HLA-F*01:04, HLA-F*01:05, and HLA-F*01:06. Some suitable HLA-G alleles include, but are not limited to, HLA-G*0101 (HLA-G*01:01:01:01), HLA-G*01:02, HLA-G*01:03 (HLA-G*01:03:01:01), HLA-G*01:04 (HLA-G*01:04:01:01), HLA-G*01:06, HLA-G*01:07, HLA-G*01:08, HLA-G*01:09: HLA-G*01:10, HLA-G*01:11, HLA-G*01:12, HLA-G*01:14, HLA-G*01:15, HLA-G*01:16, HLA-G*01:17, HLA-G*01:18: HLA-G*01:19, HLA-G*01:20, and HLA-G*01:22. Consensus sequences for those HLA-E, -F, and -G alleles without all, or substantially all, of the leader, transmembrane and cytoplasmic sequences are provided in FIG. 3H, and aligned with consensus sequences of the above-mentioned HLA-A, -B, and -C alleles provided in FIGS. 3E-3G and in FIG. 3I.

Any of the above-mentioned HLA-E, F and/or G alleles may comprise a substitution at one or more of positions 84, 139 and/or 236 as shown in FIG. 3I for the consensus sequences. In an embodiment, the substitutions may be selected from: a position 84 tyrosine to alanine (Y84A) or cysteine (Y84C), or in the case of HLA-F a R84A or R84C substitution; a position 139 alanine to cysteine (A139C), or in the case of HLA-F a V139C substitution; and an alanine to cysteine at position 236 (A236C). In addition, HLA-E, -F, and/or -G sequences having at least 75% (e.g., at least 80%, at least 85%, at least 90%, at least 95%, at least 98%, at least 99%) or 100% aa sequence identity to all or part (e.g., 50, 75, 100, 150, 200, 225, 250, or 260 contiguous aas) of any of the consensus sequences set forth in FIG. 3I may also be employed (e.g., the sequences may comprise 1-25, 1-5, 5-10, 10-15, 15-20, 20-25, or 25-30 aa insertions, deletions, and/or substitutions in addition to changes at variable residues listed therein). The HLA-E, F, or G heavy chain polypeptide sequence of a -Cell-MMP or a T-Cell-MMP-epitope conjugate may comprise as substitutions either an alanine at position 84 and a cysteine at position 139, or cysteines at both position 84 and position 139 (see e.g., FIG. 3I for the location of those positions).

(v) Mouse H2K

An MHC Class I heavy chain polypeptide of a T-Cell-MMP or a T-Cell-MMP-epitope conjugate may comprise an aa sequence of MOUSE H2K (SEQ ID NO:24) (MOUSE H2K in FIG. 3D), or a sequence having at least 75%, at least 80%, at least 85%, at least 90%, at least 95%, at least 98%, at least 99%, or 100% aa sequence identity to all or part (e.g., 50, 75, 100, 150, 200, 225, 250, or 260 contiguous aas) of that sequence (e.g., it may comprise 1-30, 1-5, 5-10, 10-15, 15-20, 20-25, or 25-30 aa insertions, deletions, and/or substitutions). In an embodiment, where the MOUSE H2K heavy chain polypeptide of a T-Cell-MMP or its epitope conjugate has less than 100% identity to the sequence labeled MOUSE H2K in FIG. 3D, it may comprise a substitution at one or more of positions 84, 139 and/or 236 selected from: a tyrosine to alanine at position 84 (Y84A); a tyrosine to cysteine at position 84 (Y84C); an alanine to cysteine at position 139 (A139C); and an alanine to cysteine at position 236 (A236C). In an embodiment, the MOUSE H2K heavy chain polypeptide of a T-Cell-MMP or its epitope conjugate comprises the Y84A and A236C substitutions. In an embodiment, the MOUSE H2K heavy chain polypeptide of a T-Cell-MMP or its epitope conjugate comprises the Y84C and A139C substitutions. In an embodiment, the MOUSE H2K heavy chain polypeptide of a T-Cell-MMP or its epitope conjugate comprises the Y84C, A139C and A236C substitutions.

(vi) The Effect of Amino Acid Substitutions in MHC Polypeptides on T-Cell-MMPs

(a) Substitutions at Positions 84,139 and 236

Substitution of position 84 of the MHC H chain (see FIG. 3I), particularly when it is a tyrosine residue, with a small amino acid such as alanine (Y84A) tends to open one end of the MHC binding pocket, allowing a linker (e.g., attached to an epitope peptide) to “thread” through the end of the pocket, and accordingly, permits a greater variation in the size of the epitope (e.g., longer peptides bearing epitope sequences) that can fit into the MHC pocket and be presented by the T-Cell-MMP. In an embodiment, the HLA-A heavy chain polypeptide of a T-Cell-MMP or its epitope conjugate comprises the Y84A and A236C substitutions. In an embodiment, the HLA-A heavy chain polypeptide of a T-Cell-MMP or its epitope conjugate comprises the Y84C and A139C substitutions. When amino acids 84 and 139 are both cysteines they may form an intrachain disulfide bond which can stabilize the MHC Class 1 protein and permit translation and excretion of the T-Cell-MMP by eukaryotic cells, even when not loaded with an epitope peptide. When position 84 is a C residue, it can also form an interchain disulfide bond with a linker attached to the N-terminus of a β2M polypeptide (e.g., epitope-linker sequence-mature β2M polypeptide, such as epitope-GCGGS(G₄S) linker sequence (SEQ ID NO:93)-mature β2M polypeptide, see SEQ ID NOs:57 to 61). When amino acid 236 is a cysteine it can form an interchain disulfide bond with a cysteine at amino acid 12 of a variant β2M polypeptide that comprises R12C substitution at that position. Some possible combinations of MHC Class 1 heavy chain sequence modifications that may be incorporated into a T-Cell-MMP or its epitope conjugate are shown in the Table that follows. Any combination of substitutions provided in the table at residues 84, 139 and 236 may be combined with any combination of substitutions in the epitope binding cleft, such as those described at positions 116 and 167.

(b) Substitutions at Positions 116 and 167

Any MHC Class I heavy chain sequences (including those disclosed above for: the HLA-A*0101 (HLA-A*01:01:01:01); HLA-A*0201; HLA-A*1101; HLA-A*2402; HLA-A*3303; HLA-B; HLA-C; and Mouse H2K, or the HLA-A, B, C, E, F, and/or G) may further comprise a cysteine substitution at position 116 (e.g., Y116C), providing thiol for anchoring an epitope peptide such as by reaction with a maleimide peptide and/or one of an alanine (W167A) or cysteine (W167C) at position 167. As with aa position 84 substitutions that open one end of the MHC-H binding pocket (e.g., Y84A or its equivalent), substitution of an alanine or glycine at position 167 or its equivalent (e.g., a W167A substitution or its equivalent) opens the other end of the MHC binding pocket, creating a groove that permits greater variation (e.g., longer length) of the epitope peptides that may be presented by the T-Cell-MMP-epitope conjugates. Substitutions at positions 84 and 167 or their equivalent (e.g., Y84A in combination with W167A or W167G) may be used in combination to modify the binding pocket of MHC-H chains. The placement of a cysteine at position 167 (e.g., a W1167C substitution) or its equivalent provides a thiol residue for anchoring an epitope peptide. Cysteine substitutions at positions 116 and 167 may be used separately or in combination to anchor epitopes (e.g., epitope peptides) in one or two locations (e.g., the ends of the epitope containing peptide. Substitutions at positions 116 and/or 167 may be combined with substitutions at positions 84, 139 and/or 236 described above.

SOME COMBINATIONS OF MHC CLASS 1 HEAVY CHAIN SEQUENCE MODIFICATIONS
THAT MAY BE INCORPORATED INTO A T-CELL-MMP OR ITS EPITOPE CONJUGATE

| | HLA Heavy | | Specific | |
|---|---|---|---|---|
| | Chain Sequence | | Substitutions at aa | Substitutions at |
| | From | | positions 84, 139 | positions 116 |
| Entry | FIGS. 3D-H | Sequence Identity Range [symbol] | and/or 236 | and/or 167 |
| 1 | HLA-A | 75%-99.8%, 80%-99.8%, 85%- | None; Y84C; Y84A; | None; |
| | Consensus | 99.8%, 90%-99.8%, 95%-99.8%, | A139C; A236C; | Y116C; |
| | FIG. 3E | 98%-99.8%, or99%-99.8%; or 1-25, | (Y84A & A236C); | W167A; |
| | | 1-5, 5-10, 10-15, 15-20, or 20-25 aa | (Y84C & A139C); or | W167C; or |
| | | insertions, deletions, and/or | (Y84C, A139C & | (Y116C & |
| | | substitutions (not counting | A236C) | W167C) |
| | | variable residues) | | |
| 2 | A*0101, A*0201, | 75%-99.8%, 80%-99.8%, 85%- | None; Y84C; Y84A; | None; |
| | A*0301, A*1101, | 99.8%, 90%-99.8%, 95%-99.8%, | A139C; A236C; | Y116C; |
| | A*2402, A*2301, | 98%-99.8%, or 99%-99.8%; or 1-25, | (Y84A & A236C); | W167A; |
| | A*2402, A*2407, | 1-5, 5-10, 10-15, 15-20, or 20-25 aa | (Y84C & A139C); or | W167C; or |
| | A*3303, or | insertions, deletions, | (Y84C, A139C & | (Y116C & |
| | A*3401 | and/or substitutions | A236C) | W167C) |
| 3 | HLA-B | 75%-99.8%, 80%-99.8%, 85%- | None; Y84C; Y84A; | None; |
| | Consensus | 99.8%, 90%-99.8%, 95%-99.8%, | A139C; A236C; | Y116C; |
| | FIG. 3F | 98%-99.8%, or 99%-99.8%; or 1-25, | (Y84A & A236C); | W167A; |
| | | 1-5, 5-10, 10-15, 15-20, or 20-25 aa | (Y84C & A139C); or | W167C; or |
| | | insertions, deletions, and/or | (Y84C, A139C & | (Y116C & |
| | | substitutions (not counting | A236C) | W167C) |
| | | variable residues) | | |
| 4 | B*0702, B*0801, | 75%-99.8%, 80%-99.8%, 85%- | None; Y84C; Y84A; | None; |
| | B*1502, B*3802, | 99.8%, 90%-99.8%, 95%-99.8%, | A139C; A236C; | Y116C; |
| | B*4001, B*4601, | 98%-99.8%, or 99%-99.8%; or 1-25, | (Y84A & A236C); | W167A; |
| | or B*5301 | 1-5, 5-10, 10-15, 15-20, or 20-25 aa | (Y84C & A139C); or | W167C; or |
| | | insertions, deletions, | (Y84C, A139C & | (Y116C & |
| | | and/or substitutions | A236C) | W167C) |
| 5 | HLA-C | 75%-99.8%, 80%-99.8%, 85%- | None; Y84C; Y84A; | None; |
| | Consensus | 99.8%, 90%-99.8%, 95%-99.8%, | A139C; A236C; | Y116C; |
| | FIG. 3G | 98%-99.8%, or 99%-99.8%; or 1-25, | (Y84A & A236C); | W167A; |
| | | 1-5, 5-10, 10-15, 15-20, or 20-25 aa | (Y84C & A139C); or | |
| | | insertions, deletions, and/or | (Y84C, A139C & | W167C; or |
| | | substitutions (not counting | A236C) | (Y116C & |
| | | variable residues) | | W167C) |
| 6 | C*0102, C*0303, | 75%-99.8%, 80%-99.8%, 85%- | None; Y84C; Y84A; | None; |
| | C*0304, C*0401, | 99.8%, 90%-99.8%, 95%-99.8%, | A139C; A236C; | Y116C; |
| | C*0602, C*0701, | 98%-99.8%, or 99%-99.8%; or 1-25, | (Y84A & A236C); | W167A; |
| | C*702, C*0801, | 1-5, 5-10, 10-15, 15-20, or 20-25 aa | (Y84C & A139C); or | W167C; or |
| | or C*1502 | insertions, deletions, | (Y84C, A139C & | (Y116C & |
| | | and/or substitutions | A236C) | W167C) |
| 7 | HLA-E, F, or G | 75%-99.8%, 80%-99.8%, 85%- | None; Y84C; Y84A; | None; |
| | Consensus | 99.8%, 90%-99.8%, 95%-99.8%, | A139C; A236C; | Y116C; |
| | FIG. 3H | 98%-99.8%, or 99%-99.8%; or 1-25, | (Y84A & A236C); | W167A; |
| | | 1-5, 5-10, 10-15, 15-20, or 20-25 aa | (Y84C & A139C); or | W167C; or |
| | | insertions, deletions, and/or | (Y84C, A139C & | (Y116C & |
| | | substitutions (not counting | A236C) | W167C) |
| | | variable residues) | | |
| 8 | MOUSE H2K | 75%-99.8%, 80%-99.8%, 85%- | None; Y84C; Y84A; | None; |
| | | 99.8%, 90%-99.8%, 95%-99.8%, | A139C; A236C; | Y116C; |
| | | 98%-99.8%, or 99%-99.8%; or 1-25, | (Y84A & A236C); | W167A; |
| | | 1-5, 5-10, 10-15, 15-20, or 20-25 aa | (Y84C & A139C); or | W167C; or |
| | | insertions, deletions, | (Y84C, A139C & | (Y116C & |
| | | and/or substitutions | A236C) | W167C) |

[symbol]

The Sequence Identity Range is the permissible range in sequence identity of an MHC-H polypeptide sequence incorporated into a T-Cell-MMP relative to the corresponding portion of the sequences listed in FIG. 3D-3H not counting the variable residues in the consensus sequences.

b. MHC Class I β2-Microglobins and Combinations with MHC-H Polypeptides

A β2M polypeptide of a T-Cell-MMP or its epitope conjugate can be a human β2M polypeptide, a non-human primate 032M polypeptide, a murine β2M polypeptide, and the like. In some instances, a β2M polypeptide comprises an aa sequence having at least 75%, at least 80%, at least 85%, at least 90%, at least 95%, at least 98%, at least 99%, or 100% as sequence identity to a 032M aa sequence depicted in FIG. 4. In some instances, a β2M polypeptide comprises an aa sequence having at least 75%, at least 80%, at least 85%, at least 90%, at least 95%, at least 98%, at least 99%, or 100% aa sequence identity to aas 21 to 119 of a β2M as sequence depicted in FIG. 4.

In some cases, an MHC polypeptide comprises a single aa substitution relative to a reference MHC polypeptide (where a reference MHC polypeptide can be a wt. MHC polypeptide), where the single an substitution substitutes an aa with a cysteine (Cys) residue. Such cysteine residues, when present in an MHC polypeptide of a first polypeptide of a T-Cell-MMP, or its epitope conjugate, can form a disulfide bond with a cysteine residue present in a second polypeptide chain.

In some cases, a first MHC polypeptide in a first polypeptide of a T-Cell-MMP and/or a second MHC polypeptide in a second polypeptide of a T-Cell-MMP. include a substitution of an aa with a cysteine, where the substituted cysteine in the first MHC polypeptide forms a disulfide bond with a cysteine in the second MHC polypeptide, where a cysteine in the first MHC polypeptide forms a disulfide bond with the substituted cysteine in the second MHC polypeptide, or where the substituted cysteine in the first MHC polypeptide forms a disulfide bond with the substituted cysteine in the second MHC polypeptide.

For example, in some cases, one of the following pairs of residues in an HLA β2M (see FIG. 4) and an HLA Class I heavy chain (see FIGS. 3D-3I) is substituted with cysteines (where residue numbers are those of the mature polypeptide): 1) β2M residue 12, HLA Class I heavy chain residue 236; 2) β2M residue 12, HLA Class I heavy chain residue 237; 3) β2M residue 8, HLA Class I heavy chain residue 234; 4) β2M residue 10, HLA Class I heavy chain residue 235; 5) β2M residue 24, HLA Class I heavy chain residue 236; 6) β2M residue 28, HLA Class I heavy chain residue 232; 7) β2M residue 98, HLA Class I heavy chain residue 192; 8) β2M residue 99, HLA Class I heavy chain residue 234; 9) β2M residue 3, HLA Class I heavy chain residue 120; 10) β2M residue 31, HLA Class I heavy chain residue 96; 11) β2M residue 53, HLA Class I heavy chain residue 35; 12) β2M residue 60, HLA Class I heavy chain residue 96; 13) β2M residue 60, HLA Class I heavy chain residue 122; 14) β2M residue 63, HLA Class I heavy chain residue 27; 15) β2M residue Arg3, HLA Class I heavy chain residue Gly120; 16) β2M residue His31, HLA Class I heavy chain residue Gln96; 17) β2M residue Asp53, HLA Class I heavy chain residue Arg35; 18) β2M residue Trp60, HLA Class I heavy chain residue G1n %; 19) β2M residue Trp60, HLA Class I heavy chain residue Asp122; 20) β2M residue Tyr63, HLA Class I heavy chain residue Tyr27; 21) β2M residue Lys6, HLA Class I heavy chain residue Glu232; 22) β2M residue Gln8, HLA Class I heavy chain residue Arg234; 23) β2M residue Tyr10, HLA Class I heavy chain residue Pro235; 24) β2M residue Ser11, HLA Class I heavy chain residue Gln242; 25) β2M residue Asn24, HLA Class I heavy chain residue Ala236; 26) β2M residue Ser28, HLA Class I heavy chain residue Glu232; 27) β2M residue Asp98, HLA Class I heavy chain residue His192; and 28) β2M residue Met99, HLA Class I heavy chain residue Arg234. The amino acid numbering of the MHC/HLA Class I heavy chain is in reference to the mature MHC/HLA Class I heavy chain, without a signal peptide. For example, in some cases, residue 236 of the mature HLA-A, -B, or -C aa sequence (i.e., residue 260 of the aa sequence depicted in FIGS. 3A-3C respectively) is substituted with a Cys. In some cases, residue 32 (corresponding to Arg-12 of mature β2M) of an aa sequence depicted in FIG. 4 is substituted with a Cys.

Separately, or in addition to, the pairs of cysteine residues in a β2M and HLA Class I heavy chain polypeptide that may be used to form interchain disulfide bonds between the first and second polypeptides of a T-Cell-MMP (discussed above), the HLA-heavy chain of a T-Cell-MMP or its epitope conjugate may be substituted with cysteines to form an intrachain disulfide bond between a cysteine substituted into the carboxyl end portion of the α1 helix and a cysteine in the amino end portion of the α2-1 helix. Such disulfide bonds stabilize the T-Cell-MMP and permit its cellular processing and excretion from eukaryotic cells in the absence of a bound epitope peptide (or null peptide). In one embodiment the carboxyl end portion of the α1 helix is from about aa position 79 to about as position 89 and the amino end portion of the α2-1 helix is from about aa position 134 to about as position 144 of the MHC Class I heavy chain (the aa positions are determined based on the sequence of the heavy chains without their leader sequence (see, e.g., FIGS. 3D-3H). In one such embodiment the disulfide bond is between a cysteine located at positions 83, 84, or 85 and a cysteine located at any of positions 138, 139 or 140 of the MHC Class I heavy chain. For example, a disulfide bond may be formed from cysteines incorporated into the MHC Class I heavy chain at aa 83 and a cysteine at an aa located at any of positions 138, 139 or 140. Alternatively, a disulfide bond may be formed between a cysteine inserted at position 84 and a cysteine inserted at any of positions 138, 139 or 140, or between a cysteine inserted at position 85 and a cysteine at any one of positions 138, 139 or 140. In an embodiment, the MHC Class 1 heavy chain intrachain disulfide bond is between cysteines substituted into a heavy chain sequence at positions 84 and 139 (e.g., the heavy chain sequence may be one of the heavy chain sequences set forth in FIGS. 3D-3H). As noted above, any of the MHC Class I intrachain disulfide bonds, including a disulfide bond between cysteines at 84 and 139, may be combined with interchain disulfide bonds including a bond between MHC Class 1 heavy position 236 and position 12 of a mature β2M polypeptide sequence (lacking its leader) as shown, for example, in FIG. 4.

In another embodiment, an intrachain disulfide bond may be formed in an MHC-H sequence of a T-Cell-MMP, or its epitope conjugate, between a cysteine substituted into the region between an positions 79 and 89 and a cysteine substituted into the region between aa positions 134 and 144 of the sequences given in FIGS. 3D-3H. In such an embodiment, the MHC Class I heavy chain sequence may have insertions, deletions and/or substitutions of 1 to 5 aas preceding or following the cysteines forming the disulfide bond between the carboxyl end portion of the α1 helix and the amino end portion of the α2-1 helix. Any inserted aas may be selected from the naturally occurring aas or the naturally occurring aas except proline and alanine.

In an embodiment, the β2M polypeptide of a T-Cell-MMP or its epitope conjugate comprises a mature β2M polypeptide sequence (aas 21-119) of any one of NP_004039.1, NP_001009066.1, NP_001040602.1, NP_776318.1, or NP_033865.2 (SEQ ID NOs:57 to 61).

In some cases, an HLA Class I heavy chain polypeptide of a T-Cell-MMP or its epitope conjugate comprises any one of the HLA-A, -B, -C, -E, -F, or -G sequences in FIGS. 3D-3H. Any of the heavy chain sequences may further comprise cysteine substitutions at positions 84 and 139, which may form an intrachain disulfide bond.

In an embodiment, the β2M polypeptide of a T-Cell-MMP, or its epitope conjugate, comprises a mature β2M polypeptide sequence (aas 21-119) of any one of the sequences in FIG. 4, which further comprises a R12C substitution.

In an embodiment, a T-Cell-MMP, or its epitope conjugate, comprises a first polypeptide comprising a mature β2M polypeptide sequence (e.g., aas 21-119 of any one of the sequences in FIG. 4) having a R12C substitution, and a second polypeptide comprising any one of the HLA-A, -B, -C, -E, -F, or -G sequences in FIGS. 3D-3H bearing a cysteine at position 236. In such embodiments an intrachain disulfide bond may form between the cysteines at positions 12 and 236. In addition, any of the heavy chain sequences may further comprise cysteine substitutions at positions 84 and 139, which may form an intrachain disulfide bond.

In some cases, an HLA Class I heavy chain polypeptide of a T-Cell-MMP, or its epitope conjugate, comprises the aa sequence of HLA-A*0201 (FIG. 3D). In some cases, an HLA Class I heavy chain polypeptide of a T-Cell-MMP, or its epitope conjugate, comprises the aa sequence of HLA-A*0201 having an A236C substitution (FIG. 3D). In some cases, an HLA Class I heavy chain polypeptide of a T-Cell-MMP, or its epitope conjugate, comprises the an sequence of HLA-A*0201 having a Y84A and a A236C substitution (FIG. 3D).

In an embodiment, a T-Cell-MMP, or its epitope conjugate, comprises a first polypeptide comprising aa residues 21-119 of NP_004039.1 with a R12C substitution (see FIG. 4), and a second polypeptide comprising an HLA-A*0201 (HLA-A2) sequence in FIG. 3D. In one such embodiment the HLA-A*0201 sequence has an A236C substitution. In another such embodiment, the HLA-A*0201 sequence has a Y84C and A139C substitution. In another such embodiment, the HLA-A*0201 sequence has a Y84C, A139C, and A236C substitution. As indicated, MHC-H sequences with Y84C and A139C substitutions may form a stabilizing intrachain disulfide bond, and a cysteine at position 236 of the mature MHC-H may bond to a cysteine at position 12 of a mature β2M polypeptide.

In an embodiment, a T-Cell-MMP, or its epitope conjugate, comprises a first polypeptide comprising aa residues 21-119 of NP_004039.1 with a R12C substitution (see FIG. 4), and a second polypeptide, an HLA Class I heavy chain polypeptide, comprises the aa sequence GSHSMRYFFTSVSRPGRGEPRFIAVGYVDDTQFVRFDSDAASQRMEPRAPWIEQEGPEYWDGET RKVKAHSQTHRVDL(aa cluster 1){C}(aa cluster 2)AGSHTVQRMYGCDVGSDWRFLRGYHQYAY DGKDYIALKEDLRSW(aa cluster 3){C}(aa cluster 4)HKWEAAHVAEQLRAYLEGTCVEWLRRYLE NGKETLQRTDAPKTHMTHHAVSDHEATLRCWALSFYPAEITLTWQRDGEDQTQDTELVETRPA GDGTFQKWAAVVVPSGQEQRYTCHVQHEGLPKPLTLRWEP (SEQ ID NO:76); or, the first polypeptide comprises the an sequence IQRTPKIQVY SCHPAENGKS NFLNCYVSGF HPSDIEVDLLKNGERIEKVE HSDLSFSKDW SFYLLYYTEF TPTEKDEYAC RVNHVTLSQP KIVKWDRDM (SEQ ID NO:77), and the second polypeptide comprises the an sequence, GSHSMRYFFTSVSRPGRGEPRFIAVGYVDDTQFVRFDSDAASQRMEPRAPWIEQEGPEYWDGET RKVKAHSQTHRVDL(aa cluster 1){C)(aa cluster 2)AGSHTVQRMYGCDVGSDWRFLRGYHQYAY DGKDYIALKEDLRSW(aa cluster 3){C}(aa cluster 4))HKWEAAHVAEQLRAYLEGTCVEWLRRYL ENGKETLQRTDAPKTHMTHHAVSDHEATLRCWALSFYPAEITLTWQRDGEDQTQDTEL(aa cluster 5)(C)(aa cluster 6)QKWAAVVVPSGQEQRYTCHVQHEGLPKPLTLRWEP (SEQ ID NO:78); where the cysteine residues indicated as {C} form a disulfide bond between the α1 and α2-1 helices and the (C) residue forms a disulfide bond with the mature β2M polypeptide cysteine at position 12.

Each occurrence of aa cluster 1, aa cluster 2, aa cluster 3, aa cluster 4, aa cluster 5, and aa cluster 6 is independently selected to be 1-5 an residues, wherein the aa residues are each selected independently from i) any naturally occurring (proteogenic) aa or ii) any naturally occurring aa except proline or glycine.

In an embodiment where the MHC Class I heavy chain is an HLA-A chain:
aa cluster 1 may be the amino acid sequence GTLRG or that sequence with one or two aas deleted or substituted with other naturally occurring aas (e.g., L replaced by I, V. A or F);
aa cluster 2 may be the amino acid sequence YNQSE or that sequence with one or two aas deleted or substituted with other naturally occurring aas (e.g., N replaced by Q, Q replaced by N, and/or E replaced by D);
aa cluster 3 may be the amino acid sequence TAADM or that sequence with one or two aas deleted or substituted with other naturally occurring aas (e.g., T replaced by S, A replaced by G, D replaced by E, and/or M replaced by L, V, or I);
aa cluster 4 may be the amino acid sequence AQTTK or that sequence with one or two aas deleted or substituted with other naturally occurring aas (e.g., A replaced by G, Q replaced by N, or T replaced by S, and or K replaced by R or Q);
aa cluster 5 may be the amino acid sequence VETRP or that sequence with one or two aas deleted or substituted with other naturally occurring aas (e.g., V replaced by I or L, E replaced by D, T replaced by S, and/or R replaced by K); and/or
aa cluster 6 may be the amino acid sequence GDGTF or that sequence with one or two aas deleted or substituted with other naturally occurring aas (e.g., D replaced by E, T replaced by S, or F replaced by L, W, or Y).

In some cases, the β2M polypeptide comprises the amino acid sequence:

(SEQ ID NO: 79)
IQRTPKIQVYSCHPAENGKSNFLNCYVSGFHPSDIEVDLLKNGERIEKVE
HSDLSFSKDWSFYLLYYTEFTPTEKDEYACRVNHVTLSQPKIVKWDRDM.

In some cases, the first polypeptide and the second polypeptide of a T-Cell-MMP of the present disclosure are disulfides linked to one another through: i) a Cys residue present in a linker connecting the peptide epitope and a β2M polypeptide in the first polypeptide chain (e.g., with the epitope placed in the N-terminal to the linker and the β2M sequences); and ii) a Cys residue present in an MHC Class I heavy chain in the second polypeptide chain. In some cases, the Cys residue present in the MHC Class I heavy chain is a Cys introduced as a Y84C substitution. In some cases, the linker connecting the peptide epitope and the β2M polypeptide in the first polypeptide chain is GCGGS(G₄S)n, where n is 1, 2, 3, 4, 5, 6, 7, 8, or 9 (SEQ ID NO:93) (e.g., epitope-GCGGS(G4S)n-mature β2M polypeptide). For example, in some cases, the linker comprises the an sequence GCGGSGGGGSGGG GSGGGGS (SEQ ID NO:95). As another example, the linker comprises the an sequence GCGGSGGG GSGGGGS (SEQ ID NO:96). Examples of such a disulfide-linked first and second polypeptide are depicted schematically in FIGS. 6E-6H.

5 Scaffold Polypeptides

T-Cell-MMPs and T-Cell-MMP-epitope conjugates can comprise a Fc polypeptide, or can comprise another suitable scaffold polypeptide.

Suitable scaffold polypeptides include antibody-based scaffold polypeptides and non-antibody-based scaffolds. Non-antibody-based scaffolds include, e.g., albumin, an XTEN (extended recombinant) polypeptide, transferrin, a Fc receptor polypeptide, an elastin-like polypeptide (see, e.g., Hassounch et al. (2012) Methods Enzymol. 502:215; e.g., a polypeptide comprising a pentapeptide repeat unit of (Val-Pro-Gly-X-Gly; SEQ ID NO:80), where X is any amino acid other than proline), an albumin-binding polypeptide, a silk-like polypeptide (see, e.g., Valluzzi et al. (2002) Philos Trans R Soc Lond B Biol Sci. 357:165), a silk-elastin-like polypeptide (SELP; see, e.g., Megeed et al. (2002) Adv Drug Deliv Rev. 54:1075), and the like. Suitable XTEN polypeptides include, e.g., those disclosed in WO 2009/023270, WO 2010/091122, WO 2007/103515, US 2010/018%82, and US 2009/0092582; see, also, Schellenberger et al. (2009) Nat Biotechnol. 27:1186). Suitable albumin polypeptides include, e.g., human serum albumin.

Suitable scaffold polypeptides (e.g., those with an Ig Fc multimerization sequence) will, in some cases, be half-life extending polypeptides. Thus, in some cases, a suitable scaffold polypeptide increases the in vivo half-life (e.g., the serum half-life) of the multimeric polypeptide, compared to a control multimeric polypeptide lacking the scaffold polypeptide. For example, in some cases, a scaffold polypeptide increases the in vivo half-life of the multimeric polypeptide, compared to a control multimeric polypeptide lacking the scaffold polypeptide, by at least about 10%, at least about 15%, at least about 20%, at least about 25%, at least about 50%, at least about 2-fold, at least about 2.5-fold, at least about 5-fold, at least about 10-fold, at least about 25-fold, at least about 50-fold, at least about 100-fold, or more than 100-fold. As an example, in some cases, a Fc polypeptide increases the in vivo half-life (serum half-life) of the multimeric polypeptide, compared to a control multimeric polypeptide lacking the Fc polypeptide, by at least about 10%, at least about 15%, at least about 20%, at least about 25%, at least about 50%, at least about 2-fold, at least about 2.5-fold, at least about 5-fold, at least about 10-fold, at least about 25-fold, at least about 50-fold, at least about 100-fold, or more than 100-fold.

a. Immunoglobulin Fc Polypeptides

In some cases, the first and/or the second polypeptide chains of a T-Cell-MMP or its corresponding T-Cell-MMP-epitope conjugate (multimeric polypeptide(s)) comprise a Fc scaffold polypeptide that may be modified to include one or more chemical conjugation sites within or attached (e.g., at a terminus or attached by a linker) to the polypeptide. The Fc polypeptide of a T-Cell-MMP or T-Cell-MMP-epitope conjugate can be, for example, from an IgA, IgD, IgE, IgG, or IgM, which may contain a human polypeptide sequence, a humanized polypeptide sequence, a Fc region polypeptide of a synthetic heavy chain constant region, or a consensus heavy chain constant region. In embodiments, the Fc polypeptide can be from a human IgG1 Fc, a human IgG2 Fc, a human IgG3 Fc, a human IgG4 Fc, a human IgA Fc, a human IgD Fc, a human IgE Fc, a human IgM Fc, etc. Unless stated otherwise, the Fc polypeptides used in the T-Cell-MMPs and their epitope conjugates do not comprise a transmembrane anchoring domain or a portion thereof sufficient to anchor the T-Cell-MMP or its epitope conjugate to a cell membrane. In some cases, the Fc polypeptide comprises an aa sequence having at least about 70% (e.g., at least about 75%, 80%, 85%, 90%, 95%, 98%, 99%, or 100%) aa sequence identity to an aa sequence of a Fc region depicted in FIGS. 2A-2G. In some cases, the Fc region comprises an aa sequence having at least about 70% (e.g., at least about 75%, 80%, 85%, 90%, 95%, 98%, 99%, or 100%) as sequence identity to the human IgG1 Fc polypeptide depicted in FIG. 2A. In some cases, the Fc region comprises an aa sequence having at least about 70%, (e.g., at least about 75%, 80%, 85%, 90%, 95%, 98%, 99%, or 100%) as sequence identity to the human IgG1 Fc polypeptide depicted in FIG. 2A; and comprises a substitution of N77, which is underlined and bolded; e.g., the Fc polypeptide comprises a N77A substitution. In some cases, the Fc polypeptide comprises an aa sequence having at least about 70% (e.g., at least about 75%, 80%, 85%, 90%, 95%, 98%, 99%, or 100%) as sequence identity to the human IgG2 Fc polypeptide depicted in FIG. 2A; e.g., the Fc polypeptide comprises an aa sequence having at least about 70% (e.g., at least about 75%, 80%, 85%, 90%, 95%, 98%, 99%, or 100%) as sequence identity to aas 99-325 of the human IgG2 Fc polypeptide depicted in FIG. 2A. In some cases, the Fc polypeptide comprises an aa sequence having at least about 70% (e.g., at least about 75%, 80%, 85%, 90%, 95%, 98%, 99%, or 100%) aa sequence identity to the human IgG3 Fc polypeptide depicted in FIG. 2A; e.g., the Fc polypeptide comprises an aa sequence having at least about 70% (e.g., at least about 75%, 80%, 85%, 90%, 95%, 98%, 99%, or 100%) aa sequence identity to aas 19-246 of the human IgG3 Fc polypeptide depicted in FIG. 2A. In some cases, the Fc polypeptide comprises an aa sequence having at least about 70% (e.g., at least about 75%, 80%, 85%, 90%, 95%, 98%, 99%, or 100%) as sequence identity to the human IgM Fc polypeptide depicted in FIG. 2B; e.g., the Fc polypeptide comprises an as sequence having at least about 70% (e.g., at least about 75%, 80%, 85%, 90%, 95%, 98%, 99%, or 100%) as sequence identity to aas 1-276 of the human IgM Fc polypeptide depicted in FIG. 2B. In some cases, the Fc polypeptide comprises an aa sequence having at least about 70% (e.g., at least about 75%, 80%, 85%, 90%, 95%, 98%, 99%, or 100%) aa sequence identity to the human IgA Fc polypeptide depicted in FIG. 2C; e.g., the Fc polypeptide comprises an aa sequence having at least about 70% (e.g., at least about 75%, 80%, 85%, 90%, 95%, 98%, 99%, or 100%) aa sequence identity to aas 1-234 of the human IgA Fc polypeptide depicted in FIG. 2C.

In some cases, the Fc polypeptide present in a multimeric polypeptide comprises the aa sequence depicted in FIG. 2A (human IgG1 Fc). In some cases, the Fc polypeptide present in a multimeric polypeptide comprises the as sequence depicted in FIG. 2A (human IgG1 Fc), except for a substitution of N297 (N77 of the as sequence depicted in FIG. 2A) with an aa other than asparagine. In some cases, the Fc polypeptide present in a multimeric polypeptide comprises the as sequence depicted in FIG. 2C (human IgG1 Fc comprising an N297A substitution, which is N77 of the as sequence depicted in FIG. 2A). In some cases, the Fc polypeptide present in a multimeric polypeptide comprises the as sequence depicted in FIG. 2A (human IgG1 Fc), except for a substitution of L234 (L14 of the aa sequence depicted in FIG. 2A) with an as other than leucine. In some cases, the Fc polypeptide present in a multimeric polypeptide comprises the as sequence depicted in FIG. 2A (human IgG1 Fc), except for a substitution of L235 with an aa other than leucine.

In some cases, the Fc polypeptide present in a multimeric polypeptide comprises the aa sequence depicted in FIG. 2E. In some cases, the Fc polypeptide present in a multimeric polypeptide comprises the aa sequence depicted in FIG. 2F. In some cases, the Fc polypeptide present in a multimeric polypeptide comprises the aa sequence depicted in FIG. 2G (human IgG1 Fc comprising an L234A substitution and an L235A substitution, corresponding to positions 14 and 15 of the aa sequence depicted in FIG. 2G). In some cases, the Fc polypeptide present in a multimeric polypeptide comprises the aa sequence depicted in FIG. 2A (human IgG1 Fc), except for a substitution of P331 (P111 of the aa sequence depicted in FIG. 2A) with an aa other than proline; in some cases, the substitution is a P331S substitution. In some cases, the Fc polypeptide present in a multimeric polypeptide comprises the aa sequence depicted in FIG. 2A (human IgG1 Fc), except for substitutions at L234 and L235 (L14 and L15 of the aa sequence depicted in FIG. 2A) with aas other than leucine. In some cases, the Fc polypeptide present in a multimeric polypeptide comprises the aa sequence depicted in FIG. 2A (human IgG1 Fc), except for substitutions at L234 and L235 (L14 and L15 of the aa sequence depicted in FIG. 2A) with aas other than leucine, and a substitution of P331 (P111 of the aa sequence depicted in FIG. 2A) with an aa other than proline. In some cases, the Fc polypeptide present in a multimeric polypeptide comprises the an sequence depicted in FIG. 2E (human IgG1 Fc comprising L234F, L235E, and P331S substitutions, corresponding to an positions 14, 15, and 111 of the an sequence depicted in FIG. 2E). In some cases, the Fc polypeptide present in a multimeric polypeptide is an IgG1 Fc polypeptide that comprises L234A and L235A substitutions (substitutions of L14 and L15 of the an sequence depicted in FIG. 2A with Ala), as depicted in FIG. 2G.

In some cases, the Fc polypeptide comprises an aa sequence having at least about 70% (e.g., at least about 75%, 80%, 85%, 90%, 95%, 98%, 99%, or 100%) an sequence identity to a human IgG4 Fc polypeptide depicted in FIG. 2C. In some cases, the Fc polypeptide comprises an aa sequence having at least about 70% (e.g., at least about 75%, 80%, 85%, 90%, 95%, 98%, 99%, or 100%) aa sequence identity to aas 100 to 327 of the GenBank P01861 human IgG4 Fc polypeptide depicted in FIG. 2C.

Any of the foregoing immunoglobin sequences may undergo proteolysis such that some amino acids are lost from the carboxyl terminal end. In particular, the terminal lysine provided in some of the sequences set forth in FIGS. 2A-2G (e.g., the IgG sequences in FIGS. 2A, 2B, 2F, and 2G) may be removed/lost during cellular processing of the T-Cell MMPs, and accordingly, those amino acids may not be present on some or all of the T-Cell MMP molecules as expressed.

6 Linkers

T-Cell-MMPs (and their T-Cell-MMP-epitope conjugates) can include one or more independently selected linker peptides interposed between, for example, any one or more of: i) an MHC polypeptide and an Ig Fc polypeptide, where such a linker is referred to herein as a “L1 linker”; ii) an MHC polypeptide and a MOD, where such a linker is referred to herein as a “L2 linker”; iii) a first MOD and a second MOD, where such a linker is referred to herein as a “L3 linker” (e.g., between a first variant 4-1BBL polypeptide and a second variant 4-1BBL polypeptide; or between a second variant 4-1BBL polypeptide and a third variant 4-1BBL polypeptide); iv) a conjugation site or a peptide antigen (conjugated “epitope” peptide) and an MHC Class I polypeptide (e.g., β2M); v) an MHC Class I polypeptide and a dimerization polypeptide (e.g., a first or a second member of a dimerizing pair); and vi) a dimerization polypeptide (e.g., a first or a second member of a dimerizing pair) and an IgFc polypeptide.

Suitable linkers (also referred to as “spacers”) can be readily selected and can be of any of a number of suitable lengths, such as from 1 aa to 25 aa, from 3 an to 20 aa, from 2 an to 15 aa, from 3 aa to 12 aa, from 4 aa to 10 aa, from 5 aa to 9 aa, from 6 aa to 8 aa, or from 7 aa to 8 aa. In embodiments, a suitable linker can be 1, 2, 3, 4, 5, 6, 7, 8, 9, 10, 11, 12, 13, 14, 15, 16, 17, 18, 19, 20, 21, 22, 23, 24, or 25 aa in length. In some cases, a linker has a length of from 25 aa to 50 aa, e.g., from 25 to 30, from 30 to 35, from 35 to 40, from 40 to 45, or from 45 to 50 an in length.

Exemplary linkers include glycine polymers (G).; glycine-serine polymers (including, for example, (GS), (GSGGS) (SEQ ID NO:81) and (GGGS) (SEQ ID NO:82), any of which may be repeated from 1 to 10 times (e.g., repeated 1, 2, 3, 4, 5, 6, 7, 8, 9, or 10 times); glycine-alanine polymers; alanine-serine polymers; and other flexible linkers known in the art. Glycine and glycine-serine polymers can both be used; both Gly and Ser are relatively unstructured and therefore can serve as a neutral tether between components. Glycine polymers access significantly more phi-psi space than even alanine, and are much less restricted than residues with longer side chains (see Scheraga, Rev. Computational Chem. 11173-142 (1992)). Exemplary linkers can also comprise an sequences including, but not limited to, GGSG (SEQ ID NO:83), GGSGG (SEQ ID NO:84), GSGSG (SEQ ID NO:85), GSGGG (SEQ ID NO:86), GGGSG (SEQ ID NO:87), GSSSG (SEQ ID NO:88), or Gly(Ser)₄ (SEQ ID NO:89), any of which may be repeated from 1 to 10 times (e.g., repeated 1, 2, 3, 4, 5, 6, 7, 8, 9, or 10 times), combinations thereof, and the like. Exemplary linkers can comprise the sequence Gly₄Ser (SEQ ID NO:90), which may be repeated from 1 to 10 times (e.g., 1, 2, 3, 4, 5, 6, 7, 8, 9, or 10 times). In one embodiment the linker comprises the an sequence AAAGG (SEQ ID NO:91), which may be repeated from 1 to 10 times.

In some cases, a linker comprises the an sequence (GGGGS) (SEQ ID NO:92), which may be repeated from 1 to 10 times (e.g., 1, 2, 3, 4, 5, 6, 7, 8, 9, or 10 times). In some cases, a linker polypeptide, present in a first polypeptide of a T-Cell-MMP or its epitope conjugate, includes a cysteine residue that can form a disulfide bond with a cysteine residue present in an epitope presenting polypeptide or a second polypeptide of a T-Cell-MMP or its epitope conjugate. In some cases, for example, the linker comprises the an sequence GCGGS(G₄S) (SEQ ID NO:93) where the G₄S unit may be repeated from 1 to 10 times (e.g., repeated 1, 2, 3, 4, 5, 6, 7, 8, 9, or 10 times), GCGASGGGGSGGGGS (SEQ ID NO:94), the sequence GCGGSGGGGSGGGGSGGGGS (SEQ ID NO:95) or the sequence GCGGSGGGGSGGGGS (SEQ ID NO:96).

Linkers, including the polypeptide linkers described above, may be present between a payload coupled to the first or second polypeptide of a T-Cell-MMP (or its epitope conjugate). In addition to the polypeptide linkers recited above, the linkers used to attach a payload or epitope (e.g., peptide) to the first and/or second polypeptide can be non-peptides. Such non-peptide linkers include polymers comprising, for example, polyethylene glycol (PEG). Other linkers, including those resulting from coupling with a bifunctional crosslinking agent, such as those recited below, may also be utilized.

7 Immunomodulatory Polypeptides (MODs)

In some cases, a MOD present in a T-Cell-MMP of the present disclosure is a wt. MOD. In other cases, a MOD present in a T-Cell-MMP of the present disclosure is a variant MOD that has reduced affinity for a Co-MOD, compared to the affinity of a corresponding wt. MOD for the Co-MOD. Some MOD polypeptides that may be incorporated into T-Cell-MMPs exhibit reduced affinity for Co-MODs. The MOD polypeptides can have from 1 aa to 10 an differences from a wt. immunomodulatory domain. For example, in some cases, a variant MOD polypeptide present in a T-Cell-MMP of the present disclosure may differ in an sequence by, for example, 1 aa. 2 aa, 3 aa, 4 aa, 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, or 20 aa (e.g., from 1aa to 5 aa, from 5 aa to 10 aa, or from 10 an to 20 aa) from a corresponding wild-type MOD. As an example, in some cases, a variant MOD polypeptide present in a T-Cell-MMP of the present disclosure has and/or includes 1, 2, 3, 4, 5, 6, 7, 8, 9, 10, 11, 12, 13, 14, 15, 16, 17, 18, 19. or 20 (e.g., from about 1 to about 20; 1 to 2; 1 to 3; 1 to 5; 2 to 4; 2 to 5; 2 to 6; 2 to 7; 2 to 8; 2 to 9; 2 to 10; 2 to 11; 2 to 12; 2 to 13; 2 to 14; 2 to 15; 2 to 16; 2 to 17; 2 to 18; 2 to 19, 2 to 20; 5 to 10; or 10 to 20) aa insertions, deletions, and/or substitutions, compared to a corresponding reference (e.g., wt.) MOD. In some cases, variant MOD polypeptides present in a T-Cell-MMP include a single an substitution compared to a corresponding reference (e.g., wt.) MOD.

As discussed above, variant MODs suitable for inclusion as domains (MOD polypeptides) in T-Cell-MMPs of the present disclosure (and/or their epitope conjugates) include those that may exhibit reduced affinity for a Co-MOD, compared to the affinity of a corresponding wt. MOD for the Co-MOD. Suitable variant MODs can be identified by, for example, mutagenesis, such as scanning mutagenesis (e.g., alanine, serine, or glycine scanning mutagenesis).

Exemplary pairs of MODs and Co-MODs include, but are not limited to, entries (a) to (r) listed in the following table:

Exemplary Pairs of MODs and Co-MODs

| | |
|---|---|
| a) 4-1BBL (MOD) and 4-1BB (Co-MOD); | l) CD83 (MOD) and CD83L (Co-MOD); |
| b) PD-L1 (MOD) and PD1 (Co-MOD); | m) HVEM (CD270) (MOD) and CD 160 |
| c) IL-2 (MOD) and IL-2 receptor (Co-MOD); | (Co-MOD); |
| d) CD80 (MOD) and CD28 (Co-MOD); | n) JAG1 (CD339) (MOD) and Notch |
| e) CD86 (MOD) and CD28 (Co-MOD); | (Co-MOD); |
| f) OX40L (CD252) (MOD) and OX40 | o) JAG1 (CD339) (MOD) and CD46 |
| (CD 134) (Co-MOD); | (Co-MOD); |
| g) Fas ligand (MOD) and Fas (Co-MOD); | p) CD70 (MOD) and CD27 (Co-MOD); |
| h) ICOS-L (MOD) and ICOS (Co-MOD); | q) CD80 (MOD) and CTLA4 (Co-MOD); |
| i) ICAM (MOD) and LFA-1 (Co-MOD); | r) CD86 (MOD) and CTLA4 (Co-MOD) |
| j) CD30L (MOD) and CD30 (Co-MOD); | and |
| k) CD40 (MOD) and CD40L (Co-MOD); | s) PD-L1 (MOD) and CD-80 (Co-MOD) |

In some cases, a variant MOD present in a T-Cell-MMP has a binding affinity for a Co-MOD that is from 100 nM to 100 μM. For example, in some cases, a variant MOD polypeptide present in a T-Cell-MMP (or its epitope conjugate) has a binding affinity for a Co-MOD that is from about 100 nM to about 150 nM, from about 100 nM to about 500 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 500 nM to about 1 μM, from about 600 nM to about 700 nM, from about 700 nM to about 800 nM, from about 800 nM to about 900 nM, from about 900 nM to about 1 μM, from about 1 μM to about 5 μM, from about 1 μM to about 25 μM from about 5 μM to about 10 μM, from about 10 μM to about 15 μM, from about 15 μM to about 20 M, from about 20 μM to about 25 μM, from about 25 μM to about 50 μM, from about 25 μM to about 100 μM, from about 50 μM to about 75 μM, or from about 75 μM to about 100 μM.

In some cases, a variant MOD present in a T-Cell-MMP exhibits reduced affinity for a cognate Co-MOD. Similarly, a T-Cell-MMP that comprises a variant MOD exhibits reduced affinity for a cognate Co-MOD as compared to the binding affinity of the wild-type MOD for its cognate co-MOD. Generally speaking, the MOD(s) present in a T-Cell-MMP will be mods that provide activating immunomodulatory signals to the T cell, including, e.g., signals that cause an increase in the number of epitope-specific T cells.

a. Wild-Type and Variant CD80 MODs

In some cases, a variant MOD polypeptide present in a T-Cell-MMP is a variant CD80 polypeptide. Wild-type CD80 binds to CD28.

A wild-type amino acid sequence of the ectodomain of human CD80 can be as follows: VIHVTK EVKEVATLSC GHNVSVEELA QTRIYWQKEK KMVLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:101).

A wild-type CD28 amino acid sequence can be as follows: MLRLLLALNL FPSIQVTGNK ILVKQSPMLV AYDNAVNLSC KYSYNLFSRE FRASLHKGLD SAVEVCVVYG NYSQQLQVYS KTGFNCDGKL GNESVTFYLQ NLYVNQTDIY FCKIEVMYPP PYLDNEKSNG TIIHVKGKHL CPSPLFPGPS KPFWVLVVVG GVLACYSLLV TVAFIIFWVR SKRSRLLHSD YMNMTPRRPG PTRKHYQPYA PPRDFAAYRS (SEQ ID NO:102). In some cases, where a T-Cell-MMP comprises a variant CD80 polypeptide, a Co-MOD is a CD28 polypeptide comprising the amino acid sequence of SEQ ID NO:102.

A wild-type CD28 amino acid sequence can also be as follows: MLRLLLALNL FPSIQVTGNK ILVKQSPMLV AYDNAVNLSW KHLCPSPLFP GPSKPFWVLV VVGGVLACYS LLVTVAFIIF WVRSKRSRLL HSDYMNMTPR RPGPTRKHYQ PYAPPRDFAA YRS (SEQ ID NO:103).

A wild-type CD28 amino acid sequence can be as follows: MLRLLLALNL FPSIQVTGKH LCPSPLFPGP SKPFWVLVVV GGVLACYSLL VTVAFIIFWV RSKRSRLLHS DYMNMTPRRP GPTRKHYQPY APPRDFAAYR S (SEQ ID NO:104).

In some cases, a variant CD80 polypeptide exhibits reduced binding affinity to CD28, compared to the binding affinity of a CD80 polypeptide comprising the amino acid sequence set forth in SEQ ID NO:102 for CD28. For example, in some cases, a variant CD80 polypeptide binds CD28 with a binding affinity that is at least 10% less (e.g., at least: 15% less, 20% less, 25% less, 30% less, 35% less, 40% less, 45% less, 50% less, 55% less, 60% less, 65% less, 70% less, 75% less, 80% less, 85% less, 90% less, 95% less, or more than 95% less) than the binding affinity of a CD80 polypeptide comprising the amino acid sequence set forth in SEQ ID NO:102 for CD28 (e.g., a CD28 polypeptide comprising the amino acid sequence set forth in one of SEQ ID NOs:102, 103, or 104).

In some cases, a variant CD80 polypeptide has a single amino acid insertion, deletion, or substitution compared to the CD80 amino acid sequence set forth in SEQ ID NO:101. In some cases, a variant CD80 polypeptide has from 2 to 10 aa insertions, deletions, or substitutions compared to the CD80 amino acid sequence set forth in SEQ ID NO:101. In some cases, a variant CD80 polypeptide has 2, 3, 4, 5, 6, 7, 8, 9, or 10 amino acid insertions, deletions, and/or substitutions compared to the CD80 amino acid sequence set forth in SEQ ID NO:101.

Variant CD80 polypeptides exhibit reduced binding affinity to CD28, compared to the binding affinity of a CD80 polypeptide comprising the amino acid sequence set forth in SEQ ID NO:101. For example, in some cases, a variant CD80 polypeptide binds CD28 with a binding affinity that is at least 10% less, at least 15% less, at least 20% less, at least 25% less, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or more than 95% less than the binding affinity of a CD80 polypeptide comprising the amino acid sequence set forth in SEQ ID NO:101 for a CD28 polypeptide (e.g., a CD28 polypeptide comprising the amino acid sequence set forth in SEQ ID NO:102 or 103), when assayed under the same conditions.

Suitable CD80 variants are described in published PCT Application WO 2019/051091, published 14 Mar. 2019 (Applicant Cue Biopharma, Inc.). See paragraphs [00170]-[00196], the disclosure of which is expressly incorporated herein by reference.

b. Wild-Type and Variant CD86 MODs

In some cases, a variant MOD polypeptide present in a T-Cell-MMP is a variant CD86 polypeptide. Wild-type CD86 binds to CD28.

The amino acid sequence of the full ectodomain of a wild-type human CD86 can be as follows:

(SEQ ID NO: 105)
APLKIQAYFNETADLPCQFANSQNQSLSELVVFWQDQENLVLNEVYLGKE
KFDSVHSKYMNRTSFDSDSWTLRLHNLQIKDKGLYQCIIHHKKPTGMIRI
HQMNSELSVLANFSQPEIVPISNITENVYINLTCSSIHGYPEPKKMSVLL
RTKNSTIEYDGIMQKSQDNVTELYDVSISLSVSFPDVTSNMTIFCILETD
KTRLLSSPFSIELEDPQPPPDHIP.

The amino acid sequence of the IgV domain of a wild-type human CD86 can be as follows:

(SEQ ID NO: 106)
APLKIQAYFNETADLPCQFANSQNQSLSELVVFWQDQENLVLNEVYLGKE
KFDSVHSKYMNRTSFDSDSWTLRLHNLQIKDKGLYQCIIHHKKPTGMIRI
HQMNSELSVL.

In some cases, a variant CD86 polypeptide exhibits reduced binding affinity to CD28, compared to the binding affinity of a CD86 polypeptide comprising the amino acid sequence set forth in SEQ ID NO:105 or SEQ ID NO:106 for CD28. For example, in some cases, a variant CD86 polypeptide binds CD28 with a binding affinity that is at least 10% less, at least 15% less, at least 20% less, at least 25% less, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or more than 95% less than the binding affinity of a CD86 polypeptide comprising the amino acid sequence set forth in SEQ ID NO:105 or SEQ ID NO:106 for CD28 (e.g., a CD28 polypeptide comprising the amino acid sequence set forth in one of SEQ ID NOs:102, 103, or 104).

In some cases, a variant CD86 polypeptide has a single aa insertion, deletion, or substitutions compared to the CD86 amino acid sequence set forth in SEQ ID NO:105. In some cases, a variant CD86 polypeptide has from 2 to 10 amino acid insertions, deletions, and/or substitutions compared to the CD86 amino acid sequence set forth in SEQ ID NO:105. In some cases, a variant CD86 polypeptide has 2, 3, 4, 5, 6, 7, 8, 9, or 10 aa insertions, deletions, and/or substitutions compared to the CD86 amino acid sequence set forth in SEQ ID NO:105.

Variant CD86 polypeptides exhibit reduced binding affinity to CD28, compared to the binding affinity of a CD86 polypeptide comprising the amino acid sequence set forth in SEQ ID NO:105 or SEQ ID NO:106. For example, in some cases, a variant CD86 polypeptide binds CD28 with a binding affinity that is at least 10% less, at least 15% less, at least 20% less, at least 25% less, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or more than 95% less than the binding affinity of a CD86 polypeptide comprising the amino acid sequence set forth in SEQ ID NO:105 or SEQ ID NO:106 for a CD28 polypeptide (e.g., a CD28 polypeptide comprising the amino acid sequence set forth in SEQ ID NO:102 or 103), when assayed under the same conditions.

Suitable CD86 variants are described in published PCT Application WO 2019/051091, published 14 Mar. 2019 (Applicant Cue Biopharma, Inc.). See paragraphs [00197]-[00228], the disclosure of which is expressly incorporated herein by reference.

c. Wild-Type and Variant 4-1BBL MODs

In some cases, a variant MOD polypeptide present in a T-Cell-MMP is a variant 4-1BBL polypeptide. Wild-type 4-1BBL binds to 4-1BB (CD137).

A wild-type 4-1BBL amino acid sequence can be as follows: MEYASDASLD PEAPWPPAPR ARACRVLP [symbol] [symbol] CPWAVSGARA SPGSAASPRL REGPELSPDD PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE(SEQ ID NO:107) NCBI Reference Sequence: NP_003802.1, where aas 29-49 are a transmembrane region.

In some cases, a variant 4-1BBL polypeptide is a variant of the tumor necrosis factor (TNF) homology domain (THD) of human 4-1BBL.

A wild-type amino acid sequence of the THD of human 4-1BBL can be, e.g., one of SEQ ID NOs:108-110, as follows:

(SEQ ID NO: 108)
PAGLLDLRQG MFAQLVAQNV LLIDGPLSWY SDPGLAGVSL
TGGLSYKEDT KELVVAKAGV YYVFFQLELR RVVAGEGSGS
VSLALHLQPL RSAAGAAALA LTVDLPPASS EARNSAFGFQ
GRLLHLSAGQ RLGVHLHTEA RARHAWQLTQ GATVLGLFRV
TPEIPAGLPS PRSE;
(SEQ ID NO: 109)
D PAGLLDLRQG MFAQLVAQNV LLIDGPLSWY SDPGLAGVSL
TGGLSYKEDT KELVVAKAGV YYVFFQLELR RVVAGEGSGS
VSLALHLQPL RSAAGAAALA LTVDLPPASS EARNSAFGFQ
GRLLHLSAGQ RLGVHLHTEA RARHAWQLTQ GATVLGLFRV
TPEIPAGLPS PRSE;
or
(SEQ ID NO: 110)
D PAGLLDLRQG MFAQLVAQNV LLIDGPLSWY SDPGLAGVSL
TGGLSYKEDT KELVVAKAGV YYVFFQLELR RVVAGEGSGS
VSLALHLQPL RSAAGAAALA LTVDLPPASS EARNSAFGFQ
GRLLHLSAGQ RLGVHLHTEA RARHAWQLTQ GATVLGLFRV
TPEIPA.

A wild-type 4-1BB amino acid sequence can be as follows: MGNSCYNIVA TLLLVLNFER TRSLQDPCSN CPAGTFCDNN RNQICSPCPP NSFSSAGGQR TCDICRQCKG VFRTRKECSS TSNAECDCTP GFHCLGAGCS MCEQDCKQGQ ELTKKGCKDC CFGTFNDQKR GICRPWTNCS LDGKSVLVNG TKERDVVCGP SPADLSPGAS SVTPPAPARE PGHSPQIISF FLALTSTALL FLLFFLTLRF SVVKRGRKKL LYIFKQPFMR PVQTTQEEDG CSCRFPEEEE GGCEL (SEQ ID NO:111). In some cases, where a T-Cell-MMP comprises a variant 4-1BBL polypeptide, a Co-MOD is a 4-1BB polypeptide comprising the amino acid sequence of SEQ ID NO:111.

Variant 4-1BBL polypeptides exhibit reduced binding affinity to 4-1BB, compared to the binding affinity of a 4-1BBL polypeptide comprising the amino acid sequence set forth in one of SEQ ID NOs:107-110. For example, in some cases, a variant 4-1BBL polypeptide binds 4-1BB with a binding affinity that is at least 10% less, at least 15% less, at least 20% less, at least 25% less, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or more than 95% less than the binding affinity of a 4-1BBL polypeptide comprising the amino acid sequence set forth in one of SEQ ID NOs:107-110 for a 4-1BB polypeptide (e.g., a 4-1BB polypeptide comprising the amino acid sequence set forth in SEQ ID NO:111), when assayed under the same conditions.

4-1BBL variants suitable for use as a MOD in a T-Cell-MMP include those comprising a sequence with at least one aa substitution and having at least 90%, at least 95%, at least 98%, or at least 99% aa sequence identity to SEQ ID NOs:108, 109 or 110. 4-1BBL variants suitable for use as a MOD in a T-Cell-MMP include those comprising a sequence with at least two aa substitutions and having at least 90%, at least 95%, at least 98%, or at least 99% aa sequence identity to SEQ ID NOs:108, 109 or 110.

4-1BBL variants suitable for inclusion in a T-Cell-MMP include those comprising a sequence with at least one aa substitution (e.g., two, three, or four insertions, deletions, and/or substitutions) include those having at least 90%, at least 95%, at least 98%, or at least 99% aa sequence identity to at least 140 (e.g., at least 160, 175, 180, or 181) contiguous aas of SEQ ID NO:108.

Suitable 4-1BBL variants are described in published PCT Application WO 2019/051091, published 14 Mar. 2019 (Applicant Cue Biopharma, Inc.). See paragraphs [00229]-[00324], the disclosure of which is expressly incorporated herein by reference.

d. Wild-Type and Variant IL-2 MODs

In some cases, a variant MOD polypeptide present in a T-Cell-MMP is a variant IL-2 polypeptide. Wild-type IL-2 binds to IL-2 receptor (IL-2R), i.e., a heterotrimeric polypeptide comprising IL-2Rα, IL-2Rβ, and IL-2Rγ.

A wild-type IL-2 amino acid sequence can be as follows: APTSSSTKKT QLQLEHLLLD LQMILNGINN YKNPKLTRML TFKFYMPKKA TELKHLQCLEEELKPLEEVL NLAQSKNFHL RPRDLISNIN VIVLELKGSE TTFMCEYADE TATIVEFLNRWITFCQSIIS TLT (UniProt, P60568, SEQ ID NO:112).

Wild-type IL2 binds to an IL2 receptor (IL2R) on the surface of a cell. An IL2 receptor is in some cases a heterotrimeric polypeptide comprising an alpha chain (IL-2Rα; also referred to as CD25), a beta chain (IL-2Rβ; also referred to as CD122) and a gamma chain (IL-2Rγ; also referred to as CD132). Amino acid sequences of human IL-2Rα, IL2Rβ, and IL-2Rγ are provided in the accompanying sequence listing as SEQ ID NO:113, SEQ ID NO:114, and SEQ ID NO:115, and are also provided in, for example, U.S. Patent Pub. No. 20200407416.

Suitable variant IL-2 polypeptide sequences include polypeptide sequences comprising an aa sequence having at least 80% (e.g., at least 85%, at least 90%, at least 95%, at least 98%, at least 99%, or 100%) aa sequence identity to at least 80 (e.g., 90, 100, 110, 120, 130 or 133) contiguous aas of SEQ ID NO:112. Potential amino acids where substitutions may be introduced include one or more of the following positions:
(i) position 15, where the aa is other than E (e.g., A);
(ii) position 16, where the aa is other than H (e.g., A, T, N, C, Q, M, V or W);
(iii) position 20 is an aa other than D (e.g., A);
(iv) position 42, where the aa is other than F (e.g., A, M, P, S, T, Y, V or H);
(v) position 45, where the aa is other than Y (e.g., A);
(vi) position 88, where the aa is other than N (e.g., A or R);
(vii) position 126, where the as is other than Q (e.g., A).

Combinations of the above substitutions include (H16X, F42X), (D20X, F42X), (E15X, D20X, F42X), (an H16X, D20X, F42X), (H16X, F42X, R88X), (H16X, F42X, Q126X), (D20X, F42X, Q126X), (D20X, F42X, and Y4X), (H16X, D20X, F42X, and Y45X), (D20X, F42X, Y45X, Q126X), (H16X, D20X, F42X, Y45X, Q126X), where X is the substituted aa, optionally chosen from the following: positions 15, 20, 45, 126—A; position 16—A or T, or also N, C, Q, M, V or W; position 42—A, or also M, P. S, T, Y, V or H; position 88—A or R.

Suitable variant IL-2 polypeptide sequences include polypeptide sequences comprising at least one insertion, deletion, or substitution and comprise an aa sequence having at least 90% (e.g., at least 95%, at least 98%, at least 99%, or 100%) an sequence identity to at least 90 (e.g., 95, 100, 110, 120, 130 or 133) contiguous aas of SEQ ID NO:112.

L-2 variants include polypeptides having at least 90% (e.g., at least 95%, 98%, or 99%) an sequence identity to at least 80 (e.g., at least 90, 100, 110, 120, or 130) contiguous aas of SEQ ID NO:112, wherein the an at position 16 is an aa other than H. In one case, the position of H16 is substituted by Asn, Cys, Gin, Met, Val, or Trp. In one case, the position of H16 is substituted by Ala. In another case, the position of H16 is substituted by Thr. Additionally, or alternatively, IL-2 variants include polypeptides having at least 90% (e.g., at least 95%, 98%, or 99%) an sequence identity to at least 80 (e.g., at least 90, 100, 110, 120, or 130) contiguous aas of SEQ ID NO:112, wherein the an at position 42 is an aa other than F. In one case, the position of F42 is substituted by Met, Pro, Ser, Thr, Trp, Tyr, Val, or His. In one case, the position of F42 is substituted by Ala.

In some cases, an IL-2 variant MOD of this disclosure exhibits decreased binding to IL-2Rα, thereby minimizing or substantially reducing the activation of Tregs by the IL-2 variant. Alternatively, or additionally, in some cases, an IL-2 variant MOD of this disclosure exhibits decreased binding to IL-2Rβ and/or IL-2Rγ such that the IL-2 variant MOD exhibits an overall reduced affinity for IL-2R. In some cases, an IL-2 variant MOD of this disclosure exhibits both properties, i.e., it exhibits decreased or substantially no binding to IL-2Rα, and also exhibits decreased binding to IL-2Rβ and/or IL-2Rγ such that the IL-2 variant polypeptide exhibits an overall reduced affinity for IL-2R. For example, IL-2 variants having substitutions at H16 and F42 have shown decreased binding to IL-2Rα and IL-2Rβ. See, Quayle et al., Clin Cancer Res; 26(8) Apr. 15, 2020, which discloses that the binding affinity of an IL-2 polypeptide with H16A and F42A substitutions for human IL-2Rα and IL-2Rβ was decreased 110- and 3-fold, respectively, compared with wild-type 1L2 binding, predominantly due to a faster off-rate for each of these interactions. TMPs comprising such variants, including variants that exhibit decreased binding to IL-2Rα and IL-2Rß, have shown the ability to preferentially bind to and activate IL-2 receptors on T cells that contain the target TCR that is specific for the peptide epitope on the TMP, and are thus less likely to deliver IL-2 to non-target T cells, i.e., T cells that do not contain a TCR that specifically binds the peptide epitope on the TMP. That is, the binding of the IL-2 variant MOD to its costimulatory polypeptide on the T cell is substantially driven by the binding of the MHC-epitope moiety rather than by the binding of the IL-2. IL-2 variants thus include polypeptides comprising an aa sequence comprising all or part of human IL-2 having a substation at position H16 and/or F42 (e.g., H16A and/or F42A substitutions).

IL-2 variants include polypeptides having at least 90% (e.g., at least 95%, 98%, or 99%) aa sequence identity to at least 80 (e.g., at least 100, 110, 120, or 130) contiguous aas of SEQ ID NO:112, wherein the aa at position 16 is an aa other than H and the aa at position 42 is other than F. In one case, the position of H16 is substituted by Ala or Thr and the position of F42 is substituted by Ala or Thr. In one case, the position of H16 is substituted by Ala and the position of F42 is substituted by Ala (an H16A and F42A variant). In a second case, the position of H16 is substituted by Thr and the position of F42 is substituted by Ala (an H16T and F42A variant). In a third case, the position of H16 is substituted by Ala and the position of F42 is substituted by Thr (an H16A and F42T variant). In a fourth case, the position of H16 is substituted by Thr and the position of F42 is substituted Thr Ala (an H16T and F42T variant). As noted above, such variants will exhibit reduced binding to both the human IL-2Rα chain and IL2Rβ chain.

In any of the wild-type or variant IL-2 sequences provided herein, the cysteine at position 125 may be substituted with an alanine (a C125A substitution). In addition to any stability provided by the substitution, it may be employed where, for example, an epitope containing peptide or payload is to be conjugated to a cysteine residue elsewhere in a T-Cell-MMP first or second polypeptide, thereby avoiding competition from the C125 of the IL-2 MOD sequence.

e. Wild-Type and Variant PD-L1 MODs

As noted above, the MOD(s) that will be present in a T-Cell-MMP generally will be MODs that provide activating immunomodulatory signals to the T cell, including, e.g., signals that cause an increase in the number of epitope-specific T cells. In some cases, however, it may be desirable to include a MOD that can provide an inhibitory/suppressing immunomodulatory signal to T cells, or may have an activating effect to T cell under some conditions, e.g., a PD-L1.

In some cases, a variant PD-L1 polypeptide (e.g., a variant of SEQ ID NO:98 or PD-L1's IgV domain, SEQ ID NO:99) exhibits reduced binding affinity to PD-1 (e.g., a PD-1 polypeptide comprising the an sequence set forth in SEQ ID NO:100), compared to the binding affinity of a PD-L1 polypeptide comprising the an sequence of wildtype PD-L1 set forth in SEQ ID NO:97 or the IgV domain provided in SEQ ID NO:98. For example, in some cases, a variant PD-L1 polypeptide binds PD-1 (e.g., a PD-1 polypeptide comprising the aa sequence set forth in SEQ ID NO:100) with a binding affinity that is at least 10% less, at least 20% less, at least 30% less, at least 40% less, at least 50% less, at least 60% less, at least 70% less, at least 80% less, at least 90% less, at least 95% less, or more than 95% less than the binding affinity of a PD-L1 polypeptide comprising the aa sequence set forth in SEQ ID NO:97 or SEQ ID NO:98. The sequences of wild type PD-L1, it's IgV domain, and PD1 are provided in the accompanying sequence listing.

Suitable PD-L1 variants are described in published PCT Application WO 2019/051091, published 14 Mar. 2019 (Applicant Cue Biopharma, Inc.). See paragraphs [00157]-[00169], the disclosure of which is expressly incorporated herein by reference. Other inhibitory/suppressing immunomodulatory, e.g., FasL also are known and may be included where an inhibitory/suppressing immunomodulatory signal is desired.

8 Additional Polypeptides

A polypeptide chain of a T-Cell-MMP or its epitope conjugate can include one or more polypeptides in addition to those described above. Suitable additional polypeptides include epitope tags and affinity domains. The one or more additional polypeptide(s) can be included as part of a polypeptide translated by cell or cell free system at the N-terminus of a polypeptide chain of a multimeric polypeptide, at the C-terminus of a polypeptide chain of a multimeric polypeptide, or internally within a polypeptide chain of a multimeric polypeptide.

9 Epitope Tags and Affinity Domains

Suitable epitope tags include, but are not limited to, hemagglutinin (HA; e.g., YPYDVPDYA (SEQ ID NO:116)); c-myc (e.g., EQKLISEEDL; SEQ ID NO:117)), and the like. Affinity domains include peptide sequences that can interact with a binding partner, e.g., such as one immobilized on a solid support, useful for identification or purification. DNA sequences encoding multiple consecutive single amino acids, such as histidine, when fused to the expressed protein, may be used for one-step purification of the recombinant protein by high affinity binding to a resin column, such as nickel SEPHAROSE®. Exemplary affinity domains include His5 (HHHHH) (SEQ ID NO:118), HisX6 (HHHHHH) (SEQ ID NO:119), C-myc (EQKLISEEDL) (SEQ ID NO:120), Flag (DYKDDDDK) (SEQ ID NO:121, StrepTag (WSHPQFEK) (SEQ ID NO:122), hemagglutinin, (e.g., HA Tag (YPYDVPDYA) (SEQ ID NO:123)), glutathione-S-transferase (GST), thioredoxin, cellulose binding domain, RYIRS (SEQ ID NO:124), Phe-His-His-Thr (SEQ ID NO:125), chitin binding domain, S-peptide, T7 peptide, SH2 domain, C-end RNA tag, WEAAAREACCRECCARA (SEQ ID NO:126), metal binding domains, e.g., zinc binding domains or calcium binding domains such as those from calcium-binding proteins, e.g., calmodulin, troponin C, calcineurin B, myosin light chain, recoverin, S-modulin, visinin, VILIP, neurocalcin, hippocalcin, frequenin, caltractin, calpain large-subunit, S100 proteins, parvalbumin, calbindin D9K, calbindin D28K, and calretinin, inteins, biotin, streptavidin, MyoD, Id, leucine zipper sequences, and maltose binding protein.

10 Epitopes

The chemical conjugation sites and chemistries described herein permit the incorporation into an unconjugated T-Cell-MMP of a molecule presenting a coronavirus epitope to form a T-Cell-MMP-epitope conjugate. Molecules that may be conjugated to an unconjugated T-Cell-MMP include those presenting a peptide epitope, phosphopeptide epitope, or glycopeptide epitope (such as those from the spike glycoprotein, nucleoprotein, membrane protein, replicase protein, non-structural protein (nsp) and the like); collectively an “epitope.” Epitopes of a T-Cell-MMP conjugate are not part of the first or second polypeptide as translated from mRNA, but may be added to a T-Cell-MMP at a chemical conjugation site. Selection of candidate MHC allele and peptide (e.g., phosphopeptide, lipopeptides or glycopeptide) epitope combinations for effective presentation to a TCR by a T-Cell-MMP-epitope conjugate can be accomplished using any of a number of well-known methods to determine if the free peptide has affinity for the specific HLA allele used to construct the T-Cell-MMP in which it will be presented as part of the epitope conjugate. It is also possible to determine if the peptide in combination with the specific heavy chain allele and β2M can affect the T-Cell in the desired manner (e.g., induction of cell activation, proliferation, anergy, or apoptosis). Applicable methods include binding assays and T-cell activation assays.

a. Cell-Based Binding Assays

As one example, cell-based peptide-induced stabilization assays can be used to determine if a candidate peptide binds an HLA class I allele intended for use in a T-Cell-MMP-epitope conjugate. The binding assay can be used in the selection of peptides for incorporation into a T-Cell-MMP-epitope conjugate using the intended allele. In this assay, a peptide of interest is allowed to bind to a TAP-deficient cell, i.e., a cell that has defective transporter associated with antigen processing (TAP) machinery, and consequently, few surface class I molecules. Such cells include, e.g., the human T2 cell line (T2 (174×CEM.T2; American Type Culture Collection (ATCC) No. CRL-1992)). Henderson et al. (1992) Science 255:1264. Without efficient TAP-mediated transport of cytosolic peptides into the endoplasmic reticulum, assembled class I complexes are structurally unstable, and retained only transiently at the cell surface. However, when T2 cells are incubated with an exogenous peptide capable of binding class I, surface peptide-HLA class I complexes are stabilized and can be detected by flow cytometry with, e.g., a pan anti-class I monoclonal antibody, or directly where the peptide is fluorescently labeled. The stabilization and resultant increased life-span of peptide-HLA complexes on the cell surface by the addition of a peptide validates their identity. Accordingly, binding of candidate peptides for presentation by various Class I HLA heavy chain alleles can be tested by genetically modifying the T2 cells to express the HLA H allele of interest.

In a non-limiting example of use of a T2 assay to assess peptide binding to HLA A*0201, T2 cells are washed in cell culture medium, and suspended at 10⁶ cells/ml. Peptides of interest are prepared in cell culture medium and serially diluted providing concentrations of 200 μM, 100 μM, 20 μM and 2 μM. The cells are mixed 1:1 with each peptide dilution to give a final volume of 200 μL and final peptide concentrations of 100 μM, 50 μM. 10 μM and 1 μM. An HLA A*0201 binding peptide, GILGFVFTL, and a non-HLA A*0201-restricted peptide, HPVGEADYF (HLA-B*3501), are included as positive and negative controls, respectively. The cell/peptide mixtures are kept at 37° C. in 5% CO₂ for ten minutes; then incubated at room temperature overnight. Cells are then incubated for 2 hours at 37° C. and stained with a fluorescently-labeled anti-human HLA antibody. The cells are washed twice with phosphate-buffered saline and analyzed using flow cytometry. The average mean fluorescence intensity (MFI) of the anti-HLA antibody staining is used to measure the strength of binding.

b. Biochemical Binding Assays

MHC Class I complexes comprising a β2M polypeptide complexed with an HLA heavy chain polypeptide of a specific allele intended for use in construction of a T-Cell-MMP can be tested for binding to a peptide of interest in a cell-free in vitro assay system. For example, a labeled reference peptide (e.g., fluorescently labeled) is allowed to bind the MHC-class I complex to form an MHC-reference peptide complex. The ability of a test peptide of interest to displace the labeled reference peptide from the complex is tested. The relative binding affinity is calculated as the amount of test peptide needed to displace the bound reference peptide. See, e.g., van der Burg et al. (1995) Human Immunol. 44:189.

As another example, a peptide of interest can be incubated with an MHC Class I complex (containing an HLA heavy chain peptide and β2M) and the stabilization of the MHC complex by bound peptide can be measured in an immunoassay format. The ability of a peptide of interest to stabilize the MHC complex is compared to that of a control peptide presenting a known T-cell epitope. Detection of stabilization is based on the presence or absence of the native conformation of the MHC complex bound to the peptide using an anti-HLA antibody. See, e.g., Westrop et al. (2009) J. Immunol. Methods 341:76; Steinitz et al. (2012) Blood 119:4073; and U.S. Pat. No. 9,205,144.

c. T-Cell Activation Assays

Whether a given peptide binds an MHC Class I complex (comprising an HLA heavy chain and a β2M polypeptide), and, when bound to the HLA complex, can effectively present an epitope to a TCR, can be determined by assessing T-cell response to the peptide-HLA complex. T-cell responses that can be measured include, e.g., interferon-gamma (IFNγ) production, cytotoxic activity, and the like.

(i) ELISPOT Assays

Suitable assays include, e.g., an enzyme linked immunospot (ELISPOT) assay where production of a product by target cells (e.g., IFNγ production by target CD8⁺ T) is measured following contact of the target with an antigen-presenting cell (APC) that presents a peptide of interest complexed with a class I MHC (e.g., HLA). Antibody to IFNγ is immobilized on wells of a multi-well plate. APCs are added to the wells, and the plates are incubated for a period of time with a peptide of interest, such that the peptide binds HLA class I on the surface of the APCs. CD8⁺ T cells specific for the peptide are added to the wells, and the plate is incubated for about 24 hours. The wells are then washed, and any IFNγ bound to the immobilized anti-IFNγ antibody is detected using a detectably labeled anti-IFNγ antibody. A colorimetric assay can be used. For example, the detectably labeled anti-IFNγ antibody can be a biotin-labeled anti-IFNγ antibody, which can be detected using, e.g., streptavidin conjugated to alkaline phosphatase. A BCIP/NBT (5-bromo-4-chloro-3-indolyl phosphate/nitro blue tetrazolium) solution is added, to develop the assay. The presence of IFNγ-secreting T cells is identified by colored spots. Negative controls include APCs not contacted with the peptide. APCs expressing various HLA heavy chain alleles can be used to determine whether a peptide of interest effectively binds to an HLA class I molecule comprising a particular HLA H chain.

(ii) Cytotoxicity Assays

Whether a given peptide binds to a particular MHC class I heavy chain allele complexed with β2M and, when bound, can effectively present an epitope to a TCR, can also be determined using a cytotoxicity assay. A cytotoxicity assay involves incubation of a target cell with a cytotoxic CD8⁺ T cell. The target cell displays on its surface an MHC class I complex comprising β2M, and an epitope-peptide and MHC heavy chain allele combination to be tested. The target cells can be radioactively labeled, e.g., with ⁵¹Cr. Whether the target cell effectively presents the epitope to a TCR on the cytotoxic CD8⁺ T cell, thereby inducing cytotoxic activity by the CD8⁺ T cell toward the target cell, is determined by measuring release of ⁵¹Cr from the lysed target cell. Specific cytotoxicity can be calculated as the amount of cytotoxic activity in the presence of the peptide minus the amount of cytotoxic activity in the absence of the peptide.

(iii) Detection of Antigen-Specific T Cells with Peptide-HLA Tetramers

As another example, multimers (e.g., tetramers) of peptide-MHC complexes are generated with fluorescent or heavy metal tags. The multimers can then be used to identify and quantify specific T cells via flow cytometry (FACS) or mass cytometry (CyTOF). Detection of epitope-specific T cells provides direct evidence that the peptide-bound HLA molecule is capable of binding to a specific TCR on a subset of antigen-specific T cells. See, e.g., Klenerman et al. (2002) Nature Reviews Immunol. 2:263.

d. Epitope-Presenting Peptides

A T-Cell-MMP-epitope conjugate comprises any of a variety of peptide epitopes derived from coronavirus components (e.g., proteins). A T-Cell-MMP-epitope conjugate may comprise a peptide that, when in an MHC/peptide complex (e.g., an HLA/peptide complex), presents an epitope to a T cell. A peptide, present in a T-Cell-MMP-epitope conjugate of the present disclosure, that, when in an MHC/peptide complex (e.g., an HLA/peptide complex), presents an epitope to a T cell, may be referred to herein as a “peptide epitope,” “T-Cell-MMP-epitope conjugate,” or, simply, an “epitope.” Peptides from post-translational modified polypeptides/proteins may also serve as epitopes, including phosphopeptides, glycopeptides and lipopeptides (e.g., peptides modified with fatty acids, isoprenoids, sterols, phospholipids, or glycosylphosphatidyl inositol).

In some cases, the coronavirus derived epitope peptide present in a T-Cell-MMP-epitope conjugate is restricted to an HLA-A, -B, -C, -E, -F or -G allele. In an embodiment, the coronavirus derived epitope peptide present in a T-Cell-MMP-epitope conjugate is restricted to an HLA protein found in any of FIGS. 3A to 3H. In an embodiment, the coronavirus derived epitope peptide present in a T-Cell-MMP-epitope conjugate is restricted to HLA-A*0101, A*0201, A*0301, A*1101, A*2301, A*2402, A*2407, A*3303, and/or A*3401. In an embodiment, the coronavirus derived epitope peptide present in a T-Cell-MMP-epitope conjugate of the present disclosure is restricted to HLA-B*0702, B*0801, B*1502, HLA-B*3501, B*3802, B*4001, HLA-B*4402, HLA-B*4403, B*4601, B*5301 and/or HLA-B*5801. In an embodiment, the coronavirus derived epitope peptide present in a T-Cell-MMP-epitope conjugate is restricted to C*0102, C*0303, C*0304, C*0401, C*0602, C*0701, C*702, C*0801, and/or C*1502. In an embodiment, the coronavirus derived epitope peptide present in a T-Cell-MMP-epitope conjugate is restricted to HLA-A*0101, A*0201, A*0301, A*1101, A*2301, and/or A*2402. In an embodiment, the coronavirus derived epitope peptide present in a T-Cell-MMP-epitope conjugate is restricted to HLA-B*0702, B*0801, HLA-B*3501, B*4001, HLA-B*4402, HLA-B*4403 and/or HLA-B*5801.

An epitope (or the epitope presenting sequence of the peptide) present in a T-Cell-MMP-epitope conjugate can be a peptide of from 4 to 25 contiguous aas (e.g., 4 aa, 5 aa, 6 aa, 7 aa, 8 aa, 9 aa, 10 aa, 11aa, 12 aa, 13 aa, 14 aa, 15 aa, 16 aa, 17 aa, 18 aa, 19 aa, 20 aa, 21 aa, 22 aa, 23 aa, 24 aa, or 25 aa. or from 7 aa to 25 aa, from 7 aa to 12 aa, from 7 an to 25 aa, from 10 an to 15 aa, from 15 an to 20 aa, or from 20 aa to 25 aa).

In an embodiment, an epitope present in a T-Cell-MMP-epitope conjugate is a peptide specifically bound by a T-cell, i.e., the epitope is specifically bound by a T-cell with a T-cell receptor specific for that epitope. An epitope-specific T cell binds an epitope having a reference amino acid sequence, but does not substantially bind an epitope that differs from the reference amino acid sequence. For example, an epitope-specific T cell binds an epitope having a reference amino acid sequence, and binds an epitope that differs from the reference amino acid sequence, if at all, with an affinity that is less than 10⁻⁶ M, less than 10⁻⁵ M, or less than 10⁻⁴ M. An epitope-specific T cell can bind an epitope for which it is specific with an affinity of at least 10⁻⁷ M, at least 10⁻⁸ M, at least 10⁻⁹ M, or at least 10⁻¹⁰ M.

(i) Epitopes Present in Coronavirus-Associated Antigens

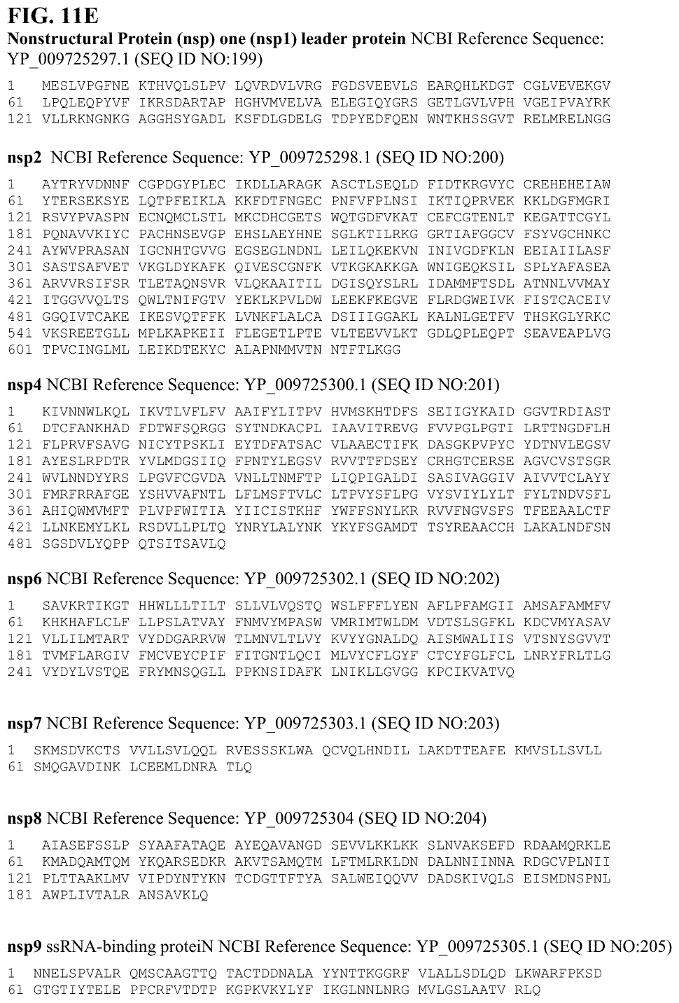

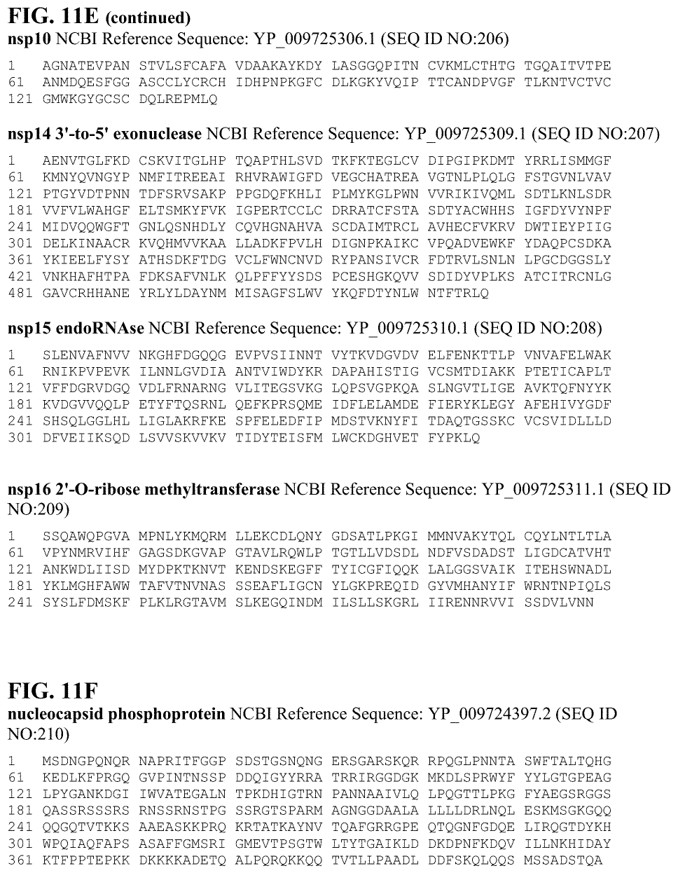

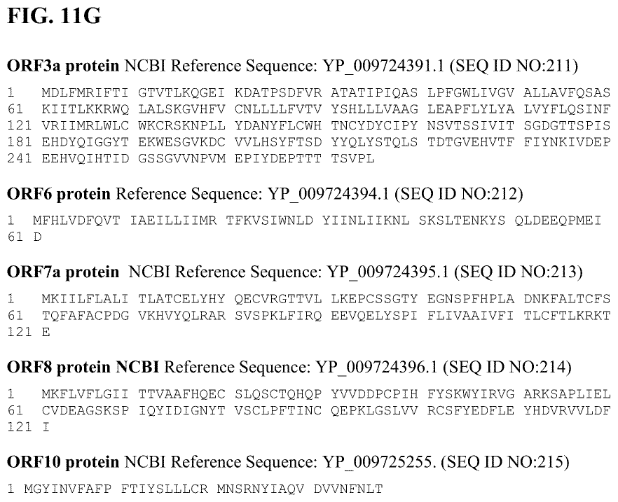

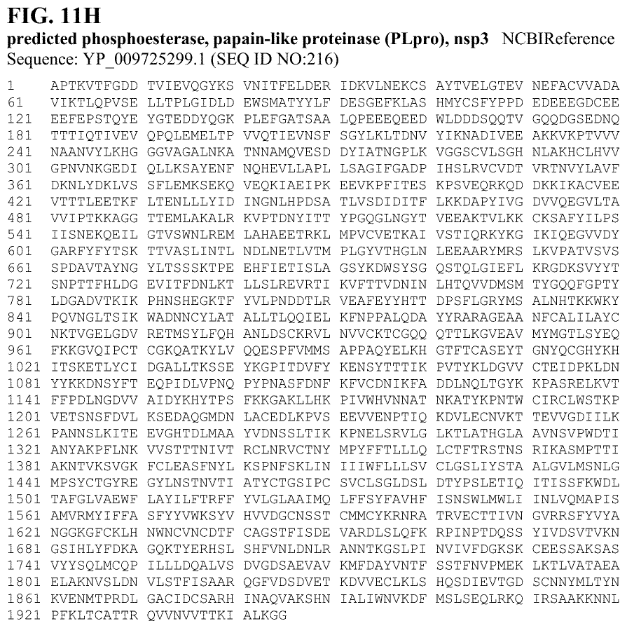

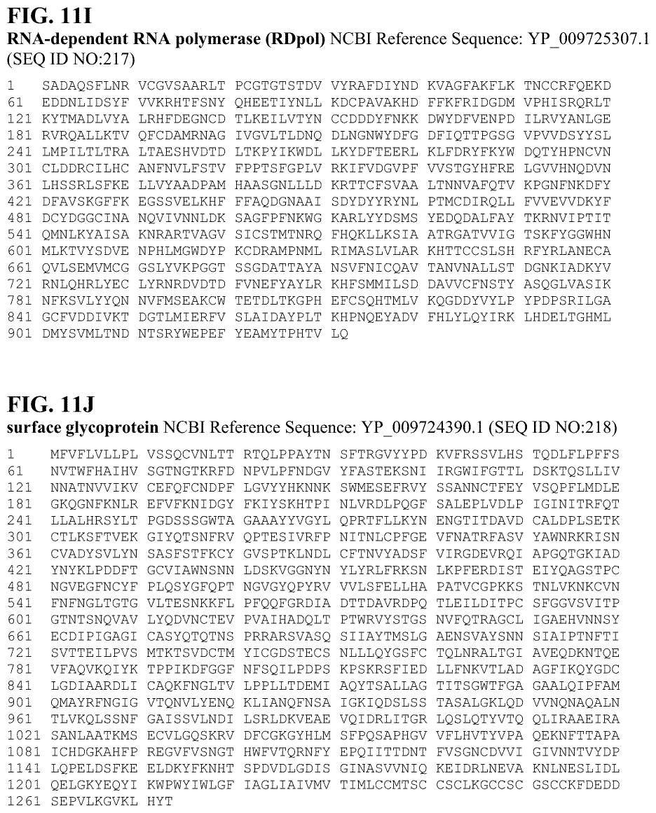

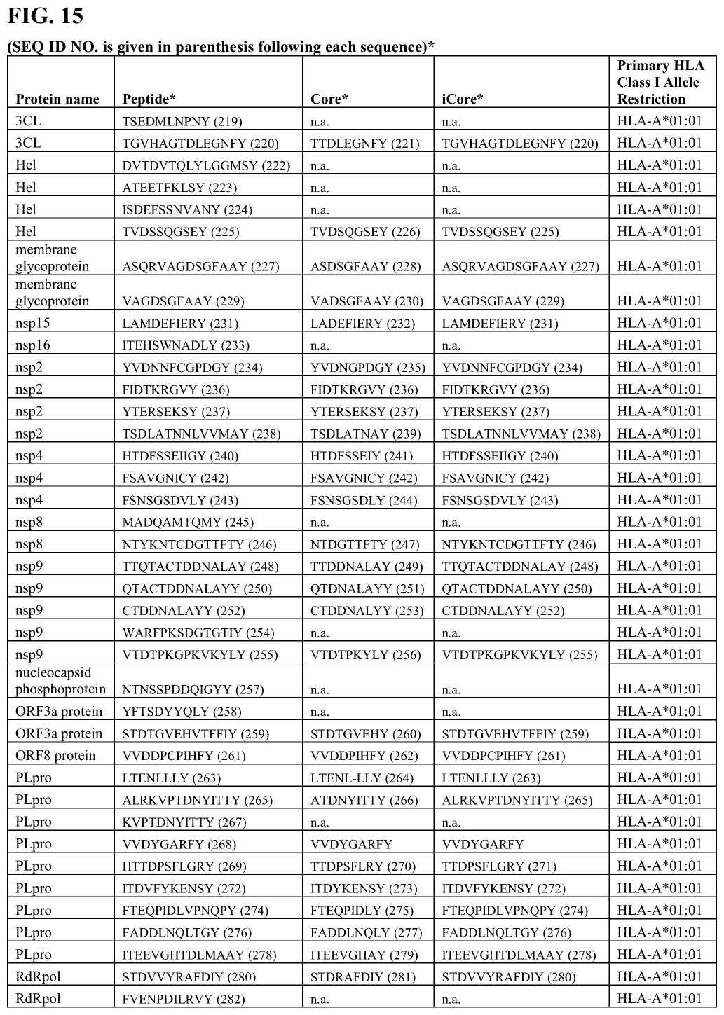

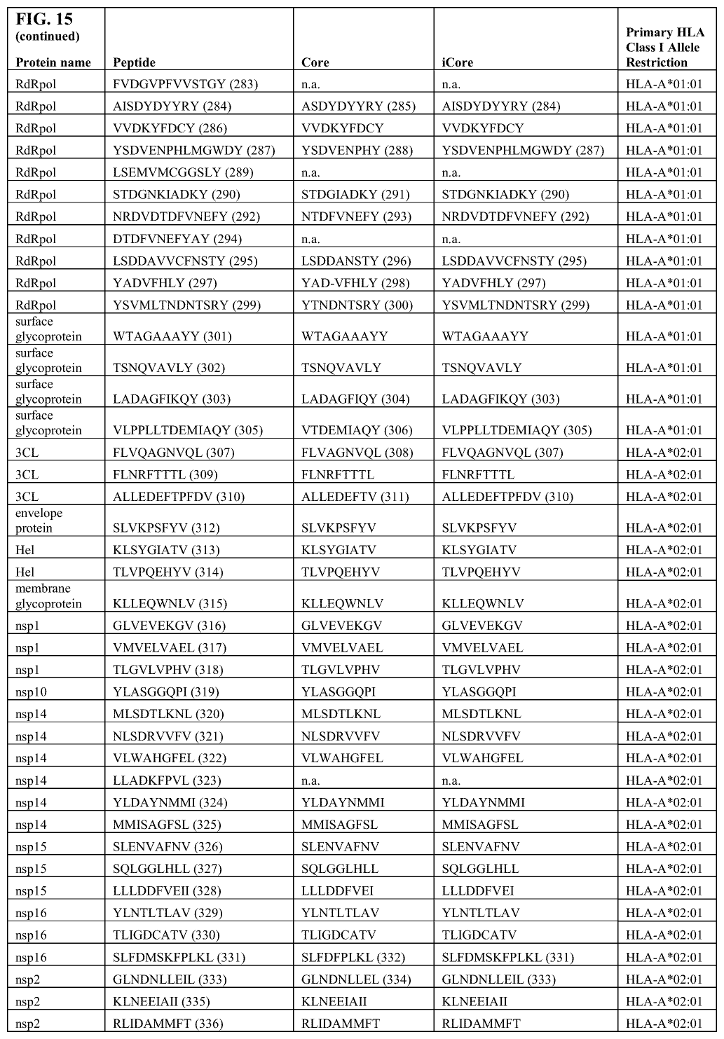

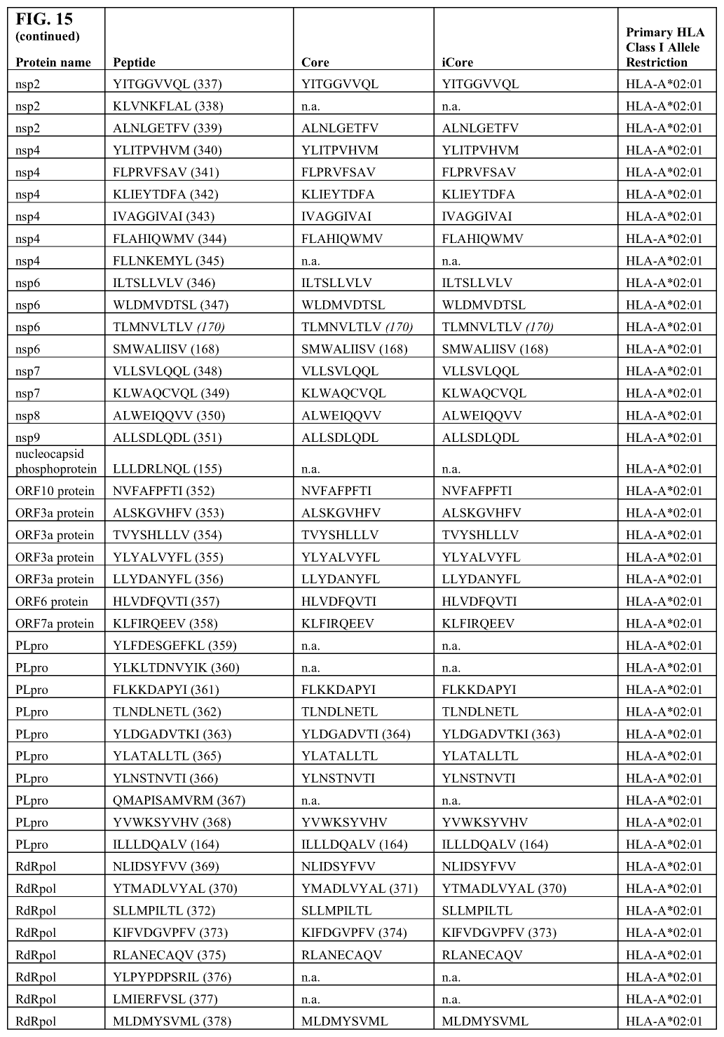

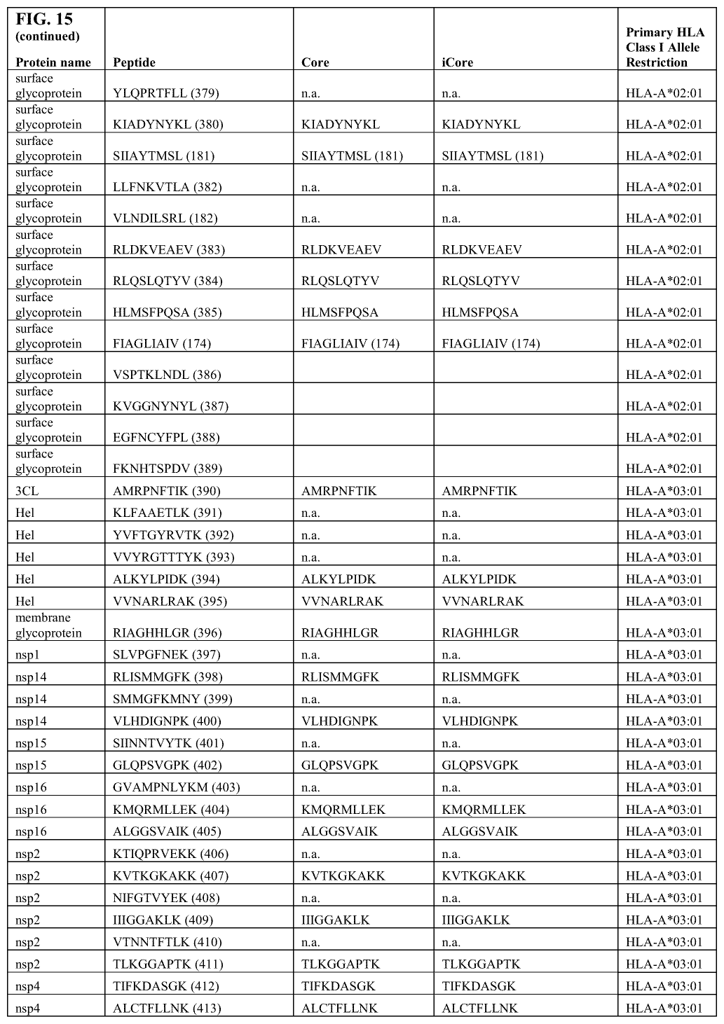

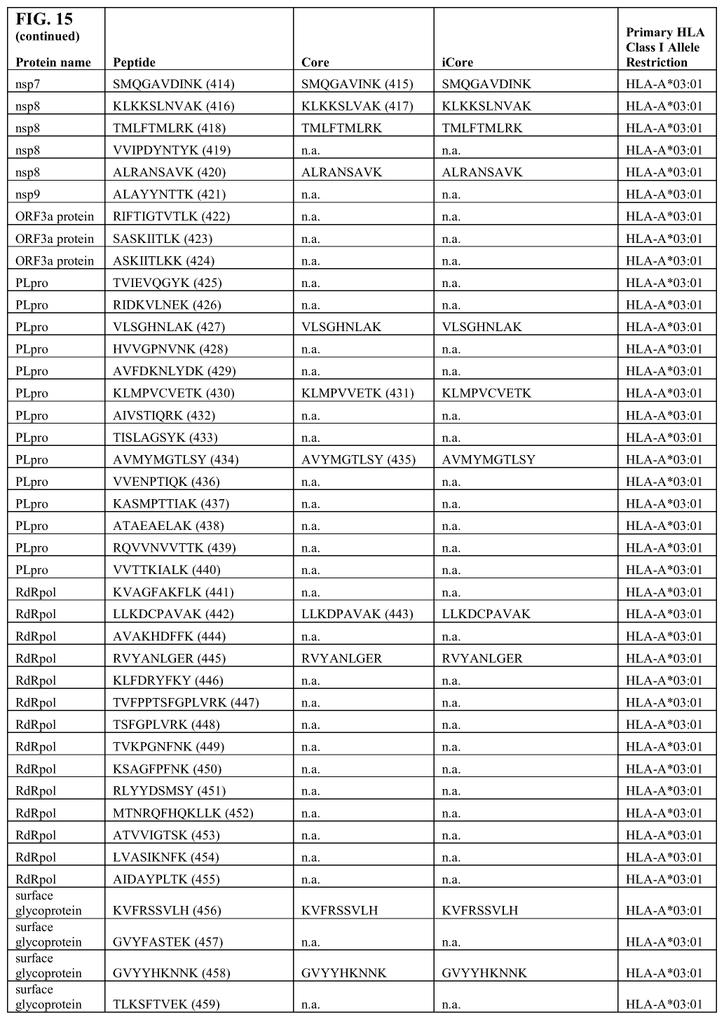

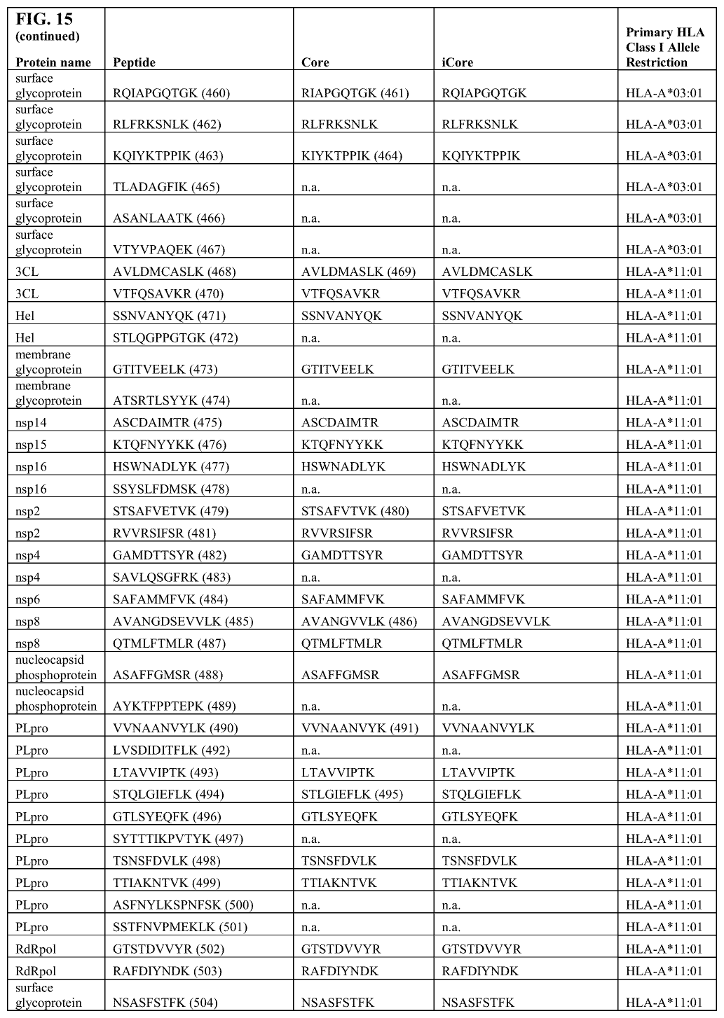

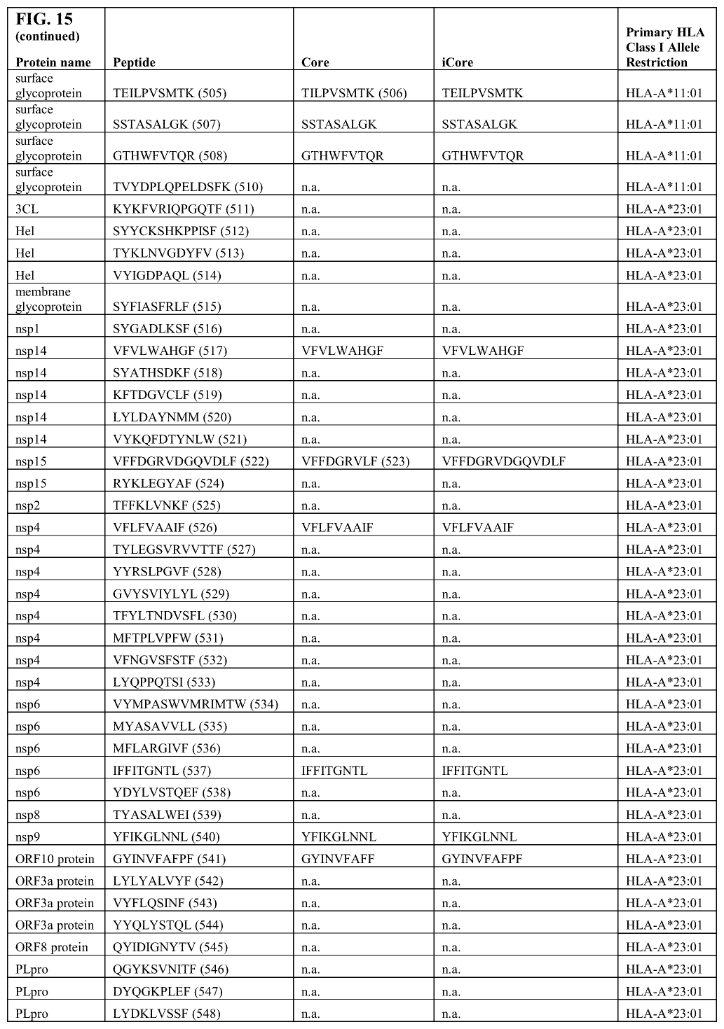

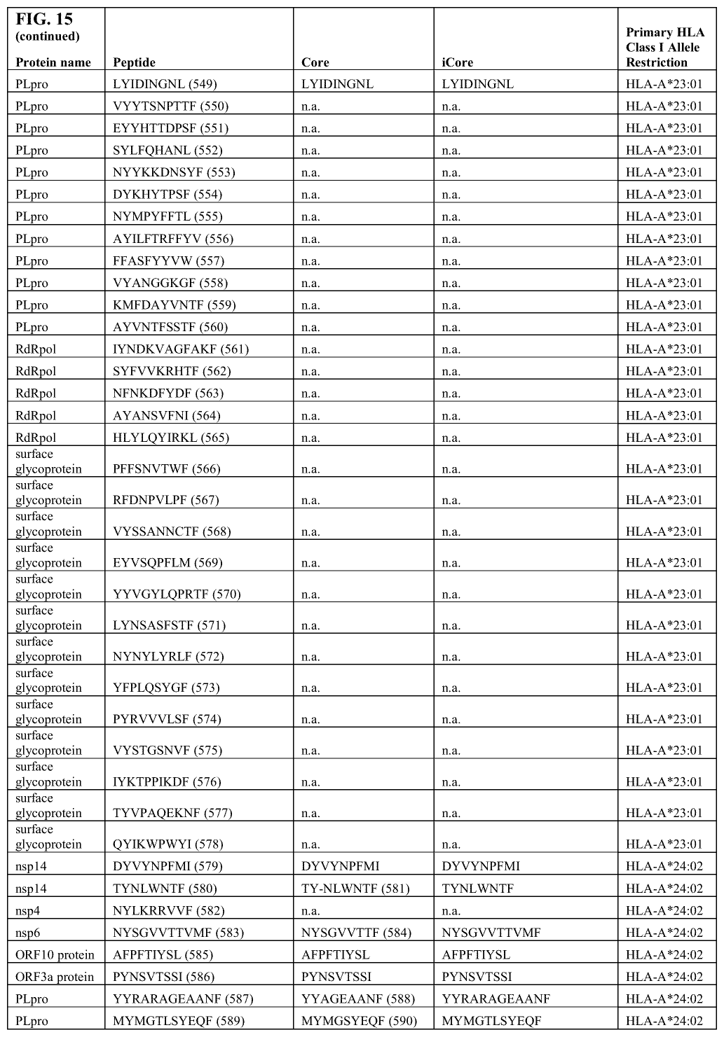

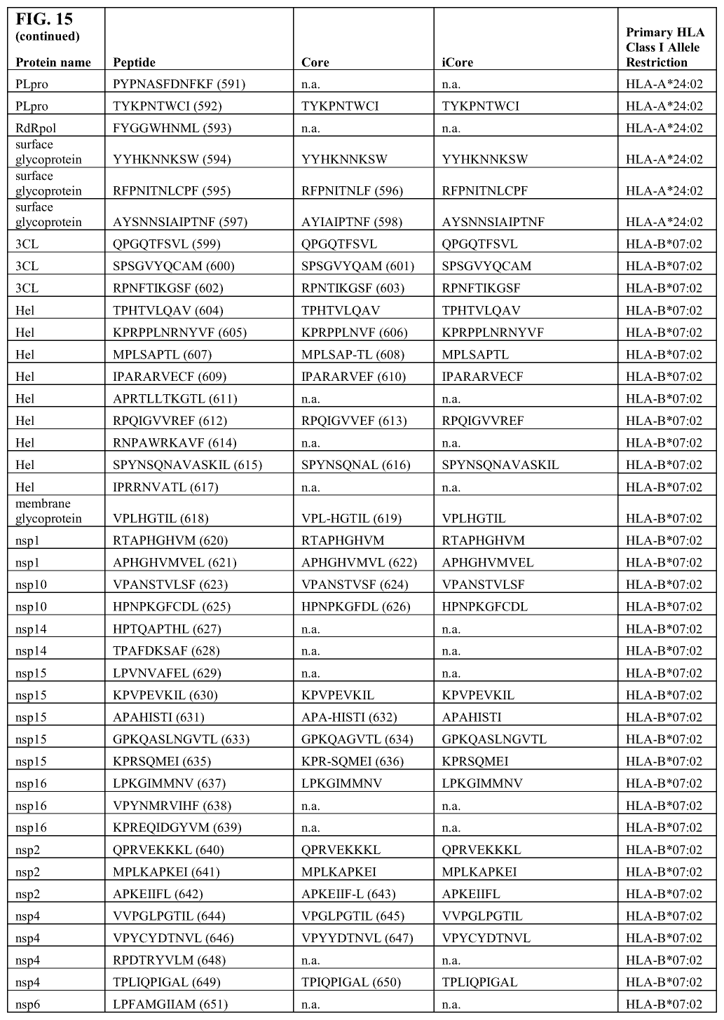

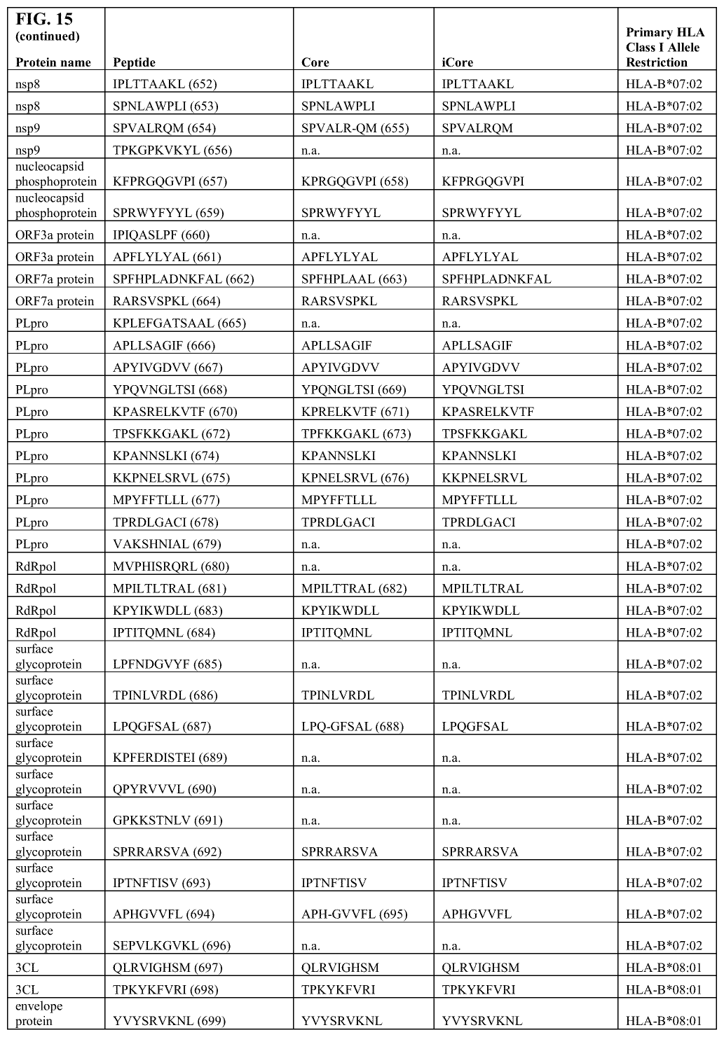

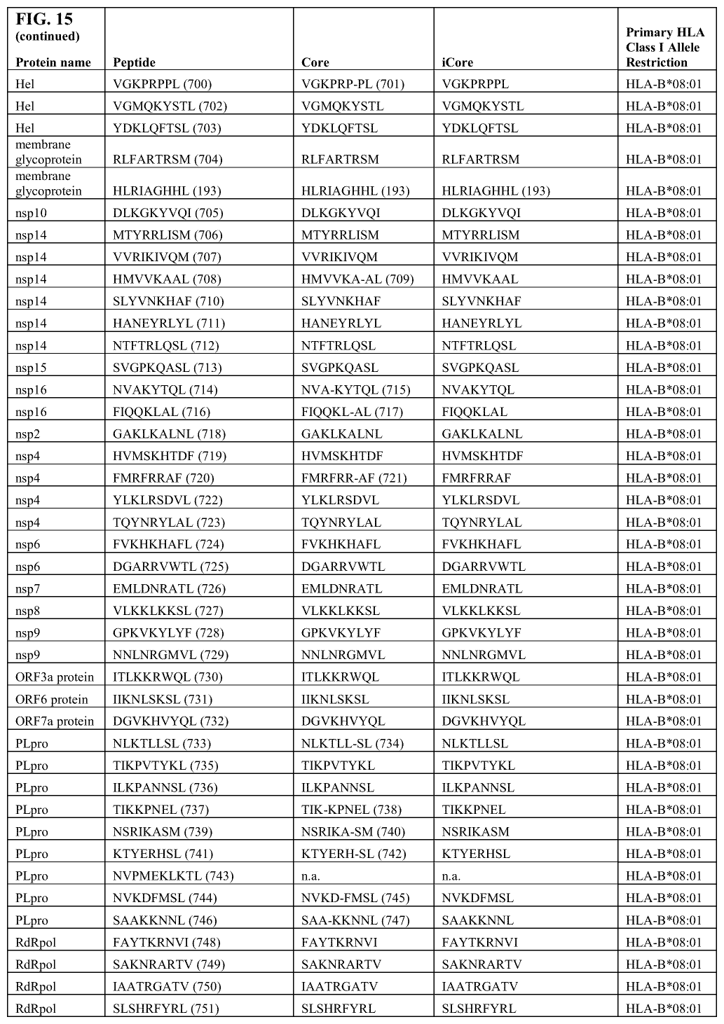

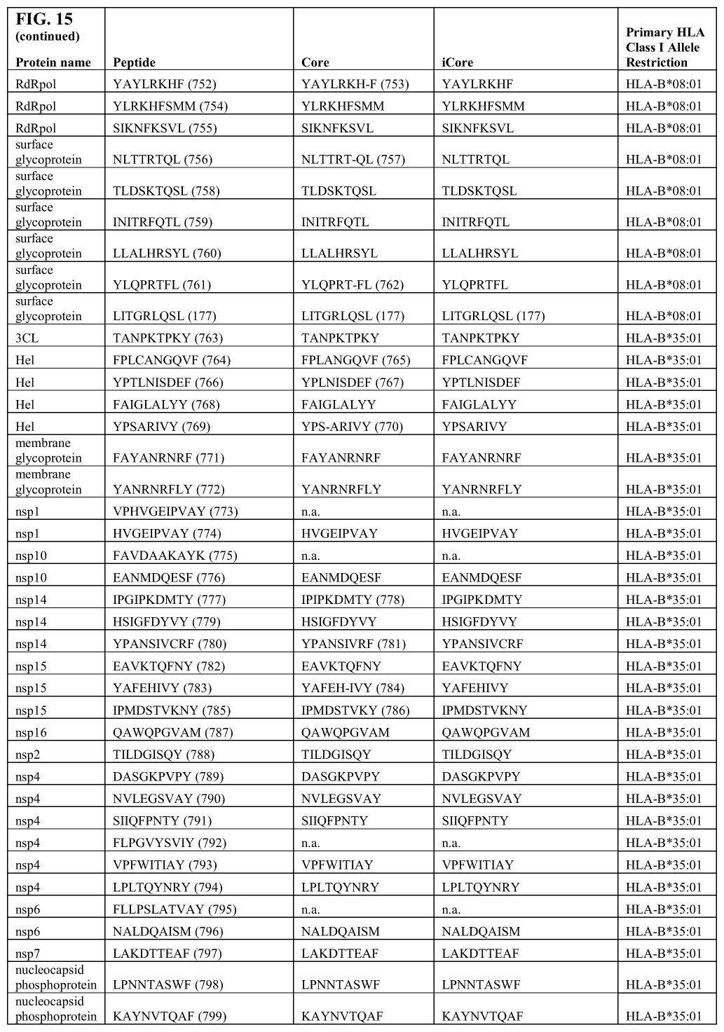

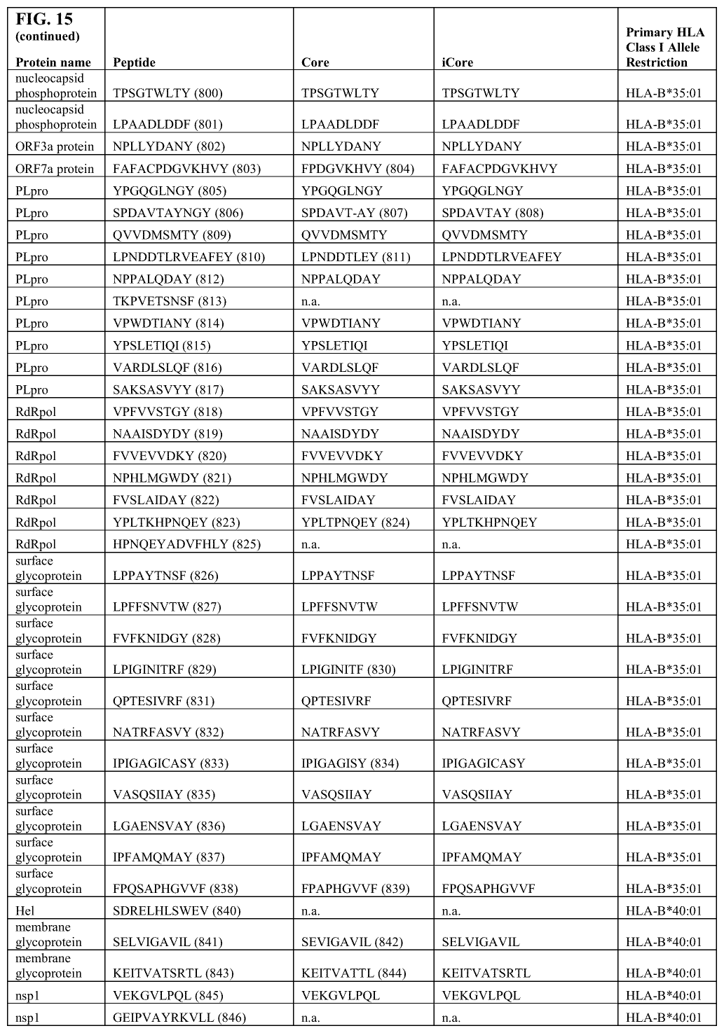

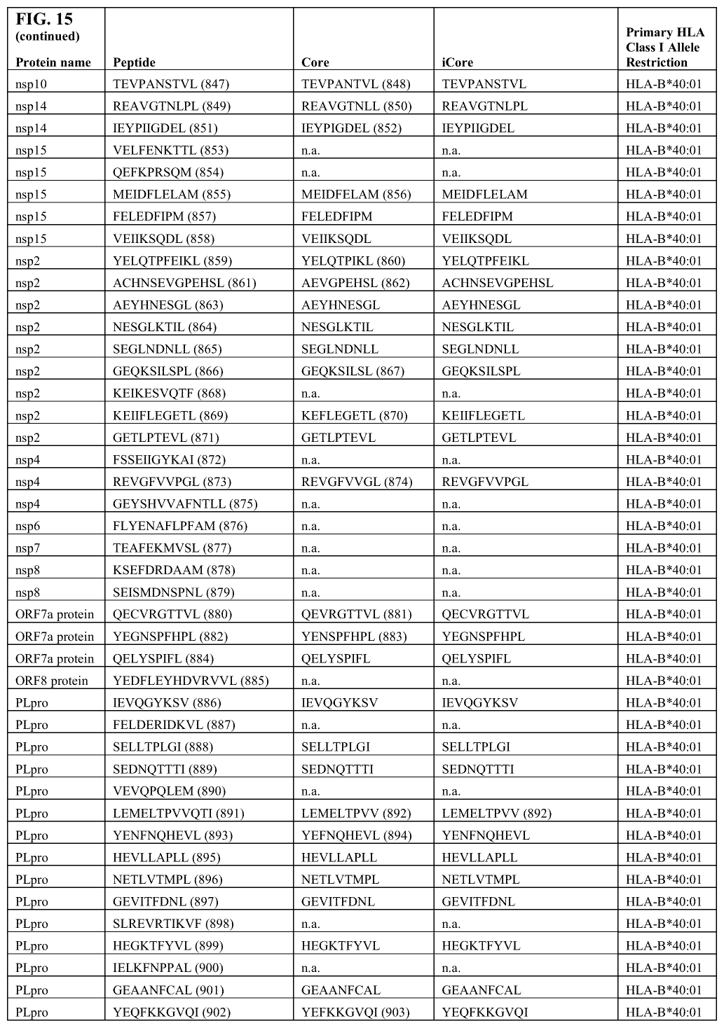

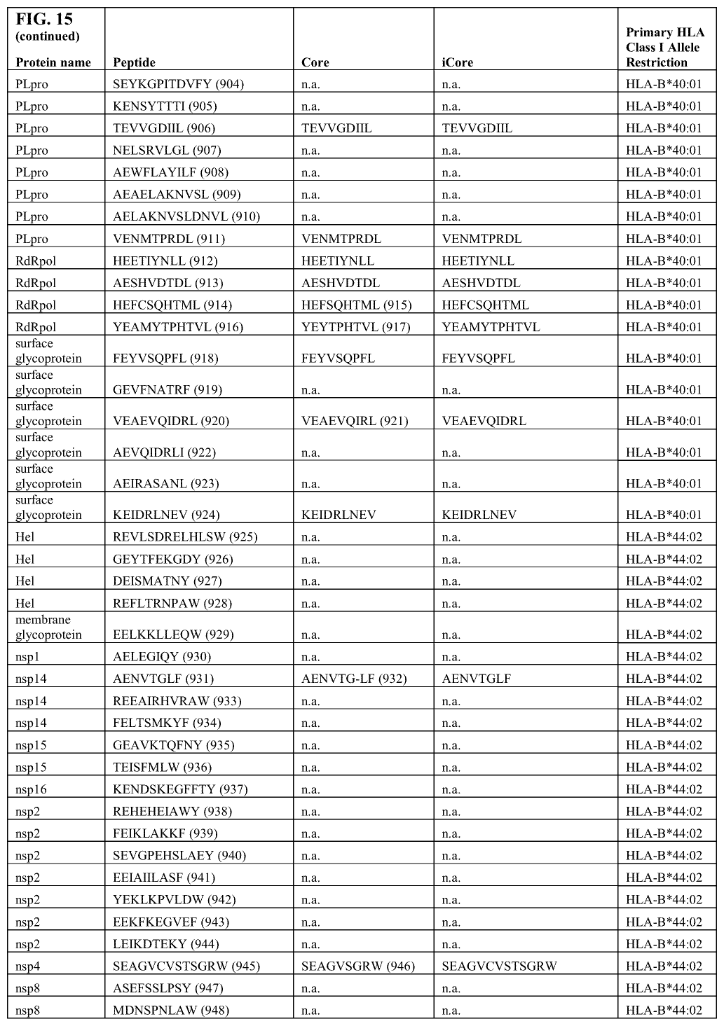

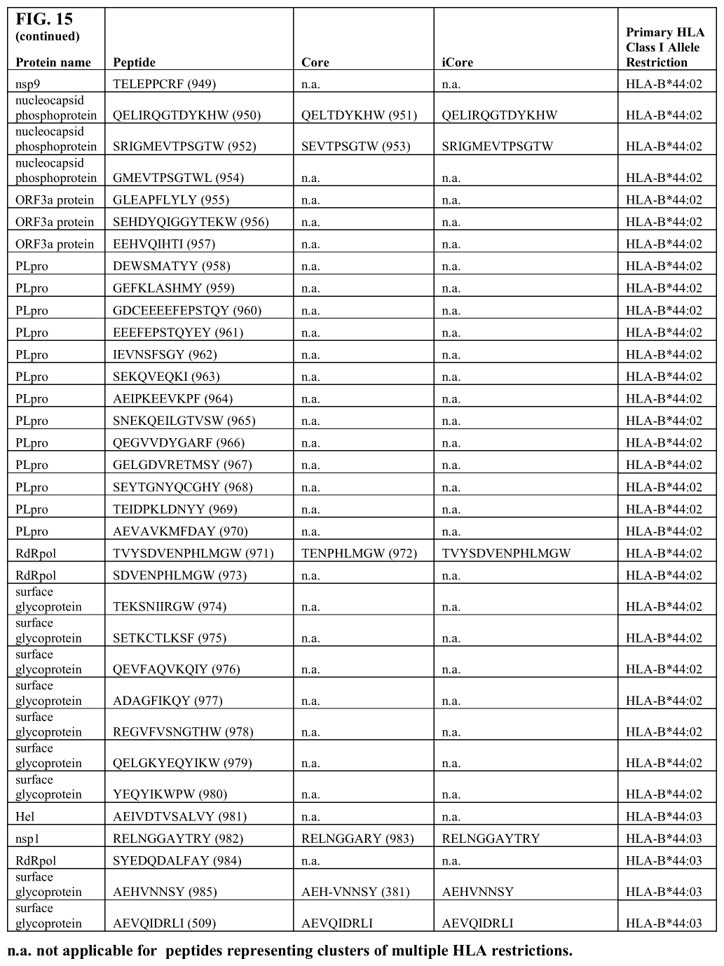

An epitope (a peptide presenting one or more epitopes) present in a T-Cell-MMP-epitope conjugate of the present disclosure is a Coronavirus peptide or derived from a coronavirus peptide (e.g., Core and iCore peptides that differ from Coronavirus peptides, see FIGS. 11A-11G). A portion of the coronavirus protein that presents one or more epitopes is referred to herein as a “Coronavirus peptide” or a “coronavirus epitope.” A coronavirus peptide present in a T-Cell-MMP-epitope conjugate can comprise a peptide having from 4 to 25 contiguous aas (e.g., 4 aa, 5 aa, 6 aa, 7 aa, 8 aa, 9 aa, 10 aa, 11 aa, 12 aa, 6-15 aa, 7-15 aa, 10-15 aa, 15-20 aa, or 20-25 aa) of an aa sequence having at least 80%, at least 90%, at least 95%, at least 98%, at least 99%, or 100% an sequence identity to any of the an sequences depicted in Table 2, FIGS. 11A to 11J, or FIG. 15. A coronavirus epitope present in a T-Cell-MMP-epitope conjugate can be a peptide having from 6 to 25 contiguous aas (e.g., 6 aa, 7 aa, 8 aa, 9 aa, 10-15 aa, 15-20 aa, or 20-25 aa) of an aa sequence having at least 80%, at least 90%, at least 95%, at least 98%, at least 99%, or 100% an sequence identity to any one of the coronavirus an sequences depicted in FIG. 15 or Table 2.

Coronavirus epitopes present in a T-Cell-MMP-epitope conjugate may be selected from those peptides presented in FIG. 15. The coronavirus epitopes present in a T-Cell-MMP-epitope conjugate may be those presented under the heading “Peptide” in FIG. 15. The coronavirus epitopes present in a T-Cell-MMP-epitope conjugate may be those presented under the heading “Core” in FIG. 15. The coronavirus epitopes present in a T-Cell-MMP-epitope conjugate may be those presented under the heading “iCore” in FIG. 15.

A coronavirus epitope present in a T-Cell-MMP-epitope conjugate can be a peptide having from 6 to 25 contiguous aas (e.g., 6 aa, 7 aa, 8 aa, 9 aa, 10-15 aa, 15-20 aa, or 20-25 aa) of an aa sequence having at least 80%, at least 90%, at least 95%, at least 98%, at least 99%, or 100% aa sequence identity to any one of the coronavirus an sequences depicted in Table 2.

Suitable coronavirus epitopes include those common to SARS-CoV and SARS-CoV-2 set forth in Table 2 (see Grifoni et al., Cell Host & Microbe 27: 1-10 (2020)).

TABLE 2

| | | SARS-CoV-2 | Corona | Start and | |
|---|---|---|---|---|---|
| SARS-CoV | HLA | (Covid-19) | virus | End | Identity |
| Sequence* | Restriction | Sequence* | Protein^{a} | Position | Percent |
| GLMWLSYFV (149) | A*02:01 | GLMWLSYFI (150) | M | 89-97 | 89 |
| TLACFVLAAV (151) | A*02:01 | TLACFVLAAV | M | 61-70 | 100 |
| ALNTPKDHI (152) | A*02:01 | ALNTPKDHI | N | 138-146 | 100 |
| GMSRIGMEV (153) | A*02:01 | GMSRIGMEV | N | 316-324 | 100 |
| LALLLLDRL (154) | A*02:01 | LALLLLDRL | N | 219-227 | 100 |
| LLLDRLNQL (155) | A*02:01 | LLLDRLNQL | N | 222-230 | 100 |
| LQLPQGTTL (156) | A*02:01 | LQLPQGTTL | N | 159-167 | 100 |
| RLNQLESKV (157) | A*02:01 | RLNQLESKM (158) | N | 226-234 | 89 |
| ALSGVFCGV (159) | A*02:01 | SLPGVFCGV (160) | Orf lab | 2942-2950 | 78 |
| CLDAGINYV (161) | A*02:01 | CLEASFNYL (162) | Orf lab | 2139-2147 | 56 |
| ILLLDQVLV (163) | A*02:01 | ILLLDQALV (164) | Orf lab | 2498-2506 | 89 |
| LLCVLAALV (165) | A*02:01 | SACVLAAEC (166) | Orf lab | 2840-2848 | 56 |
| SMWALVISV (167) | A*02:01 | SMWALIISV (168) | Orf lab | 3661-3669 | 89 |
| TLMNVITLV (169) | A*02:01 | TLMNVLTLV (170) | Orf lab | 3639-3647 | 89 |
| WLMWFIISI (171) | A*02:01 | WLMWLIINL (172) | Orf lab | 2292-2300 | 67 |
| ALNTLVKQL (173) | A*02:01 | ALNTLVKQL | S | 958-966 | 100 |
| FIAGLIAIV (174) | A*02:01 | FIAGLIAIV | S | 1220-1228 | 100 |
| KLPDDFMGCV (175) | A*02:01 | KLPDDFTGCV (176) | S | 424-433 | 90 |
| LITGRLQSL (177) | A*02:01 | LITGRLQSL | S | 996-1004 | 100 |
| NLNESLIDL (178) | A*02:01 | NLNESLIDL | S | 1192-1200 | 100 |
| RLNEVAKNL (179) | A*02:01 | RLNEVAKNL | S | 1185-1193 | 100 |
| SIVAYTMSL (180) | A*02:01 | SIIAYTMSL(181) | S | 691-699 | 89 |
| VLNDILSRL (182) | A*02:01 | VLNDILSRL | S | 976-984 | 100 |
| QFKDNVILL (183) | A*24:02 | NFKDQVILL (184) | N | 345-353 | 78 |
| GETALALLLL (185) | B*40:01 | GDAALALLLL (186) | N | 215-224 | 80 |
| MEVTPSGTWL (187) | B*40:01 | MEVTPSGTWL | N | 322-331 | 100 |
| RFFTLGSITAQPVKI | B*58:01 | RIFTIGTVTLKQGEI | Orf 3a | 6-20 | 40 |
| (188) | | (189) | | | |
| SITAQPVKI (190) | B*58:01 | TVTLKQGEI (191) | Orf 3a | 12-20 | 22 |
| HLRMAGHSL (192) | Class 1 | HLRIAGHHL (193) | M | 148-156 | 78 |
| TKQYNVTQAF (194) | Class I | TKAYNVTQAF (131) | N | 265-274 | 90 |

*(SEQ ID NO. is given in parenthesis following each sequence)
^{a}S, surface glycoprotein; M, membrane protein; N, nucleocapsid phosphoprotein, Orf lab polyprotein (see NCBI Reference Sequence: YP_009724389.1).
Restrictions defined only in HLA-transgenic mice are indicated by the italicized font

11 Payloads

A broad variety of payloads may be associated with T-Cell-MMPs and T-Cell-MMP-epitope conjugates, which may incorporate more than one type of payload conjugated (covalently) to the T-Cell-MMPs at a first or second chemical conjugation site. In addition, where the T-Cell-MMP molecules or their epitope conjugates multimerize, it may be possible to incorporate monomers labeled with different payloads into a multimer. Accordingly, it is possible to introduce one or more payloads selected, for example, from the group consisting of: therapeutic agents, antiviral agents, antibiotics, diagnostic agents or labels, and the like. It will be apparent that some payloads may fall into more than one category (e.g., a specific antiviral may fall under the general terms drug or therapeutic agent or under the narrower term antiviral agents).

As noted above, T-Cell-MMP polypeptides (e.g., a scaffold or Fc polypeptide) can be modified with crosslinking reagents to conjugate payloads and/or epitopes to chemical conjugation sites attached to or in the first or second polypeptide of the T-Cell-MMPs (e.g., at a chemical conjugation site such as an engineered cysteine or lysine). Exemplary crosslinking agents include, but are not limited to, succinimidyl 4-(N-maleimidomethyl)-cyclohexane-1-carboxylate (SMCC), sulfo-SMCC, maleimidobenzoyl-N-hydroxysuccinimide ester (MBS), sulfo-MBS or succinimidyl-iodoacetate. Introducing payloads using an excess of such crosslinking agents can result in multiple molecules of payload being incorporated into the T-Cell-MMP. Some bifunctional linkers for introducing payloads into T-Cell-MMPs and their epitope conjugates include cleavable linkers or non-cleavable linkers. In some cases, the payload linker is a protease-cleavable linker. Suitable payload linkers include, e.g., peptides (e.g., from 2 to 10 amino acids in length; e.g., 2, 3, 4, 5, 6, 7, 8, 9, or 10 amino acids in length), alkyl chains, poly(ethylene glycol), disulfide groups, thioether groups, acid labile groups, photolabile groups, peptidase labile groups, and esterase labile groups. Non-limiting examples of suitable reagents for use as payload linkers are: N-succinimidyl-[(N-maleimidopropionamido)-tetraethyleneglycol]ester (NHS-PEG4-maleimide); N-succinimidyl 4-(2-pyridyldithio)butanoate (SPDB); N-succinimidyl 4-(2-pyridyldithio)2-sulfobutanoate (sulfo-SPDB); N-succinimidyl 4-(2-pyridyldithio) pentanoate (SPP); N-succinimidyl-4-(N-maleimidomethyl)-cyclohexane-1-carboxy-(6-amidocaproate) (LC-SMCC); K-maleimidoundecanoic acid N-succinimidyl ester (KMUA); γ-maleimide butyric acid N-succinimidyl ester (GMBS); ε-maleimidocaproic acid N-hydroxysuccinimide ester (EMCS); m-maleimide benzoyl-N-hydroxysuccinimide ester (MBS); N-(α-maleimidoacetoxy)-succinimide ester (AMAS); succinimidyl-6-(β-maleimidopropionamide)hexanoate (SMPH); N-succinimidyl 4-(p-maleimidophenyl)butyrate (SMPB); N-(p-maleimidophenyl)isocyanate (PMPI); N-succinimidyl 4(2-pyridylthio)pentanoate (SPP); N-succinimidyl(4-iodo-acetyl)aminobenzoate (SIAB); 6-maleimidocaproyl (MC); maleimidopropanoyl (MP); p-aminobenzyloxycarbonyl (PAB); N-succinimidyl 4-(maleimidomethyl)cyclohexanecarboxylate (SMCC); N-succinimidyl-4-(N-maleimidomethyl)-cyclohexane-1-carboxy-(6-amidocaproate), a “long chain” analog of SMCC (LC-SMCC); 3-maleimidopropanoic acid N-succinimidyl ester (BMPS); N-succinimidyl iodoacetate (SIA); N-succinimidyl bromoacetate (SBA); and N-succinimidyl 3-(bromoacetamido)propionate (SBAP)

Control of the stoichiometry of the reaction may result in some selective modification where engineered sites with chemistry orthogonal to all other groups in the molecule is not utilized. Reagents that display far more selectivity, such as the bis-thio linkers discussed above, tend to permit more precise control of the location and stoichiometry than reagents that react with single lysine, or cysteine residues.

Where a T-Cell-MMP comprises a Fc polypeptide, the Fc polypeptide can comprise one or more covalently attached molecules of payload that are attached directly or indirectly through a linker. By way of example, where a T-Cell-MMP comprises a Fc polypeptide, the polypeptide chain comprising the Fc polypeptide can be of the formula (A)-(L)-(C), where (A) is the polypeptide chain comprising the Fc polypeptide; where (L), if present, is a linker; and where (C) is a payload. (L), if present, links (A) to (C). In some cases, the polypeptide chain comprising the Fc polypeptide can be coupled to more than one molecule of payload (e.g., 2, 3, 4, 5, or more than 5 payload molecules).

The payload may be selected from the group consisting of: therapeutic agents, drugs, diagnostic agents (e.g., labels), nucleotide or nucleoside analogs, nucleic acids or synthetic nucleic acids (e.g., antisense nucleic acids, small interfering RNA, double stranded (ds)DNA, single stranded (ss)DNA, ssRNA, dsRNA), toxins, liposomes (e.g., incorporating a therapeutic agent), nanoparticles (e.g., gold or other metal bearing nucleic acids or other molecules, lipids, particle bearing nucleic acids or other molecules), and combinations thereof.

The payload may be selected from, drugs (in active or prodrug form), therapeutic agents, antibiotics, antivirals (e.g., remdesivir), cell cycle synchronizing agents, ligands for cell surface receptor(s), immunomodulatory agents (e.g., immunosuppressants such as cyclosporine), pro-apoptotic agents, anti-angiogenic agents, cytokines, chemokines, growth factors, proteins or polypeptides, antibodies or antigen binding fragments thereof, enzymes, proenzymes, hormones and combinations thereof.

The payload may be an antiviral agent.

As discussed above, a polypeptide chain of a T-Cell-MMP or its epitope conjugate can comprise a payload including, but not limited to, therapeutic agent, linked (e.g., covalently attached) to the first or second polypeptide chain at chemical conjugation sites. The linkage between a payload and a first or second polypeptide chain of a T-Cell-MMP-epitope conjugate may be a direct or indirect linkage. Direct linkage can involve linkage directly to an amino acid side chain. Indirect linkage can be linkage via a linker. A payload (e.g., an antiviral agent) can be linked to a polypeptide chain (e.g., a Fc polypeptide) of a T-Cell-MMP-epitope conjugate via a thioether bond, an amide bond, a carbamate bond, a disulfide bond, or an ether bond.

The first and/or second polypeptide chains of a T-Cell-MMP can comprise one or more molecules of payload of photo detectable labels (e.g., dyes, fluorescent labels, phosphorescent labels, luminescent labels), contrast agents (e.g., iodine or barium containing materials), radiolabels, imaging agents, spin labels, Forster Resonance Energy Transfer (FRET)-type labels, paramagnetic labels/imaging agents (e.g., gadolinium containing magnetic resonance imaging labels), ultrasound labels and combinations thereof.

In some embodiments, the conjugate moiety comprises a label that is or includes a radioisotope. Examples of radioisotopes or other labels include, but are not limited to, ³H, ¹¹C, ¹⁴C, ¹⁵N, ³⁵S, ¹⁸F, ³²P, ³³P, ⁶⁴Cu, ⁶⁸Ga, ⁸⁹Zr, ⁹⁰Y, ⁹⁹Tc, ¹²³I, ¹²⁴I, ¹²⁵I, ¹³¹I, ¹¹¹In, ¹³¹In, ¹⁵³Sm, ¹⁸⁶Re, ¹⁸⁸Re, ²¹¹At, ²¹²Bi, and ¹⁵³Pb.

### II. Methods of Generating T-Cell-MMP Polypeptides

The present disclosure provides a method of obtaining T-Cell-MMPs (both unconjugated T-Cell-MMPs and/or T-Cell-MMP-epitope conjugates) including in dimer and other higher order aggregates, which may include one or more wt. MOD polypeptide sequences and/or one or more variant MOD polypeptide sequences that exhibit lower affinity for a Co-MOD compared to the affinity of the corresponding wt. MOD polypeptide sequence for the Co-MOD, the method comprising:
A) generating a T-Cell-MMP (or a higher order complex such as a dimer) by introducing into cells or cell-free systems one or more nucleic acids encoding an unconjugated T-Cell-MMP or each of the unconjugated T-Cell-MMPs that make up a heterodimer (e.g., a heterodimeric dimer of unconjugated T-Cell-MMPs);
wherein when the T-Cell-MMP comprises one or more nascent chemical conjugation sites, the nascent chemical conjugation site may be activated to produce an unconjugated T-Cell-MMP with chemical conjugation site (e.g., reacting sulfatase motifs with an FGE to convert a Cys residue to a fGly residue if the cells translating the T-Cell-MMP nucleic acids do not express a formylglycine generating enzyme).
   The above-mentioned method of generating T-Cell-MMPs may further comprise providing one or more nucleic acids encoding the unconjugated T-Cell-MMP, including those specifically described in the present disclosure, which may be present in a recombinant expression vector and/or operably linked to transcriptional control elements such as those functional in a eukaryotic cell. The method may be stopped at this point and the unconjugated T-Cell-MMP (e.g., unconjugated dimer T-Cell-MMP) that is unpurified (including cell lysates and unpurified media) may be obtained. Alternatively, the unconjugated T-Cell-MMP may be purified using, for example, one or more of salt precipitation (e.g., ammonium sulfate), affinity chromatography, and/or size exclusion chromatography, to produce crude (less than 60% by weight), initially refined (at least 60% by weight), partly refined (at least 80% by weight), substantially refined (at least 95% by weight), partially pure or partially purified (at least 98% by weight), substantially pure or substantially purified (at least 99% by weight), essentially pure or essentially purified (at least 99.5% by weight) or purified (at least 99.8%) or highly purified (at least 99.9% by weight) of the unconjugated T-Cell-MMP based on the total weight of protein present in the sample may be obtained by purification. Where a T-Cell-MMP-epitope conjugate is desired, the method may be continued by reacting anywhere from a crude preparation to a highly purified preparation of a T-Cell-MMP with an epitope presenting molecule as in step B:
B) providing an epitope (e.g., an epitope-presenting peptide) suitable for conjugation with the chemical conjugation site present in the unconjugated T-Cell-MMP of step A (e.g., a hydrazinyl or hydrazinyl indole modified peptide for reaction with a formyl glycine of a sulfatase motif or a maleimide containing peptide for reaction with a cysteine residue), and contacting the epitope with the T-Cell-MMP (e.g., under suitable reaction conditions) to produce a T-Cell-MMP-epitope conjugate.
   The choice of how purified the unconjugated material entered into the reaction needs to be depends on a number of factors including the conjugation reaction and conditions, the potential for side reactions, and the degree to which the final epitope conjugate will need to be purified.

The T-Cell-MMP-epitope conjugate (e.g., as a dimer or a higher order complex) may be purified by, for example, salt precipitation, size separation, and/or affinity chromatography, so that it is at least partly refined (at least 80% by weight of protein present in the sample), substantially refined (at least 95% by weight), partially pure or partially purified (at least 98% by weight), substantially pure or substantially purified (at least 99% by weight), essentially pure or essentially purified (at least 99.5% by weight), purified (at least 99.8%), or highly purified (at least 99.9% by weight) of the T-Cell-MMP-epitope conjugate based on the total weight of protein present in the sample.

Where it is desirable for a T-Cell-MMP or higher order complexes to contain a payload, the payload may be reacted with the unconjugated T-Cell-MMP or the T-Cell-MMP-epitope conjugate. The selectivity of the epitope and the payload for different conjugation sites may be controlled through the use of orthogonal chemistries and/or control of stoichiometry in the conjugation reactions. In embodiments, linkers (e.g., polypeptides or other bifunctional chemical linkers) may be used to attach the epitope and/or payloads to their conjugation sites. The payload may be, for example, an therapeutic agent such as an antiviral agent, or pro-drug form of a drug or therapeutic agent. The payload may be a retinoid. When possible, a single purification scheme that removes reagents and other materials present from the conjugation of the epitope and attachment of the payload is employed to minimize loss of the protein.

A variety of cells and cell-free systems may be used for the preparation of unconjugated T-Cell-MMPs. As discussed in the section titled “Genetically Modified Host cells,” the cells may be eukaryotic origin, and more specifically of mammalian, primate or even human origin.

The present disclosure provides a method of obtaining an unconjugated T-Cell-MMP or T-Cell-MMP-epitope conjugate (or their higher order complexes, such as dimeres) comprising one or more wt. MODs and/or variant MODs that exhibit reduced affinity for a Co-MOD compared to the affinity of the corresponding parental wt. MOD for the Co-MOD. Where a variant MOD having reduced affinity is desired, the method can comprise preparing a library of variant MOD polypeptides (e.g., that have at least one insertion, deletion or substitution) and selecting from the library of MOD polypeptides a plurality of members that exhibit reduced affinity for their Co-MOD (such as by BLI as described above). Once a variant MOD is selected a nucleic acid encoding the unconjugated T-Cell-MMP including the variant MOD is prepared and expressed. After the unconjugated T-Cell-MMP has been expressed it can be purified, and if desired conjugated to an epitope to produce the selected T-Cell-MMP-epitope conjugate. The process may be repeated to prepare a library of unconjugated T-Cell-MMPs or their epitope conjugates.

The present disclosure provides a method of obtaining a T-Cell-MMP-epitope conjugate or its higher order complexes, such as a diner) that exhibits selective binding to a T cell, the method comprising:
A) generating a library of T-Cell-MMP-epitope conjugates (or their higher order complexes) comprising a plurality of members, wherein each member comprises a different variant MOD on the T-Cell-MMP-epitope conjugate, wherein the variant MOD differs in amino acid sequence (e.g., by from 1 aa to 10 aas) from its parental wt. MOD, and wherein the T-Cell-MMP-epitope conjugate library members further comprise an epitope tag or a fluorescent label), and
B) contacting a T-Cell-MMP-epitope conjugate library member with a target T cell expressing on its surface: i) a Co-MOD that binds the parental wt. MOD; and ii) a TCR that binds to the epitope; and
C) selecting a T-Cell-MMP-epitope conjugate library member that selectively binds the target T cell relative to its binding under the same conditions to a control T cell that comprises: i) the Co-MOD that binds the parental wt. MOD; and ii) a TCR that binds to an epitope other than the epitope present in the T-Cell-MMP library member (e.g., choosing the T-Cell-MMP-epitope conjugate that has higher avidity or affinity for the target T cell than the control T cell such as by BLI as described above).
   A T-Cell-MMP-epitope conjugate library member that is identified as selectively binding to a target T cell may be isolated from the library.

When the T-Cell-MMP-epitope conjugate comprises an epitope tag or label, identifying a T-Cell-MMP-epitope conjugate selective for a target T cell may comprise detecting the epitope tag or label associated with target and control T cells by using, for example, flow cytometry. While labeled T-Cell-MMPs (e.g., fluorescently labeled) do not require modification to be detected, epitope tagged molecules may require contacting with an agent that renders the epitope tag visible (e.g., a fluorescent agent that binds the epitope tag). The affinity/avidity of the T-Cell-MMP-epitope conjugate can be determined by measuring the agent or label associated with target and control T cells (e.g., by measuring the mean fluorescence intensity using flow cytometry) over a range of concentrations. The T-Cell-MMP-epitope conjugate that binds with the highest affinity or avidity to the target T cell relative to the control T cell is understood to selectively bind to the target T cell.

MOD and Co-MOD pairs, including wt. and variant MOD and Co-MOD pairs, utilized in the methods of obtaining T-Cell-MMPs and methods of obtaining a T-Cell-MMP-epitope conjugate that exhibits selective binding to a T cell may be selected from: IL-2 and IL-2 receptor; 4-1BBL and 4-1BB; PD-L1 and PD-1; FasL and Fas; TGF-β and TGF-β receptor; CD80 and CD28; CD86 and CD28; OX40L and OX40; ICOS-L and ICOS; ICAM and LFA-1; JAG1 and Notch; JAG1 and CD46; CD70 and CD27; CD80 and CTLA4; and CD86 and CTLA4. Alternatively, they may be selected from IL-2 and IL-2 receptor; 4-1BBL and 4-1BB; PD-L1 and PD-1; FasL and Fas; CD80 and CD28; CD86 and CD28; CD80 and CTLA4; and CD86 and CTLA4. In some cases, the variant MODs present in a T-Cell-MMP, which are independently selected, comprise from 1 to 20 aa independently selected sequence variations (e.g., 1, 2, 3, 4, 5, 6, 7, 8, 9, 10, 11, 12, 13, 14, 15, 16, 17, 18, 19, or 20 aa substitutions, deletions, or insertions) compared to the corresponding parental wt. MOD.

A T-Cell-MMP (unconjugated T cell-MP or T-Cell-MMP-epitope conjugate) may comprise two or more wt. and/or variant MODs. The two or more MODs may comprise the same or different amino acid sequence. The two or more MODs may be on the same T-Cell-MMP (e.g., in tandem) of a T cell-MP-dimer. The first of two or more MODs may be on the first T-Cell-MMP of a T-Cell-MMP dimer and the second of two variant MODs may be on the second T-Cell-MMP of the dimer.

### III. Nucleic Acids

The present disclosure provides a nucleic acid comprising a nucleotide sequence encoding a T-Cell-MMP of the present disclosure. The present disclosure provides a nucleic acid comprising a nucleotide sequence encoding a T-Cell-MMP including chemical conjugation sites that are engineered into the polypeptides of the T-Cell-MMP.

The present disclosure provides nucleic acids comprising nucleotide sequences encoding the T-Cell-MMPs described herein. In some cases, the individual polypeptide chains of a T-Cell-MMP are encoded in separate nucleic acids. In some cases, all polypeptide chains of a T-Cell-MMP of the present disclosure are encoded in a single nucleic acid. In some cases, a first nucleic acid comprises a nucleotide sequence encoding a first polypeptide of a T-Cell-MMP of the present disclosure; and a second nucleic acid comprises a nucleotide sequence encoding a second polypeptide of a T-Cell-MMP of the present disclosure. In some cases, a single nucleic acid comprises a nucleotide sequence encoding a first polypeptide of a T-Cell-MMP and a second polypeptide of a T-Cell-MMP of the present disclosure.

A. Separate Nucleic Acids Encoding Individual Polypeptide Chains of a Multimeric Polypeptide

The present disclosure provides nucleic acids comprising nucleotide sequences encoding a T-Cell-MMP. As noted above, in some cases, the individual polypeptide chains of a T-Cell-MMP are encoded in separate nucleic acids. In some cases. nucleotide sequences encoding the separate polypeptide chains of a T-Cell-MMP are operably linked to transcriptional control elements, e.g., promoters, such as promoters that are functional in a eukaryotic cell, where the promoter can be a constitutive promoter or an inducible promoter.

The present disclosure provides a first nucleic acid and a second nucleic acid, where the first nucleic acid comprises a nucleotide sequence encoding a first polypeptide of a T-Cell-MMP of the present disclosure, where the first polypeptide comprises, in order from N-terminus to C-terminus: a) a first MHC polypeptide; and b) a MOD (e.g., a reduced-affinity variant MOD polypeptide as described above); and where the second nucleic acid comprises a nucleotide sequence encoding a second polypeptide of a T-Cell-MMP, where the second polypeptide comprises, in order from N-terminus to C-terminus: a) a second MHC polypeptide; and b) an Ig Fc polypeptide. Suitable epitopes, MHC polypeptides, MODs, and Ig Fc polypeptides are described above. At least one of the first and second polypeptides comprises a chemical conjugation site (or a nascent site that can be converted to a chemical conjugation site). In some cases, the nucleotide sequences encoding the first and second polypeptides are operably linked to transcriptional control elements. In some cases, the transcriptional control element is a promoter that is functional in a eukaryotic cell. In some cases, the nucleic acids are present in separate expression vectors.

The present disclosure provides a first nucleic acid and a second nucleic acid, where the first nucleic acid comprises a nucleotide sequence encoding a first polypeptide of a T-Cell-MMP, where the first polypeptide comprises a first MHC polypeptide; and where the second nucleic acid comprises a nucleotide sequence encoding a second polypeptide of a T-Cell-MMP, where the second polypeptide comprises, in order from N-terminus to C-terminus: a) a MOD (e.g., a reduced-affinity variant MOD polypeptide as described above); b) a second MHC polypeptide; and c) an Ig Fc polypeptide. Suitable MHC polypeptides, MODs, and Ig Fc polypeptides are described above. At least one of the first and second polypeptides comprises a chemical conjugation site. In some cases, the nucleotide sequences encoding the first and second polypeptides are operably linked to transcriptional control elements. In some cases, the transcriptional control element is a promoter that is functional in a eukaryotic cell. In some cases, the nucleic acids are present in separate expression vectors.

B. Nucleic Acid Encoding Two or More Polypeptides Present in a T-Cell-MMP

The present disclosure provides a nucleic acid comprising nucleotide sequences encoding at least the first polypeptide and the second polypeptide of a T-Cell-MMP. In some cases, where a T-Cell-MMP includes a first, second, and third polypeptide, the nucleic acid includes a nucleotide sequence encoding the first, second, and third polypeptides. In some cases, the nucleotide sequences encoding the first polypeptide and the second polypeptide of a T-Cell-MMP include a proteolytically cleavable linker interposed between the nucleotide sequence encoding the first polypeptide and the nucleotide sequence encoding the second polypeptide. In some cases, the nucleotide sequences encoding the first polypeptide and the second polypeptide of a T-Cell-MMP include an internal ribosome entry site (IRES) interposed between the nucleotide sequence encoding the first polypeptide and the nucleotide sequence encoding the second polypeptide. In some cases, the nucleotide sequences encoding the first polypeptide and the second polypeptide of a T-Cell-MMP include a ribosome skipping signal (or cis-acting hydrolase element, CHYSEL) interposed between the nucleotide sequence encoding the first polypeptide and the nucleotide sequence encoding the second polypeptide. Examples of nucleic acids are described below, where a proteolytically cleavable linker is provided between nucleotide sequences encoding the first polypeptide and the second polypeptide of a T-Cell-MMP; in any of these embodiments, an IRES or a ribosome skipping signal can be used in place of the nucleotide sequence encoding the proteolytically cleavable linker.

In some cases provided for herein, a first nucleic acid (e.g., a recombinant expression vector, an mRNA, a viral RNA, etc.) comprises a nucleotide sequence encoding a first polypeptide chain of a T-Cell-MMP; and a second nucleic acid (e.g., a recombinant expression vector, an mRNA, a viral RNA, etc.) comprises a nucleotide sequence encoding a second polypeptide chain of the T-Cell-MMP. In some cases, the nucleotide sequence encoding the first polypeptide and the nucleotide sequence encoding the second polypeptide are each operably linked to transcriptional control elements, e.g., promoters, such as promoters that are functional in a eukaryotic cell, where the promoter can be a constitutive promoter or an inducible promoter.

The present disclosure provides a nucleic acid comprising a nucleotide sequence encoding a recombinant polypeptide, where the recombinant polypeptide comprises, in order from N-terminus to C-terminus the elements: a) a first MHC polypeptide; b) a MOD (e.g., a reduced-affinity variant as described above); c) a proteolytically cleavable linker; d) a second MHC polypeptide; and e) an immunoglobulin (Ig) Fc polypeptide; wherein at least one of the elements comprises a chemical conjugation site that is not removed during cellular processing. The present disclosure provides a nucleic acid comprising a nucleotide sequence encoding a recombinant polypeptide, where the recombinant polypeptide comprises, in order from N-terminus to C-terminus the elements: a) a first leader peptide; b) a first MHC polypeptide; c) a MOD (e.g., a reduced-affinity variant as described above); d) a proteolytically cleavable linker; e) a second leader peptide; f) a second MHC polypeptide; and g) an Ig Fc polypeptide; wherein at least one of the elements comprises a chemical conjugation site that is not removed during cellular processing. The present disclosure provides a nucleic acid comprising a nucleotide sequence encoding a recombinant polypeptide, where the recombinant polypeptide comprises, in order from N-terminus to C-terminus, the elements: a) a first MHC polypeptide; b) a proteolytically cleavable linker; c) a MOD (e.g., a reduced-affinity variant as described above); d) a second MHC polypeptide; and e) an Ig Fc polypeptide; wherein at least one of the elements comprises a chemical conjugation site that is not removed during cellular processing. In some cases, the first leader peptide and the second leader peptide are β2M leader peptides. In some cases, the nucleotide sequence is operably linked to a transcriptional control element. In some cases, the transcriptional control element is a promoter that is functional in a eukaryotic cell.

Suitable MHC polypeptides are described above. In some cases, the first MHC polypeptide comprises a β2-microglobulin (β2M) polypeptide; and the second MHC polypeptide comprises an MHC Class I heavy chain polypeptide. In some cases, the β2M polypeptide comprises an amino acid sequence having at least about 85% (e.g., at lease about 90%, 95%, 98%, 99%, or even 100%) as sequence identity to a β2M amino acid sequence depicted in FIG. 4 (e.g., of the mature sequence lacking the signal peptide). In some cases, the MHC Class I heavy chain polypeptide is an HLA-A, HLA-B, HLA-C, HLA-E, HLA-F, or HLA-G heavy chain. In some cases, the MHC Class I heavy chain polypeptide comprises an amino acid sequence having at least 85% as sequence identity to the amino acid sequence depicted in any one of FIGS. 3A-3H. In such an embodiment the MHC Class I heavy chain polypeptide sequence may be selected such that it does not comprise a transmembrane anchoring domain or signal peptide (e.g., the heavy chain polypeptide comprises a sequence such as those in FIG. 3D lacking the transmembrane domain and signal sequence).

Suitable Fc polypeptides are described above. In some cases, the Ig Fc polypeptide is an IgG1 Fc polypeptide, an IgG2 Fc polypeptide, an IgG3 Fc polypeptide, an IgG4 Fc polypeptide, an IgA Fc polypeptide, or an IgM Fc polypeptide. In some cases, the Ig Fc polypeptide comprises an amino acid sequence having at least 85% as sequence identity to an amino acid sequence depicted in FIGS. 2A-2G.

Suitable immunomodulatory polypeptides (MODs) are described above.

In addition to any other proteolytically cleavable linkers, in some cases, the proteolytically cleavable linker comprises an amino acid sequence selected from the group consisting of: a) LEVLFQGP (SEQ ID NO:132); b) ENLYTQS (SEQ ID NO:133); c) DDDDK (SEQ ID NO:134); d) LVPR (SEQ ID NO:135); and e) GSGATNFSLLKQAGDVEENPGP (SEQ ID NO:136).

In some cases, a linker comprising a first Cys residue attached to the first MHC polypeptide is provided, and the second MHC polypeptide comprises an aa substitution to provide a second (engineered) Cys residue, such that the first and second Cys residues provide for a disulfide linkage between the linker and the second MHC polypeptide. In some cases, the first MHC polypeptide comprises an aa substitution to provide a first engineered Cys residue, and the second MHC polypeptide comprises an aa substitution to provide a second engineered Cys residue, such that the first Cys residue and the second Cys residue provide for a disulfide linkage between the first MHC polypeptide and the second MHC polypeptide. As discussed above, where disulfide bridges are provided, it is possible to use either thiol reactive agents or bis-thiol linkers to incorporate payloads or epitopes.

C. Recombinant Expression Vectors

The present disclosure provides recombinant expression vectors comprising nucleic acids of the present disclosure. In some cases, the recombinant expression vector is a non-viral vector. In some embodiments, the recombinant expression vector is a viral construct, e.g., a recombinant adeno-associated virus construct (see, e.g., U.S. Pat. No. 7,078,387), a recombinant adenoviral construct, a recombinant lentiviral construct, a recombinant retroviral construct, a non-integrating viral vector, etc.

Suitable expression vectors include, but are not limited to, viral vectors (e.g., viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, lentivirus, human immunodeficiency virus, myeloproliferative sarcoma virus, and mammary tumor virus); and the like.

Numerous suitable expression vectors are known to those of skill in the art, and many are commercially available. The following vectors are provided by way of example for eukaryotic host cells: pXT1, pSG5 (Stratagene®), pSVK3, pBPV, pMSG, and pSVLSV40 (Pharmacia). However, any other vector may be used so long as it is compatible with the host cell.

Depending on the host/vector system utilized, any of a number of suitable transcription and translation control elements, including constitutive and inducible promoters, transcription enhancer elements, transcription terminators, etc., may be used in the expression vector (see, e.g., Bitter et al. (1987), Methods in Enzymology, 153:516-544).

In some embodiments, a nucleotide sequence encoding a DNA-targeting RNA and/or a site-directed modifying polypeptide is operably linked to a control element, e.g., a transcriptional control element, such as a promoter. The transcriptional control element may be functional in either a eukaryotic cell, e.g., a mammalian cell; or a prokaryotic cell (e.g., bacterial or archaeal cell). In some embodiments, a nucleotide sequence encoding a DNA-targeting RNA and/or a site-directed modifying polypeptide is operably linked to multiple control elements that allow expression of the nucleotide sequence encoding the DNA-targeting RNA and/or site-directed modifying polypeptide in both prokaryotic and eukaryotic cells.

Non-limiting examples of suitable eukaryotic promoters (promoters functional in a eukaryotic cell) include those from cytomegalovirus (CMV) immediate early, herpes simplex virus (HSV) thymidine kinase, early and late SV40, long terminal repeats (LTRs) from retrovirus, and mouse metallothionein-I. Selection of the appropriate vector and promoter is well within the level of ordinary skill in the art. The expression vector may also contain a ribosome binding site for translation initiation and a transcription terminator. The expression vector may also include appropriate sequences for amplifying expression.

### IV. Genetically Modified Host Cells

The present disclosure provides a genetically modified host cell, where the host cell is genetically modified with a nucleic acid of the present disclosure.

Suitable host cells include eukaryotic cells, such as yeast cells, insect cells, and mammalian cells. In some cases, the host cell is a cell of a mammalian cell line. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2™), CHO cells (e.g., ATCC Nos. CRL-9618™, CCL-61™, CRL9096), 293 cells (e.g., ATCC No. CRL-1573™), Vero cells, NIH 3T3 cells (e.g., ATCC No. CRL-1658), Huh-7 cells, BHK cells (e.g., ATCC No. CCL-10™), PC12 cells (ATCC No. CRL-1721™), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, and the like.

In some cases, the host cell is a mammalian cell that has been genetically modified such that it does not synthesize endogenous MHC β2M and/or such that it does not synthesize endogenous MHC Class I heavy chains (MHC-H). In addition to the foregoing, host cells expressing formylglycine generating enzyme (FGE) activity are discussed above for use with T-Cell-MMPs comprising a sulfatase motif, and such cells may advantageously be modified such that they do not express at least one, if not both, of the endogenous MHC β2M and MHC-H proteins.

### V. Compositions and Formulations

The present disclosure provides compositions, including pharmaceutical compositions, comprising one or more T-Cell-MMP-epitope conjugates, wherein the pharmaceutical compositions may comprise one or more pharmaceutically acceptable excipients as provided below. The present disclosure also provides compositions, including pharmaceutical compositions, comprising a nucleic acid or a recombinant expression vector of the present disclosure.

A. Compositions Comprising T-Cell-MMP-Epitope Conjugates

A composition of the present disclosure can comprise, in addition to a TMP of the present disclosure, one or more additional components that provide desirable properties such as stability, solubility, etc., e.g., salts, solubilizing agents; surfactants, protease inhibitors, etc., a variety of which are known in the art and need not be discussed in detail herein. Pharmaceutically acceptable excipients for biologics have been amply described in a variety of patents and other publications, including, for example, “Remington: The Science and Practice of Pharmacy”, 19th Ed. (1995), or latest edition, Mack Publishing Co; A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds 7th ed., Lippincott, Williams, & Wilkins; and Handbook of Pharmaceutical Excipients (2000) A. H. Kibbe et al., eds., 3rd ed. Amer. Pharmaceutical Assoc.

A pharmaceutical composition can comprise a T-Cell-MMP epitope conjugate of the present disclosure, and a pharmaceutically acceptable excipient. In some cases, a subject pharmaceutical composition will be suitable for administration to a subject, e.g., will be sterile. For example, in some embodiments, a subject pharmaceutical composition will be suitable for administration to a human subject, e.g., where the composition is sterile and is free of detectable pyrogens and/or other toxins and/or such detectable pyrogens and/or other toxins are below permissible limits.

Where a T-Cell-MMP epitope conjugate is administered as an injectable (e.g., subcutaneously, intraperitoneally, intramuscularly, and/or intravenously) directly into a tissue, a formulation can be provided as a ready-to-use dosage form, a non-aqueous form (e.g., a reconstitutable storage-stable powder) or an aqueous form, such as liquid composed of pharmaceutically acceptable carriers and excipients. The protein-containing formulations may also be provided so as to enhance serum half-life of the subject protein following administration. For example, the protein may be provided in a liposome formulation, prepared as a colloid, or other conventional techniques for extending serum half-life. A variety of methods are available for preparing liposomes, as described in, e.g., Szoka et al. 1980 Ann. Rev. Biophys. Bioeng. 9:467, U.S. Pat. Nos. 4,235,871, 4,501,728 and 4,837,028. The preparations may also be provided in controlled release or slow-release forms.

Other examples of formulations suitable for parenteral administration include isotonic sterile injection solutions, anti-oxidants, bacteriostats, and solutes that render the formulation isotonic with the blood of the intended recipient, suspending agents, solubilizers, thickening agents, stabilizers, and preservatives. For example, a subject pharmaceutical composition can be present in a container, e.g., a sterile container, such as a syringe. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets.

The concentration of a T-Cell-MMP-epitope conjugate in a formulation can vary widely (e.g., from less than about 0.1%, usually at or at least about 2% to as much as 20% to 50% or more by weight) and will usually be selected primarily based on fluid volumes, viscosities, and patient-based factors in accordance with the particular mode of administration selected and the patient's needs.

The present disclosure provides a container comprising a composition of the present disclosure, e.g., a liquid composition. The container can be, e.g., a syringe, an ampoule, and the like. In some cases, the container is sterile. In some cases, both the container and the composition are sterile.

The present disclosure provides compositions, including pharmaceutical compositions, comprising a T-Cell-MMP epitope conjugate. A composition can comprise: a) a T-Cell-MMP-epitope conjugate; and b) an excipient, as described above for the T-Cell-MMP epitope conjugates. In some cases, the excipient is a pharmaceutically acceptable excipient.

In some cases, a T-Cell-MMP-epitope conjugate is present in a liquid composition. Thus, the present disclosure provides compositions (e.g., liquid compositions, including pharmaceutical compositions) comprising a T-Cell-MMP-epitope conjugate of the present disclosure. In some cases, a composition comprises: a) a T-Cell-MMP-epitope conjugate of the present disclosure; and b) saline (e.g., 0.9% or about 0.9% NaCl). In some cases, the composition is sterile. In some cases, the composition is suitable for administration to a human subject, e.g., where the composition is sterile and is free of detectable pyrogens and/or other toxins and/or such detectable pyrogens and/or other toxins are below permissible limits. Thus, the present disclosure provides a composition comprising: a) a T-Cell-MMP-epitope conjugate; and b) saline (e.g., 0.9% or about 0.9% NaCl), where the composition is sterile and is free of detectable pyrogens and/or other toxins and/or such detectable pyrogens and/or other toxins are below permissible limits.

Suitable formulations (e.g., pharmaceutical formulations) include the above-described compositions, where the compositions include a pharmaceutically acceptable excipient. In some cases, a suitable formulation comprises: a) T-Cell-MMP coronavirus epitope conjugate; and b) a pharmaceutically acceptable excipient. Some suitable pharmaceutically acceptable excipients are described above.

### VI. Methods of Modulating T-Cell Activity

The present disclosure provides a method of selectively modulating the activity of a T cell, the method comprising contacting the T cell with a MODs on a T-Cell-MMP-epitope conjugate, and in some instances the payload the T-Cell-MMP-epitope conjugate may be carrying. Where the T-Cell-MMP has been conjugated to an epitope (i.e. it is a T-Cell-MMP-epitope conjugate), contacting the conjugate to a T-cell can result in epitope-specific T-cell modulation. In some cases, the contacting occurs in vivo (e.g., in a mammal such as a human, dog, cat, pig, horse, or primate). In some cases, the contacting occurs in vitro. In some cases, the contacting occurs ex vivo. In some cases, the T-cell is a CD8+ T-cell, CD4+ T-cell, a NK-T-cell, or a Treg cell as described below under Treatment Methods. In some cases, the T-cell is a CD8+ T-cell as described below under Treatment Methods.

The present disclosure provides a method of selectively modulating the activity of an epitope-specific T-cell, the method comprising contacting the T-Cell with a T-Cell-MMP-epitope conjugate bearing the epitope recognized by the epitope-specific T-Cell, where contacting the T-Cell with a T-Cell-MMP-epitope conjugate selectively modulates the activity of the epitope-specific T-Cell. In some cases, the contacting occurs in vitro. In some cases, the contacting occurs in vivo. In some cases, the contacting occurs ex vivo.

In some cases, e.g., where the target T-cell is a CD8⁺ T-cell, the T-Cell-MMP-epitope conjugate comprises Class I MHC polypeptides (e.g., β2-microglobulin and Class I MHC heavy chain).

Where a T-Cell-MMP-epitope conjugate of the present disclosure includes a MOD that is an activating polypeptide, contacting the T-cell with the T-Cell-MMP-epitope conjugate activates the epitope-specific T-cell. In some instances, the epitope-specific T-cell is a T-cell that is specific for a coronavirus epitope (e.g., peptide, phosphopeptide, or glycopeptide epitopes such as those from a spike glycoprotein, nucleoprotein, membrane protein, replicase protein, non-structural protein (nsp) and the like), and contacting the epitope-specific T-cell with the T-Cell-MMP-epitope conjugate increases cytotoxic activity of the T-cell toward a coronavirus infected cell. In some instances, the epitope-specific T-cell is a T-cell that is specific for a coronavirus epitope, and contacting the epitope-specific T-cell with the T-Cell-MMP-epitope conjugate increases the number of the epitope-specific T-cells.

In some instances, the epitope-specific T-cell is a T-cell that is specific for an epitope present on a coronavirus-infected cell, and contacting the epitope-specific T-cell with the T-Cell-MMP-epitope conjugate increases cytotoxic activity of the T-cell toward the coronavirus-infected cell. In some instances, the epitope-specific T-cell is a T-cell that is specific for an epitope present on a coronavirus-infected cell, and contacting the epitope-specific T-cell with the T-Cell-MMP-epitope conjugate increases the number of the epitope-specific T-cells.

Where a T-Cell-MMP-epitope conjugate includes a MOD that is an inhibiting polypeptide, contacting the T-cell with the multimer inhibits the epitope-specific T-cell.

The present disclosure provides a method of modulating an immune response in an individual, the method comprising administering to the individual an effective amount of a T-Cell-MMP-epitope conjugate of the present disclosure. As noted above, administering the T-Cell-MMP-epitope conjugate induces an epitope-specific T cell response (e.g., a coronavirus epitope-specific T-cell response) and an epitope-non-specific T cell response, where the ratio of the epitope-specific T cell response to the epitope-non-specific T cell response is at least 2:1, as measured by the ratio of the increase of the number of epitope-specific T cells to the increase in the number of epitope non-specific T cells, which can be readily determined by known methods. In some cases, the ratio of the epitope-specific T cell response to the epitope-non-specific T cell response is at least 5:1. In some cases, the ratio of the epitope-specific T cell response to the epitope-non-specific T cell response is at least 10:1. In some cases, the ratio of the epitope-specific T cell response to the epitope-non-specific T cell response is at least 25:1. In some cases, the ratio of the epitope-specific T cell response to the epitope-non-specific T cell response is at least 50:1. In some cases, the ratio of the epitope-specific T cell response to the epitope-non-specific T cell response is at least 100:1.

In some cases, the individual is a human. In some cases, the modulating increases a cytotoxic T-cell response to a coronavirus-infected cell, e.g., a cell expressing a coronavirus antigen that displays the same epitope displayed by the peptide epitope present in the T-Cell-MMP-epitope conjugate. In some cases, the administering is intravenous, subcutaneous or intramuscular.

The present disclosure also provides a method of detecting, in a mixed population of cells (e.g., a mixed population of T cells) obtained from an individual, the presence of a target T cell that binds a coronavirus epitope of interest, the method comprising: a) contacting in vitro the mixed population of cells (e.g., mixed population of T cells) with a T-Cell-MMP-coronavirus epitope conjugate of the present disclosure; and b) detecting activation and/or proliferation of T cells in response to said contacting, wherein activated and/or proliferated T cells indicates the presence of the target T cell.

### VI. Methods of Selectively Delivering a Costimulatory Polypeptide (MOD)

The present disclosure provides a method of delivering one or more independently selected MODs and/or a reduced-affinity variant of naturally occurring MODs (such as a variant disclosed herein) to a selected T-cell or a selected T-cell population, e.g., in a manner such that a TCR specific for a given coronavirus epitope is targeted. The present disclosure provides a method of delivering a MOD, or a variant (e.g., a reduced-affinity variant) of a naturally occurring MOD disclosed herein, selectively to a target T-cell bearing a TCR specific for the coronavirus epitope present in a T-Cell-MMP-epitope conjugate of the present disclosure. The method comprises contacting a population of T-cells with a T-Cell-MMP-epitope conjugate of the present disclosure. The population of T-cells can be a mixed population that comprises: i) the target T-cell; and ii) non-target T-cells that are not specific for the epitope (e.g., T-cells that are specific for an epitope(s) other than the epitope to which the epitope-specific T-cell binds). The epitope-specific T-cell is specific for the coronavirus epitope-presenting peptide present in the T-Cell-MMP-epitope conjugate and binds to the peptide HLA complex or peptide MHC complex provided by the T-Cell-MMP-epitope conjugate. Accordingly, contacting the population of T-cells with the T-Cell-MMP-epitope conjugate delivers the costimulatory polypeptide (e.g., a wild-type MOD or a reduced-affinity variant of the wild-type MOD, as described herein) selectively to the T-cell(s) that are specific for the epitope present in the T-Cell-MMP-epitope conjugate. In some cases, the population of T cells is in vitro. In some cases, the population of T cells is in vivo in an individual. In some cases, the method comprises administering the T-Cell-MMP-epitope conjugate to the individual. In some case, the T cell is a cytotoxic T cell. In some cases, the mixed population of T cells is an in vitro population of mixed T cells obtained from an individual, and the contacting step results in activation and/or proliferation of the target T cell(s), generating a population of activated and/or proliferated target T cells; in some of these instances, the method further comprises administering the population of activated and/or proliferated target T cells to the individual.

Thus, the present disclosure provides a method of delivering a MOD (such as IL-2), or a variant such as a reduced-affinity variant of a naturally occurring MOD (such as an IL-2 variant) disclosed herein, or a combination of both, selectively to a target T-cell, the method comprising contacting a mixed population of T-cells with a T-Cell-MMP-epitope conjugate of the present disclosure. The mixed population of T-cells comprises the target T-cell and non-target T-cells. The target T-cell is specific for the epitope present within the T-Cell-MMP-epitope conjugate. Contacting the mixed population of T-cells with a T-Cell-MMP-epitope conjugate delivers the MOD(s) present within the T-Cell-MMP-epitope conjugate to the target T-cell.

### VIII. Treatment Methods

The present disclosure provides a method of selectively modulating the activity of an epitope-specific T-cell in an individual (e.g., treat an individual), the method comprising administering to the individual an amount of a T-Cell-MMP conjugated to a coronavirus epitope (a T-Cell-MMP-coronavirus epitope conjugate) of the present disclosure. Also provided is a T-Cell-MMP-coronavirus epitope conjugate for use in a method of treatment of a human or animal body. In some cases, a treatment method of the present disclosure comprises administering to an individual in need thereof a T-Cell-MMP-coronavirus epitope conjugate of the present disclosure. Conditions that can be treated include alpha-, beta-, gamma-, and delta-coronavirus infections. In an embodiment, the condition that can be treated is a Bat-SL-CoV, SARS-CoV, SARS-CoV-2 (Covid-19), or MERS-CoV infection. In an embodiment, the condition that can be treated is a SARS-CoV, SARS-CoV-2, or MERS-CoV infection. In an embodiment, the condition that can be treated is a SARS-CoV or SARS-CoV-2 infection. In an embodiment, the condition that can be treated is a SARS-CoV infection. In an embodiment, the condition that can be treated is a SARS-CoV-2 (Covid-19) infection.

In some cases, a T-cell-MMP-epitope conjugate of the present disclosure, when administered to an individual in need thereof, induces both an epitope-specific T-cell response and an epitope non-specific T-cell response. In other words, in some cases, a T-cell-MMP-epitope conjugate of the present disclosure, when administered to an individual in need thereof, induces an epitope-specific T-cell response by modulating the activity of a first T-cell that displays both: i) a TCR specific for the epitope present in the T-Cell-MMP; and ii) a Co-MOD that binds to the MOD present in the T-Cell-MMP-epitope conjugate; and induces an epitope non-specific T-cell response by modulating the activity of a second T-cell that displays: i) a TCR specific for an epitope other than the epitope present in the T-Cell-MMP; and ii) a Co-MOD that binds to the MOD present in the T-Cell-MMP. The ratio of the epitope-specific T-cell response to the epitope-non-specific T-cell response, when measured as the ratio of the increase of the number of epitope-specific T cells to the increase in the number of epitope non-specific T cells (as discussed above), is at least 2:1, at least 5:1, at least 10:1, at least 15:1, at least 20:1, at least 25:1, at least 50:1, or at least 100:1. The ratio of the epitope-specific T-cell response to the epitope-non-specific T-cell response is from about 2:1 to about 5:1, from about 5:1 to about 10:1, from about 10:1 to about 15:1, from about 15:1 to about 20:1, from about 20:1 to about 25:1, from about 25:1 to about 50:1, from about 50:1 to about 100:1, or more than 100:1. “Modulating the activity” of a T-cell can include one or more of: i) activating a cytotoxic (e.g., CD8⁺) T-cell (e.g., to proliferate); ii) inducing cytotoxic activity of a cytotoxic (e.g., CD8⁺) T-cell; iii) inducing production and release of a cytotoxin (e.g., a perforin; a granzyme; a granulysin) by a cytotoxic (e.g., CD8⁺) T-cell; and iv) increasing the number of epitope-specific T cells.

Combining a MOD with reduced affinity for its Co-MOD permits the affinity of the MHC bound coronavirus epitope for a TCR to dominate the binding interactions and provide for enhanced selectivity (for T cells specific to the coronavirus epitope) of a T-Cell-MMP-coronavirus epitope conjugate of the present disclosure. Thus, for example, a T-Cell-MMP-coronavirus epitope conjugate binds with higher avidity to a first T-cell that displays both: i) a TCR specific for the epitope present in the T-Cell-MMP-epitope conjugate; and ii) a Co-MOD that binds to the MOD present in the T-Cell-MMP-epitope conjugate, compared to the avidity to which it binds to a second T-cell that displays: i) a TCR specific for an epitope other than the epitope present in the T-Cell-MMP-epitope conjugate; and ii) a Co-MOD that binds to the MOD present in the T-Cell-MMP-epitope conjugate.

The present disclosure provides a method of selectively modulating the activity of a coronavirus epitope-specific T-cell in an individual, the method comprising administering to the individual an effective amount of a T-Cell-MMP-epitope conjugate of the present disclosure, where the T-Cell-MMP-epitope conjugate selectively modulates the activity of the epitope-specific T-cell in the individual. Selectively modulating the activity of an epitope-specific T-cell can treat a disease or disorder in the individual. Thus, the present disclosure provides a treatment method comprising administering to an individual in need thereof an effective amount of a T-Cell-MMP-epitope conjugate.

In some cases, the MOD is an activating polypeptide, and the T-Cell-MMP-epitope conjugate activates an epitope-specific T-cell that recognizes a coronavirus epitope. In some cases, the T cells are cytotoxic T-cells (CD8⁺ cells) and the T-Cell-MMP-coronavirus epitope conjugate increases the activity of the T-cell specific for a coronavirus infected cell. Activation of CD8+ cells can include increasing: proliferation of the cells; the production of cytokines and/or cytotoxic materials (e.g., granulysin), the release of cytokines such as interferon 7; and/or the release of cytotoxic materials (e.g., a perforin; a granzyme; a granulysin).

In some cases, a T-Cell-MMP-coronavirus epitope conjugate reduces proliferation and/or activity of an epitope restricted regulatory T cell or Treg (e.g. CD8+ Tregs which are FoxP3⁺, CD8⁺ T cells). In some cases, e.g., where a T-Cell-MMP-epitope conjugate comprises an inhibitory MOD (e.g., PD-L1, FasL, and the like), the T-Cell-MMP-epitope conjugate reduces the proliferation and/or activity of a Treg and may result in apoptosis of the T reg.

T-Cell-MMP-coronavirus epitope conjugates may comprise a viral lipopeptide, glycopeptide or phosphopeptide epitope, and may be administered to an individual in need thereof to treat a coronavirus infection in the individual, where the infectious agent expresses the epitope present in the T-Cell-MMP-epitope conjugate. Accordingly, the present disclosure provides a method of treating an infection in an individual, the method comprising administering to the individual an effective amount of a viral lipopeptide, glycopeptide or phosphopeptide epitope containing T-Cell-MMP-epitope conjugate of the present disclosure.

In some instances, the epitope-specific T-cell is a T-cell that is specific for an epitope present on a coronavirus-infected cell, and contacting the epitope-specific T-cell with the T-Cell-MMP-epitope conjugate increases cytotoxic activity of the T-cell toward the coronavirus-infected cell. In some instances, the epitope-specific T-cell is a T-cell that is specific for an epitope present on a coronavirus-infected cell, and contacting the epitope-specific T-cell with the T-Cell-MMP-epitope conjugate increases the number of epitope-specific T-cells. Accordingly, the present disclosure provides a method of treating a coronavirus infection in an individual, the method comprising administering to the individual an effective amount of a T-Cell-MMP-coronavirus epitope conjugate of the present disclosure, where the T-Cell-MMP-epitope conjugate comprises a T-cell epitope that is a viral epitope, and where the T-Cell-MMP-epitope conjugate comprises a stimulatory MOD. In some cases, an effective amount of a T-Cell-MMP-epitope conjugate is an amount that, when administered in one or more doses to an individual in need thereof, reduces the number of coronavirus-infected cells in the individual. For example, in some cases, an “effective amount” of a T-Cell-MMP-epitope conjugate is an amount that, when administered in one or more doses to an individual in need thereof, reduces the number of coronavirus-infected cells in the individual by at least 10%, at least 15%, at least 20%, at least 25%, at least 30%, at least 40%, at least 50%, at least 60%, at least 70%, at least 80%, at least 90%, or at least 95%, compared to the number of coronavirus-infected cells in the individual before administration of the T-Cell-MMP-epitope conjugate, or in the absence of administration with the T-Cell-MMP-epitope conjugate. In some cases, an effective amount of a T-Cell-MMP-epitope conjugate is an amount that, when administered in one or more doses to an individual in need thereof, reduces the number of coronavirus-infected cells in the individual to undetectable levels.

In some cases, an “effective amount” of a T-Cell-MMP-epitope conjugate is an amount that, when administered in one or more doses to an individual in need thereof, increases survival time of the individual. For example, in some cases, an effective amount of a T-Cell-MMP-epitope conjugate is an amount that, when administered in one or more doses to an individual in need thereof, increases survival time of the individual by at least 1 month, at least 2 months, at least 3 months, from 3 months to 6 months, from 6 months to 1 year, from 1 year to 2 years, from 2 years to 5 years, from 5 years to 10 years, or more than 10 years, compared to the expected survival time of the individual in the absence of administration with the T-Cell-MMP-epitope conjugate.

In some cases, an effective amount of a T-Cell-MMP-epitope conjugate is an amount that, when administered in one or more doses to individuals in a population of individuals in need thereof, increases average survival time of the population. For example, in some cases, an effective amount of a T-Cell-MMP-epitope conjugate of the present disclosure is an amount that, when administered in one or more doses to individuals in a population of individuals suffering from a coronavirus infection, increases the average survival time of the population of individuals receiving the T-Cell-MMP-epitope conjugate by at least 1 month, at least 2 months, at least 3 months, from 3 months to 6 months, from 6 months to 1 year, from 1 year to 2 years, from 2 years to 5 years, from 5 years to 10 years, or more than 10 years, compared to the average survival time of the individuals not receiving the T-Cell-MMP-epitope conjugate; wherein the population is an age, gender, weight, smoking status, and/or disease state (disease such as COPD, cystic fibrosis, asthma etc. and degree of progression) matched population.

In some cases, because T-Cell-MMP-epitope conjugates also are able to prime naïve T cells, the pharmaceutical compositions of this disclosure also may be used to treat persons who are not infected but at risk of infection, i.e., the pharmaceutical compositions can be injected to cause a human or non-human to prime and activate epitope specific T cells and/or develop memory T cells that will be therapeutically useful in the event of a SARS-CoV-2 infection. Accordingly, in some cases, an effective amount of a pharmaceutical composition comprising a T-Cell-MMP-epitope conjugate is an amount that, when administered in one or more doses to individuals in a population who do not have an infection and are at risk of infection, and/or to individuals who are at greater risk of severe illness from infection than the general population, causes a human or non-human to prime and activate epitope specific T cells and/or develop memory T cells that will be therapeutically useful in the event of a SARS-CoV-2 infection. See e.g., CDC guidelines at: www.cdc.gov/coronavirus/2019-ncov/need-extra-precautions/people-with-medical-conditions.html.

As noted above, in some cases, in carrying out a subject treatment method, a T-Cell-MMP-coronavirus epitope conjugate is administered to an individual in need thereof unformulated or formulated as a pharmaceutical composition.

### IX. Dosages and Routes of Administration

A. Dosages

A suitable dosage of a T-Cell-MMP-epitope conjugate can be determined by an attending physician, or other qualified medical personnel, based on various clinical factors. As is well known in the medical arts, dosages for any one patient depend upon many factors, including the patient's size, body surface area, age, the particular T-Cell-MMP-epitope conjugate to be administered, sex of the patient, time, route of administration, general health, and other drugs being administered concurrently. A T-Cell-MMP-epitope conjugate may be administered in amounts between 1 ng/kg body weight and 20 mg/kg body weight per dose, e.g., between 0.1 mg/kg body weight to 10 mg/kg body weight, e.g., between 0.5 mg/kg body weight to 5 mg/kg body weight; however, doses below or above this exemplary range are envisioned, especially considering the aforementioned factors. If the regimen is a continuous infusion, it can also be in the range of 1 μg to 10 mg per kilogram of body weight per minute. A T-Cell-MMP-epitope conjugate can be administered in an amount of from about 1 mg/kg body weight to 50 mg/kg body weight, e.g., from about 1 mg/kg body weight to about 5 mg/kg body weight, from about 5 mg/kg body weight to about 10 mg/kg body weight, from about 10 mg/kg body weight to about 15 mg/kg body weight, from about 15 mg/kg body weight to about 20 mg/kg body weight, from about 20 mg/kg body weight to about 25 mg/kg body weight, from about 25 mg/kg body weight to about 30 mg/kg body weight, from about 30 mg/kg body weight to about 35 mg/kg body weight, from about 35 mg/kg body weight to about 40 mg/kg body weight, or from about 40 mg/kg body weight to about 50 mg/kg body weight.

In some cases, a suitable dose of a T-Cell-MMP-epitope conjugate is from 0.01 μg to 100 mg per kg of body weight, from 0.1 μg to 10 mg per kg of body weight, from 1 μg to 1 mg per kg of body weight, from 10 μg to 100 mg per kg of body weight, from 100 μg to 10 mg per kg of body weight, or from 100 μg to 1 mg per kg of body weight. Persons of ordinary skill in the art can easily estimate repetition rates for dosing based on measured residence times and concentrations of the administered agent in bodily fluids or tissues. Following successful treatment, it may be desirable to have the patient undergo maintenance therapy to prevent the recurrence of the disease state, wherein a T-Cell-MMP-epitope conjugate is administered in maintenance doses, ranging from 0.01 μg to 100 g per kg of body weight, from 0.1 μg to 10 g per kg of body weight, from 1 μg to 1 g per kg of body weight, from 10 μg to 100 mg per kg of body weight, from 100 μg to 10 mg per kg of body weight, or from 100 μg to 1 mg per kg of body weight.

Those of skill will readily appreciate that dose levels can vary as a function of the specific T-Cell-MMP-epitope conjugate, the severity of the symptoms and the susceptibility of the subject to side effects. Preferred dosages for a given compound are readily determinable by those of skill in the art by a variety of means.

In some cases, multiple doses of a T-Cell-MMP-epitope conjugate, a nucleic acid or a recombinant expression vector are administered. The frequency of administration of a T-Cell-MMP-epitope conjugate, a nucleic acid, or a recombinant expression can vary depending on any of a variety of factors, e.g., severity of the symptoms, etc. For example, in some cases, a T-Cell-MMP-epitope conjugate, a nucleic acid, or a recombinant expression vector is administered once every year, once every 2-6 months, once per month, once every three weeks, or more frequently. When administered to persons who are uninfected and at risk of infection in order to cause priming and/or expansion of epitope-specific T cells and/or induce T cell memory, the administration can comprise an initial dose followed by one or more subsequent doses that are administered within a month, within one to two months, within two to four months, within six months, within six to twelve months, or longer than twelve months after the prior dose.

B. Routes of Administration

An active agent (a T-Cell-MMP-epitope conjugate of the present disclosure) is administered to an individual using any available method and route suitable for drug delivery, including in vivo and ex vivo methods, as well as systemic and localized routes of administration.

Conventional and pharmaceutically acceptable routes of administration include intratumoral, peritumoral, intramuscular, intralymphatic, intratracheal, intracranial, subcutaneous, intradermal, topical, intravenous, intraarterial, rectal, nasal, oral, and other enteral and parenteral routes of administration. Routes of administration may be combined, if desired, or adjusted depending upon the T-Cell-MMP-epitope conjugate and/or the desired effect. As noted above, a T-Cell-MMP-epitope conjugate of the present disclosure, can be administered in a single dose or in multiple doses.

In some embodiments, a T-Cell-MMP-epitope conjugate is administered intravenously. In some embodiments, a T-Cell-MMP-epitope conjugate is administered intramuscularly. In some embodiments, a T-Cell-MMP-epitope conjugate is administered subcutaneously.

C. Subjects Suitable for Treatment

Subjects suitable for treatment with a method or T-Cell-MMP-coronavirus epitope conjugate include individuals who have a Betacoronavirus infection such as SARS or MERS, or who are at risk of incurring a SARS or MERS infection, e.g., a SARS-CoV-2 infection, and/or are at greater risk of severe illness from such infection (e.g., e.g., a SARS-CoV-2 infection) than the general population. Suitable subjects include individuals who have been diagnosed as having SARS-CoV-2 (Covid-19), individuals who have been treated for coronavirus infection (e.g., SARS, SARS-CoV-2, or MERS) but who failed to respond to the treatment, and individuals who have been treated for coronavirus infection and who initially responded but subsequently became refractory to the treatment. Individuals who are at greater risk of severe illness from an infection such as a SARS-CoV-2 infection than the general population include individuals having one or more underlying medical conditions selected from the group consisting of chronic kidney disease, COPD (chronic obstructive pulmonary disease), Down Syndrome, heart conditions, such as heart failure, coronary artery disease, or cardiomyopathies, an immunocompromised state (weakened immune system) from solid organ transplant, obesity (body mass index [BMI] of 30 kg/m2 or higher but <40 kg/m²), severe obesity (BMI ≥40 kg/m²), pregnancy, sickle cell disease, a history of smoking, Type 2 diabetes mellitus, asthma (moderate-to-severe), cerebrovascular disease (affects blood vessels and blood supply to the brain), cystic fibrosis, hypertension or high blood pressure, an immunocompromised state (weakened immune system) from blood or bone marrow transplant, immune deficiencies, HIV, use of corticosteroids, or use of other immune weakening medicines, neurologic conditions such as dementia, liver disease, overweight (BMI >25 kg/m², but <30 kg/m²), pulmonary fibrosis (having damaged or scarred lung tissues), thalassemia or other blood disorders, and Type 1 diabetes mellitus.

### X. Certain Aspects

While the present technology has been described with reference to the specific aspects thereof, it should be understood by those skilled in the art that various changes may be made, and equivalents may be substituted without departing from the true spirit and scope of this disclosure. In addition, many modifications may be made to adapt a particular situation, material, composition of matter, process, and/or process step or steps, to the objective, spirit and scope of the present disclosure. All such modifications are intended to be within the scope of the claims appended hereto.
1. A T-Cell-MMP-epitope conjugate comprising:
   a) a first polypeptide having an N-terminus and a C-terminus, the first polypeptide comprising,
      i) a first major histocompatibility complex polypeptide (Class I MHC polypeptide) having an N-terminus and a C-terminus, and an optional linker at its N-terminus and/or C-terminus;
   b) a second polypeptide having an N-terminus and a C-terminus, the second polypeptide comprising (e.g., in order from N-terminus to C-terminus),
      i) a second MHC polypeptide (Class I MHC);
      ii) optionally an immunoglobulin (Ig) Fc polypeptide or a non-Ig polypeptide scaffold, and
      iii) an optional linker at the N-terminus and/or the C-terminus of the second polypeptide;
   c) one or more first polypeptide chemical conjugation sites attached to (e.g., at the N- or C-terminus) or within the first polypeptide, and/or one or more second polypeptide chemical conjugation sites attached to (e.g., at the N- or C-terminus) or within the second polypeptide;
   d) one or more (e.g., two or more) immunomodulatory polypeptides (MODs), wherein at least one of the one or more MODs is
      A) at the C-terminus of the first polypeptide,
      B) at the N-terminus of the second polypeptide,
      C) at the C-terminus of the second polypeptide,
      D) at the C-terminus of the first polypeptide and at the N-terminus of the second polypeptide or
      E) within the first or second polypeptide; and
   e) a coronavirus (e.g., SARS-CoV, SARS-CoV-2, MERS, etc.) epitope (e.g., peptide epitope) covalently bound, directly or indirectly (e.g., through a linker) to at least one of the one or more first polypeptide chemical conjugation sites or the one or more second polypeptide chemical conjugation sites, the coronavirus epitope (e.g., peptide epitope) comprising four (4) or more (e.g., 5, 6, 7, 8, 9, 10, 11, 12, 13, 14, 15 or 16, or 6 or more) contiguous amino acids of any of the coronavirus protein or peptide sequences set forth in FIGS. 11A to 11J, FIG. 15 and/or Table 2;

wherein each of the one or more MODs is an independently selected wild-type or variant MOD.

The T-Cell-MMP-epitope conjugate of aspect 1 may be subject to the proviso that neither the first nor the second polypeptide comprises an MHC-H polypeptide explicitly disclosed (e.g., as a sequence) in International Appln. PCT/US2018/049803, which published as WO 2019/051127. The T-Cell-MMP-epitope conjugate of aspect 1 may be subject to the proviso that it does not include or comprise a T-Cell-MMP and/or T-Cell-MMP-epitope conjugate disclosed in International Appln. PCT/US2018/049803.
2. A T-Cell-MMP-epitope conjugate comprising:
   a) a first polypeptide having an N-terminus and a C-terminus, the first polypeptide comprising,
      i) a first major histocompatibility complex polypeptide (Class I MHC polypeptide) having an N-terminus and a C-terminus, and an optional linker at its N-terminus and/or C-terminus;
   b) a second polypeptide having an N-terminus and a C-terminus, the second polypeptide comprising (e.g., in order from N-terminus to C-terminus),
      i) a second MHC polypeptide;
      ii) optionally an immunoglobulin (Ig) Fc polypeptide or a non-Ig polypeptide scaffold, and
      (iii) an optional linker at the N-terminus and/or the C-terminus of the second polypeptide;
   c) one or more first polypeptide chemical conjugation sites attached to (e.g., at the N- or C-terminus) or within the first polypeptide, and/or one or more second polypeptide chemical conjugation sites attached to (e.g., at the N- or C-terminus) or within the second polypeptide;
   d) one or more (e.g., two or more) immunomodulatory polypeptides (MODs), wherein at least one of the one or more MODs is
      A) at the C-terminus of the first polypeptide,
      B) at the N-terminus of the second polypeptide,
      C) at the C-terminus of the second polypeptide,
      D) at the C-terminus of the first polypeptide and at the N-terminus of the second polypeptide or
      E) within the first or second polypeptide; and
   e) a coronavirus peptide epitope covalently bound, directly or indirectly (e.g., through a linker) to at least one of the one or more first polypeptide chemical conjugation sites or the one or more second polypeptide chemical conjugation sites, the coronavirus peptide epitope comprising four (4) or more (e.g., 5, 6, 7, 8, 9, 10, 11, 12, 13, 14, 15 or 16, or 6 or more) contiguous amino acids of any of the coronavirus protein or peptide sequences set forth in FIGS. 11A to 11J, FIG. 15 and/or Table 2;
   wherein each of the one or more MODs is an independently selected wild-type or variant MOD; and
   wherein the first or second polypeptide comprises an MHC-H polypeptide sequence having at least 85% (e.g., at least 90%, at least 95%, at least 97%, at least 98% at least 99% or 100%) sequence identity to at least a portion (e.g., 20-250, 20-100, 30-100, 40-120, 50-150, 70-170, 100-150, 100-200, 150-200, 200-250 contiguous aas, more than 250 contiguous aas, or all) of an MHC-H chain polypeptide selected from the group consisting of: HLA-A*0101 (SEQ ID NO:20), HLA-A*0201 (SEQ ID NO:23), HLA-A*0301 (SEQ ID NO:31), HLA-A*1101 (SEQ ID NO:28), HLA-A*2301 (SEQ ID NO:32), HLA-A*2402 (SEQ ID NO:29), HLA-A*2407 (SEQ ID NO:33), HLA-A*3303 (SEQ ID NO:30), HLA-A*3401 (SEQ ID NO:34), HLA-B*0702 (SEQ ID NO:36), HLA-B*0801 (SEQ ID NO:37), HLA-B*1502 (SEQ ID NO:38), HLA-B*3501 (SEQ ID NO:127), HLA-B*3802 (SEQ ID NO:39), HLA-B*4001 (SEQ ID NO:40), HLA-B*4402 (SEQ ID NO:128), HLA-B*4403 (SEQ ID NO:129), HLA-B*4601 (SEQ ID NO:41), HLA-B*5301 (SEQ ID NO:42), HLA-B*5801 (SEQ ID NO:130), HLA-C*0102 (SEQ ID NO:44), HLA-C*0303 (SEQ ID NO:45), HLA-C*0304 (SEQ ID NO:46), HLA-C*0401 (SEQ ID NO:47), HLA-C*0602 (SEQ ID NO:48), HLA-C*0701 (SEQ ID NO:49), HLA-C*0702 (SEQ ID NO:50), HLA-C*0801 (SEQ ID NO:51), and HLA-C*1502 (SEQ ID NO:52, an HLA-E polypeptide (SEQ ID NO: 54), an HLA-F polypeptide (SEQ ID NO: 55), and an HLA-G polypeptide (SEQ ID NO:56).
      The T-Cell-MMP-epitope conjugate of aspect 2 may be subject to the proviso that neither the first nor the second polypeptide comprises an MHC-H polypeptide explicitly disclosed (e.g., as a sequence) in International Appln. PCT/US2018/049803, which published as WO 2019/051127. The T-Cell-MMP-epitope conjugate of aspect 2 may be subject to the proviso that it does not include or comprise a T-Cell-MMP and/or T-Cell-MMP-epitope conjugate disclosed in International Appln. PCT/US2018/049803.
3. A T-Cell-MMP-epitope conjugate comprising:
   a) a first polypeptide having an N-terminus and a C-terminus, the first polypeptide comprising,
      i) a first major histocompatibility complex (MHC) polypeptide having an N-terminus and a C-terminus, and an optional linker at its N-terminus and/or C-terminus;
   b) a second polypeptide having an N-terminus and a C-terminus, the second polypeptide comprising (e.g., in order from N-terminus to C-terminus),
      i) a second MHC polypeptide;
      ii) optionally an immunoglobulin (Ig) Fc polypeptide or a non-Ig polypeptide scaffold, and
      (iii) an optional linker at the N-terminus and/or the C-terminus of the second polypeptide;
   c) one or more first polypeptide chemical conjugation sites attached to (e.g., at the N- or C-terminus) or within the first polypeptide, and/or one or more second polypeptide chemical conjugation sites attached to (e.g., at the N- or C-terminus) or within the second polypeptide;
   d) one or more (e.g., two or more) immunomodulatory polypeptides (MODs), wherein at least one of the one or more MODs is
      A) at the C-terminus of the first polypeptide,
      B) at the N-terminus of the second polypeptide,
      C) at the C-terminus of the second polypeptide,
      D) at the C-terminus of the first polypeptide and at the N-terminus of the second polypeptide or
      E) within the first or second polypeptide; and
   e) a coronavirus peptide epitope covalently bound, directly or indirectly (e.g., through a linker) to at least one of the one or more first polypeptide chemical conjugation sites or the one or more second polypeptide chemical conjugation sites, the coronavirus peptide epitope comprising four (4) or more (e.g., 5, 6, 7, 8, 9, 10, 11, 12, 13, 14, 15 or 16, or 6 or more) contiguous amino acids of any of the coronavirus protein or peptide sequences set forth in FIGS. 11A to 11J, FIG. 15 and/or Table 2;
   wherein each of the one or more MODs is an independently selected wild-type or variant MOD; and
   wherein the first or second polypeptide comprises an MHC-H polypeptide sequence having at least 85% (e.g., at least 90%, at least 95%, at least 97%, at least 98% at least 99% or 100%) sequence identity to at least a portion (e.g., 20-250, 20-100, 30-100, 40-120, 50-150, 70-170, 100-150, 100-200, 150-200, 200-250 contiguous aas, more than 250 contiguous aas, or all) of an MHC-H chain polypeptide selected from the group consisting of: HLA-A*0101 (SEQ ID NO:20), HLA-A*0201 (SEQ ID NO:23), HLA-A*0301 (SEQ ID NO:31), HLA-A*1101 (SEQ ID NO:28), HLA-A*2301 (SEQ ID NO:32), HLA-A*2402 (SEQ ID NO:29), HLA-B*0702 (SEQ ID NO:36), HLA-B*0801 (SEQ ID NO:37), HLA-B*3501 (SEQ ID NO:127). HLA-B*4001 (SEQ ID NO:40), HLA-B*4402 (SEQ ID NO:128), HLA-B*4403 (SEQ ID NO:129), HLA-B*5801 (SEQ ID NO:130).
      The T-Cell-MMP-epitope conjugate of aspect 3 may be subject to the proviso that neither the first nor the second polypeptide comprises an MHC-H polypeptide explicitly disclosed (e.g., as a sequence) in International Appln. PCT/US2018/049803, which published as WO 2019/051127. The T-Cell-MMP-epitope conjugate of aspect 3 may be subject to the proviso that it does not include or comprise a T-Cell-MMP and/or T-Cell-MMP-epitope conjugate disclosed in International Appln. PCT/US2018/049803.
4. The T-Cell-MMP-epitope conjugate of any one of aspects 1-3, wherein the first polypeptide comprises:
   a first MHC polypeptide without a linker (e.g., a polypeptide linker) on its N-terminus and C-terminus,
   a first MHC polypeptide bearing a linker (e.g., a polypeptide linker) on its N-terminus,
   a first MHC polypeptide bearing a linker (e.g., a polypeptide linker) on its C-terminus, or
   a first MHC polypeptide bearing a linker (e.g., a polypeptide linker) on its N-terminus and C-terminus.
5. The T-Cell-MMP-epitope conjugate of any one of aspects 1 to 4, wherein at least one of the one or more first polypeptide chemical conjugation sites is:
   a) attached to (e.g., at the N- or C-terminus), or within, the sequence of the first MHC polypeptide, where the first MHC polypeptide is without a linker on its N- and C-termini;
   b) attached to (e.g., at the N- or C-terminus), or within, the sequence of the first MHC polypeptide where the first MHC polypeptide comprises a linker on its N- and C-terminus;
   c) attached to (e.g., at the N- or C-terminus) or within, the sequence of the linker on the N-terminus of the first MHC polypeptide; and/or
   d) attached to (e.g., at the N- or C-terminus) or within, the sequence of the linker on the C-terminus of the first MHC polypeptide.
6. The T-Cell-MMP-epitope conjugate of any one of aspects 1 to 5, wherein the first and second MHC polypeptides are Class I MHC polypeptides, and the first MHC polypeptide comprises:
   a beta-2-microglobulin (“β2M”) polypeptide (see, e.g., FIG. 4) having an N-terminus and a C-terminus with or without a linker on its N- and/or C-termini,
   a β2M polypeptide bearing a linker on its N-terminus,
   a β2M polypeptide bearing a linker on its C-terminus, or
   a β2M polypeptide bearing a linker on its N-terminus and C-terminus.
7. The T-Cell-MMP-epitope conjugate of aspect 6, wherein at least one of the one or more first polypeptide chemical conjugation sites is:
   a) attached to (e.g., at the N- or C-terminus) or within the sequence of the β2M polypeptide without a linker on its N- and/or C-termini;
   b) attached to (e.g., at the N- or C-terminus) or within the sequence of the β2M polypeptide where the β2M polypeptide comprises a linker on its N- and C-termini;
   c) attached to (e.g., at the N- or C-terminus) or within the sequence of the linker on the N-terminus of the β2M polypeptide; and/or
   d) attached to (e.g., at the N- or C-terminus) or within, the sequence of the linker on the C-terminus of the β2M polypeptide.
8. The T-Cell-MMP-epitope conjugate of any one of aspects 1 to 7, comprising:
   a second MHC polypeptide without a linker on its N-terminus and C-terminus,
   a second MHC polypeptide bearing a linker on its N-terminus,
   a second MHC polypeptide bearing a linker on its C-terminus, or
   a second MHC polypeptide bearing a linker on its N-terminus and C-terminus.
9. The T-Cell-MMP-epitope conjugate of aspect 8, wherein the second polypeptide further comprises an immunoglobulin (Ig) Fc polypeptide or a non-Ig polypeptide scaffold.
10. The T-Cell-MMP-epitope conjugate of aspect 9, wherein the second polypeptide comprises, in order from N-terminus to C-terminus: a second MHC polypeptide bearing a linker on its C-terminus followed by an immunoglobulin (Ig) Fc polypeptide or a non-Ig polypeptide scaffold; or a second MHC polypeptide bearing a linker on its N-terminus and/or C-terminus followed by an immunoglobulin (Ig) Fc polypeptide or a non-Ig polypeptide scaffold.
11. The T-Cell-MMP-epitope conjugate of any one of aspects 1 to 10, wherein at least one of the one or more second polypeptide chemical conjugation sites is:
   a) attached to (e.g., at the N- or C-terminus) or within the sequence of the second MHC polypeptide, wherein the second MHC polypeptide is without a linker on its N- and C-termini;
   b) attached to (e.g., at the N- or C-terminus) or within the sequence of the second MHC polypeptide wherein the second MHC polypeptide comprises a linker on its N- and/or C-terminus;
   c) attached to (e.g., at the N- or C-terminus) or within the sequence of the linker on the N-terminus of the second MHC polypeptide;
   d) attached to (e.g., at the N- or C-terminus) or within the sequence of the linker on the C-terminus of the second MHC polypeptide; and/or
   e) attached to (e.g., at the N- or C-terminus) or within the sequence of an immunoglobulin (Ig) Fc polypeptide or a non-Ig polypeptide scaffold when the second MHC polypeptide is followed by an immunoglobulin (Ig) Fc polypeptide or a non-Ig polypeptide scaffold.
12. The T-Cell-MMP-epitope conjugate of any one of aspects 1 to 11, wherein the second MHC polypeptide comprises: an MHC Class I heavy chain (“MHC-H,” see, e.g., FIGS. 3A-3I) polypeptide having an N-terminus and a C-terminus without a linker on its N- and C-termini, an MHC-H polypeptide bearing a linker on its N-terminus, an MHC-H polypeptide bearing a linker on its C-terminus, or an MHC-H polypeptide bearing a linker on its N-terminus and C-terminus.
13. The T-Cell-MMP-epitope conjugate of any one of aspects 6 to 12, wherein at least one of the one or more first polypeptide chemical conjugation sites is:
   a) attached to (e.g., at the N- or C-terminus), or within, the sequence of the β2M polypeptide without a linker on its N- and C-termini;
   b) attached to (e.g., at the N- or C-terminus), or within, the sequence of the β2M polypeptide where the β2M polypeptide comprises a linker on its N- and C-termini;
   c) attached to (e.g., at the N- or C-terminus), or within, the sequence of the linker on the N-terminus of the β2M polypeptide; and/or
   d) attached to (e.g., at the N- or C-terminus), or within, the sequence of the linker on the C-terminus of the β2M polypeptide.
14. The T-Cell-MMP-epitope conjugate of any one of aspects 6 to 12, wherein at least one of the one or more first polypeptide chemical conjugation sites replaces and/or is inserted between any of the amino terminal 15 amino acids of a mature β2M polypeptide sequence lacking its signal sequence (e.g., a β2M polypeptide sequence shown in FIG. 4).
15. The T-Cell-MMP-epitope conjugate of any one of aspects 1 to 14, wherein the second polypeptide comprises an Ig Fc polypeptide.
16. The T-Cell-MMP-epitope conjugate of aspect 15, wherein the Ig Fc polypeptide is an IgG1 Fc polypeptide, an IgG2 Fc polypeptide, an IgG3 Fc polypeptide, an IgG4 Fc polypeptide, an IgA Fc polypeptide, or an IgM Fc polypeptide (e.g., an immunoglobulin sequence in any of FIGS. 2A to 2G).
17. The T-Cell-MMP-epitope conjugate of aspect 16, wherein the Ig Fc polypeptide comprises an amino acid sequence having at least 85% amino acid sequence identity (e.g., at least 90%, 95%, 98% or 99% identity, or even 100% identity) to an amino acid sequence depicted in one of FIGS. 2A-2D, or a portion of a sequence (at least about 50, 75, 100, 125 or 150 amino acids in length) in one of FIGS. 2A-2D corresponding to the IgFc polypeptide.
18. The T-Cell-MMP-epitope conjugate of aspect 17, wherein the IgFc polypeptide is an IgG1 Fc polypeptide.
19. The T-Cell-MMP-epitope conjugate of aspect 18, wherein the IgG1 Fc polypeptide comprises one or more amino acid substitutions selected from N297A, L234A, L235A, L234F, L235E, and P331S.
20. The T-Cell-MMP-epitope conjugate of aspect 19, wherein the IgG1 Fc polypeptide comprises L234A and L235A substitutions.
21. The T-Cell-MMP-epitope conjugate of any one of aspects 1 to 20, wherein at least one (e.g., at least two, or each) of the one or more MODs is a wild-type or variant MOD selected independently from the group consisting of CD7, CD80, CD86, PD-L1, PD-L2, 4-1BBL, OX40L, Fas ligand (FasL), ICOS-L, ICAM, IL-2, TGF-β, CD30L, CD40, CD70, CD83, HVEM, lymphotoxin beta receptor, 3/TR6, ILT3, ILT4, HVEM, ILCD70, JAG1, and TGF-β.
22. The T-Cell-MMP-epitope conjugate of any one of aspects 1 to 21, wherein at least one (e.g., at least two) of the one or more MODs is a wild-type or variant MOD selected independently from the group consisting of: IL-2, 4-1BBL, PD-L1, CD80, and CD86.
23. The T-Cell-MMP-epitope conjugate of any one of aspects 1 to 21, wherein the T-Cell-MMP-epitope conjugate comprises one or more independently selected wild type or variant IL-2 MODs.
24. The T-Cell-MMP-epitope conjugate of any one of aspects 1 to 21, wherein the T-Cell-MMP-epitope conjugate comprises one or more independently selected wild type or variant 4-1BBL MODs.
25. The T-Cell-MMP-epitope conjugate of any one of aspects 1 to 21, wherein the T-Cell-MMP-epitope conjugate comprises one or more independently selected wild type or variant PD-L1 MODs.
26. The T-Cell-MMP-epitope conjugate of any one of aspects 1 to 21, wherein the T-Cell-MMP-epitope conjugate comprises one or more independently selected wild type or variant CD80 MODs.
27. The T-Cell-MMP-epitope conjugate of any one of aspects 1 to 21, wherein the T-Cell-MMP-epitope conjugate comprises one or more independently selected wild type or variant CD86 MODs.
28. The T-Cell-MMP-epitope conjugate of any one of aspects 1 to 27, wherein the T-Cell-MMP-epitope conjugate comprises one or more independently selected wild-type and/or variant MOD polypeptides; wherein at least one of the one or more variant MOD polypeptides exhibits a reduced affinity to a Co-MOD (its Co-MOD) compared to the affinity of a corresponding wild-type MOD for the Co-MOD (e.g., the ratio of i) the binding affinity of a control T-Cell-MMP-epitope conjugate where the control comprises a wild-type MOD to a Co-MOD to ii) the binding affinity of a T-Cell-MMP-epitope conjugate comprising a variant of the wild-type MOD to the Co-MOD, when measured by BLI (as described above), is at least 1.5:1, at least 2:1, at least 5:1, at least 10:1, at least 15:1, at least 20:1, at least 25:1, at least 50:1, at least 100:1, at least 500:1, at least 102:1, at least 5×102:1, at least 103:1, at least 5×103:1, at least 104:1, at least 105:1, or at least 106:1). 29. The T-Cell-MMP-epitope conjugate of aspect 28, wherein the variant MOD polypeptides comprise from 1, 2, 3, 4, 5, 6, 7, 8, 9, or 10 amino acid substitutions, insertions, or deletions relative to a corresponding wild-type immunomodulatory polypeptide, or comprise an amino acid sequence having at least 85% amino acid sequence identity (e.g., at least 90%, 95%, 98% or 99% identity, or even 100% identity) to an amino acid sequence of the corresponding wild-type MOD, or a portion of the sequence of the corresponding wild-type MOD (e.g., at least about 50, 75, 100, 125 or 150 contiguous amino acids of the wild-type MOD in length).
30. The T-Cell-MMP-epitope conjugate of any one of aspects 1 to 29, wherein the first MHC polypeptide comprises a β2M polypeptide; and wherein the second MHC polypeptide comprises an MHC Class I heavy chain polypeptide.
31. The T-Cell-MMP-epitope conjugate of any one of aspects 6 to 30, wherein the β2M polypeptide comprises an amino acid sequence having at least 85% amino acid sequence identity (e.g., at least 90%, 95%, 98% or 99% identity, or even 100% identity) to one of the amino acid sequences set forth in FIG. 4, or at least 60 contiguous amino acids of a mature sequence β2M polypeptide in FIG. 4 (e.g., at least about 60, 70, 80, or 90 amino acids).
32. The T-Cell-MMP-epitope conjugate of any one of aspects 6 to 31, wherein the β2M polypeptide comprises, consists essentially of, or consists of a sequence of at least 20, 30, 40, 50, 60, 70, 80, 90 or 99 contiguous amino acids having identity with at least a portion of one of the amino acid sequences set forth in FIG. 4 (e.g., a sequence having 20-99, 20-40, 30-50, 40-60, 40-90, 50-70, 60 to 80, 60-99, 70-90, or 79-99 contiguous amino acids with identity to a sequence of mature β2M polypeptide lacking its signal sequence set forth in FIG. 4).
33. The T-Cell-MMP-epitope conjugate of any one of aspects 12 to 32, wherein the MHC Class I heavy chain polypeptide comprises all or part of an HLA-A, HLA-B, HLA-C, HLA-E, HLA-F or HLA-G heavy chain (e.g., from FIGS. 3A to 3I, HLA-B*3501 (SEQ ID NO:127), HLA-B*4402 (SEQ ID NO:128), HLA-B*4403 (SEQ ID NO:129), HLA-B*5301 (SEQ ID NO:42), or HLA-B*5801 (SEQ ID NO:130).
34. The T-Cell-MMP-epitope conjugate of aspect 33, wherein the MHC Class I heavy chain polypeptide sequence comprises an amino acid sequence having at least 85% amino acid sequence identity (e.g., at least 90%, 95%, 98% or 99% identity, or even 100% identity) to all or part (e.g., 20-250, 20-40, 20-100, 30-50, 40-60, 40-90, 50-70, 60-80, 60-90, 70-100, 80-120, 100-150, 120-200, 150-200, 200-250, or more than 250 contiguous amino acids) of one of the amino acid sequences set forth in one of FIGS. 3D-3I, HLA-B*3501 (SEQ ID NO:127), HLA-B*4402 (SEQ ID NO:128), HLA-B*4403 (SEQ ID NO:129), HLA-B*5301 (SEQ ID NO:42), or HLA-B*5801 (SEQ ID NO:130).
35. The T-Cell-MMP-epitope conjugate of aspect 33, wherein the MHC Class I heavy chain polypeptide sequence comprises all or part (e.g., 20-250, 20-40, 20-100, 30-50, 40-60, 40-90, 50-70, 60-80, 60-90, 70-100, 80-120, 100-150, 120-200, 150-200, 200-250, or more than 250 contiguous amino acids) of an HLA-A polypeptide sequence having greater than 85%, 90%, 95%, or 98% sequence identity to an HLA-A allele sequence set forth in FIG. 3E.
36. The T-Cell-MMP-epitope conjugate of aspect 35, wherein the MHC Class I heavy chain polypeptide sequence comprises all or part (e.g., 20-250, 20-40, 20-100, 30-50, 40-60, 40-90, 50-70, 60-80, 60-90, 70-100, 80-120, 100-150, 120-200, 150-200, 200-250, or more than 250 contiguous amino acids) of an HLA-A polypeptide sequence having greater than 90%, 95%, 98% or 99% sequence identity to the HLA-A allele consensus sequence set forth in FIG. 3E (excluding positions X1 to X45 with defined amino acid variations in the consensus sequence).
37. The T-Cell-MMP-epitope conjugate of aspect 33, wherein the MHC Class I heavy chain polypeptide sequence comprises all or part (e.g., 20-250, 20-40, 20-100, 30-50, 40-60, 40-90, 50-70, 60-80, 60-90, 70-100, 80-120, 100-150, 120-200, 150-200, 200-250, or more than 250 contiguous amino acids) of an HLA-B polypeptide sequence having greater than 85%, 90%, 95%, or 98% sequence identity to an HLA-B allele sequence set forth in FIG. 3F, HLA-B*3501 (SEQ ID NO:127), HLA-B*4402 (SEQ ID NO:128), HLA-B*4403 (SEQ ID NO:129), or HLA-B*5801 (SEQ ID NO:130).
38. The T-Cell-MMP-epitope conjugate of aspect 37, wherein the MHC Class I heavy chain polypeptide sequence comprises all or part (e.g., 20-250, 20-40, 20-100, 30-50, 40-60, 40-90, 50-70, 60-80, 60-90, 70-100, 80-120, 100-150, 120-200, 150-200, 200-250, or more than 250 contiguous amino acids) of an HLA-B polypeptide sequence having greater than 90%, 95%, 98% or 99% sequence identity to the HLA-B allele consensus sequence set forth in FIG. 3F (excluding positions X1 to X34 with defined amino acid variations in the consensus sequence)
39. The T-Cell-MMP-epitope conjugate of aspect 33, wherein the MHC Class I heavy chain polypeptide sequence comprises all or part (e.g., 20-250, 20-40, 20-100, 30-50, 40-60, 40-90, 50-70, 60-80, 60-90, 70-100, 80-120, 100-150, 120-200, 150-200, 200-250, or more than 250 contiguous amino acids) of an HLA-C polypeptide sequence having greater than 85%, 90%, 95%, or 98% sequence identity to an HLA-C allele sequence set forth in FIG. 3G.
40. The T-Cell-MMP-epitope conjugate of aspect 39, wherein the MHC Class I heavy chain polypeptide sequence comprises all or part (e.g., 20-250, 20-40, 20-100, 30-50, 40-60, 40-90, 50-70, 60-80, 60-90, 70-100, 80-120, 100-150, 120-200, 150-200, 200-250, or more than 250 contiguous amino acids) of an HLA-C polypeptide sequence having greater than 90%, 95%, 98% or 99% sequence identity to the HLA-C allele consensus sequence set forth in FIG. 3G (excluding positions X1 to X37 with defined amino acid variations in the consensus sequence).
41. The T-Cell-MMP-epitope conjugate of aspect 33, wherein the MHC Class I heavy chain polypeptide sequence comprises all or part (e.g., 20-250, 20-40, 20-100, 30-50, 40-60, 40-90, 50-70, 60-80, 60-90, 70-100, 80-120, 100-150, 120-200, 150-200, 200-250, or more than 250 contiguous amino acids) of an HLA-E, F, or G polypeptide sequence having greater than 85%, 90%, 95%, or 98% sequence identity to an HLA-E, F, or G allele sequence set forth in FIG. 3H.
42. The T-Cell-MMP-epitope conjugate of aspect 41, wherein the MHC Class I heavy chain polypeptide sequence comprises all or part (e.g., 20-250, 20-40, 20-100, 30-50, 40-60, 40-90, 50-70, 60-80, 60-90, 70-100, 80-120, 100-150, 120-200, 150-200, 200-250, or more than 250 contiguous amino acids) of an HLA-E polypeptide sequence having greater than 90%, 95%, 98% or 99% sequence identity to an HLA-E allele consensus sequence set forth in FIG. 3H (excluding positions indicated by an “X” with defined amino acid variations in the consensus sequence).
43. The T-Cell-MMP-epitope conjugate of aspect 41, wherein the MHC Class I heavy chain polypeptide sequence comprises all or part (e.g., 20-250, 20-40, 20-100, 30-50, 40-60, 40-90, 50-70, 60-80, 60-90, 70-100, 80-120, 100-150, 120-200, 150-200, 200-250, or more than 250 contiguous amino acids) of an HLA-F polypeptide sequence having greater than 90%, 95%, 98% or 99% sequence identity to an HLA-F allele consensus sequence set forth in FIG. 3H (excluding positions indicated by an “X” with defined amino acid variations in the consensus sequence).
44. The T-Cell-MMP-epitope conjugate of aspect 41, wherein the MHC Class I heavy chain polypeptide sequence comprises all or part (e.g., 20-250, 20-40, 20-100, 30-50, 40-60, 40-90, 50-70, 60-80, 60-90, 70-100, 80-120, 100-150, 120-200, 150-200, 200-250, or more than 250 contiguous amino acids) of an HLA-G polypeptide sequence having greater than 90%, 95%, 98% or 99% sequence identity to an HLA-G allele consensus sequence set forth in FIG. 3H (excluding positions indicated by an “X” with defined amino acid variations in the consensus sequence).
45. The T-Cell-MMP-epitope conjugate of any one of aspects 12 to 44, wherein the MHC Class I heavy chain polypeptide sequence comprises a disulfide bond between a cysteine at the carboxyl end portion of the MHC heavy chain α1 helix and a cysteine in the amino end portion of the MHC heavy chain α2-1 helix, and/or a cysteine or a cysteine substitution at any one or more (two, three, four, etc.) of amino acid residues 2, 5, 7, 84, 59, 116, 139, 167, 168, 170, or 171.
46. The T-Cell-MMP-epitope conjugate of aspect 45, wherein the carboxyl end portion of the MHC heavy chain α1 helix is from about amino acid position 79 to about amino acid position 89 and the amino end portion of the MHC heavy chain α2-1 helix is from about amino acid position 134 to amino acid position 144 of the MHC Class I heavy chain, wherein the amino acid positions are determined based on the sequence of the heavy chains without their leader sequence (see, e.g., FIGS. 3D to 3I).
47. The T-Cell-MMP-epitope conjugate of any one of aspects 45 to 46, wherein the disulfide bond is between a cysteine located at positions 83, 84, or 85 and a cysteine located at position 138, 139 or 140 (e.g., from position 83 to position 138, 139 or 140, from position 84 to position 138, 139 or 140, or from position 85 to position 138, 139 or 140).
48. The T-Cell-MMP-epitope conjugate of any one of aspects 45 to 47, wherein the disulfide bond is between a cysteine located at position 84 and a cysteine located at position 139.
49. The T-Cell-MMP-epitope conjugate of any one of aspects 45 to 48, wherein the MHC Class I heavy chain sequence may have insertions, deletions and/or substitutions of 1 to 5 aas (e.g., 1, 2, 3 or 4 aas) preceding and/or following either or both cysteines forming the disulfide bond between the carboxyl end portion of the α1 helix and the amino end portion of the α2-1 helix.
50. The T-Cell-MMP-epitope conjugate of aspect 49, wherein, when substitutions and/or insertions are present, the amino acids may be selected from any naturally occurring amino acid, or any naturally occurring amino acid except glycine and/or proline.
51. The T-Cell-MMP-epitope conjugate of any one of aspects 33 to 50, wherein the MHC Class I heavy chain polypeptide amino acid sequence at positions 1 to 79 has at least 85% amino acid sequence identity (e.g., at least 90%, 95%, 98% or 99% identity, or even 100% identity) to the corresponding portion of at least one sequence set forth in FIGS. 3D to 3H (e.g., the sequence has 1, 2, 3, 4, 5, 6, 7, 8, 9 or 10 amino acid insertions, deletions, or substitutions relative to a sequence in FIGS. 3D to 3H).
52. The T-Cell-MMP-epitope conjugate of any one of aspects 33 to 50, wherein the MHC Class I heavy chain polypeptide amino acid sequence from position 89 to 134 (inclusive of those positions) has at least 85% amino acid sequence identity (e.g., at least 90%, 95%, 98% or 99% identity, or even 100% identity) to the corresponding portion of at least one sequence set forth in FIGS. 3D to 3H (e.g., the sequence has 1, 2, 3, 4, 5, or 6 amino acid insertions, deletions, or substitutions relative to a sequence in FIGS. 3D to 3H).
53. The T-Cell-MMP-epitope conjugate of any one of aspects 33 to 52, wherein the MHC Class I heavy chain polypeptide amino acid sequence from position 144 to 230 (inclusive of those positions) has at least 85% amino acid sequence identity (e.g., at least 90%, 95%, 98% or 99% identity, or even 100% identity) to the corresponding portion of at least one sequence set forth in FIGS. 3D to 3H (e.g., the sequence has 1, 2, 3, 4, 5, 6, 7, 8, 9, 10, 11, 12, or 13 amino acid insertions, deletions, or substitutions relative to a sequence in FIGS. 3D to 3H).
54. The T-Cell-MMP-epitope conjugate of any one of aspects 33 to 53, wherein the MHC Class I heavy chain polypeptide amino acid sequence from positions 242 to 274 (inclusive of those positions) has at least 85% amino acid sequence identity (e.g., at least 90%, 95%, 98% or 99% identity, or even 100% identity) to the corresponding portion of at least one sequence set forth in FIGS. 3D to 3H (e.g., the sequence has 1, 2, 3, or 4 amino acid insertions, deletions, or substitutions relative to a sequence in FIGS. 3D to 3H).
55. The T-Cell-MMP-epitope conjugate of any one of aspects 1 to 54, wherein the first polypeptide and the second polypeptide are non-covalently associated.
56. The T-Cell-MMP-epitope conjugate of any one of aspects 1 to 54, wherein the first polypeptide and the second polypeptide are covalently linked to one another.
57. The T-Cell-MMP-epitope conjugate of aspect 56, wherein the covalent linkage is via a disulfide bond.
58. The T-Cell-MMP-epitope conjugate of any one of aspects 1 to 57, comprising two or more, three or more, or four or more independently selected MODs.
59. The T-Cell-MMP-epitope conjugate of any one of aspects 1 to 58, comprising a peptide linker between any two or more, three or more, or four or more of the two or more (e.g., two, three or four) wild-type and/or variant MODs.
60. The T-Cell-MMP-epitope conjugate of any one of aspects 1 to 59, wherein the first polypeptide comprises a peptide linker between the first MHC polypeptide and at least one wild-type or variant MOD.
61. The T-Cell-MMP-epitope conjugate of any one of aspects 1 to 60, wherein the second polypeptide comprises a peptide linker between the second MHC polypeptide and at least one wild-type or variant MOD.
62. The T-Cell-MMP-epitope conjugate of any one of aspects 1 to 61, comprising at least one peptide linker in the first and/or second polypeptide chain, wherein the linker has a length of from 5 amino acids to 30 amino acids (e.g., 5-10, 10-20, or 20-30 amino acids).
63. The T-Cell-MMP-epitope conjugate of aspect 62, wherein the linker comprises a peptide of the formula AAAGG or GGGGS, which may be repeated from one to ten times (e.g., from 1 to 4, 3 to 6, or 4 to 8 times).
64. The T-Cell-MMP-epitope conjugate of any one of aspects 1 to 63, wherein the first and second chemical conjugation sites are independently selected from:
   a) peptide sequences that act as an enzymatic modification sequence (e.g., a sulfatase motif);
   b) non-natural amino acids and/or selenocysteines;
   c) engineered amino acid chemical conjugation sites;
   d) carbohydrate or oligosaccharide moieties; and/or
   e) IgG nucleotide binding sites.
65. The T-Cell-MMP-epitope conjugate of any one of aspects 1 to 64, wherein at least one of the one or more first and second chemical conjugation sites comprises an enzymatic modification sequence.
66. The T-Cell-MMP-epitope conjugate of any one of aspects 1 to 65, wherein at least one of the one or more first or second chemical conjugation sites is a sulfatase motif.
67. The T-Cell-MMP-epitope conjugate of aspect 66, wherein the sulfatase motif comprises the sequence X1Z1X2Z2X3Z3, wherein
   Z1 is cysteine or serine;
   Z2 is either a proline or alanine residue;
   Z3 is a basic amino acid (arginine, lysine, or histidine, usually lysine), or an aliphatic amino acid (alanine, glycine, leucine, valine, isoleucine, or proline, usually A, G, L, V, or I);
   X1 is present or absent and, when present, can be any amino acid, though usually an aliphatic amino acid, a sulfur-containing amino acid, or a polar, uncharged amino acid (i.e., other than an aromatic amino acid or a charged amino acid), usually L, M, V. S or T, more usually L, M, S or V, with the proviso that, when the sulfatase motif is at the N-terminus of the target polypeptide, X1 is present; and
   X2 and X3 independently can be any amino acid, though usually an aliphatic amino acid, a polar, uncharged amino acid, or a sulfur containing amino acid (i.e., other than an aromatic amino acid or a charged amino acid), usually S, T, A, V, G or C, more usually S, T, A, V or G.
68. The T-Cell-MMP-epitope conjugate of aspect 67, comprising one or more fGly amino acid residues in the amino acid sequence of the first polypeptide or the second polypeptide.
69. The T-Cell-MMP-epitope conjugate of any one of aspects 1 to 68, wherein at least one of the one or more first or second chemical conjugation site is a Sortase A enzyme site comprising the amino acid sequence LP(X5)TG, LP(X5)TA, or LPETGG positioned at the C-terminus of the first and/or second polypeptide and wherein X5 is any amino acid.
70. The T-Cell-MMP-epitope conjugate of any one of aspects 1 to 69, wherein at least one of the one or more first or second chemical conjugation sites is a Sortase A enzyme site comprising at least one oligoglycine (e.g., (G)_{2, 3, 4, or 5}) at the amino terminus of the first and/or second polypeptides, and/or at least one oligo alanine (e.g., (A)_{2, 3, 4, or 5}) at the amino terminus of the first and/or second polypeptides.
71. The T-Cell-MMP-epitope conjugate of any one of aspects 1 to 70, wherein at least one of the one or more first or second chemical conjugation sites is a transglutaminase site.
72. The T-Cell-MMP-epitope conjugate of aspect 71, wherein at least one of the one or more transglutaminase sites is selected from the group consisting of: LQG, LLQGG, LLQG, LSLSQG, and LLQLQG.
73. The T-Cell-MMP-epitope conjugate of any one of aspects 1 to 72, wherein at least one of the one or more first and second chemical conjugation sites comprises a selenocysteine or an amino acid sequence containing one or more independently selected non-natural amino acids.
74. The T-Cell-MMP-epitope conjugate of aspect 73, wherein at least one of the one or more non-natural amino acids is selected from the group consisting of para-acetylphenylalanine, para-azido phenylalanine and propynyl-tyrosine.
75. The T-Cell-MMP-epitope conjugate of any one of aspects 1 to 74, wherein at least one of the one or more first and second chemical conjugation sites comprises an engineered amino acid site (e.g., a cysteine engineered into the first or second polypeptide).
76. The T-Cell-MMP-epitope conjugate of any one of aspects 1 to 75, wherein at least one of the one or more first and second chemical conjugation sites comprises one or more sulfhydryl or amine groups (e.g., a cysteine substitution at any one or more (two, three, four, etc.) of amino acid residues 2, 5, 7, 59, 84, 116, 139, 167, 168, 170, or 171).
77. The T-Cell-MMP-epitope conjugate of aspect 76, wherein at least one of the one or more sulfhydryl or amine groups results from the presence of a lysine or cysteine in the first and or second polypeptide.
78. The T-Cell-MMP-epitope conjugate of any one of aspects 1 to 77, wherein at least one of the one or more first and second chemical conjugation sites comprises an independently selected carbohydrate, monosaccharide, disaccharide and/or oligosaccharide.
79. The T-Cell-MMP-epitope conjugate of any one of aspects 1 to 78, wherein at least one of the one or more first and second chemical conjugation sites comprises one or more IgG nucleotide antibody binding sites.
80. The T-Cell-MMP-epitope conjugate of any one of aspects 1 to 79, wherein:
   a) the first polypeptide comprises a β2M polypeptide sequence,
   b) the second polypeptide comprises (e.g., in order from N-terminus to C-terminus),
      i) an MHC-H polypeptide and an immunoglobulin (Ig) Fc polypeptide;
   c) one or more first polypeptide chemical conjugation sites within the β2M polypeptide or on a peptide linker are attached (located) at the N-terminal to the β2M polypeptide sequence; and
   d) one or more MODs, wherein at least one of the one or more MODs is
      A) at the C-terminus of the first polypeptide,
      B) at the N-terminus of the second polypeptide,
      C) at the C-terminus of the second polypeptide,
      D) at the C-terminus of the first polypeptide and at the N-terminus of the second polypeptide and/or
      E) within the first or second polypeptide;
   wherein each of the one or more MODs is an independently selected wild-type or variant MOD; and
   wherein the first and second polypeptides are optionally joined by an interpeptide covalent bond.
      The T-Cell-MMP-epitope conjugate of aspect 80, may be subject to the proviso that neither the first nor the second polypeptide comprises an MHC-H polypeptide explicitly disclosed (e.g., as a sequence) in International Appln. PCT/US2018/049803, which published as WO 2019/051127.
      The T-Cell-MMP-epitope conjugate of aspect 80 may also be subject to the proviso that it does not include or comprise a T-Cell-MMP and/or T-Cell-MMP-epitope conjugate disclosed in International Appln. PCT/US2018/049803.
81. The T-Cell-MMP-epitope conjugate of aspect 80, comprising at least one MOD at:
   A) the C-terminus of the first polypeptide;
   B) the N-terminus of the second polypeptide; and/or
   C) the C-terminus of the second polypeptide.
82. The T-Cell-MMP-epitope conjugate of aspect 80,
   wherein the first and second polypeptides are joined by a disulfide bond between the MHC-H polypeptide and the β2M polypeptide; or
   wherein the first and second polypeptides are joined by a disulfide bond between the MHC-H polypeptide and a peptide linker attached at the N-terminal to the β2M polypeptide sequence.
83. The T-Cell-MMP-epitope conjugate of any one of aspects 80 to 82, wherein the disulfide bond between the MHC-H polypeptide and the β2M polypeptide is between an aa of the MHC-H polypeptide at about position 236 (e.g., A236C) and an aa of the β2M polypeptide at about position 12 (e.g., R12C).
84. The T-Cell-MMP-epitope conjugate of any one of aspects 80 to 83, wherein at least one chemical conjugation site comprises a cysteine engineered into the first or second polypeptide.
85. The T-Cell-MMP-epitope conjugate of any one of aspects 80 to 84, wherein at least one chemical conjugation site is a cysteine engineered into the β2M polypeptide sequence at position 2, 44, 50, 77, 85, 88, 91 or 98 (e.g., a Q2C, E44C, E50C, E77C, V85V, S88C, K91C, or D98C substitution) in a mature β2M polypeptide.
86. The T-Cell-MMP-epitope conjugate of any one of aspects 80 to 85, wherein the chemical conjugation site is a cysteine engineered into the β2M polypeptide sequence as a Q2C or E44C substitution of a mature β2M polypeptide (e.g., the polypeptide of SEQ ID NOs: 57-60, which lacks the signal sequence).
87. The T-Cell-MMP-epitope conjugate of any one of aspects 84 to 86, wherein the epitope is conjugated through the cysteine engineered into the first or second polypeptide.
88. The T-Cell-MMP-epitope conjugate of any one of aspects 80 to 87, wherein the epitope is conjugated to a chemical conjugation site through a linker, selected from a peptide or non-peptide polymer.
89. The T-Cell-MMP-epitope conjugate of aspect 88, wherein the epitope is conjugated through a linker that comprises a peptide having a length of from 4 amino acids to 30 amino acids (e.g., 4-10, 10-20 or 20-30 amino acids) including, but not limited to, polypeptides comprising from 1-10 repeating units of: glycine (polyG); glycine-serine polymer repeating units (including, for example, (GS), (GSGGS), (GGGS), (GGSG), (GGSGG), (GSGSG), (GSGGG), (GGGSG), (GSSSG), and (GGGGS)n); glycine-alanine polymer repeating units such as (AAAGG); alanine-serine polymers; cysteine containing linkers (e.g., GCGASGGGGSGGGGS, GCGGSGGGGSGGGGSGGGGS, GCGGSGGGGSGGGGS, or GCGGS(G4S)n, where n is an integer of at least one (e.g., 1, 2, 3, 4, 5, 6, 7, 8, 9, or 10).
90. The T-Cell-MMP-epitope conjugate of aspect 89, wherein the epitope is conjugated through a linker that comprises a peptide of the formula (AAAGG) or (GGGGS), which may be repeated from 1 to 8 times (e.g., 1, 2, 3, 4, 5, 6, 7, or 8 times, or in a range selected from 1 to 4, 3 to 6, or 4 to 8 times).
91. The T-Cell-MMP-epitope conjugate of any one of aspects 84 to 90, wherein the epitope is conjugated through an engineered cysteine by a maleimide group that is incorporated into the epitope peptide or a linker (e.g., a GGGGS sequence repeated from 1 to 8 times) attached to the epitope peptide.
92. The T-Cell-MMP-epitope conjugate of any one of aspects 88 to 91, wherein the epitope conjugated to the T-Cell-MMP has the structure, from N-terminus to C-terminus: (epitope)-(peptide linker)-(optional alkyl linker)-(maleimide) or (epitope)-(peptide linker)-(lysine)-(alkyl linker bound to the epsilon amino group of the lysine)-(maleimide).
93. The T-Cell-MMP-epitope conjugate of any one of aspects 80 to 92, wherein the MHC-H polypeptide comprises a cysteine at positions 84 and 139 (e.g., Y84C and A139C substitutions) that form an intrachain disulfide bond (e.g., stabilizing the protein for expression).
94. A T-Cell-MMP-epitope conjugate of any one of aspects 1 to 93, wherein the T-Cell-MMP-epitope conjugate comprises a structure selected from structures A, B, C, D, E, F, G, H, I, J, K. or L of FIG. 6, wherein the first polypeptide and the second polypeptide are each organized from N-terminus to C-terminus as in the selected structure.
95. The T-Cell-MMP-epitope conjugate of any one of aspects 1 to 92 comprising at least one variant MOD, wherein:
   (a) the T-Cell-MMP-epitope conjugate binds to a first T-cell with an affinity that is at least 25% higher (1.25 times higher) than the affinity with which the T-Cell-MMP-epitope conjugate binds to a second T-cell,
      wherein the first T-cell expresses on its surface a Co-MOD and a TCR that binds the epitope with an affinity of at least 10⁻⁷ M (e.g., 10⁻⁸ or 10⁻⁹ M), and
      wherein the second T-cell expresses on its surface the Co-MOD but does not express on its surface a TCR that binds the epitope with an affinity of at least 10⁻⁷ M (e.g., an affinity less than 10⁻⁷ M, such as 10⁻⁶ or 10⁻⁵ M); or
   (b) wherein the T-Cell-MMP-epitope conjugate binds to a first T-cell with an affinity that is at least 10% (e.g., at least 15%, at least 20%, at least 25%, at least 30%, at least 40%, at least 50%, at least 60%, at least 70%, at least 80%, at least 90%), or at least 2-fold (e.g., at least 2.5-fold, at least 5-fold, at least 10-fold, at least 15-fold, at least 20-fold, at least 25-fold, at least 50-fold, at least 100-fold, or more than 100-fold), higher than the affinity to which it binds the second T-cell,
      wherein the first T-cell displays both i) a TCR specific for the epitope present in the T-Cell-MMP-epitope conjugate, and ii) a Co-MOD that binds to the MOD present in the T-Cell-MMP-epitope conjugate, and
      wherein the second T-cell displays: i) a TCR specific for an epitope other than the epitope present in the T-Cell-MMP-epitope conjugate; and ii) a Co-MOD that binds to the MOD present in the T-Cell-MMP-epitope conjugate.
96. The T-Cell-MMP-epitope conjugate of any one of aspects 1 to 95, wherein the epitope is a peptide that is not post-translationally modified (e.g., it is not a glycopeptide, phosphopeptide, or lipopeptide).
97. The T-Cell-MMP-epitope conjugate of any one of aspects 1 to 95, wherein the epitope is a peptide that has a post-translational modification (e.g., it is a glycopeptide, phosphopeptide, or lipopeptide reflective of having been post-translationally modified).
98. The T-Cell-MMP-epitope conjugate of any one of aspects 1 to 97, wherein the coronavirus peptide epitope comprises 4 to 25 contiguous aas (e.g., a range of 4-10 aas, 7-12 aas, 10-15 aas, 15-20 aas, 20-25 aas, or 5, 6, 7, 8, 9, 10, 11, 12, 13, 14, 15, 16, 17, 18, 19, 20, 21, 22, 23, 24, or 25 contiguous aas) of a protein set forth in FIGS. 11A to 11J, and wherein the T-Cell-MMP-epitope conjugate optionally comprises one or more IL-2 or variant IL-2 MODs having reduced affinity for the IL-2 receptor.
99. The T-Cell-MMP-epitope conjugate of any one of aspects 1 to 97, wherein:
   i) the coronavirus epitope comprises a sequence having 0, 1 or 2 as substitutions, deletions or insertions in an HLA-A*0201 or an HLA-A*2402 restricted epitope peptide set forth in Table 2;
   ii) the first or second polypeptide comprises an MHC-H polypeptide sequence having at least 85% (e.g., at least 90%, at least 95%, at least 97%, at least 98% at least 99% or 100%) sequence identity to a portion (e.g., 20-250, 20-100, 30-100, 40-120, 50-150, 70-170, 100-150, 100-200, 150-200, 200-250 contiguous aas, or more than 250 contiguous aas) or all of an MHC-H chain polypeptide selected from the group consisting of: HLA-A*0201 (SEQ ID NO:23), and HLA-A*2402 (SEQ ID NO:29) (see Table 2); and
   iii) the T-Cell-MMP-epitope conjugate optionally comprises one or more IL-2 or variant IL-2 MODs having reduced affinity for the IL-2 receptor.
100. The T-Cell-MMP-epitope conjugate of any one of aspects 1 to 97, wherein:
   i) the coronavirus epitope comprises a sequence having 0, 1 or 2 as substitutions, deletions or insertions in an HLA-B*4001 or HLA-B*5801 restricted epitope peptide set forth in Table 2;
   ii) the first or second polypeptide comprises an MHC-H polypeptide sequence having at least 85% (e.g., at least 90%, at least 95%, at least 97%, at least 98% at least 99% or 100%) sequence identity to a portion (e.g., 20-250, 20-100, 30-100, 40-120, 50-150, 70-170, 100-150, 100-200, 150-200, 200-250 contiguous aas, or more than 250 contiguous aas) or all of an MHC-H chain polypeptide selected from the group consisting of: HLA-B*4001 (SEQ ID NO:40), HLA-B*5801 (SEQ ID NO:130) (see Table 2); and
   iii) the T-Cell-MMP-epitope conjugate optionally comprises one or more IL-2 or variant IL-2 MODs having reduced affinity for the IL-2 receptor.
101. The T-Cell-MMP-epitope conjugate of any one of aspects 1 to 97, wherein:
   i) the coronavirus epitope comprises a sequence having 0, 1 or 2 as substitutions, deletions or insertions in an HLA*A restricted epitope peptide (i.e., under the heading “Peptide,” “Core,” or “iCore”) set forth in FIG. 15;
   ii) the first or second polypeptide comprises an MHC-H polypeptide sequence having at least 85% (e.g., at least 90%, at least 95%, at least 97%, at least 98% at least 99% or 100%) sequence identity to a portion (e.g., 20-250, 20-100, 30-100, 40-120, 50-150, 70-170, 100-150, 100-200, 150-200, 200-250 contiguous aas, or more than 250 contiguous aas) or all of an MHC-H chain polypeptide selected from the group consisting of: HLA-A*0101 (SEQ ID NO:20), HLA-A*0201 (SEQ ID NO:23), HLA-A*0301 (SEQ ID NO:31), HLA-A*1101 (SEQ ID NO:28), HLA-A*2301 (SEQ ID NO:32), and HLA-A*2402 (SEQ ID NO:29) (see FIG. 15); and
   iii) the T-Cell-MMP-epitope conjugate optionally comprises one or more IL-2 or variant IL-2 MODs having reduced affinity for the IL-2 receptor.
102. The T-Cell-MMP-epitope conjugate of any one of aspects 1 to 97, wherein:
   i) the coronavirus epitope comprises a sequence having 0, 1 or 2 as substitutions, deletions or insertions in an HLA*B restricted epitope peptide set forth in FIG. 15;
   ii) the first or second polypeptide comprises an MHC-H polypeptide sequence having at least 85% (e.g., at least 90%, at least 95%, at least 97%, at least 98% at least 99% or 100%) sequence identity to a portion (e.g., 20-250, 20-100, 30-100, 40-120, 50-150, 70-170, 100-150, 100-200, 150-200, 200-250 contiguous aas, or more than 250 contiguous aas) or all of an MHC-H chain polypeptide selected from the group consisting of HLA-B*0702 (SEQ ID NO:36), HLA-B*0801 (SEQ ID NO:37), HLA-B*3501 (SEQ ID NO:127), HLA-B*4001 (SEQ ID NO:40), HLA-B*4402 (SEQ ID NO:128), and HLA-B*4403 (SEQ ID NO:129) (see FIG. 15); and
   iii) the T-Cell-MMP-epitope conjugate optionally comprises one or more IL-2 or variant IL-2 MODs having reduced affinity for the IL-2 receptor.
103. The T-Cell-MMP-epitope conjugate of any one of aspects 1 to 102, wherein the T-Cell-MMP-epitope conjugate is in the form of a dimer.
104. The T-Cell-MMP-epitope conjugate of aspect 103, wherein the T-Cell-MMP-epitope conjugate comprises an (Ig) Fc polypeptide or a non-Ig polypeptide scaffold through which the T-Cell-MMP-epitope conjugate dimerizes, wherein the dimer is optionally stabilized by one or two disulfide bonds between the Ig Fc or non-Ig polypeptide chains.
105. The T-Cell-MMP-epitope conjugate of any one of aspects 1 to 104, further comprising one or more independently selected payloads covalently bound to one or more first and/or second chemical conjugation sites either directly or indirectly through a spacer or linker, wherein the spacer or linker is optionally cleavable (e.g., in an endosome of a mammalian cell).
106. The T-Cell-MMP-epitope conjugate of aspect 105, wherein the payload is conjugated via a linker having from 1 to 20 (e.g., 1-2, 2-4, 5-10 or 10-20) independently selected alpha, beta, delta, or gamma amino acids, or a combination thereof; or wherein the linker is a peptide of the formula poly-glycine poly-alanine, a random poly-(glycine/alanine) copolymer, or poly(GGGGS)n where n is 1, 2, 3, 4, 5, 6, 7,or 8.
107. The T-Cell-MMP-epitope conjugate of aspect 105, wherein the payload is attached to a chemical conjugations site by a spacer, wherein the spacer results from the action of a homofunctional (e.g., homobifunctional) crosslinker or a heterofunctional (e.g., heterobifunctional) crosslinker.
108. The T-Cell-MMP-epitope conjugate of any one of aspects 1 to 107, wherein the chemical conjugation site to which the epitope is covalently bound to create the T-Cell-MMP-epitope conjugate is not located in or immediately adjacent to (within one amino acid) an amino acid sequence having 100% amino acid identity to:
   the Fc polypeptide sequence in FIGS. 2A-2G;
   the MHC Class I heavy chain polypeptide sequences in FIGS. 3A-3H; or
   the β-2 microglobulin polypeptide sequences in FIG. 4.
109. The T-Cell-MMP-epitope conjugate of any one of aspects 1 to 107, wherein the chemical conjugation site to which the epitope is covalently bound to create the T-Cell-MMP-epitope conjugate is not located in or immediately adjacent to (within one amino acid) a 10, 20, 30, 40, or 50 amino acid long sequence having 100% amino acid identity to any portion of any one of:
   the Fc polypeptide sequence in FIGS. 2A-2G;
   the MHC Class I heavy chain polypeptide sequences in FIGS. 3A-3H; or
   the β-2 microglobulin polypeptide sequences in FIG. 4.
110. The T-Cell-MMP-epitope conjugate of any one of aspects 1 to 107, wherein the chemical conjugation site to which the epitope is covalently bound to create the T-Cell-MMP-epitope conjugate is not an amino acid appearing in a 5, 6, 7, 8, 9, 10, 20, 30, 40, 50, 60, or 70 amino acid long sequence having 100% amino acid identity to any portion of any one of:
   the Fc polypeptides in FIGS. 2A-2G;
   the MHC Class I heavy chain polypeptide sequences in FIGS. 3A-3H; or
   the β-2 microglobulin polypeptide sequences in FIG. 4.
111. The T-Cell-MMP-epitope conjugate of any one of aspects 1 to 107, wherein the chemical conjugation site to which the epitope is covalently bound to create the T-Cell-MMP-epitope conjugate is not a lysine, cysteine, serine, threonine, arginine, aspartic acid, glutamic acid, asparagine, or glutamine located in a 10, 20, 30, 40, 50, 60, or 70 amino acid long sequence having 100% amino acid identity to any portion of any one of:
   the Fc polypeptide sequence in FIGS. 2A-2G;
   the MHC Class I heavy chain polypeptide sequences in FIGS. 3A-3H; or
   the β-2 microglobulin polypeptide sequences in FIG. 4.
112. A composition comprising a T-Cell-MMP-epitope conjugate of any one of aspects 1-107.
113. A composition (formulation) comprising:
   a) the T-Cell-MMP-epitope conjugate of any one of aspects 1 to 107; and
   b) a pharmaceutically acceptable excipient.
114. A method of delivering an immunomodulatory polypeptide (MOD) to a target T-cell (e.g., a regulatory T-cell or cytotoxic T-cell) in an epitope-selective or epitope-selective/specific manner in vitro, or to an individual in vivo, comprising:
   contacting a T-Cell-MMP-epitope conjugate of any one of aspects 1-111 with the target T-cell or a mixed population of T-cells comprising the target T-cell in vitro; or
   administering the T-Cell-MMP-epitope conjugate of any one of aspects 1-111 or a composition comprising the T-Cell-MMP-epitope conjugate of any of aspects 112-113 to the individual (e.g., patient or subject);
   wherein the target T-cells are specific for the epitope present in the T-Cell-MMP-epitope conjugate.
115. A method of modulating the activity (e.g., activation or proliferation) of a target T-cell (e.g., a regulatory T-cell or cytotoxic T-cell) in an epitope-selective or epitope-selective/specific manner in vitro, or in an individual in vivo, comprising:
   contacting a T-Cell-MMP-epitope conjugate of any one of aspects 1-111 with the target T-cell or a mixed population of T-cells comprising the target T-cell in vitro; or
   administering the T-Cell-MMP-epitope conjugate of any one of aspects 1-111 or a composition comprising the T-Cell-MMP-epitope conjugate of any one of aspects 112-113 to the individual (e.g., patient or subject);
   wherein the target T-cells are specific for the epitope present in the T-Cell-MMP-epitope conjugate.
116. The method of aspect 115, wherein said modulating comprises increasing a cytotoxic T-cell response to a coronavirus-infected cell.
117. A method of treating a patient having a coronavirus-infection, the method comprising administering to the patient an effective amount of composition according to any one of aspects 112 to 113. 118. The method of aspect 117, wherein the coronavirus infection is selected from SARS-CoV, SARS-CoV-2 (Covid-19), and/or MERS.
119. The method of aspect 118, wherein the coronavirus infection is SARS-CoV and/or SARS-CoV-2 (Covid-19).
120. The method of any one of aspects 114 to 119, wherein the patient is a human.
121. The method of any one of aspects 114 to 120, wherein said administering is intramuscular or intravenous.
122. The method of any one of aspects 114 to 120, wherein said administering is rectal, nasal, oral, other enteral and/or parenteral routes of administration.
123. The method of any one of aspects 114 to 120, wherein said administering is intratumoral, peritumoral, intramuscular, intratracheal, intracranial, subcutaneous, intralymphatic, intradermal, topical, intravenous, and/or intraarterial.
124. A method of modulating an immune response in an individual, the method comprising administering to the individual an effective amount of the T-cell MMP-epitope conjugate of any one of aspects 1 to 111, wherein said administering induces an epitope-specific T cell response (e.g., a T cell response specific for the coronavirus epitope present in the T-cell-MMP-epitope conjugate) and an epitope-non-specific T cell response, wherein the ratio of the epitope-specific T cell response to the epitope-non-specific T cell response is at least 2:1.
125. The method of aspect 124, wherein the ratio of the epitope-specific T cell response to the epitope-non-specific T cell response is at least 5:1.
126. The method of aspect 124, wherein the ratio of the epitope-specific T cell response to the epitope-non-specific T cell response is at least 10:1.
127. The method of aspect 124, wherein the ratio of the epitope-specific T cell response to the epitope-non-specific T cell response is at least 25:1.
128. The method of aspect 124, wherein the ratio of the epitope-specific T cell response to the epitope-non-specific T cell response is at least 50:1 or at least 100:1.
129. The method of any one of aspects 124 to 128, wherein the individual is a human.
130. The method of any one of aspects 124 to 129, wherein said modulating comprises increasing a cytotoxic T-cell response to a coronavirus infected cell.
131. The method of any one of aspects 124 to 130, wherein said administering is intravenous, subcutaneous, intramuscular, systemic, intralymphatic, distal to a treatment site, local, or at or near a treatment site.
132. The method of any one of aspects 124 to 131, wherein the epitope non-specific T-cell response is less than the epitope non-specific T-cell response that would be induced by a control T-cell MMP-epitope conjugate comprising a corresponding wild-type immunomodulatory polypeptide.
133. A method of detecting, in a mixed population of T cells obtained from an individual, the presence of a target T cell that binds a coronavirus epitope of interest, the method comprising: a) contacting in vitro the mixed population of T cells with the T-cell MMP-epitope conjugate of any one of aspects 1 to 111, wherein the T-Cell-MMP-epitope conjugate comprises the coronavirus epitope of interest; and b) detecting activation and/or proliferation of T cells in response to said contacting, wherein activated and/or proliferated T cells indicate the presence of the target T cell.
134. A polypeptide construct comprising a polypeptide comprising a mature β2M polypeptide sequence (lacking its signal sequence) having an N-terminus and a C-terminus and at least one optional linker and a coronavirus peptide epitope that comprises 6 or more contiguous amino acids of a coronavirus sequence set forth in any one of FIGS. 11A to 11J.
135. The polypeptide of aspect 134, further comprising one or more chemical conjugation sites within or at the ends of the sequence of the mature β2M polypeptide, or covalently attached to the mature β2M polypeptide via the optional linker, wherein the coronavirus peptide epitope is covalently bound, directly or indirectly, to at least one of the one or more chemical conjugation sites.
136. The polypeptide of aspect 134 or 135, wherein the mature β2M polypeptide has a sequence with at least 85% (e.g., at least 90%, 95%, 98% or 99% identity, or even 100%) amino acid sequence identity to at least 60 contiguous aas (at least 70, 80, 90 or all aas) of a mature β2M polypeptide provided in FIG. 4.
137. The polypeptide of any one of aspects 134 to 136, wherein the β2M polypeptide sequence comprises, consists essentially of, or consists of a sequence of at least 20, 30, 40, 50, 60, 70, 80, 90 or 99 contiguous amino acids having identity with at least a portion of one of the amino acid sequences set forth in FIG. 4 (e.g., a sequence having 20-99, 20-40, 30-50, 40-60, 40-90, 50-70, 60-80, 60-99, 70-90, or 79-99 contiguous amino acids with identity to a sequence of mature β2M lacking its signal sequence set forth in FIG. 4).
138. The polypeptide of any one of aspects 134 to 137, wherein the β2M polypeptide sequence comprises a cysteine at one, two or more of amino acid positions 2, 44, 50, 77, 85, 88, 91 or 98 (e.g., a Q2C, E44C, E50C, E77C, V85V, S88C, K91C, or D98C substitution) of the mature β2M polypeptide sequence.
139. The polypeptide of aspect 138, wherein the first 12 amino acids of the β2M polypeptide sequence are IQRTPKIQVYSC.
140. The polypeptide of any one of aspects 135 to 137, wherein the chemical conjugation site is a sulfatase motif.
141. The polypeptide of aspect 140, wherein the sulfatase motif is linked directly, or indirectly via a linker, to the N-terminus of the β2M polypeptide sequence.
142. The polypeptide of any one of aspects 140 to 141, comprising a sulfatase motif, or a sulfatase motif wherein serine or cysteine of the sulfatase motif has been converted to an fGly (formylglycine) residue.
143. The polypeptide of aspect 142, wherein the coronavirus peptide epitope is covalently bound to the polypeptide through a chemical reaction with the fGly residue (e.g., the reaction of a thiosemicarbazide, aminooxy, hydrazide, or hydrazino modified coronavirus epitope polypeptide with the aldehyde of the fGly).
144. The polypeptide of any one of aspects 134 to 143, further comprising a signal sequence, or a signal sequence and a linker, wherein the signal sequence is the amino-terminal most element of the polypeptide.
145. A composition comprising a polypeptide of any one of aspects 134 to 144 optionally comprising a pharmaceutically acceptable carrier.
146. A polypeptide construct comprising:
   a mature MHC Class I heavy chain polypeptide sequence (lacking its signal sequence) and an optional linker;
   a coronavirus polypeptide that comprises 6 or more contiguous amino acids of a coronavirus sequence set forth in FIGS. 11A to 11J; and
   optionally an immunoglobulin (Ig) Fc polypeptide or a non-Ig polypeptide scaffold.
147. The polypeptide of aspect 146, further comprising one or more chemical conjugation sites within or at the ends of the sequence of the mature MHC Class I heavy chain polypeptide, or covalently attached to the mature MHC Class I heavy chain polypeptide via the optional linker, wherein the coronavirus peptide epitope is covalently bound, directly or indirectly, to at least one of the one or more chemical conjugation sites.
148. The polypeptide of aspect 146 or 147, wherein the MHC Class I heavy chain polypeptide has a sequence with at least 85% (e.g., at least 90%, 95%, 98%, 99%, or even 100%) amino acid sequence identity to any of the sequences provided in FIGS. 3D-3H; wherein identity between the MHC Class I heavy chain polypeptide and the corresponding sequences in FIGS. 3D-3H is determined without consideration of the (Ig) Fc polypeptide and any optional linker present.
149. The polypeptide of any one of aspects 146 to 148, wherein the MHC Class I heavy chain polypeptide comprises, consists essentially of, or consists of a sequence of at least 20, 30, 40, 50, 60, 70, 80, 90 or 100 contiguous amino acids having identity with at least a portion of one of the amino acid sequence set forth in any of FIGS. 3D-3H (e.g., a sequence having 20-100, 20-40, 30-50, 40-60, 40-90, 50-70, 60-80, 60-90, 70-90, or 80-100 contiguous amino acids with identity to an MHC Class I heavy chain polypeptide sequence set forth in any of FIGS. 3D-3H).
150. The polypeptide of aspect 149, wherein the MHC Class I heavy chain polypeptide comprises one, two or three sequences selected from the group consisting of:
   i) a sequence from about amino acid position 79 to about amino acid position 89;
   ii) a sequence from about amino acid position 134 to about amino acid position 144; and
   iii) a sequence from about amino acid position 231 to about amino acid position 241 of the MHC Class I heavy chain sequences set forth in any of FIGS. 3D-3H.
151. The polypeptide of aspect 150, wherein the MHC Class I heavy chain polypeptide comprises:
   i) the sequence from about amino acid position 79 to about amino acid position 89; and
   ii) the sequence from about amino acid position 134 to about amino acid position 144;
   wherein one of positions 83, 84, or 85 has been substituted with a cysteine that forms an intrachain disulfide bond with a cysteine substituted at one of positions 138, 139, or 140. 152. The polypeptide of any one of aspects 150 to 151, wherein the polypeptide comprises an MHC Class I heavy chain polypeptide sequence from about amino acid position 231 to about amino acid position 241 of the MHC Class I heavy chain sequences set forth in any of FIGS. 3D-3H wherein one of positions 235, 236 or 237 has been substituted by a cysteine.
153. The polypeptide of any one of aspects 146 to 152, wherein any one or more of the linkers is selected independently from peptides of formula (AAAGG)n or (GGGGS)n, where n is from 1 to 8 (e.g., 1, 2, 3, 4, 5, 6, 7, or 8, or in a range selected from 1 to 4, 3 to 6, or 4 to 8).
154. A composition comprising a polypeptide of any one of aspects 146 to 153.
155. A composition comprising a polypeptide of any one of aspects 146 to 153 and a pharmaceutically acceptable carrier.
156. A T-cell modulatory multimeric polypeptide epitope conjugate (T-Cell-MMP-epitope conjugate) comprising:
   a) a first polypeptide having an N-terminus and a C-terminus, the first polypeptide comprising,
      i) a beta-2-microglobulin (“β2M”) polypeptide having an N-terminus and a C-terminus, and an optional linker at the N-terminus and/or the C-terminus;
   b) a second polypeptide having an N-terminus and a C-terminus, the second polypeptide comprising,
      i) an MHC-H polypeptide;
      ii) an immunoglobulin (Ig) Fc polypeptide or a non-Ig polypeptide scaffold, and an optional linker at the N-terminus and/or the C-terminus of the second polypeptide;
   c) one or more first polypeptide chemical conjugation sites attached to or within the first polypeptide, and/or one or more second polypeptide chemical conjugation sites attached to or within the second polypeptide; and
   d) one or more immunomodulatory polypeptides (MODs), wherein at least one of the one or more MODs is
      A) at the C-terminus of the first polypeptide,
      B) at the N-terminus of the second polypeptide,
      C) at the C-terminus of the second polypeptide,
      D) at the C-terminus of the first polypeptide and at the N-terminus of the second polypeptide or
      E) within the first or second polypeptide; and
   e) a coronavirus peptide epitope covalently bound, directly or indirectly, to at least one of the one or more first polypeptide chemical conjugation sites or the one or more second polypeptide chemical conjugation sites, the coronavirus peptide epitope comprising six (6) or more (e.g., 8 or more) contiguous amino acids of a coronavirus protein selected from the group consisting of: surface glycoprotein; membrane protein; nucleocapsid phosphoprotein; open reading frame (Orf) 1b protein; Orf3a protein; Orf6 protein; Orf7a protein; Orf8 protein; Orf10 protein; envelope protein; membrane glycoprotein; nonstructural protein (nsp) one (nsp1); nsp2; nsp4; nsp6; nsp7, nsp8, nsp9, nsp10; nsp14; nsp15; nsp16; nucleocapsid phosphoprotein; papain-like cysteine protease (PLpro); 3C-like protease (3CL); RNA-dependent RNA polymerase (RDpol) and helicase (Hel);
   wherein each of the one or more MODs is an independently selected wild-type or variant MOD;
   wherein the MHC-H polypeptide sequence has at least 90% sequence identity to at least 200 contiguous aas of an MHC-H chain polypeptide selected from the group consisting of: HLA-A*0101 (SEQ ID NO:20), HLA-A*0201 (SEQ ID NO:23), HLA-A*0301 (SEQ ID NO:31), HLA-A*1101 (SEQ ID NO:28), HLA-A*2301 (SEQ ID NO:32), HLA-A*2402 (SEQ ID NO:29), HLA-A*2407 (SEQ ID NO:33), HLA-A*3303 (SEQ ID NO:30), HLA-A*3401 (SEQ ID NO:34), HLA-B*0702 (SEQ ID NO:36), HLA-B*0801 (SEQ ID NO:37), HLA-B*1502 (SEQ ID NO:38), HLA-B*3501 (SEQ ID NO:127), HLA-B*3802 (SEQ ID NO:39), HLA-B*4001 (SEQ ID NO:40), HLA-B*4402 (SEQ ID NO:128), HLA-B*4403 (SEQ ID NO:129), HLA-B*4601 (SEQ ID NO:41), HLA-B*5301 (SEQ ID NO:42), HLA-B*5801 (SEQ ID NO:130), HLA-C*0102, (SEQ ID NO:44), HLA-C*0303 (SEQ ID NO:45), HLA-C*0304 (SEQ ID NO:46), HLA-C*0401 (SEQ ID NO:47), HLA-C*0602 (SEQ ID NO:48), HLA-C*0701 (SEQ ID NO:49), HLA-C*0702 (SEQ ID NO:50), HLA-C*0801 (SEQ ID NO:51), and HLA-C*1502 (SEQ ID NO:52, an HLA-E polypeptide (SEQ ID NO: 54), an HLA-F polypeptide (SEQ ID NO: 55), and an HLA-G polypeptide (SEQ ID NO:56); and
   wherein optionally substantially all of the T-Cell-MMP-epitope conjugate is in the form of a dimer comprising a first T-cell-MMP-epitope conjugate and a second T-Cell-MMP-epitope conjugate associated by covalent and/or non-covalent interactions between their polypeptide scaffold sequences.
157. The T-Cell-MMP-epitope conjugate of aspect 156,
   wherein the coronavirus peptide epitope comprises six (6) or more (e.g., 8 or more) contiguous amino acids of a coronavirus protein selected from the group consisting of surface glycoprotein, membrane protein, nucleocapsid phosphoprotein, and open reading frame (Orf) 1b protein;
   wherein each of the one or more MODs is an independently selected wild-type or variant MOD; and
   wherein the MHC-H polypeptide sequence has at least 90% sequence identity to at least 200 contiguous aas of an MHC-H chain polypeptide selected from the group consisting of: HLA-A*0201 (SEQ ID NO:23), HLA-A*2402 (SEQ ID NO:29), HLA-A*1101 (SEQ ID NO:28), HLA-B*4001 (SEQ ID NO:40), and HLA-B*5801 (SEQ ID NO:130).
158. The T-Cell-MMP-epitope conjugate of aspect 156 or aspect 157, wherein the peptide is selected from the group consisting of: GLMWLSYFV, TLACFVLAAV, ALNTPKDHI, GMSRIGMEV, LALLLLDRL, LLLDRLNQL, LQLPQGTTL, RLNQLESKV, ALSGVFCGV, CLDAGINYV, ILLLDQVLV, LLCVLAALV, SMWALVISV, TLMNVITLV, WLMWFISI, ALNTLVKQL, FIAGLIAIV, KLPDDFMGCV, LITGRLQSL, NLNESLIDL, RLNEVAKNL, SIVAYTMSL, VLNDILSRL, QFKDNVILL, GETALALLLL, MEVTPSGTWL, RFFTLGSITAQPVKI, and SITAQPVKI.
158. The T-Cell-MMP-epitope conjugate of aspect 156, wherein the coronavirus peptide epitope comprises six (6) or more (e.g., 8 or more) contiguous amino acids of a coronavirus protein selected from the group consisting of: Orf3a protein; Orf6 protein; Orf7a protein; Orf8 protein; Orf10 protein; envelope protein; membrane glycoprotein; nonstructural protein (nsp) one (nsp1); nsp2; nsp4; nsp6; nsp7, nsp8, nsp9, nsp10; nsp14; nsp15; nsp16; nucleocapsid phosphoprotein; papain-like cysteine protease (PLpro); 3C-like protease (3CL); RNA-dependent RNA polymerase (RDpol) and helicase (Hel).
159. The T-Cell-MMP-epitope conjugate of aspect 158, wherein the coronavirus peptide epitope comprises six (6) or more, or eight (8) or more, contiguous amino acids of any one of the peptides set forth in FIG. 15 and/or Table 2.
160. The T-Cell-MMP-epitope conjugate of aspect 159, wherein the MHC-H polypeptide sequence has at least 90% sequence identity to 200 aas of an MHC-H chain polypeptide selected from the group consisting of: HLA-A*0101 (SEQ ID NO:20), HLA-A*0201 (SEQ ID NO:23), HLA-A*0301 (SEQ ID NO:31), HLA-A*1101 (SEQ ID NO:28), HLA-A*2301 (SEQ ID NO:32), HLA-A*2402 (SEQ ID NO:29), HLA-B*0702 (SEQ ID NO:36), HLA-B*0801 (SEQ ID NO:37), HLA-B*3501 (SEQ ID NO:127), HLA-B*4001 (SEQ ID NO:40), HLA-B*4402 (SEQ ID NO:128), HLA-B*4403 (SEQ ID NO:129), and HLA-B*5801 (SEQ ID NO:130).
161. The T-Cell-MMP-epitope conjugate of any one of aspects 156 to 160, wherein the one or more MODs are wild type MODs or variant MODs selected independently from the group consisting of IL-2, 4-1BBL, PD-L1, CD70, CD80, CD86, ICOS-L, OX-40L, FasL, JAG1, TGF-β, ICAM, and PD-L2, and variants thereof.
162. The T-Cell-MMP-epitope conjugate of any one of aspects 156 to 161, wherein the one or more MODs are wild type MODs or variant MODs selected independently from the group consisting of IL-2, 4-1BBL, CD80, CD86, and variants thereof.
163. The T-Cell-MMP-epitope conjugate of any one of aspects 156 to 162, wherein the second polypeptide comprises an immunoglobulin (Ig) Fc polypeptide scaffold.
164. The T-Cell-MMP-epitope conjugate of any one of aspects 156 to 163, wherein the first MHC polypeptide comprises:
   a β2M polypeptide bearing a linker on its N-terminus,
   a β2M polypeptide bearing a linker on its C-terminus, or
   a β2M polypeptide bearing a linker on its N-terminus and C-terminus.
165. The T-Cell-MMP-epitope conjugate of any one of aspects 156 to 164, wherein the first and second chemical conjugation sites are independently selected from:
   a) amino acid chemical conjugation sites;
   b) non-natural amino acids and/or selenocysteines;
   c) peptide sequences that act as enzymatic modification sequences;
   d) carbohydrate or oligosaccharide moieties; and/or
   e) IgG nucleotide binding sites.
166. The T-Cell-MMP-epitope conjugate of any one of aspects 156 to 165, wherein the coronavirus peptide epitope is covalently bound, directly or indirectly through a linker to the β2M polypeptide sequence.
167. The T-Cell-MMP-epitope conjugate of any one of aspects 156 to 166, wherein the coronavirus peptide is bound to an amino acid chemical conjugation site.
168. The T-Cell-MMP-epitope conjugate of aspect 167, wherein the amino acid chemical conjugation site to which the epitope is attached is a cysteine engineered into the β2M polypeptide or an amino acid of a linker at the N-terminus of the β2M polypeptide.
169. The T-Cell-MMP-epitope conjugate of aspect 168, wherein the epitope is attached to a cysteine engineered into the β2M polypeptide sequence as a substitution selected from Q2C, E44C, E50C, E77C, V85V, S88C, K91C, and/or D98C; and wherein the β2M polypeptide has a sequence with at least 90% sequence identity to at least 80 contiguous amino acids of a mature β2M polypeptide set forth in any of SEQ ID NOs: 57-61.
170. The T-Cell-MMP-epitope conjugate of any one of aspects 156 to 169, wherein the coronavirus peptide epitope comprises from 6 to 25 contiguous aas, or 8 to 16 contiguous aas, of one coronavirus protein, and wherein the T-Cell-MMP-epitope conjugate optionally comprises one or more IL-2 or variant IL-2 MODs having reduced affinity for the IL-2 receptor.
171. The T-Cell-MMP-epitope conjugate of any one of aspects 1-3, wherein:
   i) the coronavirus epitope comprises a sequence having 0, 1 or 2 aa substitutions, deletions or insertions in an HLA*A restricted epitope peptide selected from the group consisting of GLMWLSYFV, TLACFVLAAV, ALNTPKDHI, GMSRIGMEV, LALLLLDRL, LLLDRLNQL, LQLPQGTTL, RLNQLESKV, ALSGVFCGV, CLDAGINYV, ILLLDQVLV, LLCVLAALV, SMWALVISV, TLMNVITLV, WLMWFIISI, ALNTLVKQL, FIAGLIAIV, KLPDDFMGCV, LITGRLQSL, NLNESLIDL, RLNEVAKNL, SIVAYTMSL, VLNDILSRL, and QFKDNVILL;
   ii) the MHC-H polypeptide sequence has at least 90% (e.g., at least 95%, at least 97%, at least 98% at least 99% or 100%) sequence identity to a portion (e.g., 200-250 contiguous aas, or more than 250 contiguous aas) or all of an MHC-H chain polypeptide selected from the group consisting of: HLA-A*0201 (SEQ ID NO:23), and HLA-A*2402 (SEQ ID NO:29); and
   iii) the T-Cell-MMP-epitope conjugate optionally comprises one or more IL-2 or variant IL-2 MODs having reduced affinity for the IL-2 receptor.
172. The T-Cell-MMP-epitope conjugate of any one of aspects 156 to 158, wherein:
   i) the coronavirus epitope comprises a sequence having 0, 1 or 2 aa substitutions, deletions or insertions in an HLA*B restricted epitope peptide selected from the group consisting of GETALALLLL, MEVTPSGTWL, RFFTLGSITAQPVKI, and SITAQPVKI;
   ii) the MHC-H polypeptide sequence has at least 90% (e.g., at least 95%, at least 97%, at least 98% at least 99% or 100%) sequence identity to a portion (e.g., 200-250 contiguous aas, or more than 250 contiguous aas) or all of an MHC-H chain polypeptide selected from the group consisting of: HLA-B*4001 (SEQ ID NO:40) and HLA-B*5801 (SEQ ID NO:130); and
   iii) the T-Cell-MMP-epitope conjugate optionally comprises one or more IL-2 or variant IL-2 MODs having reduced affinity for the IL-2 receptor.
173. The T-Cell-MMP-epitope conjugate of any one of aspects 156 to 172, comprising one or more, or two or more, IL-2 and/or variant IL-2 MODs having reduced affinity for the IL-2 receptor.
174. A T-Cell-MMP-epitope conjugate of any one of aspects 156 to 173, wherein the T-Cell-MMP-epitope conjugate has a structure selected from structure A, B, C, D, E, F, G, H, I, J, K, or L of FIG. 6.
175. The T-Cell-MMP-epitope conjugate of any one of aspects 156 to 174, wherein the T-Cell-MMP-epitope conjugate forms a dimer through covalent or non-covalent interactions between the (Ig) Fc polypeptide or the non-Ig polypeptide scaffold.
176. A composition comprising:
   a) the T-Cell-MMP-epitope conjugate of any one of aspects 156 to 175; and
   b) a pharmaceutically acceptable excipient.
177. The use of the T-Cell-MMP-epitope conjugate of any one of aspects 156 to 175, for the manufacture of a medicament for administering to an individual in need thereof an effective amount of the T-Cell-MMP-epitope conjugate.
178. The use of a T-Cell-MMP-epitope conjugate of any one of aspects 156 to 175 for the manufacture of a medicament for use in a method of delivering an immunomodulatory polypeptide (MOD) to a target T-cell in an epitope-selective or epitope-selective/specific manner in vitro, or to an individual in vivo, comprising:
   contacting the medicament with the T-Cell in vitro, or
   administering the medicament to the individual;
   wherein the target T-cells are specific for the epitope present in the T-Cell-MMP-epitope conjugate.
179. A method of treating a patient or individual, the method comprising administering to the patient or individual an effective amount of the T-Cell-MMP-epitope conjugate of any one of aspects 156 to 175. 180. The method of aspect 179, wherein the patient or individual is being treated for a SARS CoV infection and/or SARS-CoV-2 (Covid-19) infection.
   The subject matter of this disclosure including any of aspects 1 to 180 may be subject to the proviso that the T-Cell-MMP-epitope conjugates do not comprise an MHC-H polypeptide explicitly disclosed (e.g., as a sequence) in International Appln. PCT/US2018/049803, which published as WO 2019/051127. The subject matter of this disclosure including any of aspects 1 to 180 may be subject to the proviso that the T-Cell-MMP-epitope conjugates do not include or comprise a T-Cell-MMP and/or T-Cell-MM-epitope conjugate disclosed in International Appln. PCT/US2018/049803.

### XI. Examples

### Example 1

This example describes and provides for the preparation of a T-Cell-MMP having a first polypeptide (see FIG. 9 A) containing a sulfatase motif (bolded but not underlined) that can be acted on by an FGE to provide a fGly chemical conjugation site and a second polypeptide. The first and second polypeptides taken together form a T-Cell-MMP into which an epitope can be conjugated, and which can be dimerized through the IgFc regions. At B, FIG. 9 shows a second polypeptide of a T-Cell-MMP having tandem IL-2 MODs attached to the amino end of a human MHC Class I HLA-A heavy chain polypeptide followed by a human IgG1 Fc polypeptide.

The polypeptides are prepared by assembling the coding sequences of the first and second polypeptides in expression cassettes that include constitutive or inducible promoter elements for driving the expression of mRNA molecules encoding the first and second polypeptides along with polyadenylation and stop codons. The expression cassettes are assembled into separate vectors (plasmid, viral etc.), or a single vector, for transient expression from a suitable cell line (e.g., CHO, HEK, Vero, COS, yeast etc.). Alternatively, the assembled cassettes are stably integrated into such cells for constitutive or induced expression of the first and second polypeptides.

1A. First Polypeptides

The first polypeptide of this example comprises from the N-terminus to the C-terminus a) a leader sequence, b) a sulfatase motif to introduce an fGly chemical coupling site, c) an optional linker, and d) a β2M polypeptide. Following the action of a FGE, the first peptide has a cysteine in the motif converted to a formylglycine (fGly) residue.

Within the above-mentioned first peptide, the first 20 aas serve as the signal sequence and are removed during cellular processing during maturation of the polypeptide. The residues of the sulfatase motif (X1, Z1, X2, Z2, X3, and Z3), here LCTPSR, are described in Section I.A above flanked by the linker sequence GGGGS (SEQ ID NO:92) to emphasize that linkers may be placed before and/or after the motif. The map also indicates by double underlining the location of a potential amino acid substitution at position 12 in the β2M polypeptide changing an arginine to a cysteine (R12C).

1B. Second Polypeptides

The second polypeptide of this example comprises from N-terminus to C-terminus a) a leader sequence, b) a MOD polypeptide(s), c) an optional linker, d) an MHC Class 1 heavy chain polypeptide, e) an optional linker, and f) an immunoglobulin Fc region.

The mRNAs encode the second polypeptide polypeptides having the overall structure: signal sequence-linker-tandem IL-2 (IL2 polypeptide-optional linker-IL2 polypeptide)-linker-MHC Class 1 heavy chain polypeptide-linker-immunoglobulin heavy chain Fc polypeptide where the signal sequence is a 20 aa human IL2 signal sequence. The polypeptide also contains a human HLA-A polypeptide and a human IgG1 Fc polypeptide. Indicated below the map are the locations of potential amino acid substitutions including the location of the Y84C, A139C, and the A236C cysteine substitutions. The Y84C and A139C substitutions permit a stabilizing disulfide bond to form between the region near the carboxyl end of the HLA α1 helix and the region around the amino terminus of the HLA α2-1 helix. The cysteine resulting from the A236C substitution can form an interchain disulfide bond with a cysteine at, for example, position 12 of the β2M polypeptide in the first polypeptide. Below the map appears an exemplary peptide sequence for a second polypeptide including the leader sequence.

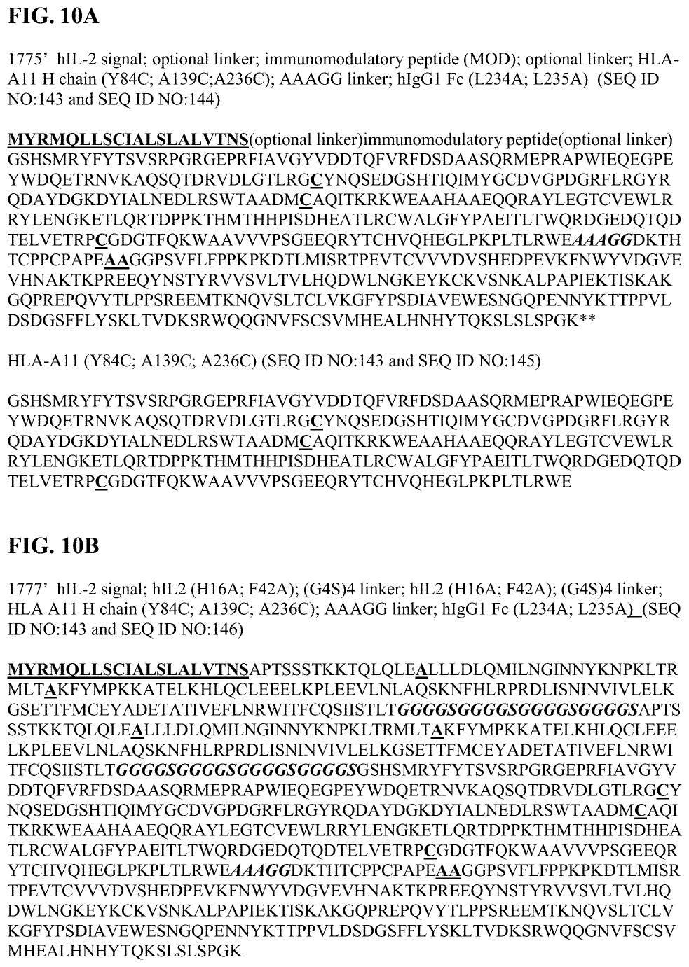

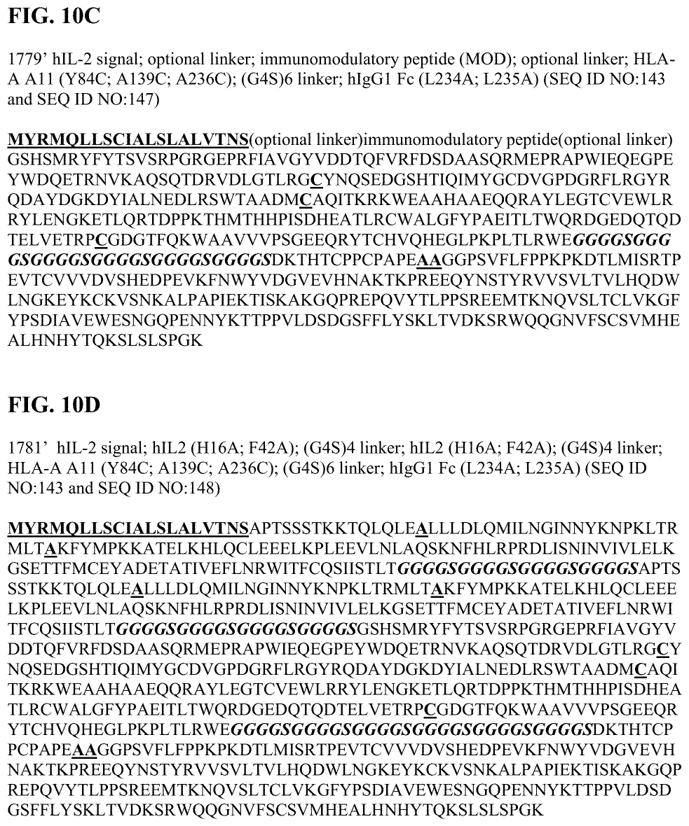

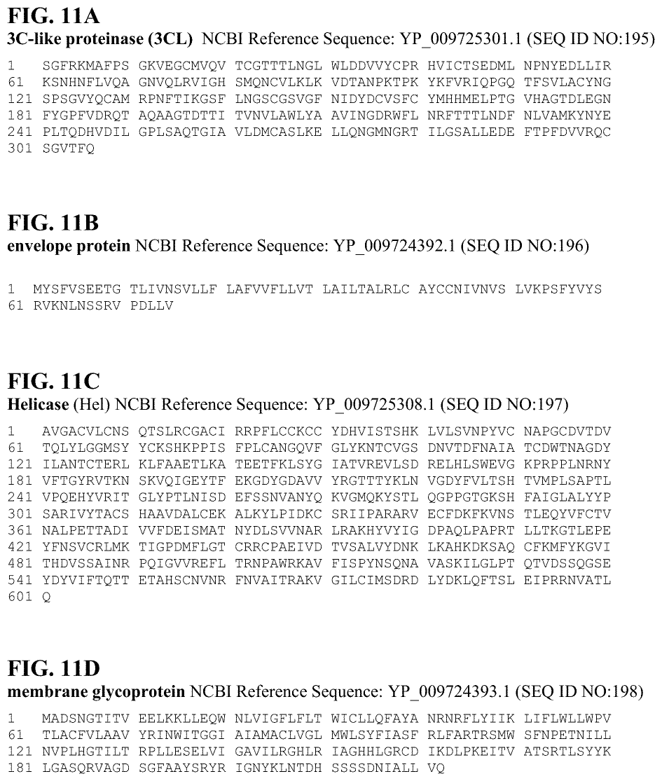

Additional polypeptides that could be used to prepare T-Cell-MMPs and their epitope conjugates are provided in FIG. 10. In addition, although exemplified principally with the A11 MHC Class I heavy chain HLA-A*1101, any of the other MHC heavy chains set forth in FIGS. 3A to 3D, or portions thereof, could have been employed, including, but not limited to, HLA-A*0201; HLA-A*2401; HLA-A*2402 (SEQ ID NO:16) and HLA-A*3303 (SEQ ID NO:17).

1C. Expression and Maturation of the First Second Polypeptides

As indicated above, first and second polypeptides are prepared by transient or stable expression in a suitable cell line (e.g., a eukaryotic or mammalian cell line). Processing in the cell removes the signal sequence and forms a fGly residue when the cells employed for polypeptide expression also express an FGE that is capable of converting a cysteine or serine of the sulfatase motif to a formylglycine (fGly) residue.

T-Cell-MMPs can be processed by cells as a complex that includes the first and second polypeptides and a bound (non-covalently associated) epitope or null polypeptide. The introduction of the disulfide bond in the HLA heavy chain polypeptide between the region at the carboxyl end of the α1 helix and the region at the amino terminus of the α2-1 helix permits expression in the absence of an epitope polypeptide associated with the first and second polypeptides. In addition, as the T-Cell-MMP complexes do not contain a membrane anchor region, the complex is released from the expressing cell in soluble form.

Cell culture media containing the expressed T-Cell-MMP is collected after suitable levels of the expressed T-Cell-MMP have been attained. Where the cells used for expression did not have FGE activity, the T-Cell-MMPs are treated with an FGE capable of forming the fGly residue at the sulfatase motif. Isolation and concentration of the T-Cell-MMP from the media (e.g., serum free media) is conducted using, for example, chromatographic methods to produce a purified T-Cell-MMP having a fGly chemical conjugation site at or near the amino terminus of the first polypeptide of the complex. The resulting T-Cell-MMP has the general structure shown in FIG. 5, part B, where the MHC-1 in the first polypeptide is the β2M polypeptide, the second polypeptide “MOD” is the pair of IL2 polypeptides, the MHC-2 is an HLA-A polypeptide, and Fc is a IGg1 heavy chain constant region. The disulfide bond between the first and second polypeptides results from the cysteines arising from the β2M polypeptide R12C and HLA-A A236C substitutions.

1.D. Preparation of T-Cell-MMP-Epitope Conjugates

Epitope polypeptides are conjugated to the fGly-containing T-Cell-MMP prepared above by forming on the epitope peptide a group capable of reacting with the fGly aldehyde. While thiosemicarbazide, aminooxy, hydrazide, or hydrazino aldehyde reactive groups can be utilized, this example is illustrated by the use of a hydrazinyl group (e.g., attached to an indole) where the epitope peptide is covalently bound, directly or indirectly, to the nitrogen of the indole ring. Contacting the epitope peptide with the fGly containing polypeptide of the T-Cell-MMP results in the T-Cell-MMP and epitope becoming covalently linked, thereby forming the T-Cell-MMP-epitope conjugates.

1.E. Epitopes for T-Cell-MMP Conjugates

Non-limiting examples of coronavirus epitopes that can be used to form T-Cell-MMP-epitope conjugates include those recited in Table 2 and FIG. 15.

### Example 2

Example 2 illustrates the ability to produce T-Cell-MMPs and conjugate them to a peptide resulting in a protein that is not aggregated (a dimer of T-cell-MMPs), displays suitable stability for use at 37° C., and can be purified. This example and Examples 3 and 5 are conducted with a CMV epitope peptide and/or other epitope peptides; however, coronavirus epitope peptides can equally be utilized to form epitope conjugates providing similar results. In the Example two immunomodulatory proteins were prepared by cellular expression in Expi-CHO cells using transient transfection using an expression vector containing a nucleic acid construct encoding the proteins. The proteins were purified over Protein A (MabSelect SuRe™; GE), followed by further purification by size exclusion chromatography.

The first immunomodulatory protein, having structure A set forth in FIG. 12 (generically termed an IL-2 Control Construct), is not a T-Cell-MMP or an epitope conjugate thereof. The protein acts as a control for the T-Cell-MMP of the present disclosure. That control protein comprises a first polypeptide having a 9 as cytomegalovirus (CMV) epitope at the N-terminus of a β2M polypeptide sequence:

(SEQ ID NO: 137)
NLVPMVATV [symbol] [symbol] IQRTPKIQVYSCHPAENGKSNFLNC
YVSGFHPSDIEVDLLKNGERIEKVEHSDLSFSKDWSFYLLYYTEFTPTE
KDEYACRVNHVTLSQPKIVKWDRDM.
Linkers are shown in bold and italics.

The second polypeptide of the control protein has, from N-terminus to C-terminus: two copies of an IL-2 immunomodulatory sequence (with H16A F42A substitutions) in tandem; a linker; an HLA-A*0201 polypeptide (with Y84A and A236C substitutions); a linker; and a human IgG1-Fc polypeptide having a LALA (L234A/L235A, see, e.g., FIG. 2G) substitution:

(SEQ ID NO: 138)
APTSSSTKKTQLQLEALLLDLQMILNGINNYKNPKLTRMLTAKFYMPKK
ATELKHLQCLEEELKPLEEVLNLAQSKNFHLRPRDLISNINVIVLELKG
SETTFMCEYADETATIVEFLNRWITFCQSIISTLT [symbol] [symbol]
APTSSSTKKTQLQLEALLLDLQMILNGINNYKNPKLTRM
LTAKFYMPKKATELKHLQCLEEELKPLEEVLNLAQSKNFHLRPRDLISN
INVIVLELKGSETTFMCEYADETATIVEFLNRWITFCQSIISTLT [symbol] [symbol]
GSHSMRYFFTSVSRPGRGEPRFIAVGYVD
DTQFVRFDSDAASQRMEPRAPWIEQEGPEYWDGETRKVKAHSQTHRVDL
GTLRGAYNQSEAGSHTVQRMYGCDVGSDWRFLRGYHQYAYDGKDYIALK
EDLRSWTAADMAAQTTKHKWEAAHVAEQLRAYLEGTCVEWLRRYLENGK
ETLQRTDAPKTHMTHHAVSDHEATLRCWALSFYPAEITLTWQRDGEDQT
QDTELVETRPCGDGTFQKWAAVVVPSGQEQRYTCHVQHEGLPKPLTLRW
E [symbol] DKTHTCPPCPAPEAAGGPSVFLFPPKPKDTLMISRTPEVTCVV
VDVSHEDPEVKFNWYVDGVEVHNAKTKPREEQYNSTYRVVSVLTVLHQD
WLNGKEYKCKVSNKALPAPIEKTISKAKGQPREPQVYTLPPSREEMTKN
QVSLTCLVKGFYPSDIAVEWESNGQPENNYKTTPPVLDSDGSFFLYSKL
TVDKSRWQQGNVFSCSVMHEALHNHYTQKSLSLSPGK.
Linkers are shown in bold and italics.

The second immunomodulatory protein, having structure B set forth in FIG. 12 is an IL-2 containing T-Cell-MMP having tandem IL-2 polypeptide sequences. The T-Cell-MMP comprises a first polypeptide having a linker bearing cysteine at aa 44 of the mature β2M polypeptide (E44C substitution marked as C*) that acts as a chemical conjugation site located at the N-terminus of the β2M polypeptide sequence: IQRTPKIQVYSCHPAENGKSNFLNCYVSGFHPSDIEVDLLKNGC*RIEKVEHSDLSFSKD WSFYLLYYTEFTPTEKDEYACRVNHVTLSQPKIVKWDRDM (SEQ ID NO:139).

The second polypeptide of the T-Cell-MMP has, from N-terminus to C-terminus: two copies of an IL-2 immunomodulatory sequence (with H16A F42A substitutions) in tandem; a linker; an HLA-A*0201 polypeptide (with Y84C, A139C, and A236C substitutions); a linker; and a human IgG1-Fc polypeptide having a LALA (L234A/L235A, see, e.g., FIG. 2G) substitution:

(SEQ ID NO: 140)
APTSSSTKKTQLQLEALLLDLQMILNGINNYKNPKLTRMLTAKFYMPKK
ATELKHLQCLEEELKPLEEVLNLAQSKNFHLRPRDLISNINVIVLELKG
SETTFMCEYADETATIVEFLNRWITFCQSIISTLT [symbol] [symbol]
APTSSSTKKTQLQLEALLLDLQMILNGINNYKNPKLTRM
LTAKFYMPKKATELKHLQCLEEELKPLEEVLNLAQSKNFHLRPRDLISN
INVIVLELKGSETTFMCEYADETATIVEFLNRWITFCQSIISTLT [symbol] [symbol]
GSHSMRYFFTSVSRPGRGEPRFIAVGYVD
DTQFVRFDSDAASQRMEPRAPWIEQEGPEYWDGETRKVKAHSQTHRVDL
GTLRGCYNQSEAGSHTVQRMYGCDVGSDWRFLRGYHQYAYDGKDYIALK
EDLRSWTAADMCAQTTKHKWEAAHVAEQLRAYLEGTCVEWLRRYLENGK
ETLQRTDAPKTHMTHHAVSDHEATLRCWALSFYPAEITLTWQRDGEDQT
QDTELVETRPCGDGTFQKWAAVVVPSGQEQRYTCHVQHEGLPKPLTLRW
E [symbol] DKTHTCPPCPAPEAAGGPSVFLFPPKPKDTLMISRTPEVTCVV
VDVSHEDPEVKFNWYVDGVEVHNAKTKPREEQYNSTYRVVSVLTVLHQD
WLNGKEYKCKVSNKALPAPIEKTISKAKGQPREPQVYTLPPSREEMTKN
QVSLTCLVKGFYPSDIAVEWESNGQPENNYKTTPPVLDSDGSFFLYSKL
TVDKSRWQQGNVFSCSVMHEALHNHYTQKSLSLSPGK.

Linkers are shown in bold and italics. The Y84C and A139C substitutions are shown as forming a disulfide bond between the α1 and α2 segments (helices) of the Class I heavy chain.

As shown in FIG. 12 at C, the expressed and purified proteins were subject to reducing SDS PAGE gel electrophoresis with both the control and T-Cell-MMP providing a light chain (first polypeptide) and heavy chain (second polypeptide).

### Example 3

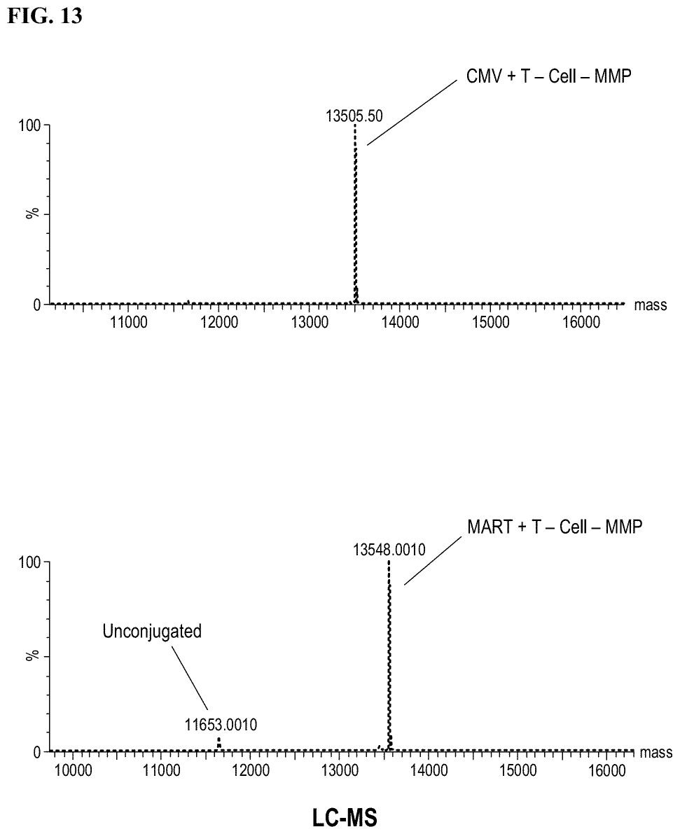

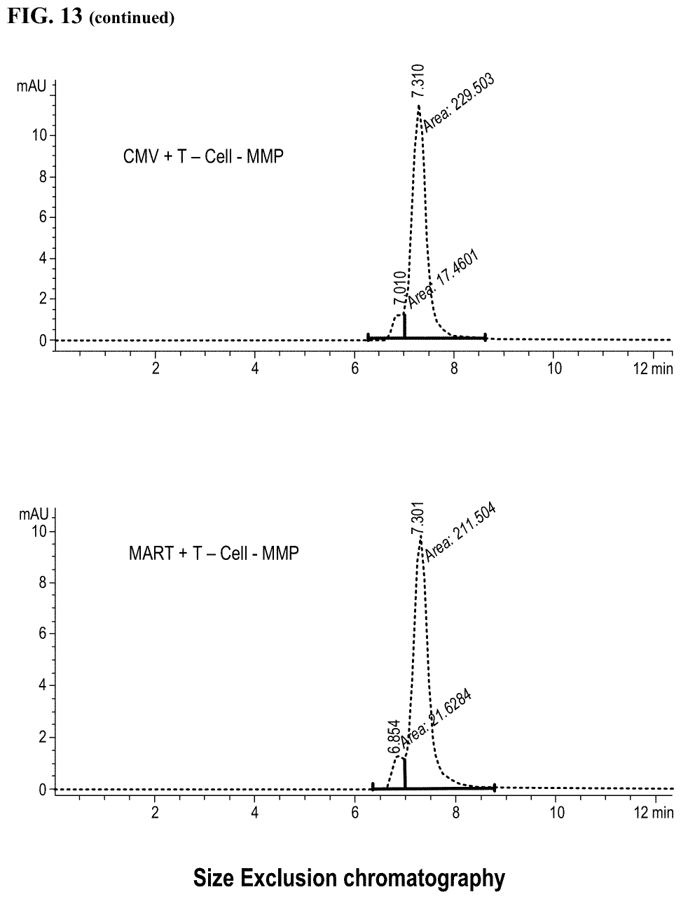

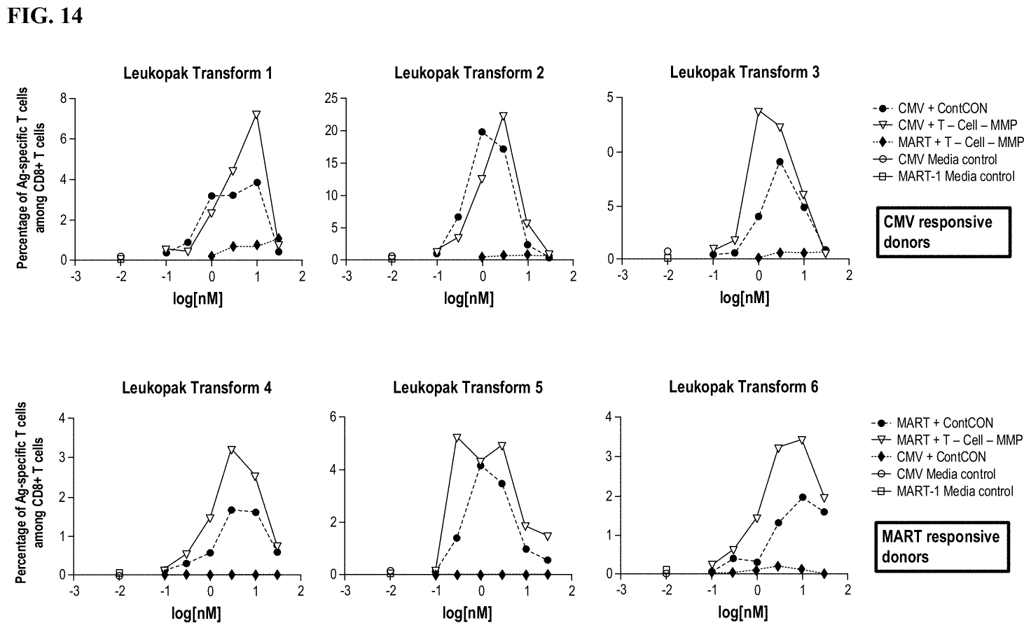

A T-Cell-MMP similar to Example 2 structure B was expressed and purified as described in Example 2. The purified T-Cell-MMP (labeled “IL-2 T-Cell-MMP”), which has an engineered cysteine residue as a chemical conjugation site, was conjugated to a CMV polypeptide (“CMV+ T-Cell-MMP”) or a melanoma antigen MART-1 (“MART+ T-Cell-MMP”) via a maleimide reactive linker attached to the peptide. The T-Cell-MMP-epitope conjugates were subjected to LCMS. The upper LCMS plot in FIG. 13 shows the IL-2 T-Cell-MMP+CMV light chain parent ion at 13,505.50 mass units with substantially complete conjugation. The lower LCMS plot in FIG. 13 shows the IL-2 T-Cell-MMP+MART light chain parent ion at 13,548.0010 mass units, with a minor amount of unconjugated T-Cell-MMP at 11,653.0010 mass units. Size exclusion chromatography indicates that at least 93% of the T-Cell-MMP conjugated to the CMV polypeptide and 91% of the T-Cell-MMP conjugated to the MART-1 polypeptide is in the form of an unaggregated dimer comprised of two first and two second polypeptides (see FIGS. 13 and 14 for reference).

### Example 4

Control constructs (see FIG. 12 structure A) comprising a first polypeptide having at the N-terminus of a β2M polypeptide sequence either a CMV epitope peptide as in Example 2 (“CMV+Cont-CON”) or a MART-1 epitope peptide (“MART+ContCON”) were prepared by cell expression and purification as described in Examples 2 and 3.

T-Cell-MMP-epitope-conjugates with tandem IL-2 MODs having a conjugated CMV peptide (“CMV+ T-Cell-MMP”) or conjugated MART-1 peptide (“MART+ T-Cell-MMP”) were prepared as in Example 3.

Ficoll-Paque® samples of leukocytes from three CMV responsive donors (Leukopak Transforms 1-3) and from three MART-1 (MART) responsive donors (Leukopak Transforms 4-6) were used. Responsiveness of the donor cells was determined based on the ability to expand CMV or MART-1 specific T-Cells upon CMV or MART-1 peptide stimulation in the presence of IL-2 as determined by flow cytometry. For the test shown in FIG. 14 the leukocytes were suspended at 2.5×10⁶ cells per ml in Immunocult media containing the indicated amounts of the control constructs or T-Cell-MMPs. As an additional control, cells grown without stimulation were stained with CMV or MART-1 tetramers purchased from MBL International Corp. After 10 days in culture the number of cells responsive to CMV or MART-1 were assessed by Flowcytometry. The results indicate that both the CMV+ContCON (having a CMV epitope expressed as part of the protein) and CMV+IL-2 T-Cell-MMPs (having a conjugated CMV epitope peptide) stimulate the expansion of CMV responsive CD8+ T-Cells in CMV responsive donors in a concentration dependent manner. Similarly, both the MART-1 control construct (having a MART-1 epitope expressed as part of the protein) and MART-1 IL-2 T-Cell-MMPs (having a conjugated MART-1 epitope peptide) stimulate the expansion of MART-1 responsive CD8+ T-Cells in MART-1 responsive donors in a concentration dependent manner.

The antigen specificity in the responses are evidenced by the fact that CMV Control Construct and IL-2 T-Cell-MMP molecules did not stimulate expansion of MART-1 responsive CD8+ T-cells. Likewise, MART-1 Control Construct and IL-2 T-Cell-MMP molecules did not stimulate expansion of CMV responsive CD8+ T-cells. Accordingly, the presence of IL-2 polypeptide sequences present in each of the molecules were not responsible for nonspecific expansion of the leukocytes.

1. A T-cell modulatory multimeric polypeptide epitope conjugate (T-Cell-MMP-epitope conjugate) comprising:
a) a first polypeptide having an N-terminus and a C-terminus, the first polypeptide comprising,
i) a beta-2-microglobulin (“β2M”) polypeptide having an N-terminus and a C-terminus, and an optional linker at the N-terminus and/or the C-terminus;
b) a second polypeptide having an N-terminus and a C-terminus, the second polypeptide comprising,
i) an MHC-H polypeptide;
ii) an immunoglobulin (Ig) Fc polypeptide or a non-Ig polypeptide scaffold, and an optional linker at the N-terminus and/or the C-terminus of the second polypeptide;
c) one or more first polypeptide chemical conjugation sites attached to or within the first polypeptide, and/or one or more second polypeptide chemical conjugation sites attached to or within the second polypeptide; and
d) one or more immunomodulatory polypeptides (MODs), wherein at least one of the one or more MODs is
A) at the C-terminus of the first polypeptide,
B) at the N-terminus of the second polypeptide,
C) at the C-terminus of the second polypeptide,
D) at the C-terminus of the first polypeptide and at the N-terminus of the second polypeptide or
E) within the first or second polypeptide;
e) a coronavirus peptide epitope covalently bound, directly or indirectly, to at least one of the one or more first polypeptide chemical conjugation sites or the one or more second polypeptide chemical conjugation sites, the coronavirus peptide epitope comprising six (6) or more contiguous amino acids of a coronavirus protein selected from the group consisting of: surface glycoprotein; membrane protein; nucleocapsid phosphoprotein; open reading frame (Orf) 1b protein; Orf3a protein; Orf6 protein; Orf7a protein; Orf8 protein; Orf10 protein; envelope protein; membrane glycoprotein; nonstructural protein (nsp) one (nsp1); nsp2; nsp4; nsp6; nsp7, nsp8, nsp9, nsp10; nsp14; nsp15; nsp16; nucleocapsid phosphoprotein; papain-like cysteine protease (PLpro); 3C-like protease (3CL); RNA-dependent RNA polymerase (RDpol) and helicase (Hel);
wherein each of the one or more MODs is an independently selected wild-type or variant MOD;
wherein the MHC-H polypeptide sequence has at least 90% sequence identity to at least 200 contiguous aas of an MHC-H chain polypeptide selected from the group consisting of: HLA-A*0101 (SEQ ID NO:20), HLA-A*0201 (SEQ ID NO:23), HLA-A*0301 (SEQ ID NO:31), HLA-A*1101 (SEQ ID NO:28), HLA-A*2301 (SEQ ID NO:32), HLA-A*2402 (SEQ ID NO:29), HLA-A*2407 (SEQ ID NO:33), HLA-A*3303 (SEQ ID NO:30), HLA-A*3401 (SEQ ID NO:34), HLA-B*0702 (SEQ ID NO:36), HLA-B*0801 (SEQ ID NO:37), HLA-B*1502 (SEQ ID NO:38), HLA-B*3501 (SEQ ID NO:127), HLA-B*3802 (SEQ ID NO:39), HLA-B*4001 (SEQ ID NO:40), HLA-B*4402 (SEQ ID NO:128), HLA-B*4403 (SEQ ID NO:129), HLA-B*4601 (SEQ ID NO:41), HLA-B*5301 (SEQ ID NO:42), HLA-B*5801 (SEQ ID NO:130), HLA-C*0102, (SEQ ID NO:44), HLA-C*0303 (SEQ ID NO:45), HLA-C*0304 (SEQ ID NO:46), HLA-C*0401 (SEQ ID NO:47), HLA-C*0602 (SEQ ID NO:48), HLA-C*0701 (SEQ ID NO:49), HLA-C*0702 (SEQ ID NO:50), HLA-C*0801 (SEQ ID NO:51), and HLA-C*1502 (SEQ ID NO:52, an HLA-E polypeptide (SEQ ID NO: 54), an HLA-F polypeptide (SEQ ID NO: 55), and an HLA-G polypeptide (SEQ ID NO:56); and
wherein optionally substantially all of the T-Cell-MMP is in the form of a dimer comprising a first T-cell MMP and a second T-Cell-MMP associated by covalent and/or non-covalent interactions between their polypeptide' scaffold sequences.

2. The T-Cell-MMP-epitope conjugate of claim 1,
wherein the coronavirus peptide epitope comprises six (6) or more contiguous amino acids of a coronavirus protein selected from the group consisting of: surface glycoprotein; membrane protein; nucleocapsid phosphoprotein; and open reading frame (Orf) 1b protein;
wherein each of the one or more MODs is an independently selected wild-type or variant MOD; and
wherein the MHC-H polypeptide sequence has at least 90% sequence identity to at least 200 contiguous aas of an MHC-H chain polypeptide selected from the group consisting of HLA-A*0201 (SEQ ID NO:23), HLA-A*2402 (SEQ ID NO:29), HLA-A*1101 (SEQ ID NO:28), HLA-B*4001 (SEQ ID NO:40), and HLA-B*5801 (SEQ ID NO:130).

3. The T-Cell-MMP-epitope conjugate of claim 2, wherein the peptide is selected from the group consisting of: GLMWLSYFV, TLACFVLAAV, ALNTPKDHI, GMSRIGMEV, LALLLLDRL, LLLDRLNQL, LQLPQGTTL, RLNQLESKV, ALSGVFCGV, CLDAGINYV, ILLLDQVLV, LLCVLAALV, SMWALVISV, TLMNVITLV, WLMWFIISI, ALNTLVKQL, FIAGLIAIV, KLPDDFMGCV, LITGRLQSL, NLNESLIDL, RLNEVAKNL, SIVAYTMSL, VLNDILSRL, QFKDNVILL, GETALALLLL, MEVTPSGTWL, RFFTLGSITAQPVKI, and SITAQPVKI.

4. The T-Cell-MMP-epitope conjugate of claim 1, wherein the coronavirus peptide epitope comprises six (6) or more contiguous amino acids of a coronavirus protein selected from the group consisting of: Orf3a protein; Orf6 protein; Orf7a protein; Orf8 protein; Orf10 protein; envelope protein; membrane glycoprotein; nonstructural protein (nsp) one (nsp1); nsp2; nsp4; nsp6; nsp7, nsp8, nsp9, nsp10; nsp14; nsp15; nsp16; nucleocapsid phosphoprotein; papain-like cysteine protease (PLpro); 3C-like protease (3CL); RNA-dependent RNA polymerase (RDpol) and helicase (Hel).

5. The T-Cell-MMP-epitope conjugate of claim 1, wherein the coronavirus peptide epitope comprises six (6) or more, or eight (8) or more, contiguous amino acids of any one of the peptides set forth in SEQ ID NO 131, 149-194 or 219-195.

6. The T-Cell-MMP-epitope conjugate of claim 5, wherein the MHC-H polypeptide sequence has at least 90% sequence identity to 200 aas of an MHC-H chain polypeptide selected from the group consisting of: HLA-A*0101 (SEQ ID NO:20), HLA-A*0201 (SEQ ID NO:23), HLA-A*0301 (SEQ ID NO:31), HLA-A*1101 (SEQ ID NO:28), HLA-A*2301 (SEQ ID NO:32), HLA-A*2402 (SEQ ID NO:29), HLA-B*0702 (SEQ ID NO:36), HLA-B*0801 (SEQ ID NO:37), HLA-B*3501 (SEQ ID NO:127), HLA-B*4001 (SEQ ID NO:40), HLA-B*4402 (SEQ ID NO:128), HLA-B*4403 (SEQ ID NO:129), and HLA-B*5801 (SEQ ID NO:130).

7. The T-Cell-MMP-epitope conjugate of claim 1, wherein the one or more MODs are wild type MODs or variant MODs selected independently from the group consisting of IL-2, 4-1BBL, PD-L1, CD70, CD80, CD86, ICOS-L, OX-40L, FasL, JAG1, TGF-β, ICAM, and PD-L2, and variants thereof.

8. The T-Cell-MMP-epitope conjugate of claim 1, wherein the one or more MODs are wild type MODs or variant MODs selected independently from the group consisting of IL-2, 4-1BBL, CD80, CD86, and variants thereof.

9. The T-Cell-MMP-epitope conjugate of claim 1, wherein the second polypeptide comprises an immunoglobulin (Ig) Fc polypeptide scaffold.

10. The T-Cell-MMP-epitope conjugate of claim 1, wherein the first MHC polypeptide comprises:
a β2M polypeptide bearing a linker on its N-terminus,
a β2M polypeptide bearing a linker on its C-terminus, or
a β2M polypeptide bearing a linker on its N-terminus and C-terminus.

11. The T-Cell-MMP-epitope conjugate of claim 1, wherein the first and second chemical conjugation sites are independently selected from:
a) amino acid chemical conjugation sites;
b) non-natural amino acids and/or selenocysteines;
c) peptide sequences that act as enzymatic modification sequences;
d) carbohydrate or oligosaccharide moieties; and/or
e) IgG nucleotide binding sites.

12. The T-Cell-MMP-epitope conjugate of claim 11, wherein the coronavirus peptide epitope is covalently bound, directly or indirectly through a linker to the β2M polypeptide sequence.

13. The T-Cell-MMP-epitope conjugate of claim 12, wherein the coronavirus peptide is bound to an amino acid chemical conjugation site.

14. The T-Cell-MMP-epitope conjugate of claim 13, wherein the amino acid chemical conjugation site to which the epitope is attached is a cysteine engineered into the β2M polypeptide or an amino acid of a linker at the N-terminus of the β2M polypeptide.

15. The T-Cell-MMP-epitope conjugate of claim 14, wherein the epitope is attached to a cysteine engineered into the β2M polypeptide sequence as a substitution selected from Q2C, E44C, E50C, E77C, V85V, S88C, K91C, and/or D98C; and wherein the β2M polypeptide has a sequence with at least 90% sequence identity to at least 80 contiguous amino acids of a mature β2M polypeptide set forth in any of SEQ ID NOs: 57-61.

16. The T-Cell-MMP-epitope conjugate of claim 15, wherein the coronavirus peptide epitope comprises from 6 to 25 contiguous aas of one coronavirus protein, and wherein the T-Cell-MMP-epitope conjugate optionally comprises one or more IL-2 or variant IL-2 MODs having reduced affinity for the IL-2 receptor.

17. The T-Cell-MMP-epitope conjugate of claim 5, wherein:
A
i) the coronavirus epitope comprises a sequence having 0, 1 or 2 aa substitutions, deletions or insertions in an HLA*A restricted epitope peptide selected from the group consisting of GLMWLSYFV, TLACFVLAAV, ALNTPKDHI, GMSRIGMEV, LALLLLDRL, LLLDRLNQL, LQLPQGTTL, RLNQLESKV, ALSGVFCGV, CLDAGINYV, ILLLDQVLV, LLCVLAALV, SMWALVISV, TLMNVITLV, WLMWFIISI, ALNTLVKQL, FIAGLIAIV, KLPDDFMGCV, LITGRLQSL, NLNESLIDL, RLNEVAKNL, SIVAYTMSL, VLNDILSRL, and QFKDNVILL;
ii) the MHC-H polypeptide sequence has at least 90% sequence identity to 200-250 contiguous aa or all of an MHC-H chain polypeptide selected from the group consisting of: HLA-A*0201 (SEQ ID NO:23), and HLA-A*2402 (SEQ ID NO:29); and
iii) the T-Cell-MMP-epitope conjugate optionally comprises one or more IL-2 or variant IL-2 MODs having reduced affinity for the IL-2 receptor; or
B
i) the coronavirus epitope comprises a sequence having 0.1 or 2 aa substitutions, deletions or insertions in an HLA*B restricted epitope peptide selected from the group consisting of GETALALLLL, MEVTPSGTWL, RFFTLGSITAQPVKI, and SITAQPVKI;
ii) the MHC-H polypeptide sequence has at least 90% sequence identity to 200-250 continuous aas or all of an MHC-H chain polypeptide selected from the group consisting of: HLA-B*4001 (SEQ ID NO:40) and HLA-B*5801 (SEQ ID NO:130); and
iii) the T-Cell-MMP-epitope conjugate optionally comprises one or more IL-2 or variant IL-2 MODs having reduced affinity for the IL-2 receptor.

18. (canceled)

19. The T-Cell-MMP-epitope conjugate of claim 14, comprising one or more, or two or more, IL-2 and/or variant IL-2 MODs having reduced affinity for the IL-2 receptor.

20. (canceled)

21. The T-Cell-MMP-epitope conjugate of claim 1, wherein the T-Cell-MMP-epitope conjugate forms a dimer through covalent or non-covalent interactions between the (Ig) Fc polypeptide or the non-Ig polypeptide scaffold.

22. A composition comprising:
a) the T-Cell-MMP-epitope conjugate of claim 14; and
b) a pharmaceutically acceptable excipient.

23-24. (canceled)

25. A method of treating a patient or individual, the method comprising administering to the patient or individual an effective amount of the T-Cell-MMP-epitope conjugate of claim 14.

26. The method of claim 25, wherein the patient or individual is being treated for a SARS CoV infection and/or SARS-CoV-2 (Covid-19) infection.
